(19) United States
(12) Patent Application Publication
(10) Pub. No.: US 2025/0129169 A1
(43) Pub. Date: Apr. 24, 2025

(54) SURROGATE CYTOKINE POLYPEPTIDES

(71) Applicant: The Board of Trustees of the Leland Stanford Junior University, Stanford, CA (US)

(72) Inventors: Kenan Christopher GARCIA, Palo Alto, CA (US); Michelle YEN, Palo Alto, CA (US); Junming REN, Palo Alto, CA (US); Qingxiang LIU, Palo Alto, CA (US)

(21) Appl. No.: 18/835,511

(22) PCT Filed: Feb. 3, 2023
(86) PCT No.: PCT/US2023/062005

§ 371 Date: Aug. 2, 2024

Related U.S. Application Data
(60) Provisional application No. 63/306,882, filed on Feb. 4, 2022.

(51) Int. Cl. C07K 16/28; A61P 31/14; G01N 33/68

(52) U.S. Cl. CPC C07K 16/2866; A61P 31/14; G01N 33/6854; C07K 2317/31; C07K 2317/569; C07K 2317/622; G01N 2333/715

(57) ABSTRACT

The present disclosure relates to to compositions and methods relating to cytokine agonists and their engineered polypeptides. The engineered polypeptides have specificity to receptors in immune systems including IL-2/15, Type I IFN and IL-10. The present disclosure also relates to methods for identifying surrogate cytokine agonists and to a system for engineering ligands that can compel formation of non-naturally-occurring cytokine receptor heterodimers. The present disclosure also relates to methods and system for identifying surrogate agonists for cell surface receptors including dimeric and trimeric receptors.

### CROSS-REFERENCE TO RELATED APPLICATION

The present application claims priority to U.S. Provisional Patent Application Ser. No. 63/306,882, filed on Feb. 4, 2022. The content of the above-referenced application is herein expressly incorporated by reference in its entirety, including any drawings.

### FIELD

The technology relates generally to the field of immunology. More particularly, the technology relates to methods and compositions for the discovery and identification of surrogate cytokine agonists for modulating transduction mediated by IL-2, IL-10, IL-15 and Type I IFN. The technology also relate to platforms for the generation and screening of agonists for naturally- and non-naturally occurring combinations of receptors.

### INCORPORATION OF THE SEQUENCE LISTING

The material in the accompanying Sequence Listing is hereby incorporated by reference into this application. The accompanying Sequence Listing file, named 078430-539002WO_Sequence Listing_ST26.xml, was created on Feb. 1, 2023, and is 190,297 bytes.

### BACKGROUND

Cytokines are garnering increasing interest as therapeutics given their powerful actions in the immune system, as well as other systems that regulate human biology. However, the process of therapeutic discovery for cytokines is generally limited to exploration of the intrinsic biological properties of the natural cytokine ligands, through modifications such as affinity maturation, half-life extension and/or tissue targeting (Berraondo et al., 2018; Mansurov et al., 2021; Overwijk et al., 2021). More recently, cytokine engineering strategies have succeeded in demonstrating that cytokine pleiotropy can be mitigated by selective structure-based engineering and protein design (Glassman et al., 2021b; Mendoza et al., 2019; Mitra et al., 2015; Saxton et al., 2021). However, unlike multi-pass transmembrane proteins such as GPCRs and ion channels, cytokine systems that signal through Type I single-pass transmembrane receptors are not amenable to medicinal chemistry types of high-throughput approaches (Shoichet and Kobilka, 2012). This is due to two principal reasons.

First, cytokines are globular proteins that function to bind to receptor extracellular domains and dimerize them (Spangler et al., 2015; Stroud and Wells, 2004). The cytokine forms large protein-protein contact surfaces with the receptor ECDs to supply the binding energy needed to bridge two receptor subunits. In contrast, small molecules bind within pockets in GPCR and ion channel transmembrane helices. Thus, cytokine receptor systems are not amenable to small molecule library-based screening campaigns. Furthermore, cytokines themselves are single-domain four-helix bundle proteins that present structural limitations for ligand engineering (Silva et al., 2019), which is generally limited to interface mutagenesis (Glassman et al., 2021a; Levin et al., 2012; Mitra et al., 2015).

Second, cytokine signaling has generally been assumed to be “on or off,” in contrast to tunable GPCR (i.e. biased) signaling (Smith et al., 2018). Thus, cytokine agonist therapeutics have largely been limited to variations of the natural cytokine. However, recent studies have shown that the orientation and proximity of dimeric receptor assemblies can profoundly influence signaling output and that cytokine receptor signaling is ‘tunable’ (Mohan et al., 2019; Moraga et al., 2015). Furthermore, antibodies can, in some instances, act as cytokine agonists by dimerizing the cytokine receptors into appropriate signaling geometries (Harris et al., 2021; Moraga et al., 2015).

Therefore, there is a need to bridge the gap between medicinal chemistry library approaches that can identify biased agonists, and traditional cytokine engineering approaches that do not access the full scope of cytokine receptor signaling plasticity. There is also a need for new cytokine agonists and methods for their discovery.

### SUMMARY

The present disclosure relates generally to the development of engineered polypeptides that are surrogate cytokine receptors comprising single-chain and two-chain bispecific ligands. The present disclosure also relates to cells, nucleic acid constructs, expression constructs, compositions, pharmaceutical composition including the engineered polypeptides as well as methods for identifying surrogate cytokine agonists.

In some aspects of the disclosure, provided herein are engineered polypeptides including a single-chain bispecific ligand wherein a first specificity of the ligand is to IFNAR1 and a second specificity of the ligand is to IFNAR2 and wherein the engineered polypeptide is a cytokine agonist. In some embodiments, the single-chain bispecific ligand comprises a first nanobody specific to IFNAR1 and a second nanobody specific to IFNAR2. In some embodiments, the single-chain bispecific ligand comprises an amino acid sequence at least 80% identical to the amino acid sequence set forth in SEQ. ID. No.: 51-69. In some embodiments, the single-chain bispecific ligand includes an amino acid sequence at least 90% identical to the amino acid sequence set forth in SEQ. ID. No.: 51-69. In some embodiments, the single-chain bispecific ligand includes an amino acid sequence at least 95% identical to the amino acid sequence set forth in SEQ. ID. No.: 51-69. In some embodiments, the single-chain bispecific ligand includes an amino acid sequence that is the amino acid sequence set forth in SEQ. ID. No.: 51-69. In some embodiments, the first nanobody includes an amino acid sequence that is the amino acid sequence set forth in SEQ. ID. No.: 36-44. In some embodiments, the second nanobody comprises an amino acid sequence that is the amino acid sequence set forth in SEQ. ID. No.: 45-50. In some embodiments, the single-chain bispecific ligand is a dimerizing-ligand for an IFNAR1/IFNAR2 receptor heterodimer. In some embodiments, the engineered polypeptide is capable of inducing phosphorylation of STAT1, or STAT2 or STAT3 or a combination thereof in vitro. In some embodiments, the engineered polypeptide is capable of inducing phosphorylation of STAT1, or STAT2 or STAT3 or a combination thereof in vivo.

In some embodiments, the engineered polypeptide is capable of inhibiting SARS-CoV-2 replication. In some embodiments, the engineered polypeptide is capable of inhibiting SARS-COV-2 replication in vitro. In some embodiments, the engineered polypeptide is capable of inhibiting SARS-COV-2 replication in vivo. In some embodiments, the engineered polypeptide is capable of inhibiting SARS-COV-2 replication in a cell without inducing the expression of pro-inflammatory cytokines. In some embodiments, the engineered polypeptide is capable of inhibiting SARS-COV-2 replication in a cell without inducing the expression of anti-proliferative cytokines. In some embodiments, the cell is a mammalian cell. In some embodiments, the mammalian cell is a human cell. In some embodiments, the cell is a mouse cell.

In some embodiments of the engineered polypeptides of the disclosure, wherein the first nanobody is specific to IFNAR1 and the second nanobody is specific to IFNAR2, the first nanobody and the second nanobody are linked by a linker. In some embodiments, the linker is a peptide linker.

In yet another aspect, provided herein are cells including the engineered polypeptide of the disclosure.

In a further aspect, provided herein are compositions including the engineered polypeptide of the disclosure.

Also provided herein, are pharmaceutical compositions including a pharmaceutically acceptable excipient and the engineered polypeptide of the disclosure.

In yet further aspect, the disclosure provides nucleic acids or molecules encoding any of the engineered polypeptide of the disclosure.

Further aspects of the present disclosure, provided herein are expression constructs including any of the engineered polypeptide of the disclosure.

In other aspects, provided herein are methods for identifying surrogate cytokine agonists, the method includes providing nanobodies or scFvs against a first target cytokine receptor and against a second target cytokine receptor, and linking a nanobody or scFv against the first target cytokine receptor with a nanobody or scFv against the second target cytokine receptor thereby identifying a surrogate cytokine agonist. In some embodiments, the methods further include screening for induction of downstream signaling activity. In some embodiments, the method includes screening for induction of STAT1, STAT2, STAT3, STAT5, STAT6, Akt, S6, or ERK activity or combinations thereof.

Also provided herein are methods for identifying surrogate agonists for a cell surface receptor wherein the cell surface receptor includes a first component and a second component, and wherein the method includes assembling one or more antibody domains to form a bispecific ligand such that a first specificity of the ligand is to the first component and a second specificity of the ligand is to the second component; and identifying surrogate agonists for the receptor. In some embodiments, the cell surface receptor is a dimeric receptor. In some embodiments, the dimeric receptor is an RTK, a cytokine or an IgSF receptor. In some embodiments, the receptor is a trimeric receptor and includes a third component. In some embodiments, the trimeric receptor is a death receptor. Examples of death receptors include but are not limited to TNF receptor-1, CD95 (Fas), TRAMP, TRAIL-R1, and TRAIL-R2.

In non-limiting exemplary embodiments of the methods for identifying surrogate agonists for a cell surface receptor, the antibody domains that are used to assemble the ligands are VHHs or scFvs. In some embodiments, the ligand is a single chain homodimer. In some embodiments, the ligand is a heterodimer. In some embodiments, the ligand is an Fc fusion. In some embodiments, the ligand is a multi-chain agonist comprised of fusions to oligomeric zippers.

In some embodiments of the methods for identifying surrogate agonists, the surrogate agonist is a cytokine agonist. In some embodiments, the surrogate agonist is an IFN agonist.

In some embodiments, the methods further includes screening the surrogate IFN for activity by analyzing the differential or preferential induction of interferon stimulated genes (ISGs). Examples of ISGs include, but are not limited to, MX1, OAS1, IFIT1, IFITM1, TRAIL, CXCL10, ISG15, CH25CH, cGAS, BST2, and NCOA7ISG.

In some embodiments, the methods further comprise employing a screening of the differential induction of interferon stimulated genes (ISGs) as a metric for surrogate IFN activity. In some embodiments, the ligand is a dimerizing ligand for the receptor. In some embodiments, the ligand homodimerizes the first, second and third component of the receptor.

Also provided herein is a platform for the generation and screening of agonists. In some embodiments, the agonists dimerize naturally-occurring receptors. In some embodiments, the agonists dimerize non-naturally occurring combinations of receptors.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1G show a schematic illustration of the platform of the present disclosure for the generation and screening of bispecific IL-2Rβ-γ_{c} surrogate agonists. (A) Approach to developing IL-2 receptor agonists based on construction of bispecific VHH or scFv specific for IL-2Rβ and γC. (B) Schematic representation of VHH and scFv binding to diverse epitopes along the IL-2Rβ or γ_{c} extracellular domains (left), combinatorial matrix to generate a collection of β-γ dimerizing ligands (middle), and representation of VHH-VHH or VHH-scFv fusion constructs connected by short linkers in Forward or Reverse orientations (right). (C) Schematic pipeline of protein expression and activity screening. Bispecific VHH were produced by gene synthesis of VHH monomers and cloning, expressed at 2 mL scale in Expi293 cells, and purified via their 6-His tags on Ni²⁺ affinity resin followed by size exclusion chromatography (SEC) and SDS-PAGE analysis. Protein activity was measured via a pSTAT5 phosphoflow assay on YT-1 cells. (D) Heatmap of pSTAT5 activity evoked by bispecific antibody pairings. YT-1 cells were stimulated with saturating ligand concentration for 20 min., fixed and permeabilized, then stained with α-STAT5 (pY694)-AlexaFluor647 and analyzed via flow cytometry. (E) Affinity to IL-2Rβ does not predict STAT5 activity. Each circle represents a bispecific molecule, with pSTAT5 Eₘₐₓ (normalized to hIL-2) plotted against the affinity of its IL-2Rβ-specific VHH. Data were fit by linear regression, with R2=0.0335. (F) Affinity to γ_{C} does not predict STAT5 activity. Each circle represents a bispecific molecule, with pSTAT5 Eₘₐₓ (normalized to hIL-2) plotted against the affinity of its γ_{C}-specific VHH or scFv. Data were fit by linear regression, with R2=0.0004. (G) Overall receptor binding affinity (IL-2Rβ×γ_{C} K_{D}) is not predictive of STAT5 activity. Bispecific molecules with identical IL-2Rβ×γ_{C} antibody usage are depicted in the same color. Data were fit by linear regression, with R2=0.0005. This shows that a given VHH β-γ_{C} pair does not have the same activity in the forward and reverse (γ_{c}-β) orientations.

FIGS. 2A-2H show data on SPR validation of IL-2Rβ- and γ_{c}-VHHs and agonist screening in YT-1 cells. (A) Biotinylated human IL-2Rβ ECD was immobilized on a streptavidin (SA) sensor chip, and varying concentrations of IL-2Rβ VHH were applied to determine binding parameters using SPR. Sensorgrams are shown on the left, and steady state binding is plotted on the right. Binding affinities (K_{D}) were determined by fitting to steady state response values. (B) Biotinylated human γ_{C} ECD was immobilized on a SA chip, and varying concentrations of γ_{c} VHH were flowed over the chip to determine binding. Sensorgrams (left) and steady state binding (right) are displayed as in (A). (C) Steady-state binding affinities between VHH/scFv and IL-2R, as determined by SPR. Values for scFv affinities are derived from patent US20160367664A1 (Wang et al., 2016). (D) Table of pSTAT5 Eₘₐₓ values for the indicated VHH pairings. (E) Tuning IL-2 activity via generation of acid-base VHH zipper pairings. (F) Tuning IL-2 activity via generation of 2:1 β:γ_{c} or γ_{c}:β stoichiometries. VHH binding modules are linked together in a tripartite (β-γ_{c}-β or γ_{c}-β-γ_{c}) manner. Graph axes are represented as in (E). (G) Dose-response relationship of pSTAT5 geometric mean flurorescence intensities (gMFI) of selected agonists. (H) Corresponding histograms showing STAT5 activity in YT-1 cells treated with human IL-2 vs. IL-2 surrogate agonists at saturating concentration.

FIGS. 3A-3F depict the profiling, signaling properties IL-2 surrogate ligands. (A) Kinetics of pSTAT5, pERK, and pAkt signaling evoked by IL-2 or surrogate agonists. YT-1 cells were serum-starved for 1-2 hr., then stimulated with 50 nM ligand for 0.5-15 min. at 37° C., fixed and permeabilized, then stained with fluorescently-conjugated phospho-antibodies before reading on a flow cytometer. (B) Dose-response relationship of pSTAT5, pERK, and pAkt activity evoked by IL-2 or surrogate agonists. Serum-starved YT-1 cells were stimulated with varying concentration of ligand for 3 min., then processed as in (A) for phosphoflow analysis. (C) Classification of signal strength for IL-2 surrogate agonists, with relative strength of activity encoded by colored gradients. (D) T cell blasts were stimulated with 50 nM hIL-2 or surrogate agonist for 20 min. at 37° C., fixed and permeabilized, then stained with fluorescently conjugated antibodies against pSTAT1, pSTAT3, or pSTAT5 and read on a flow cytometer. Raw fluorescence intensities were background subtracted against that of unstimulated cells, then normalized to hIL-2 values. (E) T cell blasts were stimulated with 50 nM hIL-2 or surrogate agonist for 1 hr. at 37° C. Cells were fixed and permeabilized, then stained with fluorescently-conjugated antibodies against pSTAT5 and pS6, and read on a flow cytometer. Data are baseline subtracted and normalized as in (D). (F) Summary of ligand signaling properties across pSTAT1/3/5 and pAkt pathways, with relative strength of activity encoded by colored gradients. Data were collected in triplicate, with graphs displaying the mean±sem.

FIGS. 4A-4E depict modeled dimeric geometrics from structures of IL-2Rβ:VHH and γ_{c}:VHH complexes. (A) Side view comparisons between the human IL-2:IL-2Rβ binary complex (PDB: 2B5I) (Wang et al., 2005) and β-VHH6:IL-2Rβ binary complex. Surface representations of IL-2 and β-VHH6 are colored in purple and light blue, respectively, while IL-2Rβ is shown in ribbon representation in navy. (B) Side view comparisons of IL-2:γ_{C} and γ_{C}-VHH6:γ_{C} receptor complexes. γ_{C}-VHH6 is shown in pink, with γ_{C} colored in red (C) Crystal structure of the human IL-2:IL-2Rβ: γ_{C} ternary complex (PDB: 2B5I) (Wang et al., 2005). Side view with membrane bilayer and schematic representation of receptor transmembrane and intracellular domains (ICD) is shown at middle. Top view (below) is related to the side view by a 90° rotation about the horizontal axis. (D) Model of γ_{c}-VHH6-β-VHH6 bound to its receptors. Structures of the γ_{c}-VHH6:γ_{C} and IL-2Rβ:β-VHH6 were determined separately. The γ_{C}-VHH6-β-VHH6 linker distance was modeled in and represented by a dotted line (top), with a side views of receptor-bound model shown underneath. (E) Model of β-VHH6-γ_{c}-VHH6 bound to its receptors. The VHH-VHH linker was modeled as in (D).

FIGS. 5A-5D show transcriptional profiling of IL-2 surrogate agonists. (A) Principal component analysis (PCA) of gene expression in CD8⁺ T cells from 3 donors stimulated with IL-2 or surrogate ligands for 24 hours. Samples from a given donor lie along a horizontal line, with unstimulated samples at the right and IL-2/IL-15 treated samples at the left. The effect of various ligand stimulations is largely described by PC1. (B) Relationship between surrogate ligand pSTAT5 activity and the total number of differentially expressed genes (DEG) induced by ligand stimulation. STAT5 phosphorylation was normalized to that of hIL-2 stimulated cells. (C) Log₂ fold expression change of transcription factors which play opposing roles in CD8⁺ memory vs. effector differentiation. Opposing transcription factor pairs are diagrammed (right) with the accompanying log 2 fold changes induced by surrogate ligands (left). (D) Log₂ fold expression change of selected markers of memory and effector T cells (left). Memory T cells express CD62L (encoded by SELL), IL7 receptor, and the transcription factor TCF1 (encoded by TCF7), whereas effector CD8⁺ T cells produce abundant amounts of cytokines TNFα and IFNγ and cytolytic molecules such as granzymes A and B (right).

FIGS. 6A-6G show that the IL-2 surrogate agonists of the present disclosure support T and NK cell proliferation and cytolysis. Naïve T cells were isolated from PBMC by negative magnetic selection, preactivated for 4d with surface-bound α-CD3+soluble α-CD28, then cultured in the presence of 100 nM hIL-2 or surrogate agonist for 8d. (A) Cytokine profiling of CD8⁺ T cells was performed by stimulating cells with PMA+ionomycin in the presence of brefeldin A and monensin, followed by intracellular staining to assess IFNγ, IL-2, and TNFα production. Data represent an average of 3 replicate wells and are colored by heat map encoding the percentage of CD8⁺ cells expressing the indicated cytokine. (B) Cells were stained with surface antibodies against CD4, CD8, CCR7, and CD45RA to enumerate differentiation into T cell memory subtypes. The fraction of naïve, central memory, effector memory, and T_{EMRA} cells are represented using pie charts. (C) PBMC were cultured for 2 weeks in the presence of 100 nM hIL-2 or surrogate agonists, then stained with phenotyping markers for T and NK cells and enumerated using flow cytometry. The graph displays absolute live cell counts of CD8⁺ T, CD4⁺ T, CD16⁺NK, and CD16⁻ NK cells. (D) Pie charts of cell count data from (C) depict the fraction of T and NK cell types. (E) T cell cytolytic activity stimulated by culture with hIL-2 or surrogate agonists. Pre-activated human T cells were lentivirally transduced with A3A TCR and cultured for 10d in the presence of 100 nM hIL-2 or IL-2 surrogate agonists to generate CTLs. Cytotoxicity was measured by mixing effector T cells with a fixed number of CTV-labeled A375 melanoma target cells for 4-6 hr., then assessing apoptosis via annexin V staining. (F) NK cytolytic activity stimulated by culture with hIL-2 or surrogate agonists. Pre-activated NK cells were cultured for 4 weeks in the presence of 100 nM hIL-2 or surrogate agonists and mixed with 25,000 CTV-labeled K562 target cells per well. Following 5 hr. incubation, cells were stained with annexin V-PE, then analyzed for early apoptosis using flow cytometry. (G) Relative efficiency of NK vs. T cell cytolysis supported by surrogate IL-2 ligands. Annexin V positivity rates were normalized to hIL-2 in NK cells (F) or T cells (E) cultured with surrogate ligands, then ratioed and represented as a heat map.

FIGS. 7A-7O illustrate that Type I interferon surrogate agonists exhibit biased signaling and inhibit viral replication. (A) Schematic representation of bispecific type I IFN surrogate ligands which heterodimerize IFNAR1 and IFNAR2 (left). A collection of 11 IFNAR1 binders (1 scFv, 10 VHH) were paired with 6 IFNAR2 binders (VHH), resulting in 66 combinations of IFNAR1-IFNAR2 fusion molecules connected via a 5 a.a. linker (right). Twelve of these molecules induced pSTAT1 activity on YT-1 cells (pink shading). The IFNAR2-specific scFv “3F11” was identified from the patent U.S. Pat. No. 7,662,381B2 (Cardarelli et al., 2010). Seven of the hits, “HIS1-7,” were selected for further analysis. (β-D) Dose-response relationship of STAT1 phosphorylation evoked by IFNω or surrogate agonists. YT-1 cells (B), A549 cells (C), or PBMCs (D) were stimulated with saturating ligand concentration for 20 min., fixed and permeabilized, then stained with α-STAT1 (pY701)-AlexaFluor647 and analyzed via flow cytometry. (E) Heatmap representation of STAT1-STAT6 phosphorylation evoked by surrogate agonists in YT-1 cells at different time points and normalized to the activation induced by IFNω (F) Heatmap representation of STAT1 and STAT2 phosphorylation evoked by surrogate agonists in A549 cells at varying time points, normalized to activation induced by IFNω (G) qRT-PCR analysis of SeV RNA in A549 cells pre-treated with 10 nM surrogate ligands or IFNω for 24 hr. followed by SeV infection (MOI=0.1) for 24 hr. (H) SARS-COV-2 nLUC A549-hACE2 Antiviral Assay. A549-hACE2 cells were treated with varying concentration of surrogate ligands, IFNω or negative control (monomer VHH “A1”) for 24 hr. prior to infection with SARS-COV-2 nLUC. SARS-COV-2 nLUC replication (relative light units) for triplicate wells per VHH dilution is shown. (I-J) Heatmap representation of selected ISGs induced by surrogate ligands in A549 cells (I) or human primary bronchial/tracheal epithelial cells (J). Gene expression is normalized to the level induced by IFNω (K) qRT-PCR analysis of SeV RNA in PBMCs pre-treated with 10 nM surrogate ligands or IFNω for 24 hr. followed by SeV infection (MOI=0.5) for 24 hr. (L) Heatmap representation of selected ISGs induced by surrogate ligands in PBMCs. (M) CellTiter-GLO assay of human primary bronchial/tracheal epithelial cells treated with 10 nM surrogate ligands or IFNω for 72 hr. (N) Identification of four mouse IFN surrogate agonists with pSTAT1 activity on J774.2 cells (left) and on mouse embryonic fibroblasts (right). (O) Mouse IFN surrogates exert antiviral activity against SeV.

FIGS. 8A-8D depict signaling kinetics and gene expression driven by Type I Interferon surrogate ligands. (A) SPR sensorgrams displaying dose-dependent binding of IFNAR2 VHHs to immobilized human IFNAR2 ECD. SPR experiments were performed using the same conditions as for IL-2 specific VHHs. Binding constants were determined from kinetic fitting and summarized in (B). (C) qRT-PCR analysis of mRNA level of indicated genes in human primary bronchial/tracheal epithelial cells treated with 10 nM surrogate ligands or IFNω for 8 hr. (D) qRT-PCR analysis of mRNA level of indicated genes in PBMCs treated with 10 nM surrogate ligands or IFNω for 8 hr.

FIGS. 9A-9O embodies a surrogate agonist that enforces proximity between IL-2Rβ and IL-10Rβ activates pSTAT5 signaling in T and NK cells. (A) Schematic showing non-natural receptor pairing of IL-10Rβ/IL2-Rβ to create a synthetic JAK1/TYK2 heterodimer. (B) Thirty IL-10β/IL-2Rβ VHH pairings (with 8 a.a. linkers) in forward and reverse orientations were expressed and assayed for pSTAT5 activity in YT-1 CD25⁻ cells. Of the thirty combinations, four pairings had partial pSTAT5 activity (pink shading). (C) Modulation of ligand activity by linker length. 10Rβ1-2Rβ6 agonists with varying linker length between 0-16 amino acids were tested for pSTAT5 signaling in YT-1 cells. (D) Modulation of agonist activity by Fc-mediated dimerization. (E) Dimerization of the 10Rβ1-2Rβ6 ligand via Fc-fusion enhances pSTAT5 in primary human T cells. (F-G) CD8⁺ but not CD4⁺ T cell proliferation is driven by 10Rβ1-2Rβ6 and 10Rβ1-2Rβ6-Fc. Pre-activated human T cells were cultured with varying concentrations of 10Rβ1-2Rβ6 (pink) and 10Rβ1-2Rβ6-Fc agonists (black). Dose-response relationship of CD4+ (F) and CD8+ (G) T cells proliferation is indicated. (H) CD8+ but not CD4+ T cell differentiation is driven by 10Rβ1-2Rβ6 and 10Rβ1-2Rβ6-Fc. (I) Dose-response of 10Rβ1-2Rβ6 agonist (pink) and 10Rβ1-2R6-Fc agonist (black) on pSTAT5 in primary effector NK cells. Data (mean±SD) are from three independent replicates. (J) Effector NK cells were labelled with 5 μM CFSE for 20 min at 37° C. Histogram at 100 nM ligand concentration displays proliferation of effector NK following 3d culture. (K) NKL killing of K562 tumor cells is enhanced by treatment with 10Rβ1-2Rβ6 (pink), 10Rβ1-2Rβ6-Fc (black) and hIL-2 (red). (L-O) Degranulation and activation of NKL cells in response to 10Rβ1-2Rβ6, 10Rβ1-2Rβ6-Fc, hIL-7 and hIL-2.

FIGS. 10A-10B demonstrate that a surrogate agonist compels heterodimerization of IL-2Rβ and IL-10Rβ and shows bias for CD8+ T and NK cells. (A) SPR sensorgrams displaying dose-dependent binding of IL-10Rβ VHHs to immobilized human IL-10Rβ ECD. SPR experiments were performed using the same conditions as for IL-2 specific VHHs. Binding constants were determined from kinetic fitting and summarized in (B).

FIG. 11 shows sequences of individual VHH binding modules of human IL-2 surrogate agonists.

FIG. 12 shows sequences of active molecules from initial screen of human IL-2 surrogate agonists (FIG. 1D left, β-γc Forward orientation):IL2Rβ-VHH-γc-VHH with 2 or 8a.a. Linker. The first column denotes the name of the molecule (for e.g. MY144-F), the second column denotes the type of module combo (for e.g. R-VHH1γc-VHH4.)

FIGS. 13A-13B show sequences of active molecules from initial screen of human IL-2 surrogate agonists (FIG. 1D right, γc-β Reverse orientation) with 2 or 8 a.a. linkers.

FIGS. 14A-14B show sequences of active molecules of human IL-2 surrogate agonists assembled in alternative formats, including acid-base zippers and in a triple module orientation.

FIGS. 15A-15B show sequences of individual VHH binding modules of Human Type I IFN surrogate agonists.

FIG. 16 shows sequences of individual VHH binding modules of Mouse Type I IFN surrogate agonists.

FIGS. 17A-17B show sequences of active molecules from the initial screen (FIG. 11A) of Human Type I IFN surrogate agonists.

FIG. 18 shows sequences of active molecules from Mouse Type I IFN surrogate agonists.

FIG. 19 shows sequences of molecules selected for functional studies of Human Type I IFN surrogate agonists (see FIGS. 7A and 7β-M).

FIG. 20 shows sequences of individual VHH binding modules of Human IL-2Rβ/IL-10Rβ surrogate agonists.

FIG. 21 shows sequences of active molecules from initial screen of Human IL-2Rβ/IL-10Rβ surrogate agonists (see FIG. 9B).

FIG. 22A shows sequences of linker-modulated 10Rβ1-2Rβ6 constructs (see FIG. 9C) of Human IL-2Rβ/IL-10Rβ surrogate agonists. FIG. 22B shows sequence of 10Rβ1-2Rβ6-Fc construct (refers to FIGS. 7D-O).

### DETAILED DESCRIPTION OF THE DISCLOSURE

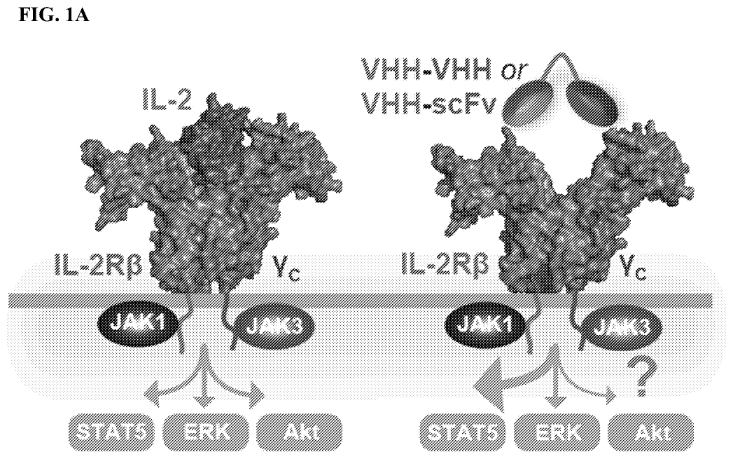

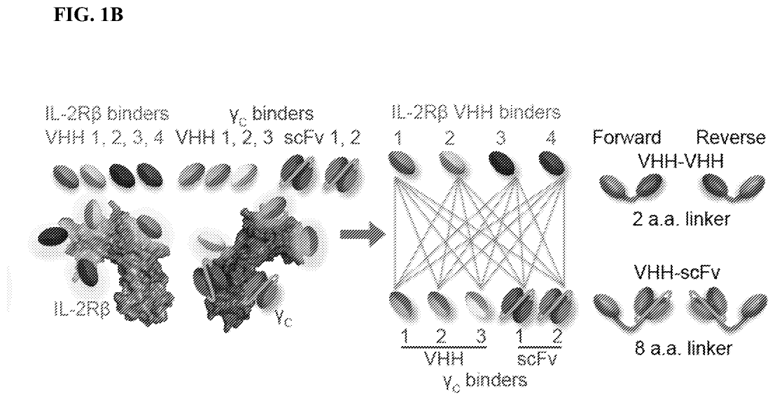

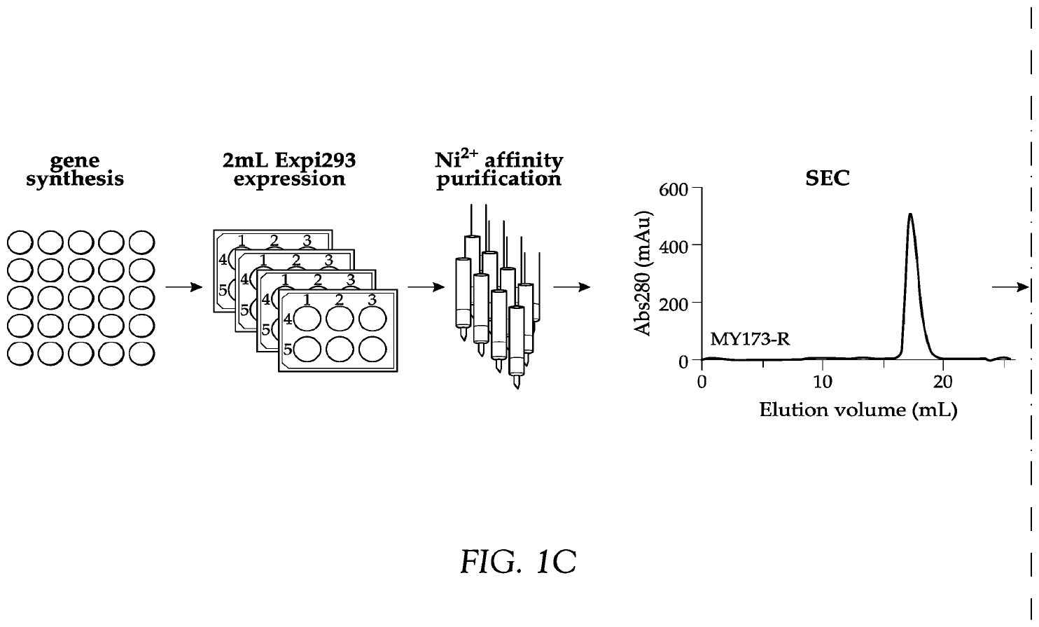

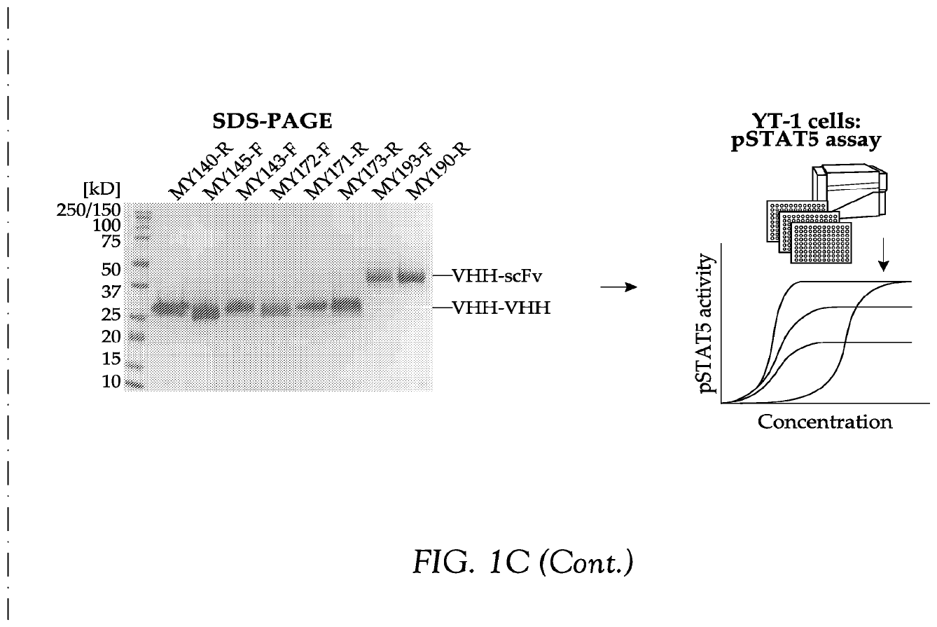

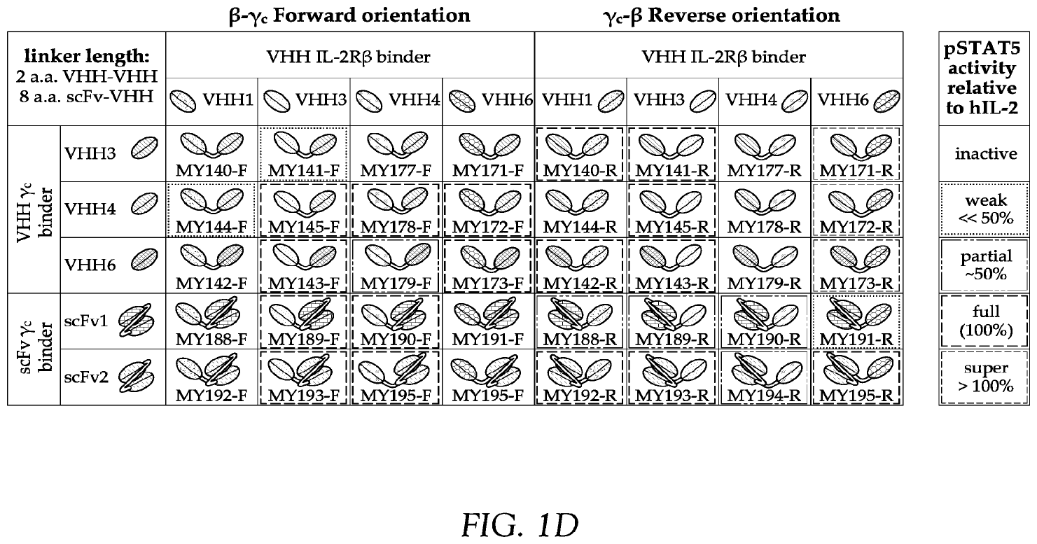

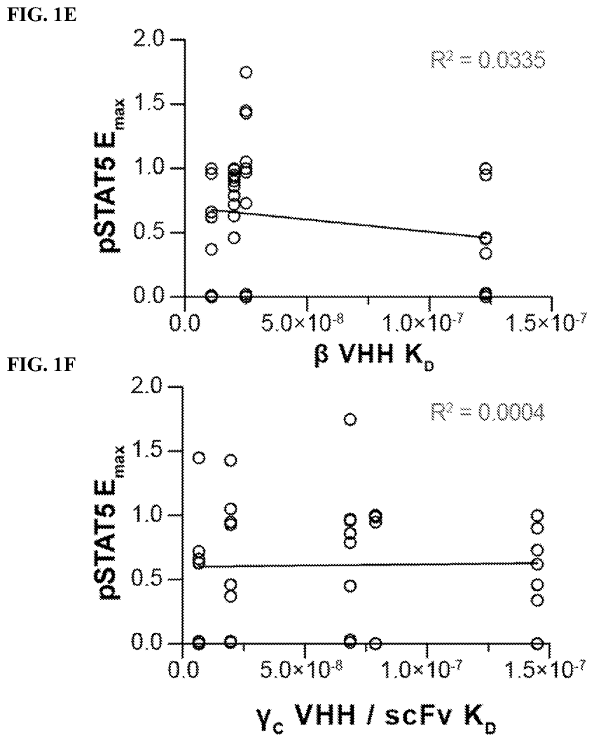

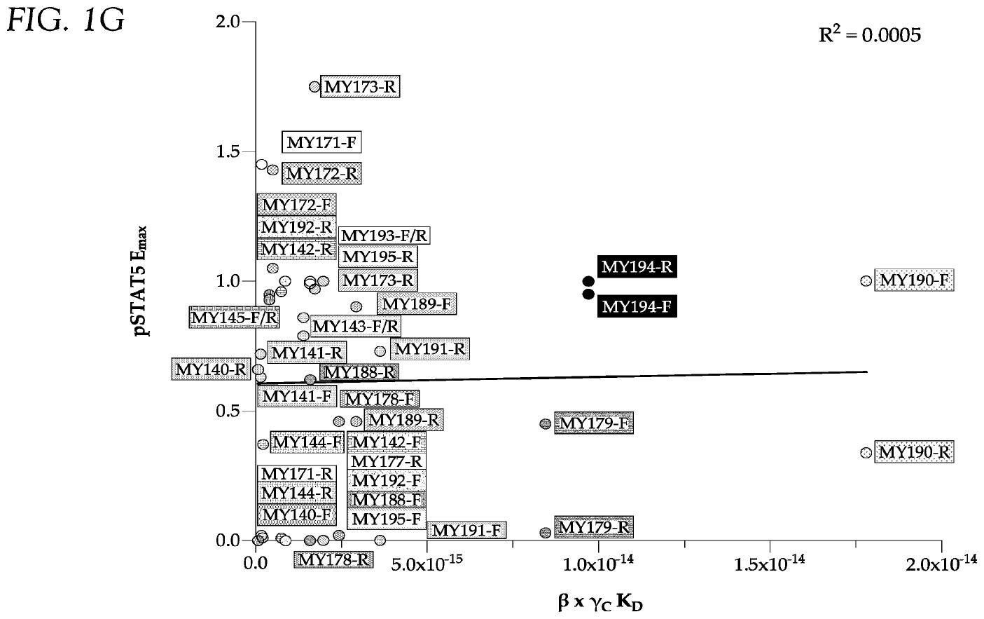

The present disclosure generally relates to compositions and methods pertaining to engineered polypeptides that are cytokine agonists. The present disclosure also relates to platforms for the generation and screening of agonists for naturally- and non-naturally occurring combinations of receptors. The engineered polypeptides of the present disclosure include ligands that have the capacity to dimerize cell surface receptors in ways that are structurally inaccessible to natural or engineered cytokines. The ligands are single chain bispecific ligands that can include one or more antibody domains. The domains (binders) can include one or more nanobodies (VHH) and/or scFvs that can be mixed and matched in modular fashion to create libraries of dimerizing ligands (FIGS. 1A and 1B). The engineered polypeptides can dimerize various receptors including, but not limited to, the IL-2/IL-15, Type I IFN and IL-10 cytokine systems.

The present disclosure also relates to methods of and systems for identifying such cytokine agonists. These methods and systems can be used for any multimeric cell surface receptors including dimeric receptors (e.g. cytokine, Receptor Tyrosine Kinase (RTK) and IgSF family), trimeric receptors (e.g. death receptors), and other systems. The methods and systems can also be used for systems with limited or nonexistent structural knowledge, or for creating surrogate ligands when the cognate ligands present biochemical challenge. The methods and systems can be used in both natural and non-natural receptor combinations and can be used to explore new receptor combinations for drug discovery.

Cytokines are powerful immune modulators that initiate signaling through receptor dimerization, but natural cytokines have structural limitations as therapeutics. Disclosed herein are strategies and methods for the discovery of surrogate cytokine agonists using modular ligands with the capability of exploring receptor dimer geometry as a pharmacological variable. The strategies and methods are amenable to high-throughput screening. As described in greater detail herein, combinatorial matrices of single chain bispecific ligands that exhibited a diverse spectrum of agonist strengths, signaling biases and functional activities that have been inaccessible through traditional cytokine engineering have been generated using VHH and scFv to, for example, human or mouse Interleukin-2/15, Type I Interferon and Interleukin-10 receptors. As described in greater detail below, this modular approach can enable the engineering of a ligand that compels the formation of heterodimers on T and NK cells, generating a non-canonical activation signal.

The section headings used herein are for organizational purposes only and are not to be construed as limiting the subject matter described.

### I. General Techniques

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

### II. Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “linker”, as used herein, refers to an amino acid or sequence of amino acids that that is optionally located between two amino acid sequences in a fusion polypeptide of the invention.

The term “specific” or “specificity”, as used herein in reference to the binding of two molecules or a molecule and a complex of molecules, refers to the specific recognition of one for the other and the formation of a stable complex, as compared to substantially less recognition of other molecules and the lack of formation of stable complexes with such other molecules. Preferably, “specific,” in reference to binding, means that to the extent that a molecule forms complexes with other molecules or complexes, it forms at least fifty percent of the complexes with the molecule or complex for which it has specificity. Generally, the molecules or complexes have areas on their surfaces or in cavities giving rise to specific recognition between the two binding moieties. Exemplary of specific binding are antibody-antigen interactions, enzyme-substrate interactions, polynucleotide hybridizations and/or formation of duplexes, cellular receptor-ligand interactions, and so forth.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, which in some embodiments relate to mammalian nucleic acid and amino acid sequences, are intended to encompass homologous and/or orthologous genes and gene products from other animals including, but not limited to other mammals, fish, amphibians, reptiles, and birds. In some embodiments, the genes, nucleic acid sequences, amino acid sequences, peptides, polypeptides and proteins are human. The term “gene” is also intended to include variants thereof.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the complement of a sequence. This definition also includes sequences that have deletions and/or additions, as well as those that have substitutions. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “pharmaceutically acceptable excipient” as used herein refers to any suitable substance that provides a pharmaceutically acceptable carrier, additive or diluent for administration of a compound(s) of interest to a subject. As such, “pharmaceutically acceptable excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics and additional therapeutic agents) can also be incorporated into the compositions.

The term “engineered” or “recombinant” polypeptide as used herein, refers to a polypeptide that has been altered through human intervention. As non-limiting examples, an engineered polypeptide can be one which: 1) has been synthesized or modified in vitro, for example, using chemical or enzymatic techniques; 2) includes conjoined polypeptide sequences that are not conjoined in nature; 3) has been engineered using molecular cloning techniques such that it lacks one or more amino acids with respect to the naturally occurring polypeptide sequence; and/or 4) has been manipulated using molecular cloning techniques such that it has one or more sequence changes or rearrangements with respect to the naturally occurring polypeptide.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value=up to 10%, up to =5%, or up to +1%.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub combination was individually and explicitly disclosed herein.

### Interleukin-2 (IL-2) and Interleukin-15 (IL-15)

Some embodiments of the disclosure relate to new cytokine ligands with properties of partial or complete agonisms of the downstream signal transduction mediated through immunoregulatoy cytokines such as interleukin-2 (IL-2) or interleukin-15 (IL-15).

Interleukin-2 (IL-2) is a stimulatory cytokine that directs proliferation and survival of T lymphocytes, natural killer (NK) cells, and B lymphocytes (Lin and Leonard, 2018). IL-2, like IL-15, signals through a receptor heterodimer composed of common gamma (γ_{c}) and IL-2Rβ which trigger signaling through JAK-STAT, MAP kinase/ERK, and PI3 kinase-Akt pathways (Leonard et al., 2019). IL-2 activates JAK1 and JAK3 kinases, which relay the signal primarily through STAT5 activity (Miyazaki et al., 1994; Russell et al., 1994; Xue et al., 2002). An important function of IL-2 is to induce CD8⁺ T cell differentiation and to direct the differentiation of naïve CD8⁺ T cells into memory and cytotoxic effector cells. IL-2 and IL-15 are also known to support NK cell expansion and arm them with cytotoxic function (Wu et al., 2017).

Common gamma chain (γ_{c}) is a shared receptor component of the γ_{c} family of cytokines, which includes IL-2/15, as well as interleukins 4, 7, 9, and 21. Its importance in immune function is underscored by the fact that loss of function mutations in Ye result in severe combined immunodeficiency in humans. The γ_{C} family cytokines collectively control the differentiation, homeostatic proliferation, and function of immune cells (Leonard et al. 2019).

### Interleukin-10 (IL-10)

Some embodiments of the disclosure also relate to new cytokine ligands with properties of partial or complete agonisms of the downstream signal transduction mediated through the cytokine interleukin-10 (IL-10).

IL-10 is an immunoregulatory cytokine that possesses both anti-inflammatory and immunostimulatory properties and is frequently dysregulated in human disease. It is a pleiotropic cytokine expressed as a non-covalently linked homodimer of ˜37 kDa and regulates multiple immune responses through actions on T cells, B cells, macrophages, and antigen presenting cells (APC). Its predominantly anti-inflammatory properties have been widely reported. IL-10 has been reported to suppress immune responses by inhibiting expression of IL-1, IL-Ib, IL-6, IL-8, TNF-α, GM-CSF, and G-CSF in activated monocytes and activated macrophages. Although IL-10 is predominantly expressed in macrophages, expression has also been detected in activated T cells, B cells, mast cells, and monocytes. In addition to suppressing immune responses, IL-10 exhibits immuno-stimulatory properties, including stimulating the proliferation of thymocytes treated with IL-2 and IL-4, enhancing the viability of B cells, and stimulating the expression of MHC class II.

Various immuno-stimulatory properties of IL-10 have been reported. IL-10 can costimulate B-cell activation, prolong B-cell survival, and contribute to class switching in B-cells. Moreover, it can costimulate natural killer (NK) cell proliferation and cytokine production and act as a growth factor to stimulate the proliferation of certain subsets of CD8⁺ T cells. It has been reported that high doses of IL-10 in humans can lead to an increased production of INFγ. IL-10 signals through a two-receptor complex consisting of two copies each of IL-10 receptor 1 (IL-10Rα) and IL-10Rβ. It has been reported that IL-10Rα binds IL-10 with a relatively high affinity (−35-200 pM), and the recruitment of IL-10Rβ to the receptor complex makes only a marginal contribution to ligand binding. However, the engagement of IL-10Rβ to the complex enables signal transduction following ligand binding. Thus, the functional receptor consists of a dimer of heterodimers of IL-10Rα and IL-10Rβ. Most hematopoietic cells constitutively express low levels of IL-10Rα, and receptor expression can often be dramatically upregulated by various stimuli. In contrast, the IL-10Rβ is expressed on most cells. The binding of IL-10 to the receptor complex activates the Janus tyrosine kinases, JAK1 and Tyk2, associated with IL-10Rα and IL-10Rβ, respectively, to phosphorylate the cytoplasmic tails of the receptors. This results in the recruitment of STAT3 to the IL-10Rα. The homodimerization of STAT3 results in its release from the receptor and translocation of the phosphorylated STAT homodimer into the nucleus, where it binds to STAT3-binding elements in the promoters of various genes. One of these genes is IL-10 itself, which is positively regulated by STAT3. STAT3 also activates the suppressor of cytokine signaling 3 (SOCS3), which controls the quality and quantity of STAT activation. SOCS3 is induced by IL-10 and exerts negative regulatory effects on various cytokine genes.

As a result of its pleiotropic activity, IL-10 has been linked to a broad range of diseases, disorders and conditions, including inflammatory conditions, immune-related disorders, fibrotic disorders and cancer. In view of the prevalence and severity of IL-10-associated diseases, disorders and conditions, novel IL-10 agents and modifications thereof would be of tremendous value in the treatment and prevention of IL-10-associated diseases, disorders and conditions.

IL-10 maintains the balance of the immune response, allowing the clearance of infection when minimizing damage to the host. It can also dampen the harmful immune responses elicited in autoimmunity and allergy. IL-10 dimerizes IL-10Rα and IL-10Rβ to elicit STAT1 and STAT3 activation (Ouyang and O'Garra, 2019).

### Type I Interferons (IFN)

Some embodiments of the disclosure relate to new cytokine ligands with properties of partial or complete agonisms of the downstream signal transduction mediated through Type I Interferons (IFN).

Type I interferons (IFNs) are a network of homologous cytokines that bind to a shared, heterodimeric cell surface receptor (has two transembrane subunits IFNAR1 and IFNAR2) and engage signaling pathways that activate innate and adaptive immune responses. The IFNs have a wide range of immunomodulatory, anti-viral, and anti-proliferative actions which are mediated by 16 different sub-types of IFN cytokines that dimerize IFNAR1/IFNAR2 to activate several STATs, principally STAT1 (Ng. et al., 2016).

### III. Compositions

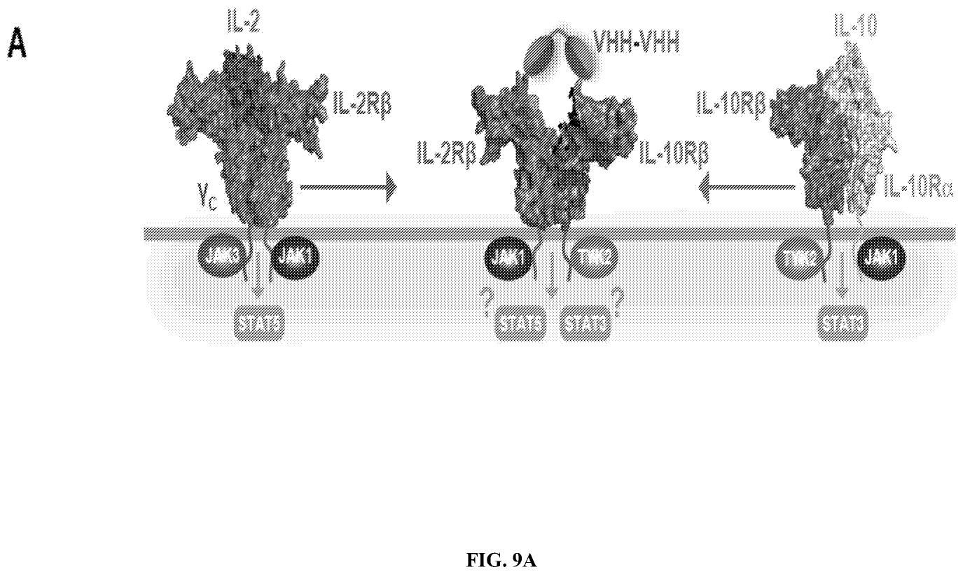

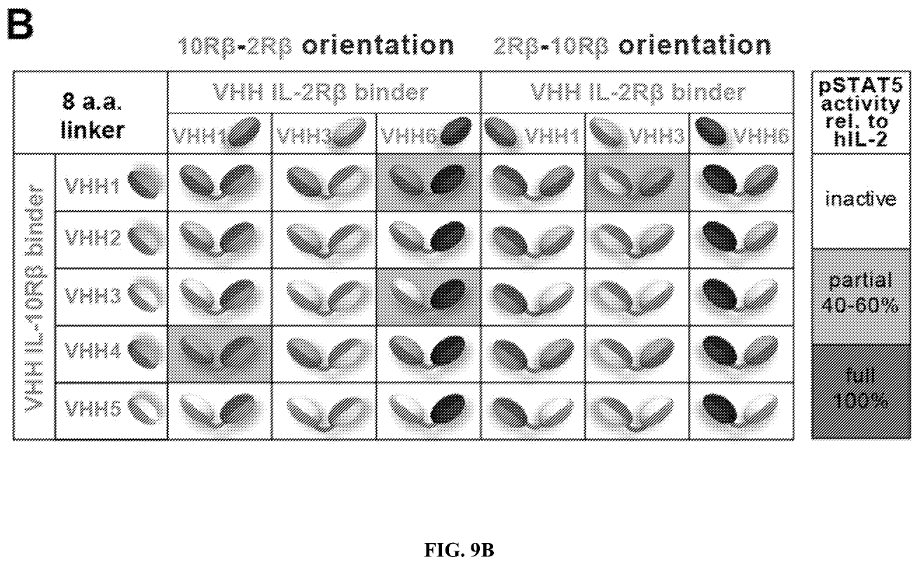

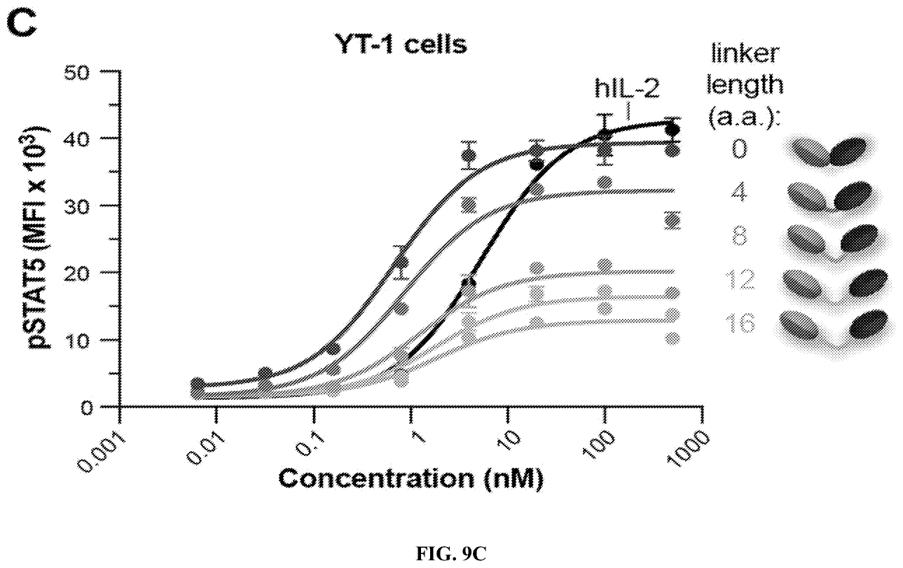

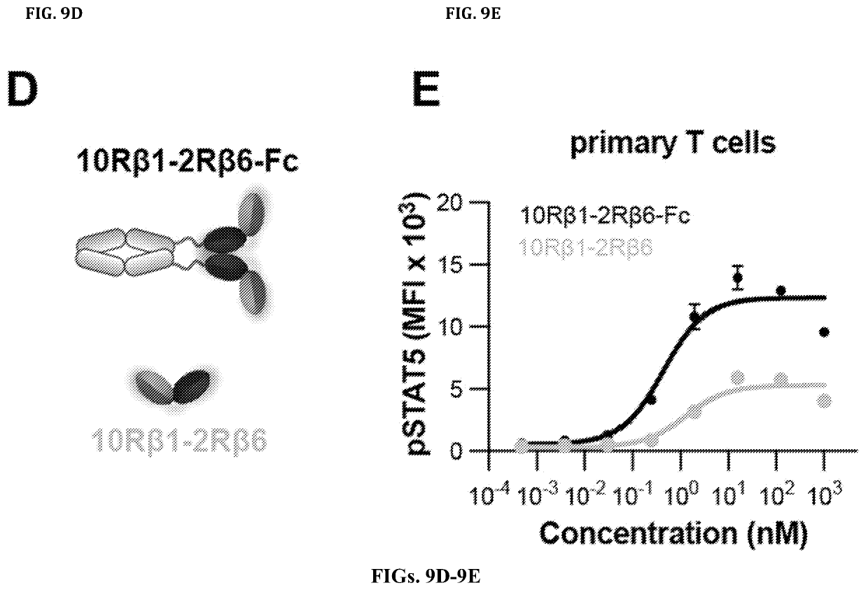

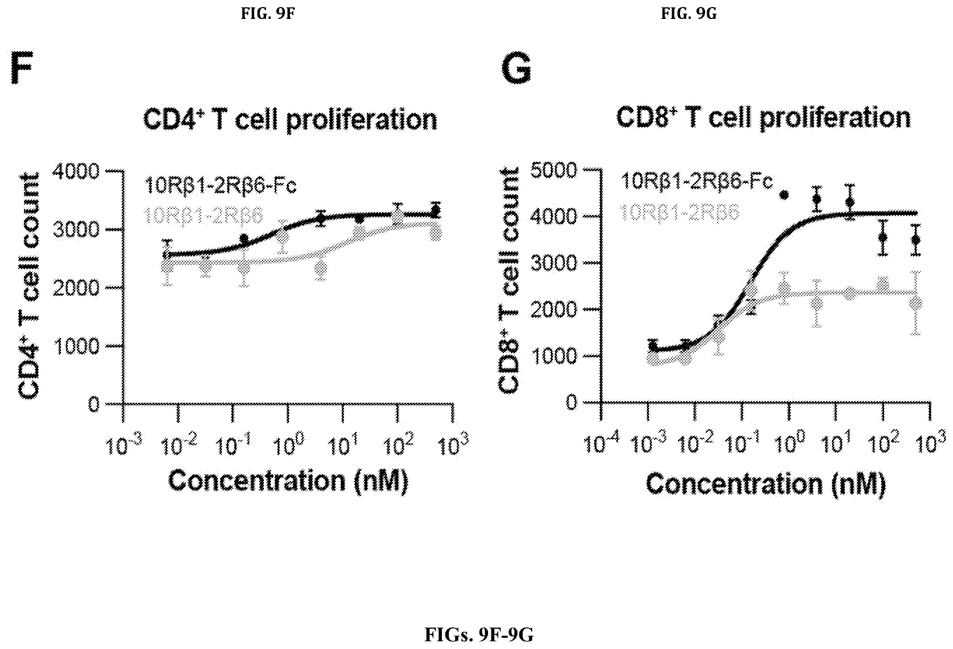

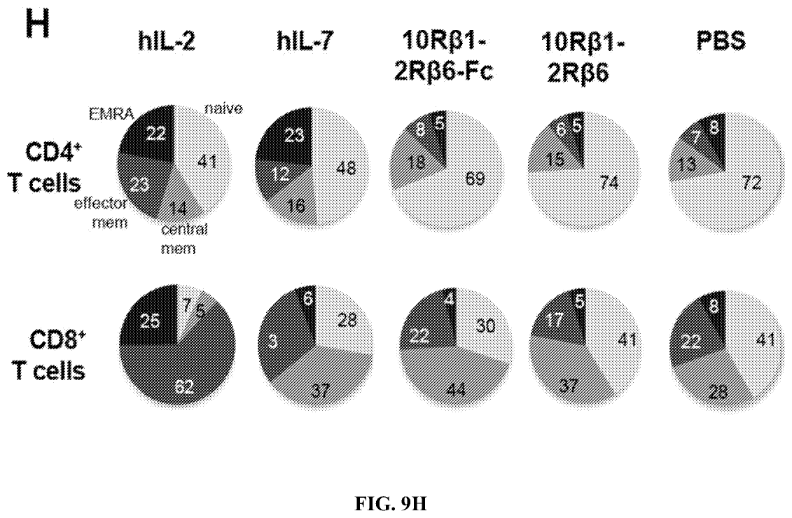

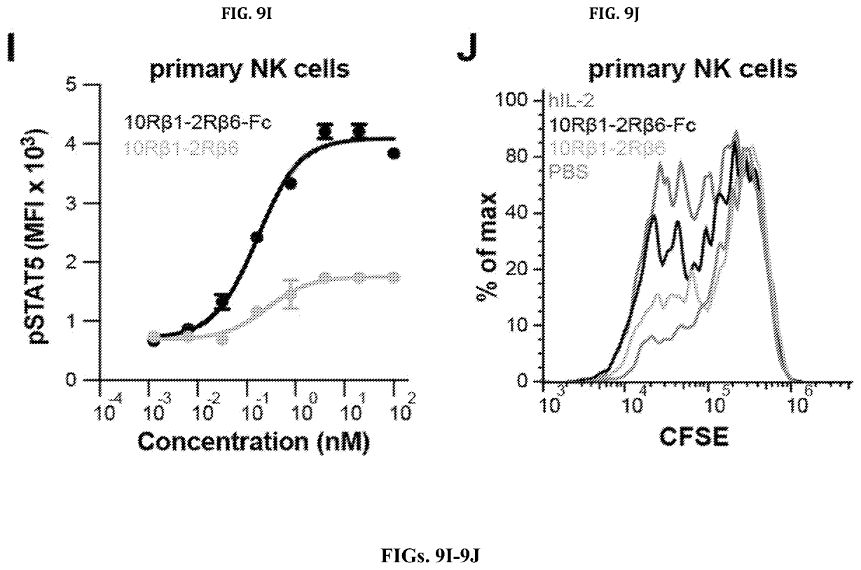

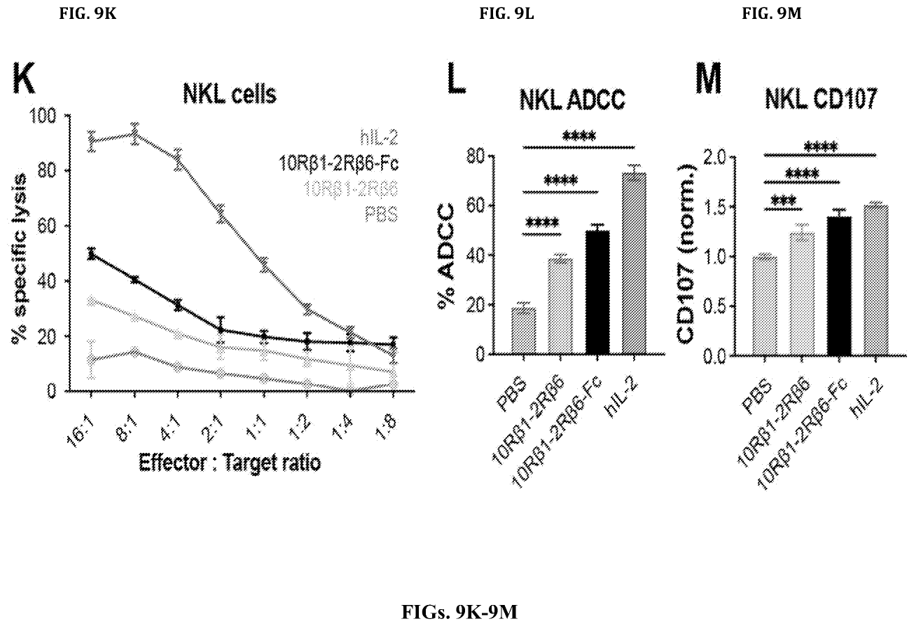

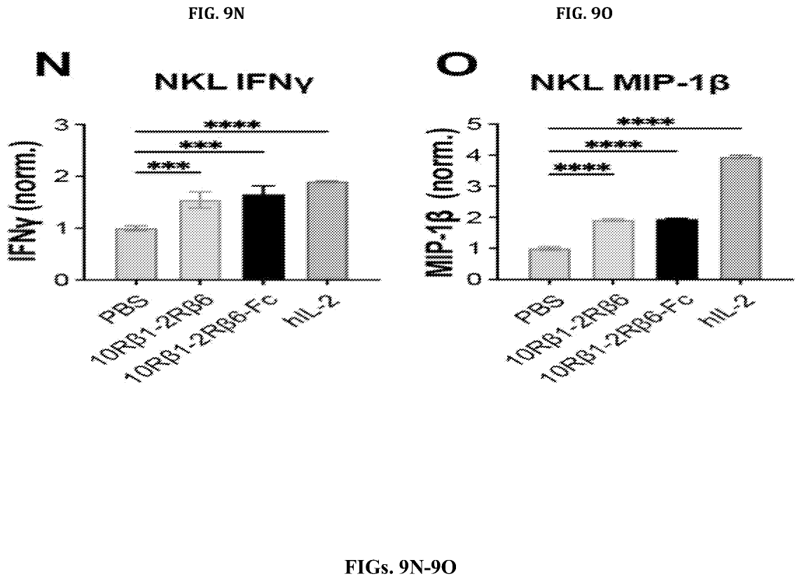

The compositions disclosed herein include surrogate cytokine agonists that comprise bispecific single-chain ligands. The single-chain ligands are made up of antibody domains (binders) that can be mixed and matched to create libraries of dimerizing ligands described throughout the disclosure, figures and Examples presented below. For instance, various combinations of antibody domains are shown in FIGS. 1D and 9B and in the Informal Sequence Listing. A person of skill in the art would appreciate that while several embodiments have the scFvs or VHHs fused together, other formats, (e.g. zippers and Fc heterodimers) may also be used.

The ligands can be assembled pursuant to methods known to those skilled in the art. In some embodiments, the ligands are heterodimers. In some embodiments, the heterodimers can be expressed as Fc fusions, which can then self-dimerize via their Fc domains to generate bispecific homodimers, as shown in FIG. 9. In some embodiments, the domains can be separately fused to acidic or basic zippers, which when co-expressed, self-assemble to generate bispecific heterodimers. In some embodiments, the ligands are heterotrimers. In some embodiments, the ligands are two-chain ligands wherein the ligand is encoded by a single polypeptide and wherein two molecules of the polypeptide self-assemble to make a homodimer.

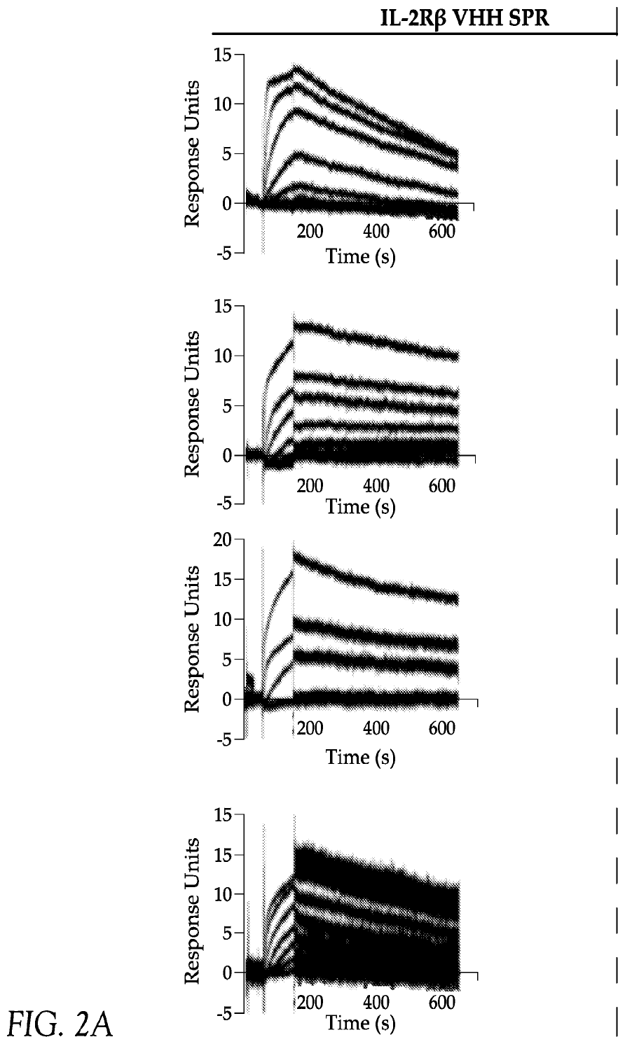

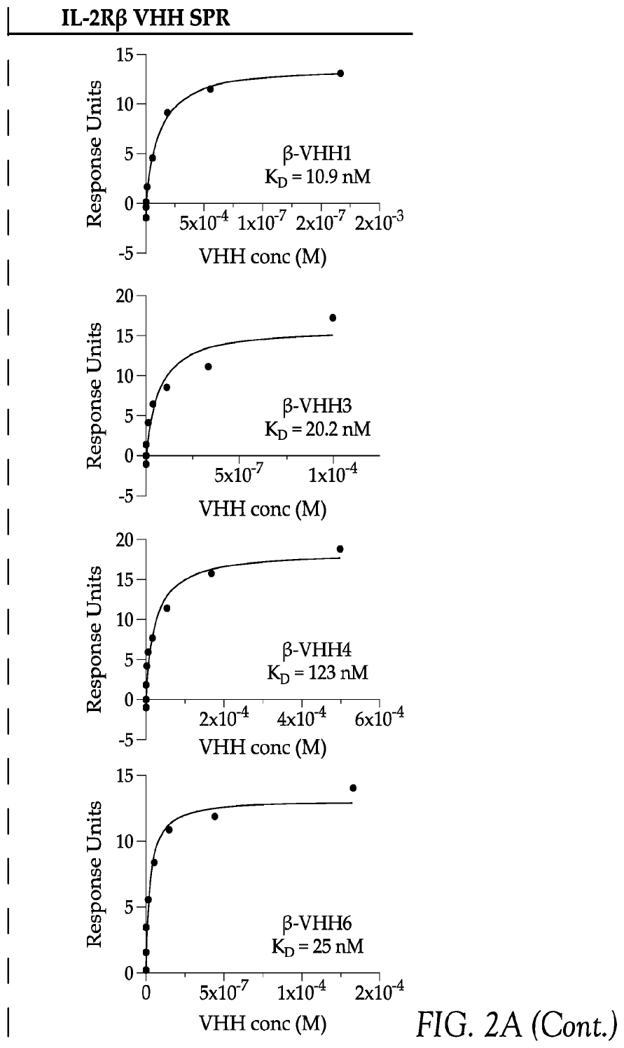

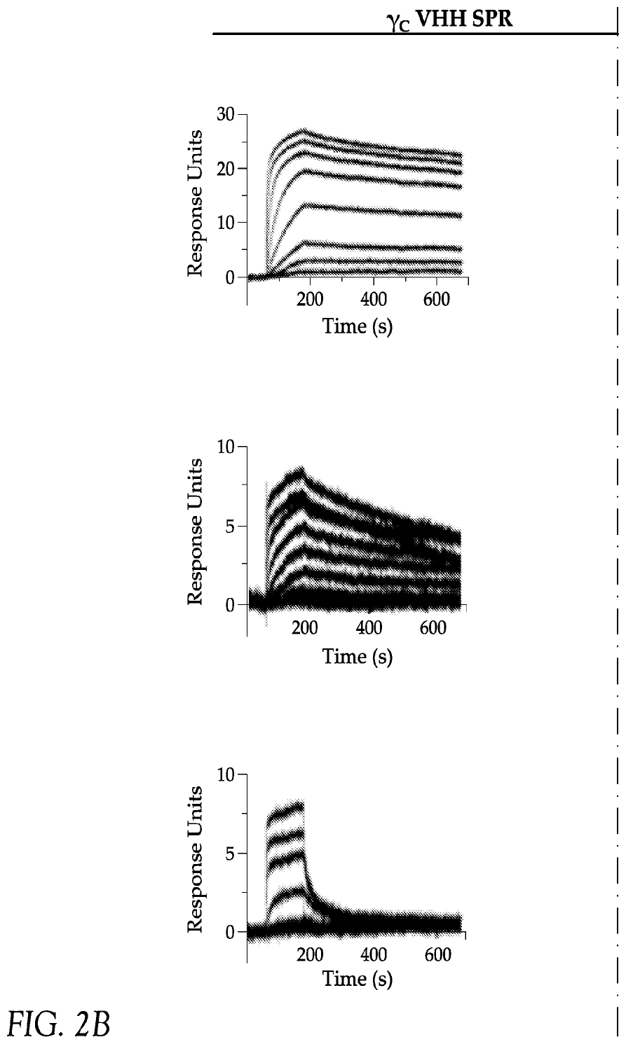

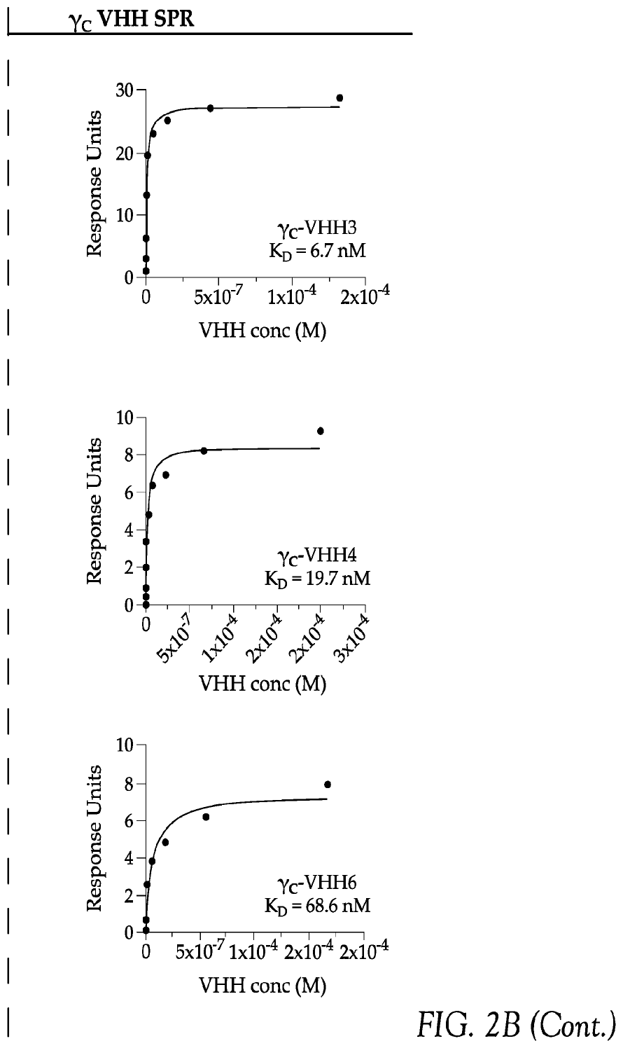

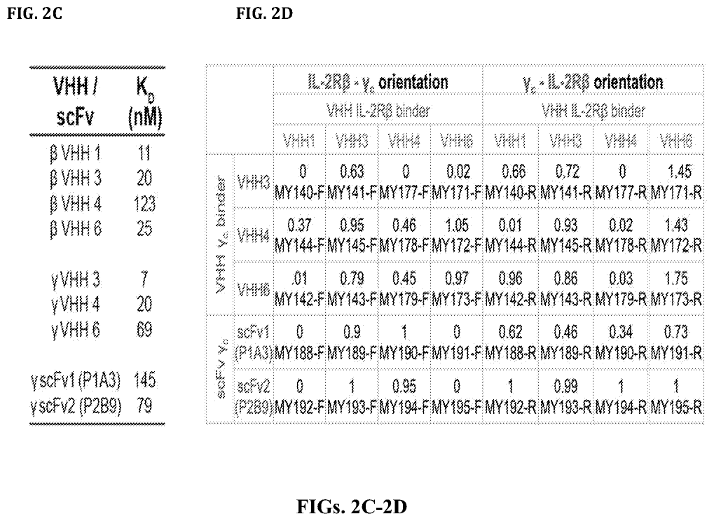

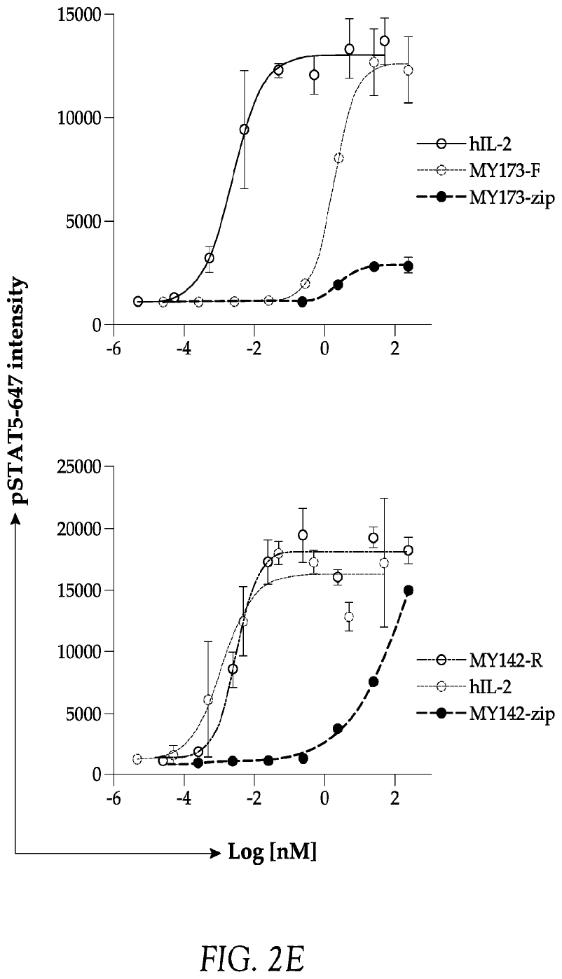

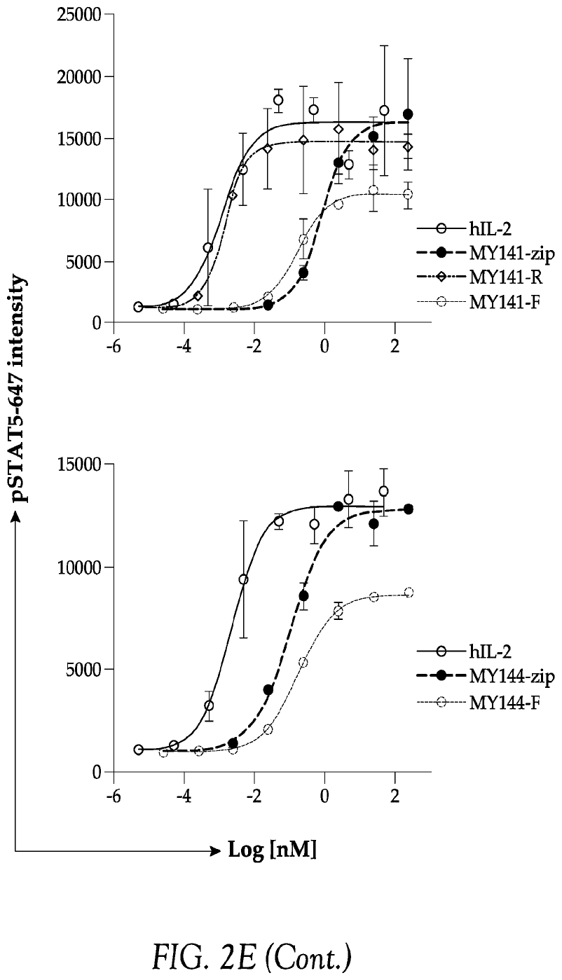

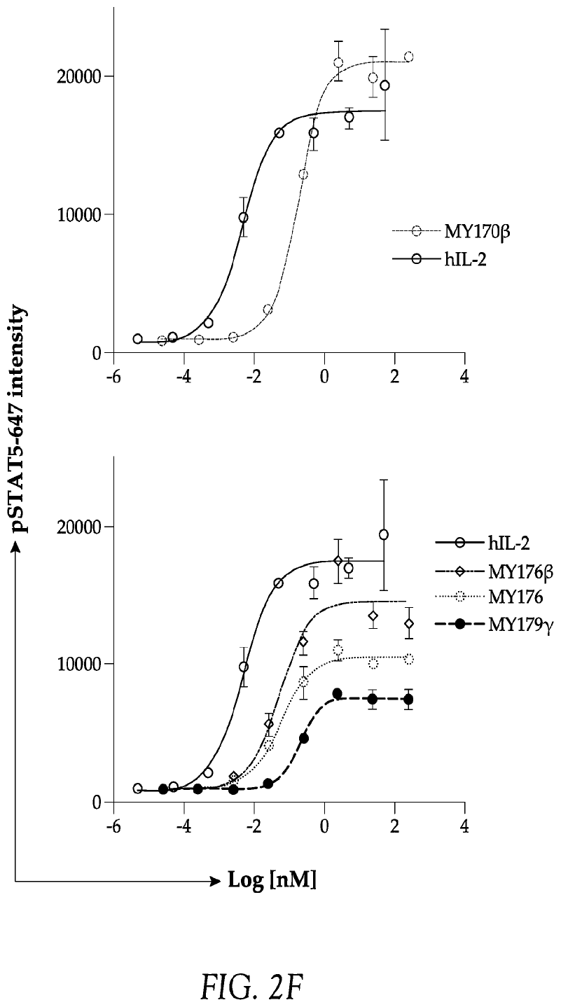

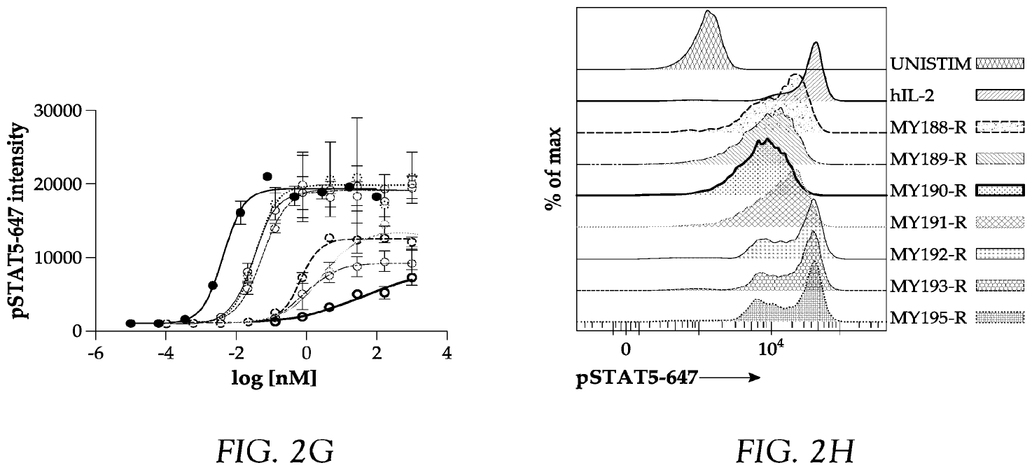

In some embodiments, the ligands are multi-chain agonists that include fusions to oligomeric zippers. Oligomeric zippers are comprised of alpha helical protein domains that self-assemble into dimers, trimers, tetramers, etc. They can be fused to proteins of interest to multimerize them into the desired stoichiometry. Oligomeric zippers are known in the art, such as described by Harbuy et al. 1993. The use of such ligands in tuning of IL-2 activity via generation of acid-base VHH zipper pairings is shown in FIG. 2E.

The ligands of the present disclosure are surrogate cytokine agonists. Measuring agonism is within the standard knowledge of a skilled artisan. For example, in some embodiments, measuring agonism may be done by stimulating cells expressing the receptor(s)-of-interest with the candidate ligand, then assaying a biological output such as proximal signaling. In some embodiments of the present disclosure, measuring agonism can be performed by assaying phosphorylation of STATs. In some embodiments, measuring agonism can be performed by assaying downstream function(s) (for example, assaying proliferation, differentiation, antiviral activity, etc. using known methods).

Engineered Polypeptides and Ligands with Specificity to IL-2Rβ and Ic

As described in greater detail below, the present disclosure relates to engineered polypeptides that are cytokine agonists. In one aspect, the present disclosure relates to an engineered polypeptide having a single chain bispecific ligand wherein a first specificity of the ligand is to IL-2Rβ and a second specificity of the ligand is to ye and wherein the engineered polypeptide is a cytokine agonist.

In some embodiments, the single-chain bispecific ligand includes one or more antibody domains (binders, modules). The term “antibody domain” or “antigen binding fragment” as used herein and throughout the present disclosure refers to an antibody fragment such as, for example, a diabody, a Fab, a Fab′, a F(ab′) 2, an Fv fragment, a disulfide stabilized Fv fragment (dsFv), a (dsFv) 2, a bispecific dsFv (dsFv-dsFv′), a disulfide stabilized diabody (ds diabody), a single-chain antibody molecule (scFv), an scFv dimer (bivalent diabody), a multispecific antibody formed from a portion of an antibody comprising one or more CDRs. The antigen-binding moiety can include naturally-occurring polypeptides or can be engineered, designed, or modified so as to provide desired and/or improved properties. As used herein, the term “VHH” or “nanobody” are used herein interchangeably to refer to variable domain of a heavy-chain antibody. A nanobody is the smallest antigen binding fragment or single variable domain derived from naturally occurring heavy chain antibody and is known to the person skilled in the art. They are derived from heavy chain only antibodies, seen in camelids (Hamers-Casterman el al. 1993; Desmyter el al. 1996). In the family of “camelids,” immunoglobulins devoid of light polypeptide chains are found. “Camelids” comprise old world camelids (Camelus bactrianus and Camelus dromedarius) and new world camelids (for example, Lama paccos, Lama glama, Lama guanicoe, and Lama vicugna). The single variable domain heavy chain antibody is herein designated as a nanobody or a VHH antibody. Nanobodies can also be derived from sharks. In some embodiments, the antibody domain is an antigen binding fragment of a VHH (nanobody) or a single-chain variable fragment (scFv). In some embodiments, the single chain bispecific ligand includes two nanobodies, a first nanobody that is specific to IL-2Rbeta and a second nanobody that is specific to γ_{c}.

In some embodiments, the single chain bispecific ligand includes three nanobodies. In some embodiments, the single chain bispecific ligand includes a first VHH specific to IL2Rβ, a second VHH that is specific to γ_{c}, and a third nanobody that is specific to IL2Rβ. In some embodiments, the single chain bispecific ligand includes a first VHH to γ_{c} and a second VHH that is specific to IL2Rβ and a third nanobody that is specific to γ_{c}. This embodiment is shown in FIG. 2F, which describes tuning IL-2 activity via generation of 2:1 β:γ_{c} orγ_{c}:β stoichiometries. VHH binding modules were linked together in a tripartite (β-γ_{c}-β or γ_{c}-β-γ_{c}) manner.

In some embodiments, the single-chain bispecific ligand has the first nanobody specific to IL-2Rβ at the N-terminus of the engineered polypeptide and the second nanobody at the C-terminus of the engineered polypeptide. In some embodiments, the first and second nanobodies are in the opposite or reverse orientation—the first nanobody is at the C-terminus of the engineered polypeptide and the second nanobody is at the N-terminus of the engineered polypeptide.

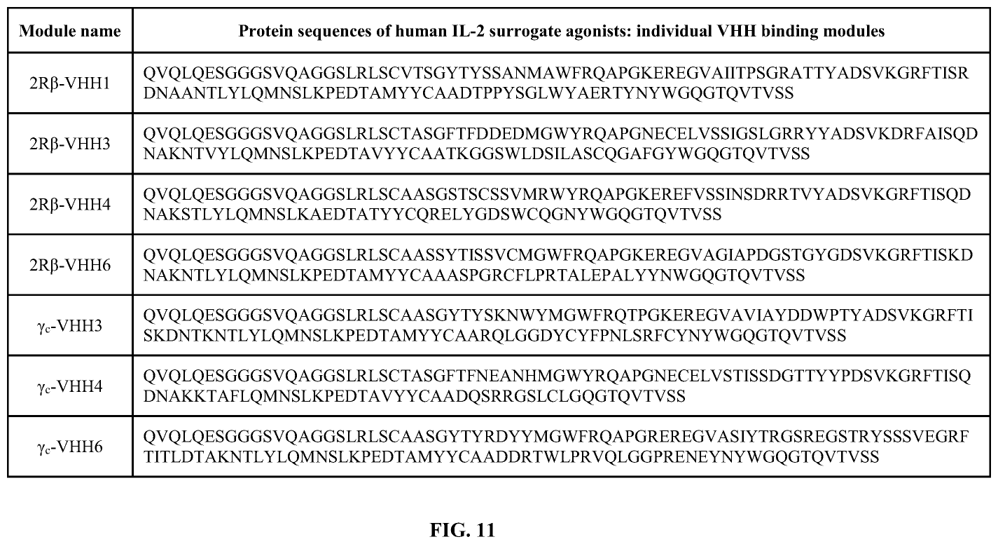

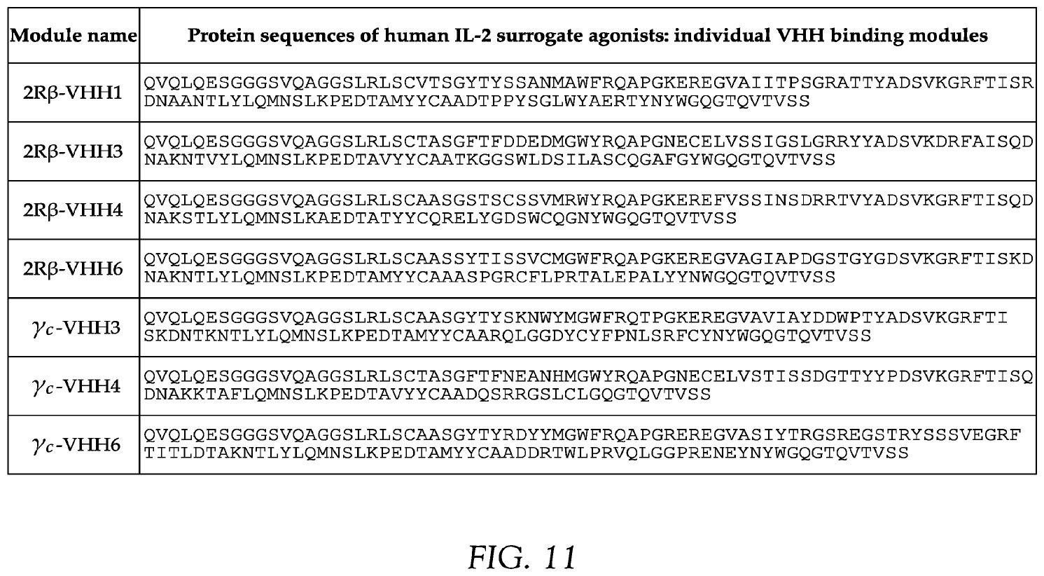

In some embodiments, the first nanobody includes an amino acid sequence having the same identity to the amino acid sequence set forth in any one of SEQ. ID. NOS.: 1-4 (FIG. 11). In some embodiments, the first nanobody has at least 80%, or 85% or 90%, or 91% or 92% or 93% or 94% or 95% or 96% or 97% or 98% or 99%, or any values in between, sequence identity to the amino acid sequence set forth in any one of SEQ. ID. NOS.: 1-4. In some embodiments, the first nanobody includes an amino acid sequence having the same identity to the amino acid sequence set forth in any one of SEQ. ID. NO: 1. In some embodiments, the first nanobody includes an amino acid sequence that has at least 80%, or 85% or 90%, or 91% or 92% or 93% or 94% or 95% or 96% or 97% or 98% or 99% sequence identity to the amino acid sequence of SEQ ID NO: 1. In some embodiments, the first nanobody includes an amino acid sequence having the same identity to the amino acid sequence set forth in any one of SEQ. ID. NO: 2. In some embodiments, the first nanobody includes an amino acid sequence that has at least 80%, or 85% or 90%, or 91% or 92% or 93% or 94% or 95% or 96% or 97% or 98% or 99% sequence identity to the amino acid sequence of SEQ ID NO: 2. In some embodiments, the first nanobody includes an amino acid sequence having the same identity to the amino acid sequence set forth in any one of SEQ. ID. NO: 3. In some embodiments, the first nanobody includes an amino acid sequence that has at least 80%, or 85% or 90%, or 91% or 92% or 93% or 94% or 95% or 96% or 97% or 98% or 99% sequence identity to the amino acid sequence of SEQ ID NO: 3. In some embodiments, the first nanobody includes an amino acid sequence having the same identity to the amino acid sequence set forth in any one of SEQ. ID. NO: 4. In some embodiments, the first nanobody includes an amino acid sequence that has at least 80%, or 85% or 90%, or 91% or 92% or 93% or 94% or 95% or 96% or 97% or 98% or 99% sequence identity to the amino acid sequence of SEQ ID NO: 4.

In some embodiments, the second nanobody includes an amino acid sequence having the same identity to the amino acid sequence set forth in any one of SEQ. ID. NOS.: 5-7 (FIG. 11). In some embodiments, the second nanobody has at least 80%, or 85% or 90%, or 91% or 92% or 93% or 94% or 95% or 96% or 97% or 98% or 99%, or any values in between, sequence identity to the amino acid sequence set forth in any one of SEQ. ID. NOS.: 5-7. In some embodiments, the second nanobody includes an amino acid sequence having the same identity to the amino acid sequence set forth in any one of SEQ. ID. NO: 5. In some embodiments, the second nanobody includes an amino acid sequence that has at least 80%, or 85% or 90%, or 91% or 92% or 93% or 94% or 95% or 96% or 97% or 98% or 99% sequence identity to the amino acid sequence of SEQ ID NO: 5. In some embodiments, the second nanobody includes an amino acid sequence having the same identity to the amino acid sequence set forth in any one of SEQ. ID. NO: 6. In some embodiments, the second nanobody includes an amino acid sequence that has at least 80%, or 85% or 90%, or 91% or 92% or 93% or 94% or 95% or 96% or 97% or 98% or 99% sequence identity to the amino acid sequence of SEQ ID NO: 6. In some embodiments, the second nanobody includes an amino acid sequence having the same identity to the amino acid sequence set forth in any one of SEQ. ID. NO: 7. In some embodiments, the second nanobody includes an amino acid sequence that has at least 80%, or 85% or 90%, or 91% or 92% or 93% or 94% or 95% or 96% or 97% or 98% or 99% sequence identity to the amino acid sequence of SEQ ID NO: 7.

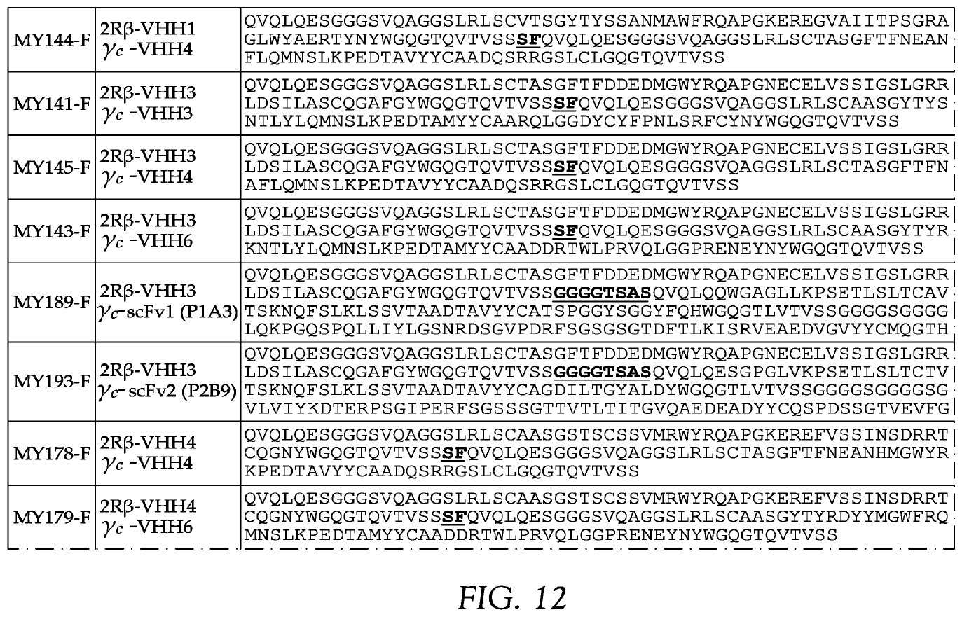

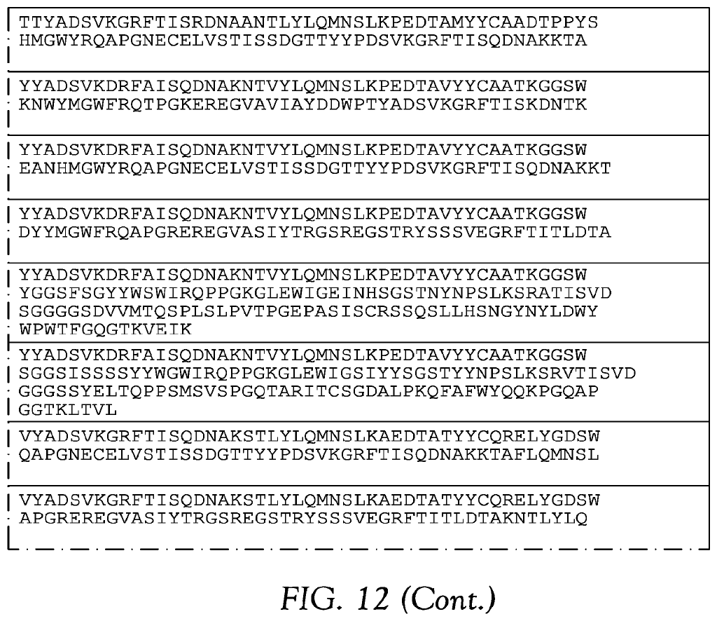

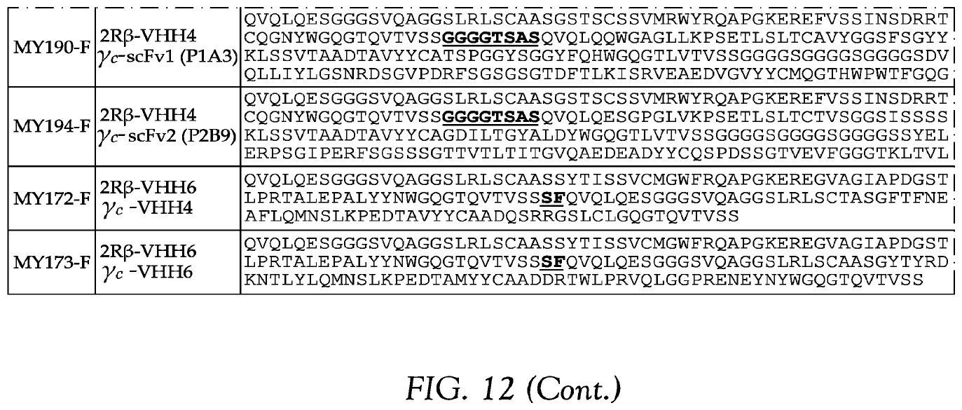

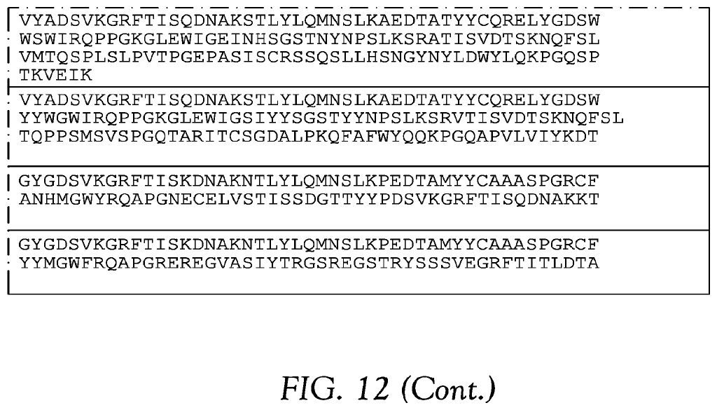

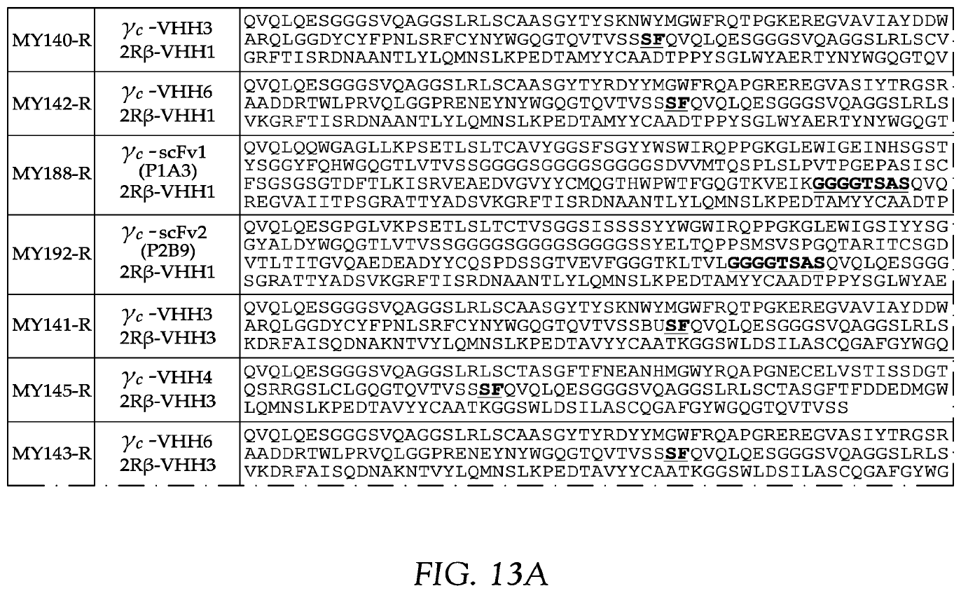

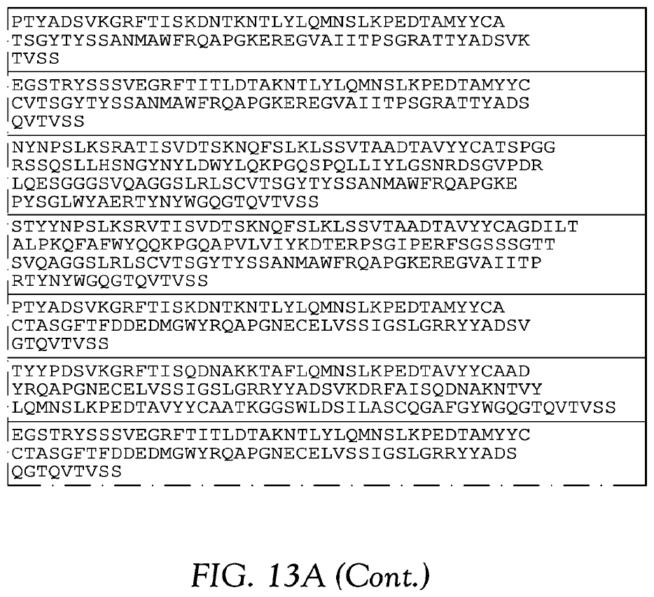

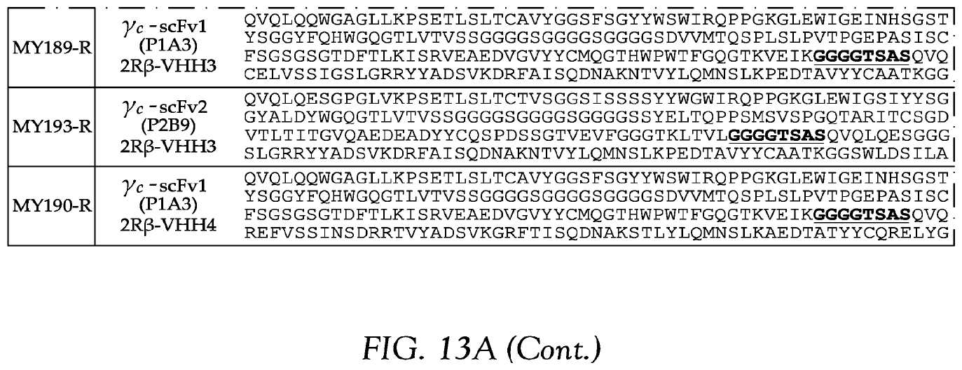

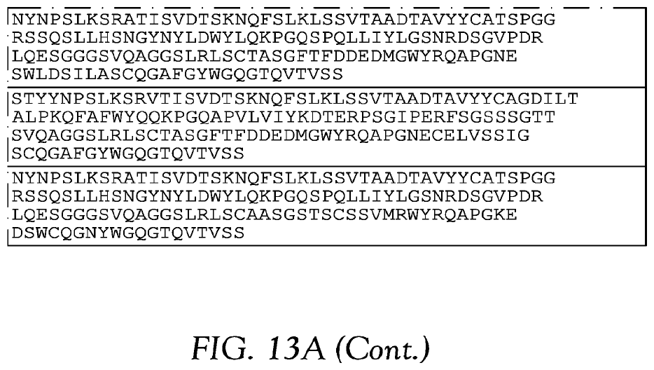

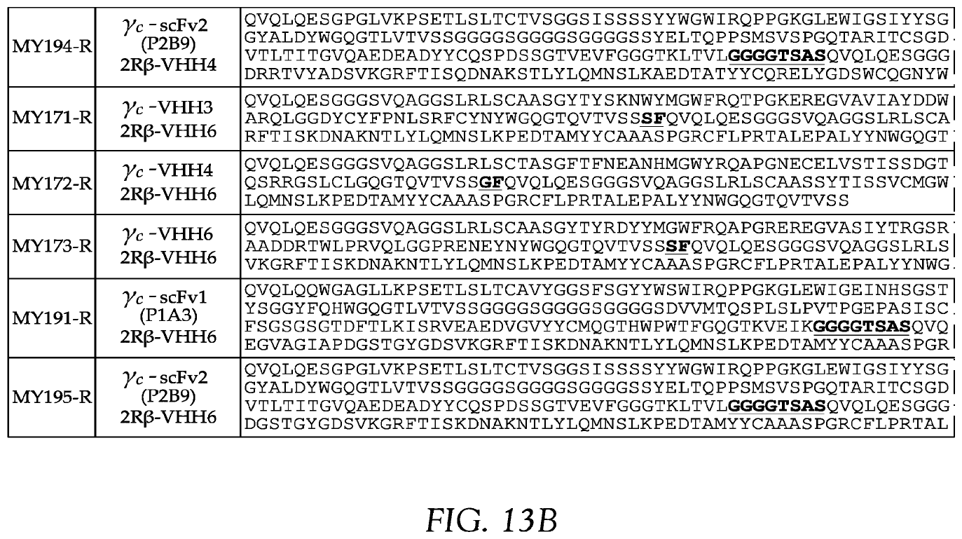

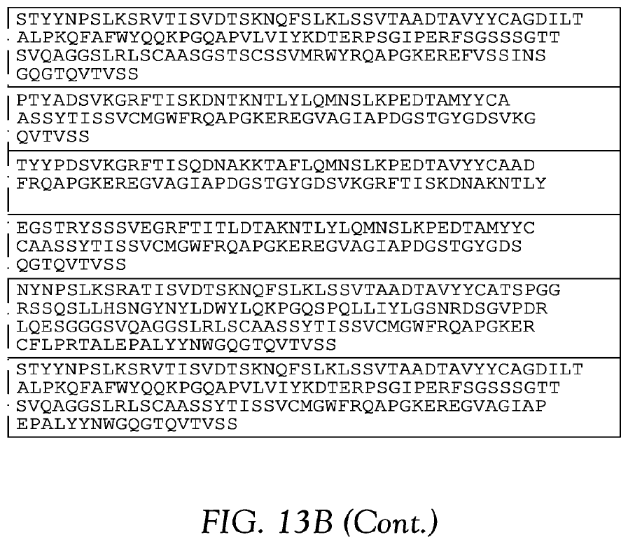

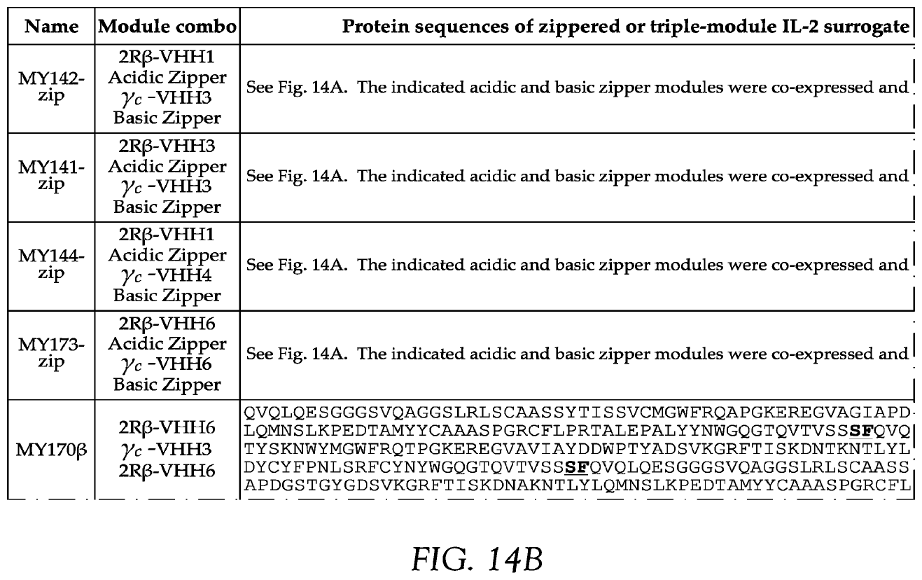

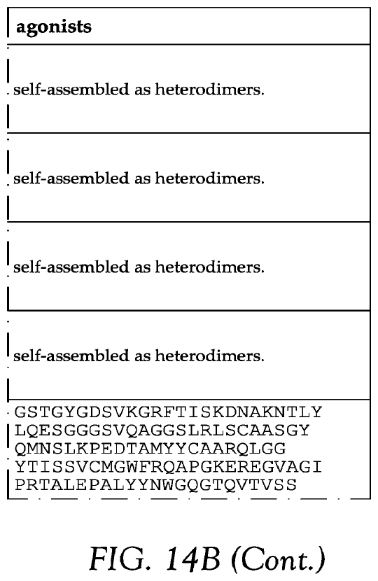

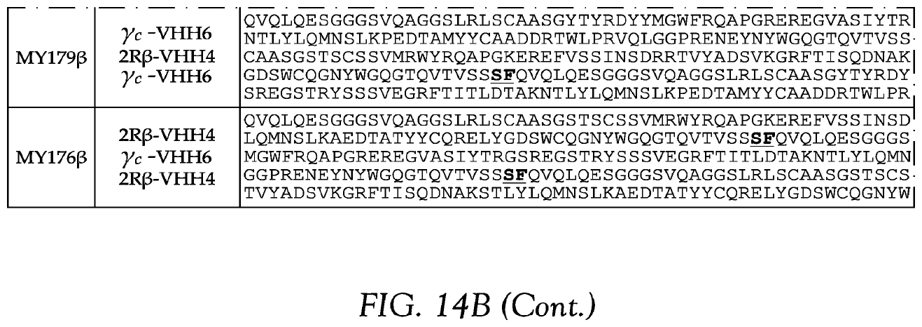

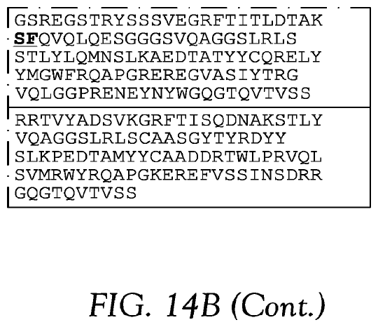

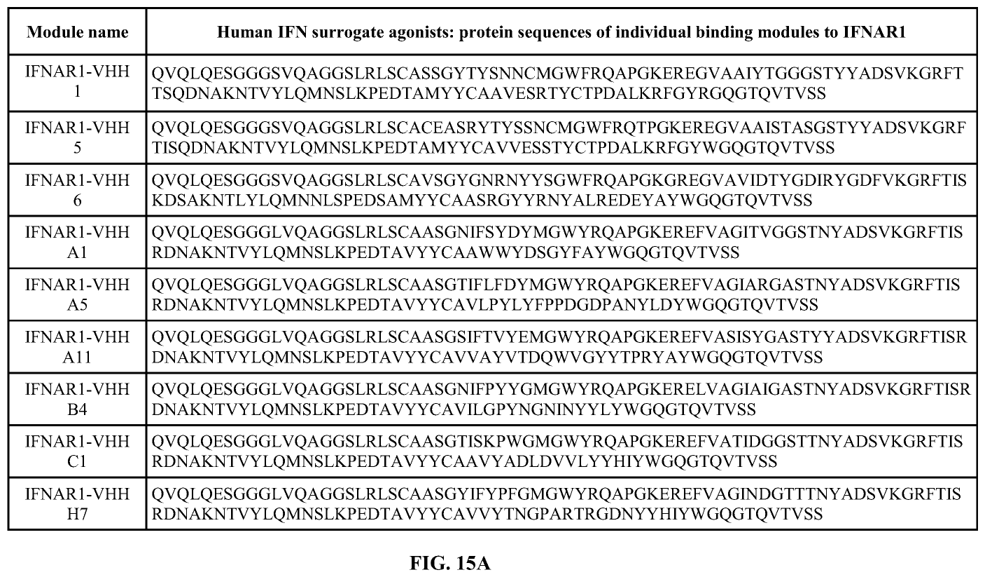

Some embodiments of the engineered polypeptide of the disclosure include a single chain bispecific ligand having an amino acid sequence identical to any of SEQ. ID. NOS.: 8-35 (FIGS. 12 and 13A-13B). In some embodiments, the single chain bispecific ligand has an amino acid sequence that has at least 80%, or 85%, or 90%, or 91%, or 92%, or 93%, or 94%, or 95%, or 96%, or 97%, or 98%, or 99%, or any values in between, sequence identity to the amino acid sequence set forth in any one of SEQ. ID. NOS.: 8-35. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 8. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 9. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 10. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 11. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 12. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 13. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 14. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 15. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 16. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 17. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 18. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 19. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 20. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 21. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 22. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 23. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 24. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 25. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 26. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 27. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 28. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 29. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 30. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 31. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 32. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 33. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 34. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 35.

In some embodiments, the single chain bispecific ligand includes one nanobody specific to IL-2Rβ and a scFv specific to γ_{c}. In some embodiments, the ligand has an amino acid sequence that is identical to any one of SEQ. ID. NOS: 22, 23, 27, 28, 29, 30, 34 or 35. In some embodiments the ligand has an amino acid sequence that has at least 80%, or 85%, or 90%, or 91%, or 92%, or 93%, or 94%, or 95%, or 96%, or 97%, or 98%, or 99% or any values in between, sequence identity to any one of SEQ. ID. NOS: 22, 23, 27, 28, 29, 30, 34 or 35. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 22. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 23. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 27. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 28. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 29. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 30. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 34. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 35.

One skilled in the art will appreciate that the complete amino acid sequence can be used to construct a back-translated gene. For example, a DNA oligomer containing a nucleotide sequence coding for a given polypeptide can be synthesized. For example, several small oligonucleotides coding for portions of the desired polypeptide can be synthesized and then ligated. The individual oligonucleotides typically contain 5′ or 3′ overhangs for complementary assembly.

In addition to generating polypeptides via expression of nucleic acid molecules that have been altered by recombinant molecular biological techniques, a subject single chain bispecific ligand in accordance with the present disclosure can be chemically synthesized. Chemically synthesized polypeptides are routinely generated by those of skill in the art.

Once assembled (by synthesis, site-directed mutagenesis or another method), the DNA sequences encoding a single chain bispecific ligand as disclosed herein will be inserted into an expression vector and operably linked to an expression control sequence appropriate for expression of the single chain bispecific ligand in the desired transformed host. Proper assembly can be confirmed by nucleotide sequencing, restriction mapping, and expression of a biologically active polypeptide in a suitable host. As is known in the art, in order to obtain high expression levels of a transfected gene in a host, the gene must be operably linked to transcriptional and translational expression control sequences that are functional in the chosen expression host.

The binding activity of the single chain bispecific ligands of the disclosure can be assayed by any suitable method known in the art. A ligand that “preferentially binds” or “specifically binds” (used interchangeably herein) to a target protein or target epitope is a term well understood in the art, and methods to determine such specific or preferential binding are also known in the art. An antibody or polypeptide is said to exhibit “specific binding” or “preferential binding” if it reacts or associates more frequently, more rapidly, with greater duration and/or with greater affinity with a particular protein or epitope than it does with alternative proteins or epitopes. A ligand “specifically binds” is “specific to” or “preferentially binds” to a target if it binds with greater affinity, avidity, more readily, and/or with greater duration than it binds to other substances. Also, a ligand “specifically binds” or “preferentially binds” to a target if it binds with greater affinity, avidity, more readily, and/or with greater duration to that target in a sample than it binds to other substances present in the sample. It is also understood by reading this definition, for example, that a ligand which specifically or preferentially binds to a first target may or may not specifically or preferentially bind to a second target. As such, “specific binding” or “preferential binding” does not necessarily require (although it can include) exclusive binding.

A variety of assay formats may be used to select a single chain bispecific ligand that specifically binds a molecule of interest. For example, solid-phase ELISA immunoassay, immunoprecipitation, Biacore™ (GE Healthcare, Piscataway, NJ), KinExA, fluorescence-activated cell sorting (FACS), Octet™ (ForteBio, Inc., Menlo Park, CA) and Western blot analysis are among many assays that may be used to identify an antibody that specifically reacts with an antigen or a receptor, or ligand binding portion thereof, that specifically binds with a cognate ligand or binding partner. Typically, a specific or selective reaction will be at least twice the background signal or noise, more typically more than 10 times background, even more typically, more than 50 times background, more typically, more than 100 times background, yet more typically, more than 500 times background, even more typically, more than 1000 times background, and even more typically, more than 10,000 times background. Also, an antibody is said to “specifically bind” an antigen when the equilibrium dissociation constant (K_{D}) is <7 nM.

The term “binding affinity” is herein used as a measure of the strength of a non-covalent interaction between two molecules, e.g., an antibody or portion thereof and an antigen. The term “binding affinity” is used to describe monovalent interactions (intrinsic activity). Binding affinity between two molecules may be quantified by determination of the dissociation constant (K_{D}). In turn, K_{D} can be determined by measurement of the kinetics of complex formation and dissociation using, e.g., the surface plasmon resonance (SPR) method (Biacore). The rate constants corresponding to the association and the dissociation of a monovalent complex are referred to as the association rate constants k_{d} (or kₒₙ) and dissociation rate constant k_{d} (or k_{off}), respectively. K_{D} is related to kₐ and k_{d} through the equation K_{D}=k_{d}/kₐ. The value of the dissociation constant can be determined directly by well-known methods, and can be computed even for complex mixtures by methods such as those set forth in Caceci et al. (1984, Byte 9:340-362). For example, the K_{D} may be established using a double-filter nitrocellulose filter binding assay such as that disclosed by Wong & Lohman (1993, Proc. Natl. Acad. Sci. USA 90:5428-5432). Other standard assays to evaluate the binding ability of antibodies or polypeptides of the present disclosure towards target antigens are known in the art, including for example, ELISAs, Western blots, RIAs, and flow cytometry analysis, and other assays exemplified elsewhere herein. The binding kinetics and binding affinity of the antibody also can be assessed by standard assays known in the art, such as Surface Plasmon Resonance (SPR), e.g. by using a Biacore™ system, or KinExA.

In some embodiments, the single chain bispecific ligand is a dimerizing ligand for an IL-2 B/γ_{c} receptor. As used herein, a “dimerizing ligand” refers to a ligand which, upon binding to its receptors, brings the receptors into appropriate signaling geometries (Harris et al., 2021; Moraga et al., 2015).

In some embodiments the single-chain bispecific ligand is capable of inducing STAT 5 phosphorylation in vitro and/or in vivo. Measuring phosphorylation of STAT5 is known to the skilled in the art and includes, for example, western blot and phospho flow cytometry. In some embodiments, assaying the phosphorylation of STAT1, or STAT2 or STAT3 is done as described in the Examples below.

In some embodiments, the single-chain bispecific ligands promote cytolytic ability against tumors in vitro and/or in vivo. In some embodiments, the cytolytic ability of the single-chain bispecfic ligands described herein can be measured using a cytolytic assay, e.g., an assay examining the ability of NK-92 cells to kill K562 or A549 leukemic cells or lung adenocarcinoma cells, respectively (Reid et al. 2002).

In some embodiments, the IL-2 B/γ_{c} receptors that are targets for the ligands/agonists of the present disclosure are mammalian receptors. In some embodiments, the receptors are human receptors.

Engineered Polypeptides and Ligands with Specificity to IL-2Rβ and IL-10R

Also provided herein are compositions having engineered polypeptides that include a single chain bispecific ligand, wherein a first specificity of the ligand is to IL-2Rβ and a second specificity of the ligand is to IL-10Rβ and wherein the engineered polypeptide is a cytokine agonist.

In some embodiments, the single-chain bispecific ligand includes one or more antibody domains (binders, modules). The term “antibody domain” or “antigen binding fragment” as used herein and throughout the present disclosure refers to an antibody fragment such as, for example, a diabody, a Fab, a Fab′, a F(ab′) 2, an Fv fragment, a disulfide stabilized Fv fragment (dsFv), a (dsFv) 2, a bispecific dsFv (dsFv-dsFv′), a disulfide stabilized diabody (ds diabody), a single-chain antibody molecule (scFv), an scFv dimer (bivalent diabody), a multispecific antibody formed from a portion of an antibody comprising one or more CDRs. The antigen-binding moiety can include naturally-occurring polypeptides or can be engineered, designed, or modified so as to provide desired and/or improved properties. As used herein, the term “VHH” or “nanobody” are used herein interchangeably to refer to variable domain of a heavy-chain antibody. A nanobody is the smallest antigen binding fragment or single variable domain derived from naturally occurring heavy chain antibody and is known to the person skilled in the art. They are derived from heavy chain only antibodies, seen in camelids (Hamers-Casterman el al. 1993; Desmyter el al. 1996). In the family of “camelids,” immunoglobulins devoid of light polypeptide chains are found. “Camelids” comprise old world camelids (Camelus bactrianus and Camelus dromedarius) and new world camelids (for example, Lama paccos, Lama glama, Lama guanicoe, and Lama vicugna). The single variable domain heavy chain antibody is herein designated as a nanobody or a VHH antibody. Nanobodies can also be derived from sharks.

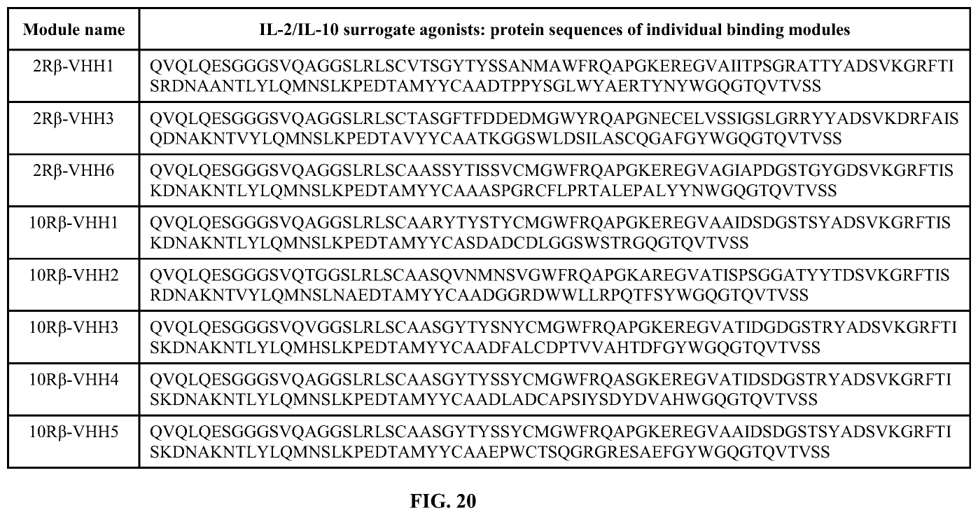

In some embodiments, the single chain bispecific ligand comprises a first nanobody specific to IL-2Rβ and a second nanobody specific to IL-10Rβ. In some embodiments, the first nanobody includes an amino acid sequence set forth in any one of SEQ. ID. NOS.: 70-72 (FIG. 20). In some embodiments, the first nanobody comprises an amino acid sequence at least 80%, 85%, 87%, 90%, 91%, 92%, 93%, 94%, 95%, 96%, 97%, 98%, or 99% identical to the amino acid sequence set forth in any one of SEQ. ID. NOS.: 70-72. In some embodiments, the first nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 70. In some embodiments, the first nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 71. In some embodiments, the first nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 72.

In some embodiments, the second nanobody includes an amino acid sequence set forth in any one of SEQ. ID. NOS: 73-77 (FIG. 20). In some embodiments, the second nanobody comprises an amino acid sequence that has at least 80%, 85%, 87%, 90%, 91%, 92%, 93%, 94%, 95%, 96%, 97%, 98%, or 99% identity to the amino acid sequence set forth in any one of SEQ. ID. NOS.: 73-77. In some embodiments, the second nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 73. In some embodiments, the second nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 74. In some embodiments, the second nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 75. In some embodiments, the second nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 76. In some embodiments, the second nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 77.

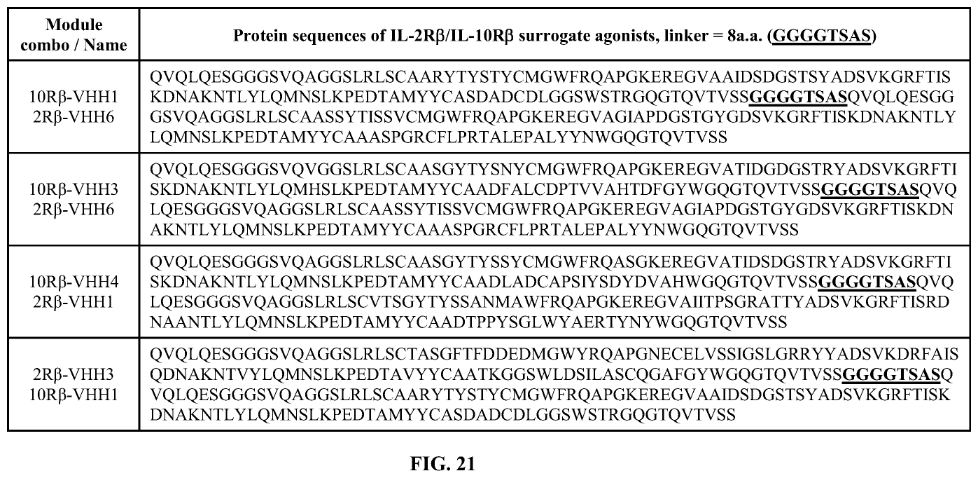

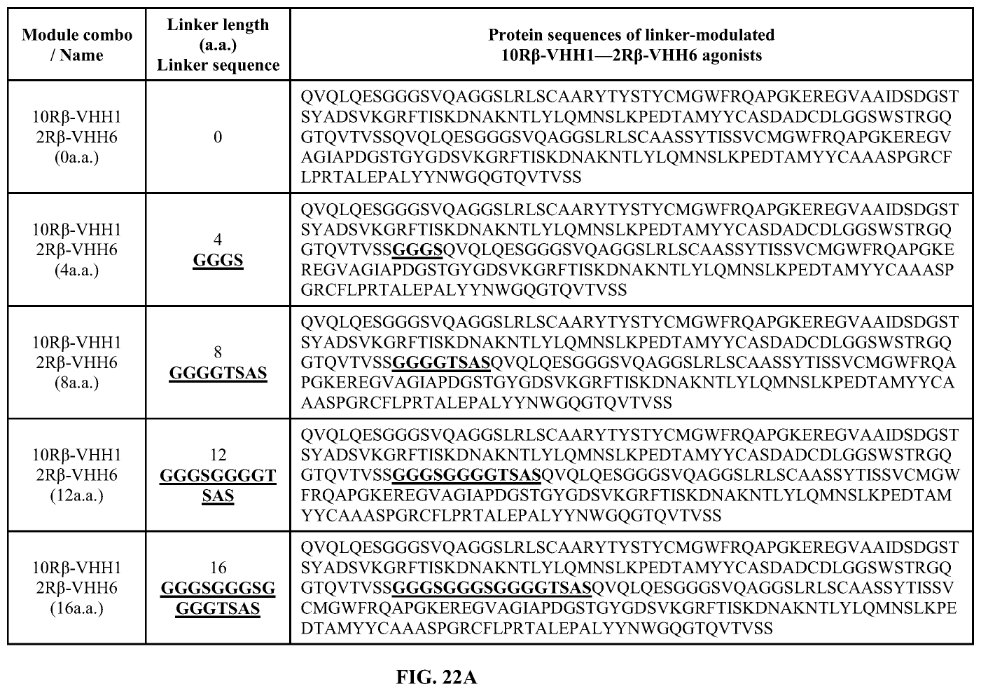

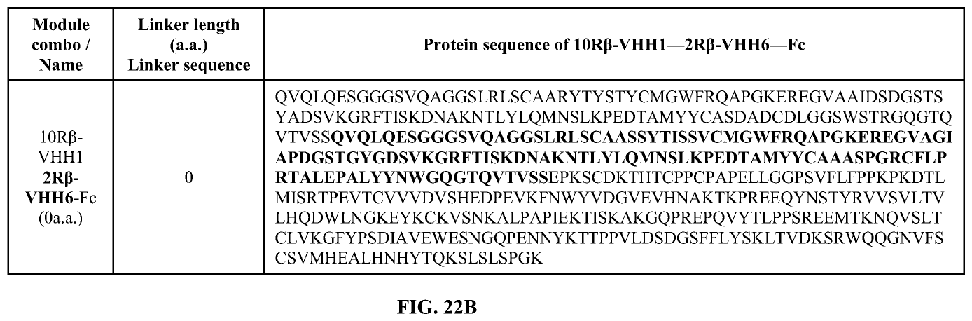

In some embodiments, the engineered polypeptides of the disclosure include a single chain bispecific ligand having an amino acid sequence identical to any of SEQ. ID. NOS.: 78-87 (FIG. 21, FIGS. 22A-22B). In some embodiments, the single chain bispecific ligand has an amino acid that has at least 80%, or 85%, or 90%, or 91%, or 92%, or 93%, or 94%, or 95%, or 96%, or 97%, or 98%, or 99%, identity to the amino acid sequence set forth in any one of SEQ. ID. NOS.: 78-87. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 78. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 79. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 80. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 81. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 82. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 83. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 84. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 85. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 86. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 87.

In some embodiments, the single chain bispecific ligands of the disclosure can be heterodimers. In other embodiments, the ligands can be homodimers.

In some embodiments, the single chain bispecific ligands are Fc fusions. single chain bispecific ligands can be fused to the Fc domain of IgG to extend its half-life, e.g. by pegylation, glycosylation, and the like as known in the art. Fc-fusion can also endow alternative Fc receptor mediated properties in vivo. The “Fc region” can be a naturally occurring or synthetic polypeptide that is homologous to an IgG C-terminal domain produced by digestion of IgG with papain. IgG Fc has a molecular weight of approximately 50 kDa. The single chain bispecific ligands can include the entire Fc region, or a smaller portion that retains the ability to extend the circulating half-life of a chimeric polypeptide of which it is a part. In addition, full-length or fragmented Fc regions can be variants of the wild-type molecule. That is, they can contain mutations that may or may not affect the function of the polypeptides. In some embodiments, the single chain bispecific ligand can have the amino acid sequence set forth in SEQ. ID. NO.: 87. In some embodiments, the ligand includes an amino acid sequence that has at least 80%, or 85%, or 90%, or 91%, or 92%, or 93%, or 94%, or 95%, or 96%, or 97%, or 98%, or 99%, identity to the amino acid sequence set forth in SEQ. ID. NO.: 87.

In some embodiments of the engineered polypeptides of the present disclosure, the single chain bispecific ligand is a dimerizing-ligand for an IL-2Rβ/IL-10Rβ receptor heterodimer. In some embodiments, the engineered polypeptide is capable of inducing phosphorylation of STAT5, or STAT3 or a combination thereof in vivo or in vitro. Methods for measuring phosphorylation of STAT5 or STAT3 is known to the skilled in the art and are described above. In some embodiments, assaying the phosphorylation of STAT5 or STAT3 is done as described in the Examples below.

In some embodiments, the IL-2Rβ/IL-10Rβ receptors that are targets for the ligands/agonists of the present disclosure are mammalian receptors. In some embodiments, the receptors are human receptors.

### Engineered Polypeptides and Ligands Specific to Type I IFN

Provided herein are compositions including an engineered polypeptide with a single chain bispecific ligand wherein a first specificity of the ligand is to IFNAR1 and a second specificity of the ligand is to IFNAR2 and wherein the engineered polypeptide is a cytokine agonist.

In some embodiments, the single-chain bispecific ligand includes one or more antibody domains (binders, modules). The term “antibody domain” or “antigen binding fragment” as used herein and throughout the present disclosure refers to an antibody fragment such as, for example, a diabody, a Fab, a Fab′, a F(ab′) 2, an Fv fragment, a disulfide stabilized Fv fragment (dsFv), a (dsFv) 2, a bispecific dsFv (dsFv-dsFv′), a disulfide stabilized diabody (ds diabody), a single-chain antibody molecule (scFv), an scFv dimer (bivalent diabody), a multispecific antibody formed from a portion of an antibody comprising one or more CDRs. The antigen-binding moiety can include naturally-occurring polypeptides or can be engineered, designed, or modified so as to provide desired and/or improved properties. As used herein, the term “VHH” or “nanobody” are used herein interchangeably to refer to variable domain of a heavy-chain antibody. A nanobody is the smallest antigen binding fragment or single variable domain derived from naturally occurring heavy chain antibody and is known to the person skilled in the art. They are derived from heavy chain only antibodies, seen in camelids (Hamers-Casterman el al. 1993; Desmyter el al. 1996). In the family of “camelids,” immunoglobulins devoid of light polypeptide chains are found. “Camelids” comprise old world camelids (Camelus bactrianus and Camelus dromedarius) and new world camelids (for example, Lama paccos, Lama glama, Lama guanicoe, and Lama vicugna). The single variable domain heavy chain antibody is herein designated as a nanobody or a VHH antibody. Nanobodies can also be derived from sharks.

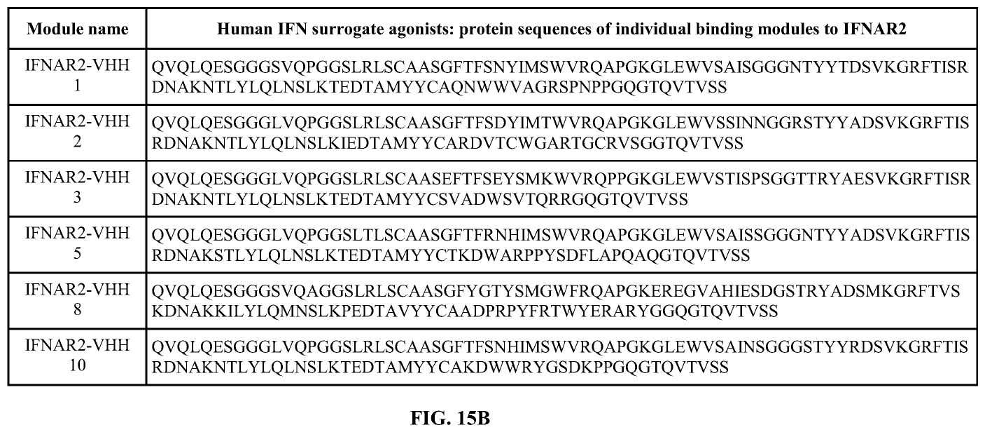

In some embodiments, the single chain bispecific ligand comprises a first nanobody specific to IFNAR1 and a second nanobody specific to IFNAR2. In some embodiments, the first nanobody comprises an amino acid sequence that is the amino acid sequence set forth in any one of SEQ. ID. NOS.: 36-44 (FIGS. 15A-15B). In some embodiments, the first nanobody comprises an amino acid sequence that has at least 80%, 85%, 87%, 89%, 90%, 91%, 92%, 93%, 94%, 95%, 96%, 97%, 98%, 99%, or any values in between, sequence identity to the amino acid sequence set forth in any one of SEQ. ID. NOS.: 36-44. In some embodiments, the first nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 36. In some embodiments, the first nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 37. In some embodiments, the first nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 38. In some embodiments, the first nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 39. In some embodiments, the first nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 40. In some embodiments, the first nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 41. In some embodiments, the first nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 42. In some embodiments, the first nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 43. In some embodiments, the first nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 44.

In some embodiments, the second the second nanobody includes an amino acid sequence set forth in any one of SEQ. ID. NOS.: 45-50. In some embodiments, the second nanobody comprises an amino acid sequence that has at least 80%, 85%, 87%, 89%, 90%, 91%, 92%, 93%, 94%, 95%, 96%, 97%, 98%, 99%, or any values in between, sequence identity to the amino acid sequence set forth in any one of SEQ. ID. NOS.: 45-50. In some embodiments, the second nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 45. In some embodiments, the second nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 46. In some embodiments, the second nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 47. In some embodiments, the second nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 48. In some embodiments, the second nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 49. In some embodiments, the second nanobody includes an amino acid sequence that has at least 80%, at least 85%, at least 90%, at least 95%, at least 96%, at least 97%, at least 98%, at least 99%, or 100% sequence identity to the amino acid sequence of SEQ ID NO: 50.

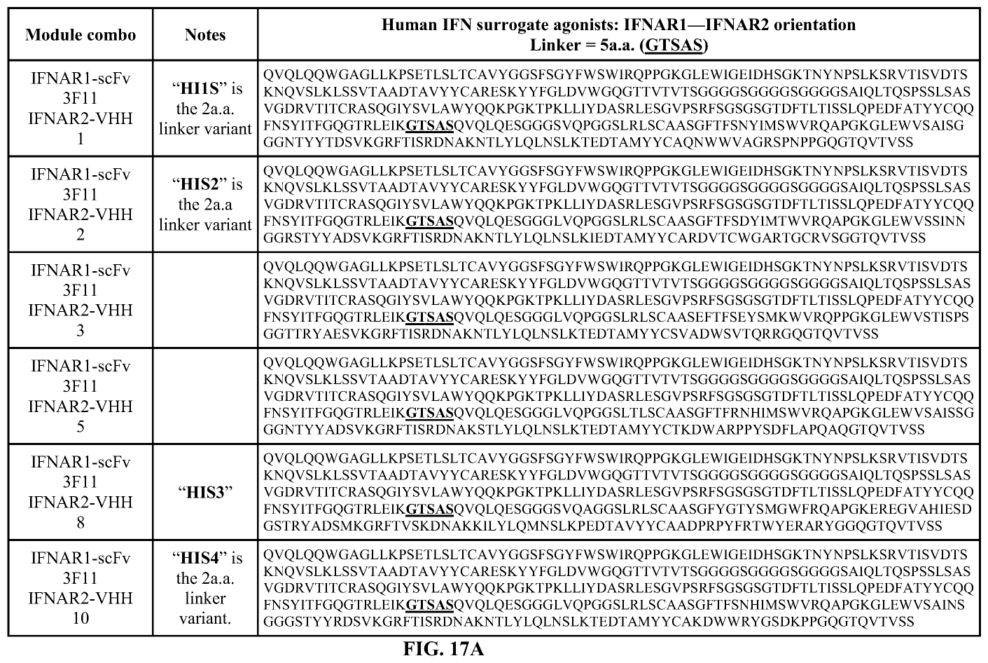

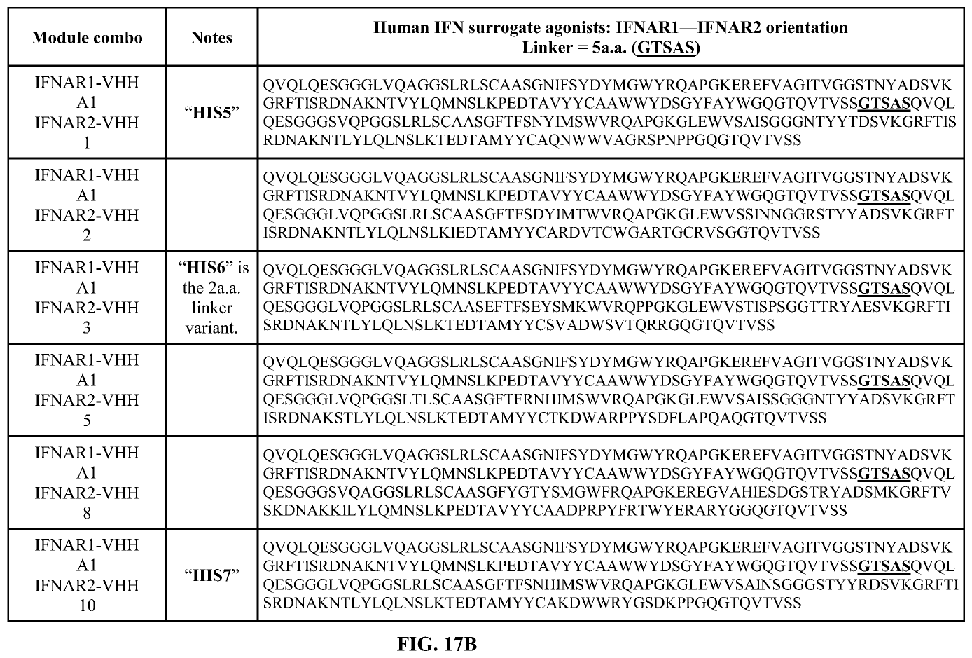

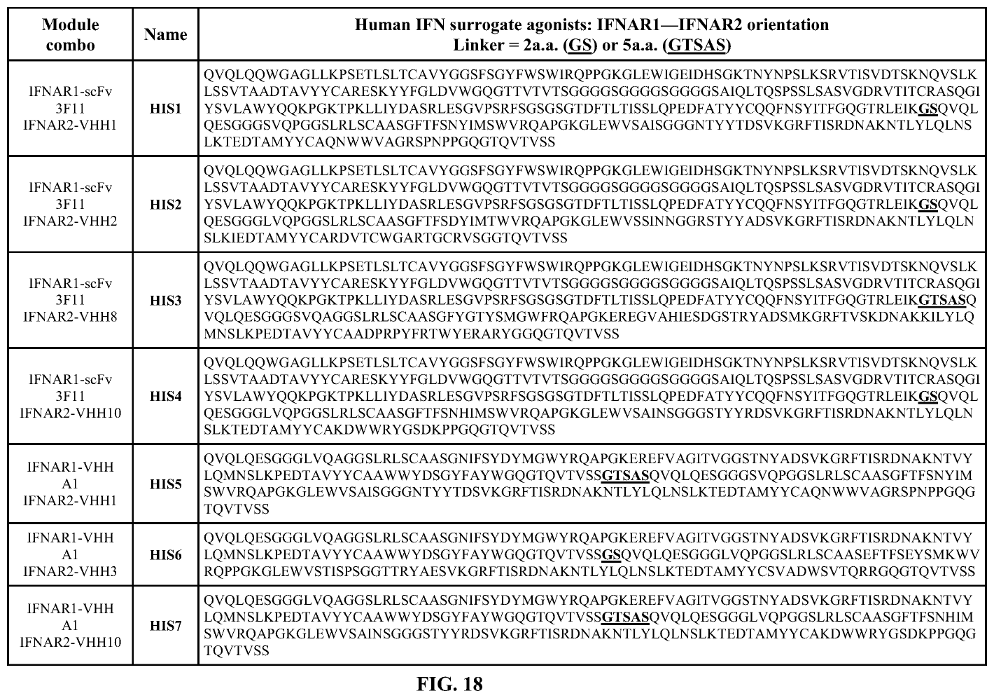

In some embodiments, the single chain bispecific ligand comprises the amino acid sequence set forth in any one of SEQ. ID. NOS.: 51-69 (FIGS. 17A-17B, FIG. 18). In some embodiments, the single chain bispecific ligand comprises an amino acid sequence that has at least 80%, or 85%, or 87%, or 90%, or 91%, or 92%, or 93%, or 94%, or 95%, or 96%, or 97%, or 98%, or 99%, or any values in between, sequence identity to the amino acid sequence set forth in any one of SEQ. ID. NOS.: 51-69. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 51. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 52. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 53. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 54. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 55. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 56. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 57. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 58. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 59. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 60. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 61. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 62. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 63. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 64. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 65. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 66. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 67. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 68. In some embodiments, the single chain bispecific ligand includes an amino acid sequence that has at least 80%, at least 85%, at least 90%, at least 95%, at least 96%, at least 97%, at least 98%, at least 99%, or 100% sequence identity to the amino acid sequence of SEQ ID NO: 69.

In some embodiments, the single chain bispecific ligand is a dimerizing-ligand for an IFNAR1/IFNAR2 receptor heterodimer. In some embodiments, the engineered polypeptide with a single chain bispecific ligand having a first specificity to IFNAR1 and a second specificity to IFNAR2 is capable of inducing phosphorylation of STAT1, or STAT2 or STAT3 or a combination thereof in vitro and/or in vivo. Methods of measuring phosphorylation of STAT1, or STAT2 or STAT3 is known to the skilled in the art and are described above. In some embodiments, assaying the phosphorylation of STAT1, or STAT2 or STAT3 is done as described in the Examples below.

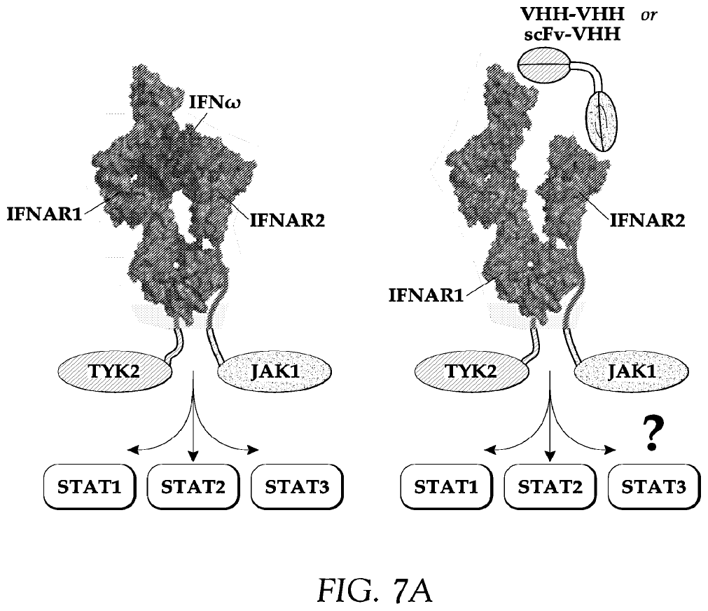

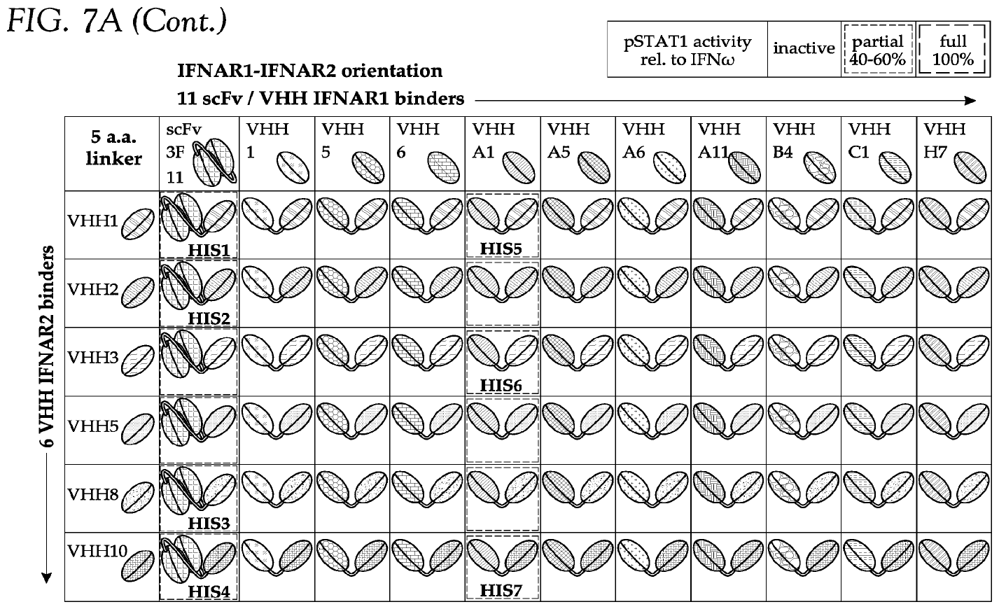

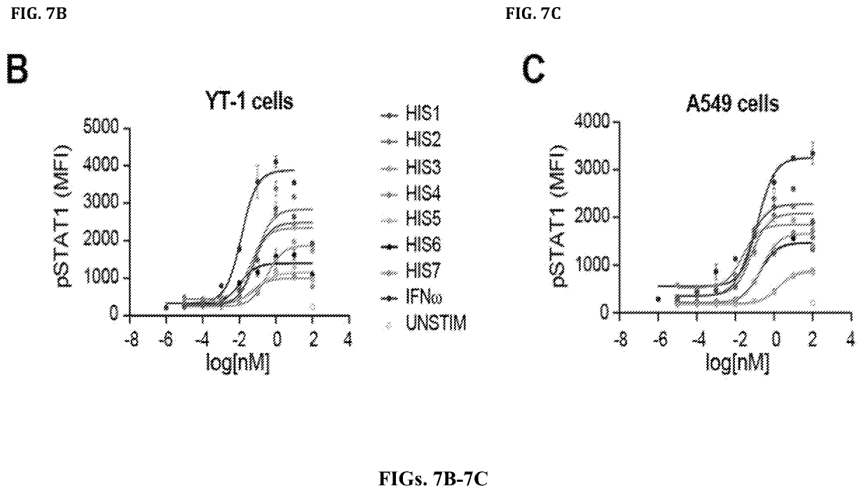

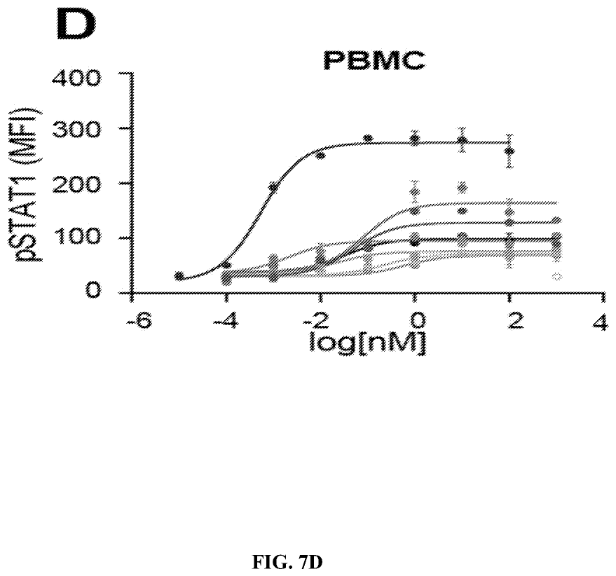

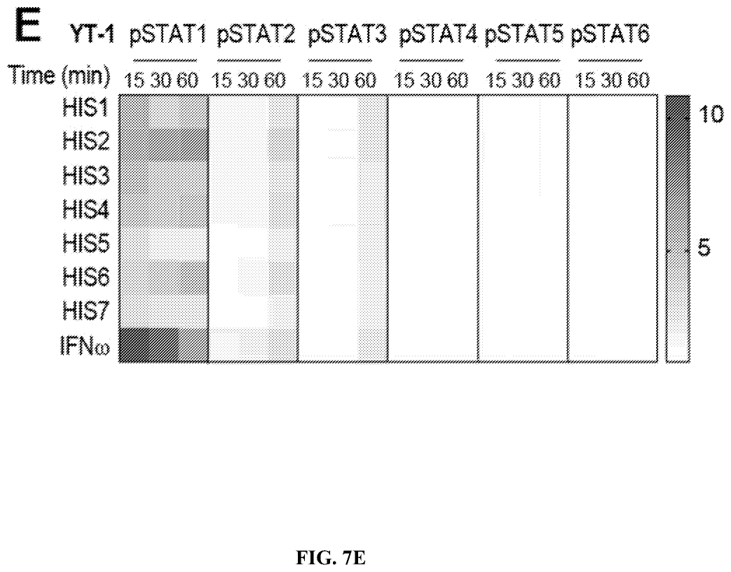

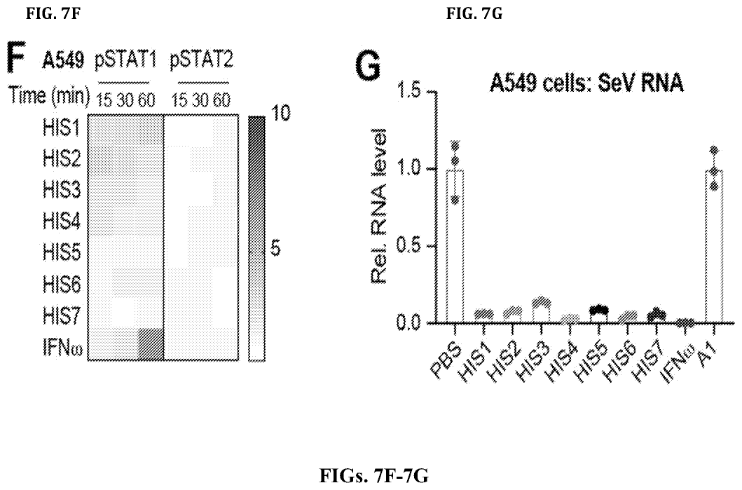

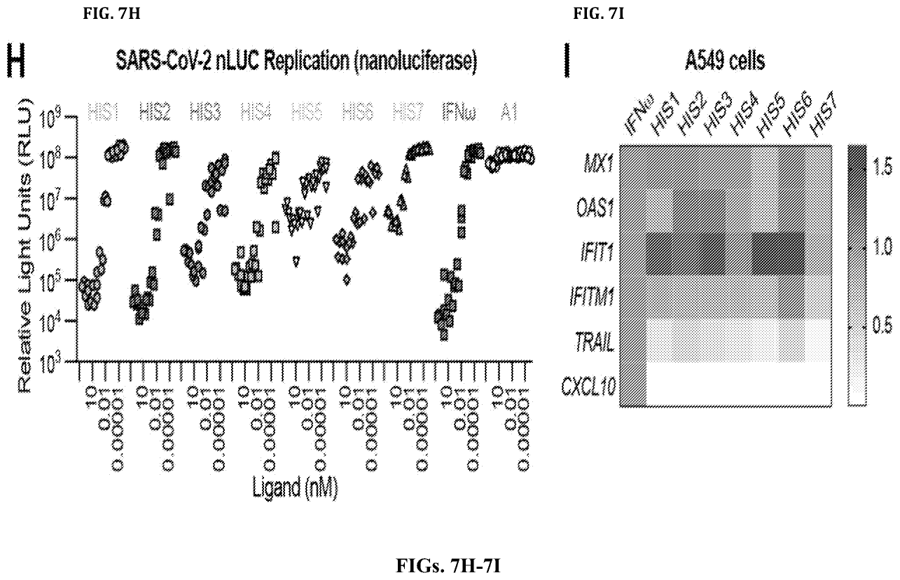

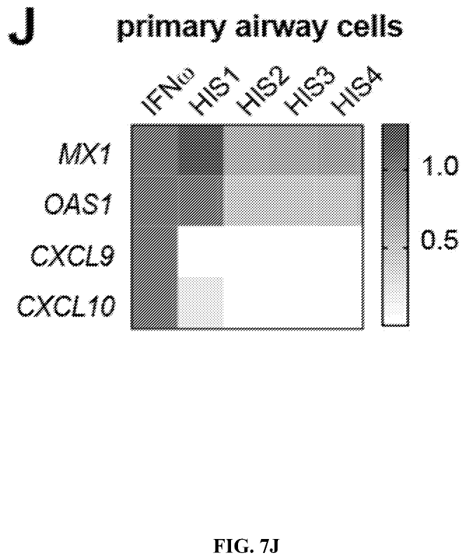

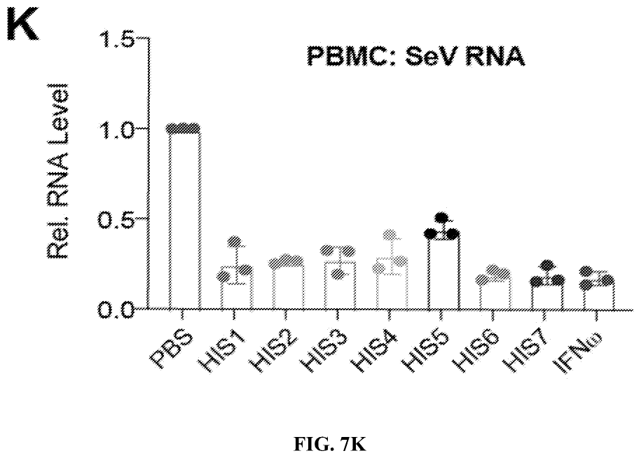

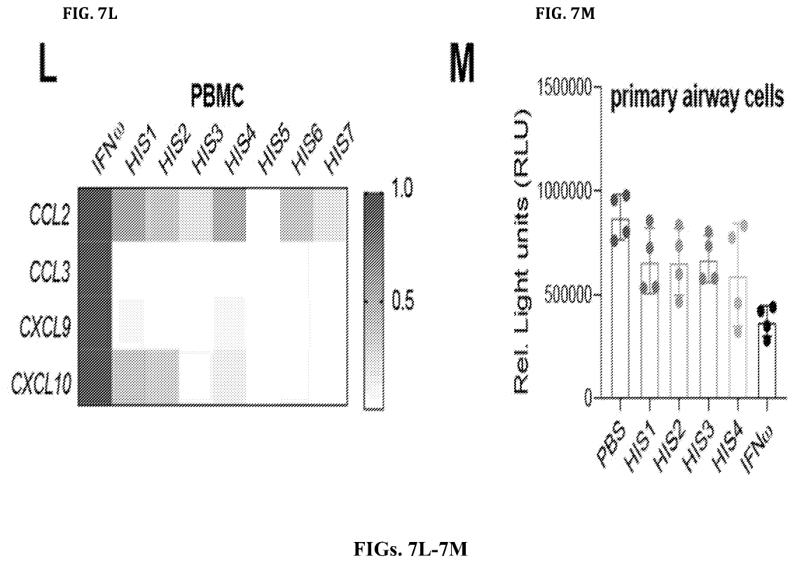

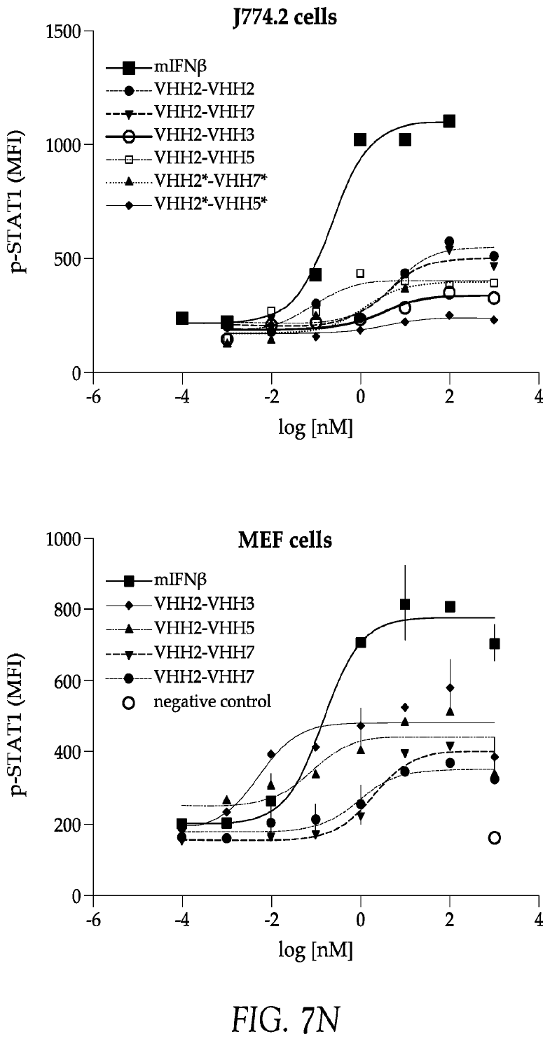

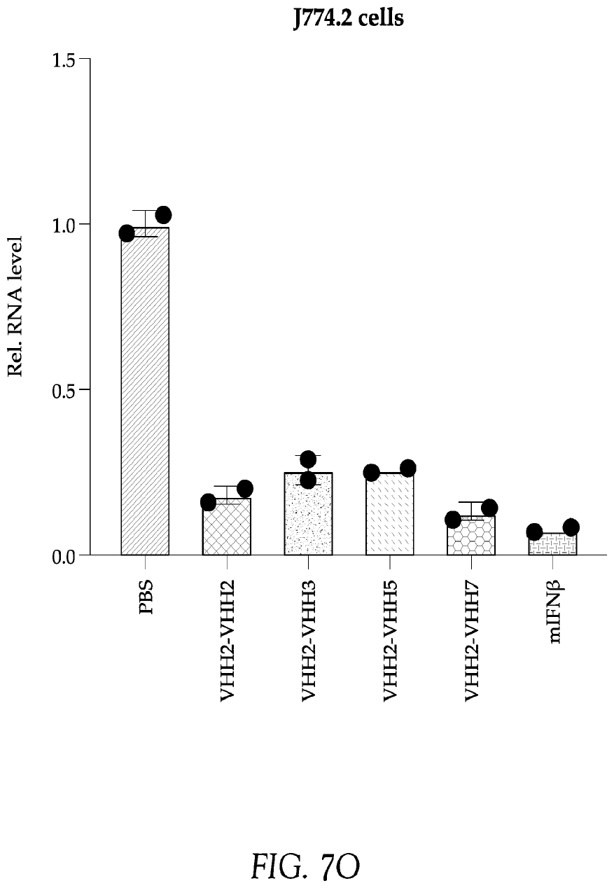

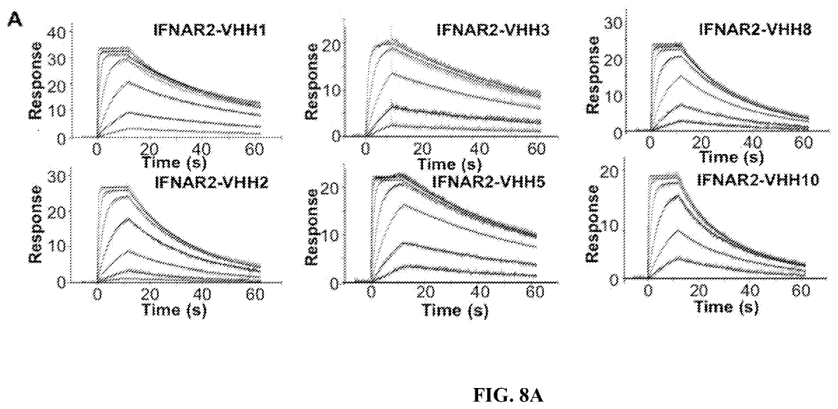

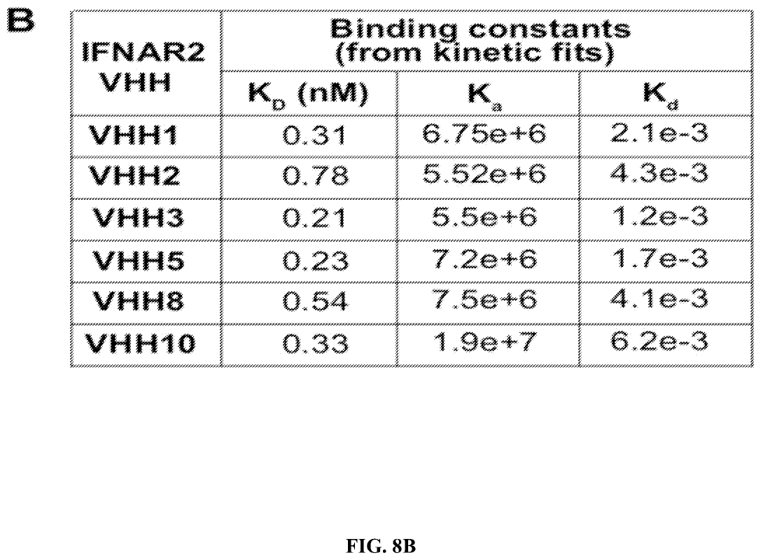

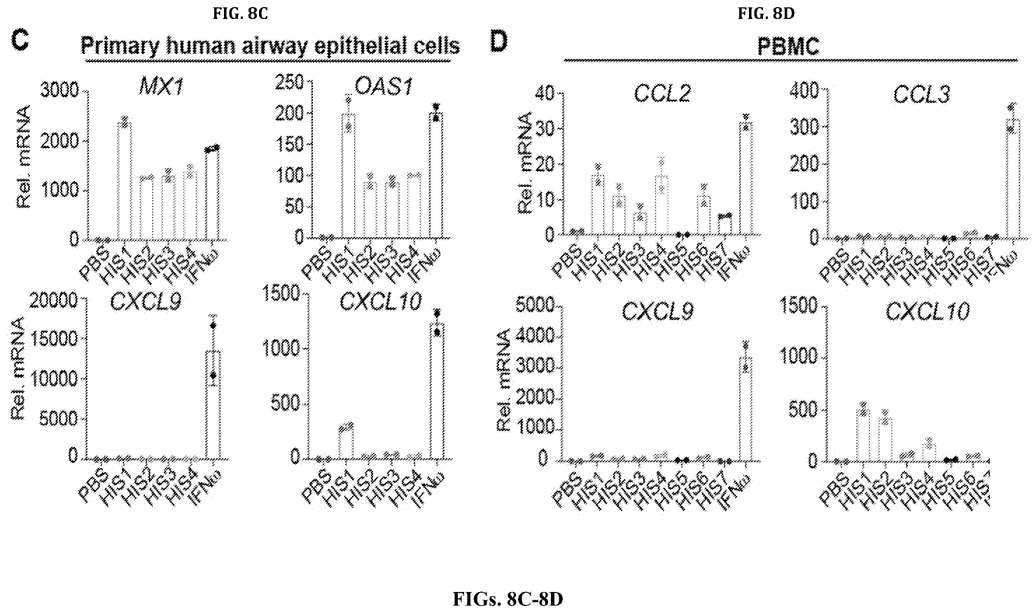

The surrogate IFNs of the disclosure exhibit anti-viral activity. Type I IFNs, for example, can exhibit antiviral ability by inducing interferon stimulated genes (ISGs). As described in the Examples, surrogate IFN ligands of the disclosure showed biased induction of ISGs (as compared with IFNω; FIGS. 7I and 8D.). In human primary airway epithelial cells, “Human Interferon Surrogates” (HIS) ligands induced high levels of the antiviral genes MX1 and OASI with minimal induction of pro-inflammatory genes CXCL9 and CXCL10 (FIG. 7J). Moreover, HIS agonists effectively inhibited SeV replication in PBMCs while barely inducing pro-inflammatory cytokine expression (FIGS. 7K-L). In addition to anti-viral activity against SeV, anti-viral activity against other viruses is also contemplated. Examples of viruses against which the surrogate IFNS of the present disclosure can exhibit anti-viral activity include, but are not limited to, Hepatitis B virus (HBV), Hepatitis C virus (HCV), Varicella-zoster virus (can cause chickenpox and shingles, VZV), Herpes Simplex Virus (can cause herpes and encephalitis, HSV), Dengue Virus (can cause dengue fever, DENV), Vesicular Stomatitis Virus (VSV), Influenza A virus (IAV), HIV-1, Human Cytomegalovirus (HCMV), Ebola Virus Disease (EVD), and Human Papilloma Virus (HPV).

The surrogate IFNs of the disclosure can be components of pharmaceutical compositions. Pharmaceutical compositions can be anti-viral compositions that can be used, for example in, but not limited to, the treatment of viral infections. Any viral infection, such as infection with e.g. retrovirus, lentivirus, hepadna virus, herpes viruses, pox viruses, human papilloma viruses, etc., is within the scope of the disclosure including hepatits B and hepatitis C infections (Li et. al, 2018.)

In some embodiments, the engineered polypeptide with a single chain bispecific ligand having a first specificity to IFNAR1 and a second specificity to IFNAR2 is capable of inhibiting SARS-COV-2 replication in vitro or in vivo. Assaying the inhibition of SARS-COV-2 replication is known to the skilled in the art (for example, Hou et al. 2020). In some embodiments, assaying the inhibition of SARS-COV-2 replication is done as follows: plates are seeded with A549-hACE2 cells. A549 is a human lung epithelial cell line stably expressing the SARS-COV-2 receptor, hACE2, to facilitate efficient infection for antiviral assays (Hou et al., 2020). Cells are infected with recombinant SARS-COV-2 engineered to express nanoluciferase at a multiplicity of infection of 0.25. After incubation, input virus is removed, cells are washed and infection medium is added. After 48 hr. of infection, levels of virus replication can be measured by Promega NanoGlo assay measured on a Promega GloMax Luminometer. Similarly treated uninfected sister plates can be generated in order to gauge potential cytotoxicity by Promega CellTiter Glo assay read on a Promega GloMax Luminometer. In some embodiments, assaying the inhibition of SARS-COV-2 replication is done as described in Example 8 below.

In some embodiments, antiviral ability can be assayed by observing the induction of expression of interferon stimulated genes (ISGs). Such genes include, but are not limited to, for example, MX1, OASI, IFIT1, IFITM1, TRAIL, CXCL10, ISG15, CH25CH, cGAS, BST2, and NCOA7. In some embodiments, the differential ISG induction can be used as a metric for screening surrogate IFNs for activity. In some embodiments, the analysis for differential ISG induction involves comparing the levels of specific ISGs and determining whether certain functional categories are preferentially reduced or weakened by surrogate ligands compared to endogenous interferons.

In some embodiments, the engineered polypeptides with a single chain bispecific ligand having a first specificity to IFNAR1 and a second specificity to IFNAR2 inhibit SARS-CoV-2 replication in a cell without inducing pro-inflammatory cytokine expression. Techniques for measuring the expression of pro-inflammatory cytokines are known to the skilled in the art. In some embodiments, pro-inflammatory cytokines include CCL2, CCL3, CXCL9, CXCL10 and others. In some embodiments, mRNA expression of pro-inflammatory cytokines is assayed. In some embodiments, polypeptide levels of pro-inflammatory cytokines are assayed as known by the skilled in the art (Metzemaekers 2018).

In some embodiments, inhibiting SARS-COV-2 replication in a cell occurs without inducing anti-proliferative cytokine expression, including BAX, BAK1, FAS, and others.

In some embodiments, inhibiting SARS-COV-2 replication in a cell occurs without inducing pro-apoptotic gene expression, such as TRAIL, ISG12, TNFSF10, and IFIT2.

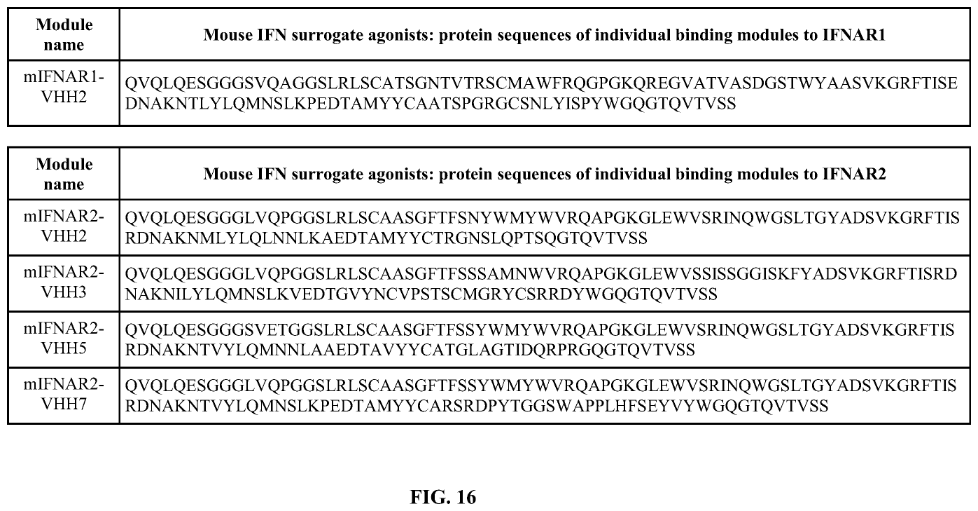

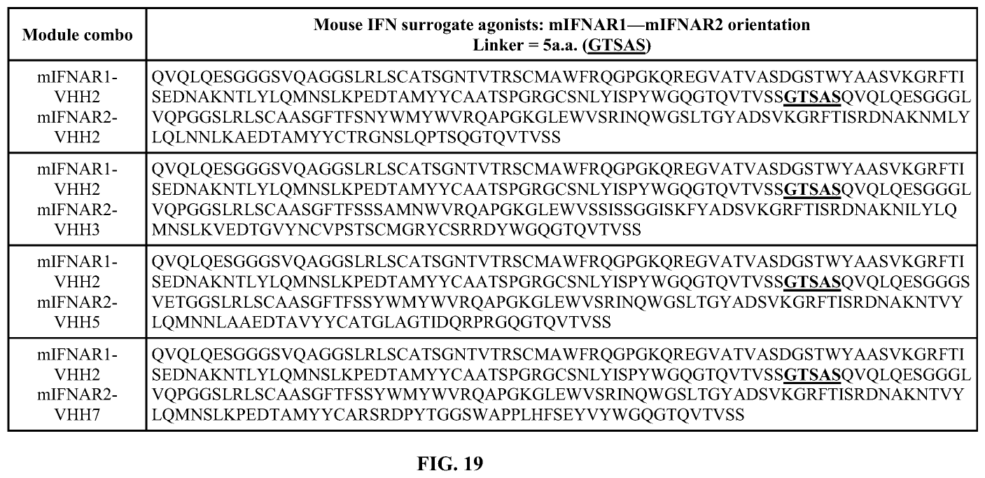

In some embodiments, the IL-2Rβ/IL-10Rβ receptors that are targets for the ligands/agonists of the present disclosure are mammalian receptors. In some embodiments, the receptors are human receptors. In some embodiments, the IFNAR1 and IFNAR2 receptors that are targets for ligands/agonists of the present disclosure are mammalian receptors. In some embodiments, the receptors are human receptors. In some embodiments, the receptors are mouse receptors. Exemplary human and mouse sequences of IFN surrogate agonists are shown in FIG. 16 and FIG. 19. Activity of mouse IFN surrogate agonists is shown in FIGS. 7N-7O.

### Linkers

In some embodiments, the antibody domains (binders) of the engineered polypeptides of the disclosure are operably joined to one another by an intervening linker. There is no particular limitation on the linkers that can be used in the chimeric polypeptides described herein. In some embodiments, the linker is a synthetic compound linker such as, for example, a chemical cross-linking agent. Non-limiting examples of suitable cross-linking agents that are available on the market include N-hydroxysuccinimide (NHS), disuccinimidylsuberate (DSS), bis(sulfosuccinimidyl) suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis [2-(succinimidooxycarbonyloxy)ethyl]sulfone (BSOCOES), and bis [2-(sulfosuccinimidooxycarbonyloxy)ethyl]sulfone (sulfo-BSOCOES).

In some embodiments, the linker is a peptide linker. In some embodiments, the linker is 1, 2, 3, 4, 5, 6, 7, 8, 9, 10, 11, 12, 13, 14, 15, 16, 17, 18, 19 or 20 amino acids in length. In some embodiments, the linker peptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker peptide sequence includes about 1 to 10, about 5 to 15, about 10 to 20, about 15 to 25, about 20 to 40, about 30 to 50, about 40 to 60, about 50 to 70 amino acid residues. In some embodiments, the linker peptide sequence includes about 40 to 70, about 50 to 80, about 60 to 80, about 70 to 90, or about 80 to 100 amino acid residues. In some embodiments, the linker peptide sequence includes about 1 to 10, about 5 to 15, about 10 to 20, about 15 to 25 amino acid residues.

In some embodiments, the bispecific ligand molecules of the present disclosure are generated by fusing IL-2Rβ and γ_{C} binders through 8 amino acids for VHH-scFv fusions and 2 amino acids for VHH-VHH fusions. In some embodiments, VHH and scFv binders to human IFNAR1 and IFNAR2, are fused via 2 amino acids or 5 amino acids linkers (FIG. 7A). A person of skill in the art readily appreciates testing different linker lengths to modulate activity. For example, FIG. 9C shows how 10Rβ1-2Rβ6 agonists with varying linker length between 0-16 amino acids were tested for pSTAT5 signaling in YT-1 cells.

In some embodiments, the linkers are flexible Gly-Ser linkers. Examples of such polypeptide linkers include but are not limited to: GS, GGS, GGGS (SEQ ID NO: 106). In some embodiments, the linker can include other amino acids such as A or T. Examples include but are not limited to GTSAS (SEQ ID NO: 107), GGGGTSAS (SEQ ID NO: 108), GGGSGGGGTSAS (SEQ ID NO: 109), GGGSGGGSGGGGTSAS (SEQ ID NO: 110).

The antibody domains or binders of the disclosure can be linked in a Forward or Reverse orientation (as shown, for example in FIG. 1B).

In some embodiments, the antibody domains are not joined by a linker. An example is the 10Rβ1-2Rβ6-Fc shown in FIG. 22.

### Pharmaceutical Compositions

The present disclosure, also provides pharmaceutical compositions including the engineered polypeptides disclosed herein. In some embodiments, the pharmaceutical compositions include, in addition to the engineered polypeptide, a pharmaceutically acceptable excipient or carrier.

The term “pharmaceutically acceptable excipient” as used herein refers to any suitable substance that provides a pharmaceutically acceptable carrier, additive or diluent for administration of a compound(s) of interest to a subject. As such, “pharmaceutically acceptable excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics) can also be incorporated into the compositions.

In some embodiments, the engineered polypeptides of the disclosure are prepared with carriers that will protect the engineered polypeptides against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acID. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811. As described in greater detail below, the recombinant polypeptides of the present disclosure may also be modified to achieve extended duration of action such as by PEGylation, acylation, Fc fusions, linkage to molecules such as albumin, etc. In some embodiments, the recombinant polypeptides can be further modified to prolong their half-life in vivo and/or ex vivo. Non-limiting examples of known strategies and methodologies suitable for modifying the recombinant polypeptides of the disclosure include (1) chemical modification of a recombinant polypeptide described herein with highly soluble macromolecules such as polyethylene glycol (“PEG”) which prevents the recombinant polypeptides from contacting with proteases; and (2) covalently linking or conjugating a recombinant polypeptide described herein with a stable protein such as, for example, albumin. Accordingly, in some embodiments, the recombinant polypeptides of the disclosure can be fused to a stable protein, such as, albumin. For example, human albumin is known as one of the most effective proteins for enhancing the stability of polypeptides fused thereto and there are many such fusion proteins reported.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.) or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the common methods of preparation are vacuum drying and freeze-drying which yields a powder of the active ingredient plus any additional desired ingredient from a previously sterile-filtered solution thereof.

Oral compositions, if used, generally include an inert diluent or an edible carrier. For the purpose of oral therapeutic administration, the active compound (e.g., engineered polypeptides, or agonists of the disclosure) can be incorporated with excipients and used in the form of tablets, troches, or capsules, e.g., gelatin capsules. Oral compositions can also be prepared using a fluid carrier. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

In the event of administration by inhalation, the subject engineered polypeptides, or agonists of the disclosure are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration of the engineered polypeptides, or agonists of the disclosure can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, the engineered polypeptides, or agonists of the disclosure can also be prepared in the form of suppositories (e.g., with conventional suppository bases such as cocoa butter and other glycerides) or retention enemas for rectal delivery.

In some embodiments, the engineered polypeptides, or agonists of the disclosure can also be administered by transfection or infection using methods known in the art, including but not limited to the methods described in McCaffrey et al. (Nature 418:6893, 2002), Xia et al. (Nature Biotechnol. 20:1006-1010, 2002), or Putnam (Am. J. Health Syst. Pharm. 53:151-160, 1996, erratum at Am. J. Health Syst. Pharm. 53:325, 1996)

### Nucleic Acids, Nucleic Acid Constructs and Vectors

Provided herein are also nucleic acids, nucleic acid constructs and vectors expressing the engineered polypeptides of the present disclosure.

The terms “nucleic acids” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR § 1.822 is used herein.

Nucleic acid molecules of the present disclosure can be nucleic acid molecules of any length, including nucleic acid molecules that are preferably between about 5 Kb and about 50 Kb, for example between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

The term “recombinant” nucleic acid molecule as used herein, refers to a nucleic acid molecule that has been altered through human intervention. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant nucleic acid molecule: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination)) of nucleic acid molecules; 2) includes conjoined nucleotide sequences that are not conjoined in nature, 3) has been engineered using molecular cloning techniques such that it lacks one or more nucleotides with respect to the naturally occurring nucleic acid molecule sequence, and/or 4) has been manipulated using molecular cloning techniques such that it has one or more sequence changes or rearrangements with respect to the naturally occurring nucleic acid sequence.

Methods for constructing a DNA sequence encoding the engineered polypeptides and expressing those sequences in a suitably transformed host include, but are not limited to, using a PCR-assisted mutagenesis technique. Mutations that consist of deletions or additions of amino acid residues to an engineered polypeptides can also be made with standard recombinant techniques. In the event of a deletion or addition, the nucleic acid molecule encoding the engineered polynucleotides is optionally digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

The complete amino acid sequence can be used to construct a back-translated gene. A DNA oligomer containing a nucleotide sequence coding for an engineered polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis or another method), the DNA sequences encoding an an engineered polypeptide will be inserted into an expression vector and operatively linked to an expression control sequence appropriate for expression of the engineered polypeptide in the desired transformed host. Proper assembly can be confirmed by nucleotide sequencing, restriction mapping, and expression of a biologically active polypeptide in a suitable host. As is well known in the art, in order to obtain high expression levels of a transfected gene in a host, the gene must be operatively linked to transcriptional and translational expression control sequences that are functional in the chosen expression host.

Some embodiments disclosed herein relate to vectors or expression cassettes including a recombinant nucleic acid molecule as disclosed herein. As used herein, the term “expression cassette” refers to a construct of genetic material that contains coding sequences and enough regulatory information to direct proper transcription and/or translation of the coding sequences in a recipient cell, in vivo and/or ex vivo. The expression cassette may be inserted into a vector for targeting to a desired host cell and/or into a subject. As such, the term expression cassette may be used interchangeably with the term “expression construct”.

Also provided herein are vectors, plasmids or viruses containing one or more of the nucleic acid molecules encoding any of the chimeric polypeptides and bispecific ligands disclosed herein. The nucleic acid molecules described above can be contained within a vector that is capable of directing their expression in, for example, a cell that has been transduced with the vector. In some embodiments of the present disclosure, the engineered polynucleotides can be expressed from vectors, preferably expression vectors. In some embodiments, the expression vectors are mammalian expression vectors. Examples of mammalian expression vectors are known to a person skilled in the art. Such vectors include but are not limited to pD649. In some embodiments, the VHHs and scFvs of the present disclosure and/or fusions thereof can be cloned into such expression vectors. Suitable vectors for use in eukaryotic are known in the art and are commercially available or readily prepared by a skilled artisan. Additional vectors can also be found, for example, in Ausubel, F. M., et al., Current Protocols in Molecular Biology, (Current Protocol, 1994) and Sambrook et al., “Molecular Cloning: A Laboratory Manual,” 2nd ED. (1989).

In some embodiments the subject polypeptides, either alone or as a part of a chimeric polypeptide, such as those described above, can be obtained by expression of a nucleic acid molecule.

It should of course be understood that not all vectors and expression control sequences will function equally well to express the DNA sequences described herein. Neither will all hosts function equally well with the same expression system. However, one of skill in the art may make a selection among these vectors, expression control sequences and hosts without undue experimentation. For example, in selecting a vector, the host must be considered because the vector must replicate in it. The vector's copy number, the ability to control that copy number, and the expression of any other proteins encoded by the vector, such as antibiotic markers, should also be considered. For example, vectors that can be used include those that allow the DNA encoding the engineered polypeptides or fragments thereof to be amplified in copy number. Such amplifiable vectors are well known in the art. They include, for example, vectors able to be amplified by DHFR amplification (see, e.g., Kaufman, U.S. Pat. No. 4,470,461, Kaufman and Sharp, “Construction of a Modular Dihydrafolate Reductase cDNA Gene: Analysis of Signals Utilized for Efficient Expression”, Mol. Cell Biol., 2, pp. 1304-19 (1982)) or glutamine synthetase (“GS”) amplification (see, e.g., U.S. Pat. No. 5,122,464 and European published application 338,841).

The vectors can be useful for autonomous replication in a host cell or may be integrated into the genome of a host cell upon introduction into the host cell, and thereby are replicated along with the host genome (e.g, non-episomal mammalian vectors). Expression vectors are capable of directing the expression of coding sequences to which they are operably linked. In general, expression vectors of utility in recombinant DNA techniques are often in the form of plasmids (vectors). However, other forms of expression vectors, such as viral vectors (e.g., replication defective retroviruses including lentivirus, adenoviruses, and adeno-associated viruses) are included also.

Exemplary recombinant expression vectors can include one or more regulatory sequences, selected on the basis of the host cells to be used for expression, operably linked to the nucleic acid sequence to be expressed.

The expression constructs or vectors can be designed for expression of an engineered polypeptide thereof in host cells.

Vector DNA can be introduced into prokaryotic or eukaryotic ceils via conventional transformation or transfection techniques. Suitable methods for transforming or transfecting host cells can be found in Sambrook el al. (1989) Molecular Cloning: A Laboratory Manual (2nd ed., Cold Spring Harbor Laboratory' Press, Plainview, N.Y.) and other standard molecular biology laboratory manuals.

The nucleic acid sequences encoding the engineered polypeptides, or agonists of the disclosure can be optimized for expression in the host cell of interest. For example, the G-C content of the sequence can be adjusted to levels average for a given cellular host, as calculated by reference to known genes expressed in the host cell. Methods for codon optimization are known in the art. Codon usages within the coding sequence of the chimeric polypeptides and bispecific antibodies disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Vectors suitable for use include T7-based vectors for use in bacteria, the pMSXND expression vector for use in mammalian cells, and baculovirus-derived vectors for use in insect cells. In some embodiments nucleic acid inserts, which encode the subject engineered polypeptides, or agonists of the disclosure in such vectors, can be operably linked to a promoter, which is selected based on, for example, the cell type in which expression is sought.

In selecting an expression control sequence, a variety of factors should also be considered. These include, for example, the relative strength of the sequence, its controllability, and its compatibility with the actual DNA sequence encoding the subject chimeric polypeptide or bispecific antibody, particularly as regards potential secondary structures. Hosts should be selected by consideration of their compatibility with the chosen vector, the toxicity of the product coded for by the DNA sequences of this disclosure, their secretion characteristics, their ability to fold the polypeptides correctly, their fermentation or culture requirements, and the ease of purification of the products coded for by the DNA sequences.

Within these parameters one of skill in the art may select various vector/expression control sequence/host combinations that will express the desired DNA sequences on fermentation or in large scale animal culture, for example, using CHO cells or COS 7 cells.

The choice of expression control sequence and expression vector, in some embodiments, will depend upon the choice of host. A wide variety of expression host/vector combinations can be employed. Non-limiting examples of useful expression vectors for eukaryotic hosts, include, for example, vectors with expression control sequences from SV40, bovine papilloma virus, adenovirus and cytomegalovirus. Non-limiting examples of useful expression vectors for bacterial hosts include known bacterial plasmids, such as plasmids from E. coli, including col El, pCRI, pER32z, pMB9 and their derivatives, wider host range plasmids, such as RP4, phage DNAs, e.g., the numerous derivatives of phage lambda, e.g., NM989, and other DNA phages, such as M13 and filamentous single stranded DNA phages. Non-limiting examples of useful expression vectors for yeast cells include the 2μplasmid and derivatives thereof. Non-limiting examples of useful vectors for insect cells include pVL 941 and pFastBac™ 1.

In addition, any of a wide variety of expression control sequences can be used in these vectors. Such useful expression control sequences include the expression control sequences associated with structural genes of the foregoing expression vectors. Examples of useful expression control sequences include, for example, the early and late promoters of SV40 or adenovirus, the lac system, the trp system, the TAC or TRC system, the major operator and promoter regions of phage lambda, for example PL, the control regions of fd coat protein, the promoter for 3-phosphoglycerate kinase or other glycolytic enzymes, the promoters of acid phosphatase, e.g., PhoA, the promoters of the yeast α-mating system, the polyhedron promoter of Baculovirus, and other sequences known to control the expression of genes of prokaryotic or eukaryotic cells or their viruses, and various combinations thereof.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans will readily appreciate numerous promoters and other regulatory elements which can be used to direct expression of nucleic acids.

In addition to sequences that facilitate transcription of the inserted nucleic acid molecule, vectors can contain origins of replication, and other genes that encode a selectable marker. For example, the neomycin-resistance (neoR) gene imparts G418 resistance to cells in which it is expressed, and thus permits phenotypic selection of the transfected cells. Those of skill in the art can readily determine whether a given regulatory element or selectable marker is suitable for use in a particular experimental context.

Viral vectors that can be used in the disclosure include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

Prokaryotic or eukaryotic cells that contain and express a nucleic acid molecule that encodes a subject engineered polypeptides, or agonists disclosed herein are also features of the disclosure. A cell of the disclosure is a transfected cell, e.g, a cell into which a nucleic acid molecule, for example a nucleic acid molecule encoding an engineered polypeptide, has been introduced by means of recombinant DNA techniques. The progeny of such a cell are also considered within the scope of the disclosure.

The precise components of the expression system are not critical. For example, an engineered polypeptide as disclosed herein can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

The expressed polypeptides can be purified from the expression system using routine biochemical procedures, and can be used, e.g., as therapeutic agents, as described herein.

In some embodiments, engineered polypeptides obtained will be glycosylated or unglycosylated depending on the host organism used to produce the chimeric polypeptides or bispecific antibodies. If bacteria are chosen as the host then the chimeric polypeptide or bispecific antibody produced will be unglycosylated. Eukaryotic cells, on the other hand, will glycosylate the chimeric polypeptides or bispecific antibodies, although perhaps not in the same way as native polypeptides is glycosylated. The engineered polypeptides produced by the transformed host can be purified according to any suitable methods known in the art. Produced engineered polypeptides can be isolated from inclusion bodies generated in bacteria such as E. coli, or from conditioned medium from either mammalian or yeast cultures producing a given engineered polypeptide using cation exchange, gel filtration, and or reverse phase liquid chromatography.

In addition or alternatively, another exemplary method of constructing a DNA sequence encoding the engineered polypeptides of the disclosure is by chemical synthesis. This includes direct synthesis of a peptide by chemical means of the protein sequence encoding for a engineered polypeptide exhibiting the properties described. This method can incorporate both natural and unnatural amino acids at positions that affect the binding affinity of the engineered polypeptide with the target protein. Alternatively, a gene which encodes the desired engineered polypeptide can be synthesized by chemical means using an oligonucleotide synthesizer. Such oligonucleotides are designed based on the amino acid sequence of the desired engineered polypeptide, and preferably selecting those codons that are favored in the host cell in which the engineered polypeptides will be produced. In this regard, it is well recognized in the art that the genetic code is degenerate that an amino acid may be coded for by more than one codon. For example, Phe (F) is coded for by two codons, TIC or TTT, Tyr (Y) is coded for by TAC or TAT and his (H) is coded for by CAC or CAT. Trp (W) is coded for by a single codon, TGG. Accordingly, it will be appreciated by those skilled in the art that for a given DNA sequence encoding a particular engineered polypeptide, there will be many DNA degenerate sequences that will code for that engineered polypeptide. For example, it will be appreciated that in addition to the DNA sequences for engineered polypeptides provided in the Sequence Listing, there will be many degenerate DNA sequences that code for the engineered polypeptides disclosed herein. These degenerate DNA sequences are considered within the scope of this disclosure. Therefore, “degenerate variants thereof” in the context of this disclosure means all DNA sequences that code for and thereby enable expression of a particular engineered polypeptide.

The DNA sequence encoding the subject engineered polypeptide, whether prepared by site directed mutagenesis, chemical synthesis or other methods, can also include DNA sequences that encode a signal sequence. Such signal sequence, if present, should be one recognized by the cell chosen for expression of the engineered polypeptide. It can be prokaryotic, eukaryotic or a combination of the two. In general, the inclusion of a signal sequence depends on whether it is desired to secrete the engineered polypeptide as disclosed herein from the recombinant cells in which it is made. If the chosen cells are prokaryotic, it generally is preferred that the DNA sequence not encode a signal sequence. If the chosen cells are eukaryotic, it generally is preferred that a signal sequence be included.

The nucleic acid molecules provided can contain naturally occurring sequences, or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g, either a sense or an antisense strand).

The nucleic acid molecules are not limited to sequences that encode polypeptides; some or all of the non-coding sequences that lie upstream or downstream from a coding sequence can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

Exemplary isolated nucleic acid molecules of the present disclosure can include fragments not found as such in the natural state. Thus, this disclosure encompasses recombinant molecules, such as those in which a nucleic acid sequence (for example, a sequence encoding a engineered polypeptide) is incorporated into a vector (e.g., a plasmid or viral vector) or into the genome of a heterologous cell (or the genome of a homologous cell, at a position other than the natural chromosomal location).

### Host Cells

Also provided herein are host cells expressing the engineered polypeptides of the disclosure. Non-limiting examples of host cells that can be used include 293 variants (Expi293FTM, Expi293TM GnTI-, 293F, 293S, etc.), ExpiCHO-STM, High Five cells, and E. coli.

### IV. Methods of the Disclosure

### Methods for Identifying Surrogate Cytokine Agonists

Provided herein, in the present disclosure, are also methods for identifying surrogate cytokine agonists wherein the methods include providing nanobodies or scFvs against a first target cytokine receptor and against a second target cytokine receptor; and linking a nanobody or scFv against the first target cytokine receptor with a nanobody or scFv against the second target cytokine receptor thereby identifying a surrogate cytokine agonist.

In some embodiments, the method further comprises screening for induction of downstream signaling activity. In some embodiments, the screening can be for the induction of STAT1, STAT2, STAT3, STAT5, STAT6, Akt, S6, or ERK activity or combinations thereof.

An exemplary workflow of the methods for identifying surrogate cytokine agonists is as follows. However, the discussion of the general methods given herein is intended for illustrative purposes only. Other alternative methods and alternatives will be apparent to those of skill in the art upon review of this disclosure, and are to be included within the spirit and purview of this application.

First a collection of small, single Ig-domain VHH and/or scFv binders are generated against a target receptor or ECD antigen. For example, in some embodiments, single Ig-domain VHH and/or scFv binders are generated against human IL-2Rβ and γ_{C}. Bactrian camels can be immunized by the appropriate antigens expressed as Fc fusions. Following the isolation of peripheral blood cells, phage-displayed VHH libraries can be constructed and subjected to bio-panning for binding to receptor ECDs as shown, for example, in FIG. 2. VHH clones can be recombinantly expressed and ELISA-based screening can be used to identify receptoro-specific binders. In some embodiments, the abilities of the binders are assessed for binding to NK cells. Bispecific ligands can then generated by fusing two binders. In some embodiments, the binders are fused by peptide linkers of varying amino acid lengths. In some embodiments, no linkers are used. In some embodiments, the binders are linked in the forward orientation. In some embodiments, the binders are linked in the reverse orientation. The binders can be utilized in a pairwise combinatorial manner. In some embodiments, these small protein constructs can be rapidly produced by gene synthesis, expressed through a transient transfection and purified. This approach rapidly generates a small library of compounds. In some embodiments, the protein constructs are between 20-50 kDa. The compounds, or surrogate dimerizing ligands are screened for the induction of downstream signaling activity as shown, for example in FIGS. 1-2, or in the Examples provided below. Specific embodiments of the generalized teaching are detailed below in Examples 1, 6 and 7.

This same general workflow can be followed for other cytokine systems presented in this disclosure. This same approach and strategy could also be applied towards any cell surface receptor pairs across both cytokine, receptor tyrosine kinase (RTK), and other dimeric systems such as IgSF family of receptors. The same platform can be also used to create surrogate agonists against trimeric receptors including death receptors, such as TNF receptor-1, CD95 (Fas), TRAMP, TRAIL-R1, or TRAIL-R2. The same platform could also be used for the generation and screening of agonists for naturally-occurring as well as non-naturally occurring combinations of receptors. An example of a non-naturally occurring receptor is detailed throughout the disclosure as well as in the Examples pertaining to the IL-2R/IL-10R combination.

### Methods for Identifying Surrogate Agonists for Cell Surface Receptors

Also provided herein are methods for identifying surrogate agonists for cell surface receptors. The cell surface receptors can be dimeric or trimeric receptors. Examples of dimeric receptors include, but are not limited to cytokine receptors, RTK receptors, and IgSF family. Examples of trimeric receptors include death receptors such as such as TNF receptor-1, CD95 (Fas), TRAMP, TRAIL-R1, or TRAIL-R2. The cell surface receptors can be naturally-occurring or non-naturally occurring. The methods of the disclosure can also pertain, as taught supra, to creating agonists that bring together non-natural combinations of receptor components.

The methods for identifying surrogate agonists include assembling one or more antibody domains to form ligands. The ligands can be monospecific or bispecific. The ligands can be monospecific but include 3 identical binding sites so as to homodimerize three copies of the receptor (in case of trimeric receptors). In some embodiments, the antibody domains are VHHs. In some embodiments the antibody domains are scFvs.

In some embodiments, the methods further comprise employing a screening of the differential induction of interferon stimulated genes (ISGs) as a metric for surrogate IFN activity. The biased induction of ISGs can be used a metric for identifying agonists that force non-natural combinations of receptors. This methodology can be a powerful technique for drug discovery.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purpose.

Although the present disclosure has been described in some detail by way of illustration and example for purposes of clarity and understanding, it will be apparent that certain changes and modifications can be practiced within the scope of the appended claims.

### EXAMPLES

### Example 1-IL-2/15 System

In the IL-2/15 system, the inventors generated a collection of small, single Ig-domain VHH binders against IL-2Rβ and γ_{C}. Human IL-2Rβ and γ_{C} ECD antigens were expressed as Fc fusions and purified, then were used to immunize Bactrian camels. Following isolation of peripheral blood cells, phage-displayed VHH libraries were constructed and subjected to three rounds of bio-panning for binding to receptor ECDs. ELISA-based screening of recombinantly expressed VHH clones identified 65 IL-2Rβ binders and 50 γ_{C} binders. Based on their CDR3 sequence diversity, 10 IL-2Rβ clones from the 4 VHH classes and 6 γ_{C} clones (each from distinct VHH classes) were selected for further evaluation. The binning into distinct VHH classes was to select for diverse epitope coverage on the receptors ECDs. This same general workflow was followed for other cytokine systems presented in this disclosure.

The inventors assessed the ability of the isolated VHHs to bind to YT-1 cells, a human NK cell line which endogenously expresses IL-2Rβ and γ_{C}. Based on strong cell surface staining and diversity of CDR3 loop sequences, four IL-2Rβ-specific VHH clones (β-VHH1, 3, 4, and 6) were chosen for SPR analysis, and bound to IL-2Rβ with steady-state affinities ranging from ˜10-125 nM (FIGS. 2A-C). VHH against γ_{C} were also used for cell binding studies on YT-1 cells, yielding three γ_{C} nanobody clones (γ_{c}-VHH3, 4, and 6) which bound to the cells in a dose-dependent fashion, and SPR experiments measuring binding to immobilized γ_{C} yielded affinities ranging from ˜7-70 nM (FIGS. 2β-C). Based on the SPR affinity measurements, we selected four IL-2Rβ binders (β-VHH1, 3, 4, and 6), three γ_{C} clones (γ_{c}-VHH3, 4, and 6), along with two γ_{C} scFv clones (P1A3, P2B9) whose sequences we identified from a patent (FIG. 2C) (Wang et al. 2016). The inventors followed a similar VHH selection and triage strategy for the IFN and IL-10 systems discussed in this disclosure.

Bispecific molecules were generated by fusing IL-2Rβ and γ_{C} binders through short, flexible Gly-Ser linkers (8 a.a. for VHH-scFv fusions and 2 a.a. for VHH-VHH fusions) in both Forward and Reverse orientations (FIG. 1B). In total, the “all by all” matrix of 4 IL-2Rβ binders×5 γ_{C} binders×2 orientations resulted in 40 molecules. These small protein constructs (˜23 kDa-40 kDa) were rapidly produced by gene synthesis, expressed through transient transfection of approximately 2 mL of Expi293 cells and purified via their 6-His tags using small Ni²⁺-agarose columns (FIG. 1C). Such an approach rapidly generated a small library of compounds.

The surrogate dimerizing ligands, after normalizing for relative concentrations, were then screened in parallel for induction of STAT5 phosphorylation in YT-1 cells (FIGS. 1D, 2G, and 2H). Of the 40 candidates, we found 28 agonists (˜70% “hit rate”), spanning from minimally active (3 ligands), ˜½ Eₘₐₓ relative to hIL-2 (5 ligands), full Eₘₐₓ (17 ligands), and supraphysiologic Eₘₐₓ (3 ligands) (FIGS. 1D and 2D). Notably, amongst the agonists, there was no discernable relationship between a ligand's Eₘₐₓ and its affinity for individual IL-2Rβ or γ_{c} receptors (FIGS. 1E and 1F). This finding highlights the influence of VHH binding epitope on the receptor ECDs, which influences the overall geometry of the signaling dimer, in addition to ligand-receptor affinity, in determining signaling output. There was also a lack of correlation between Eₘₐₓ and the product of receptor binding affinities β×γ_{C} (FIG. 1G). Even amongst ligand pairs with identical β and γ_{C} VHH, alternative (Forward or Reverse) orientations (β-γ_{C} vs. γ_{C}-β), elicited divergent activities in 15 of 20 total pairings (FIG. 1G).

### Example 2—Outputs of IL-2 Signaling

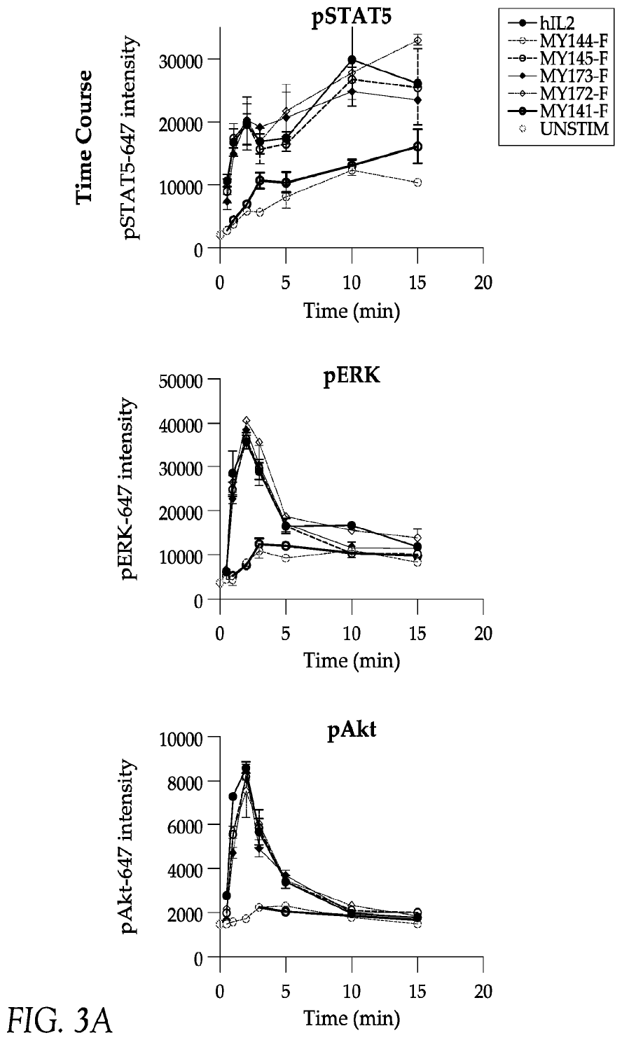

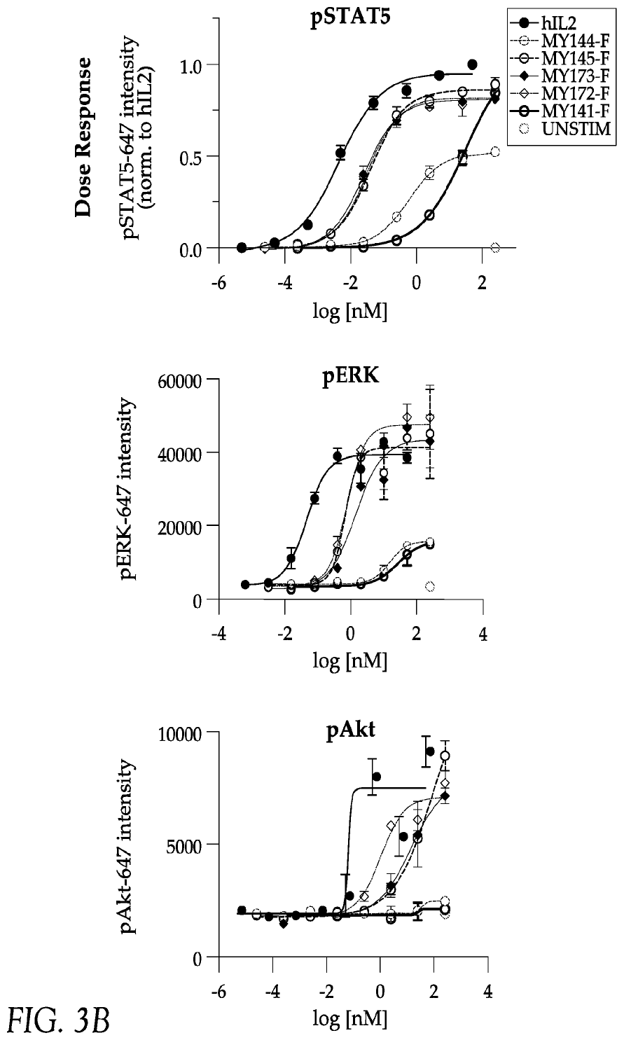

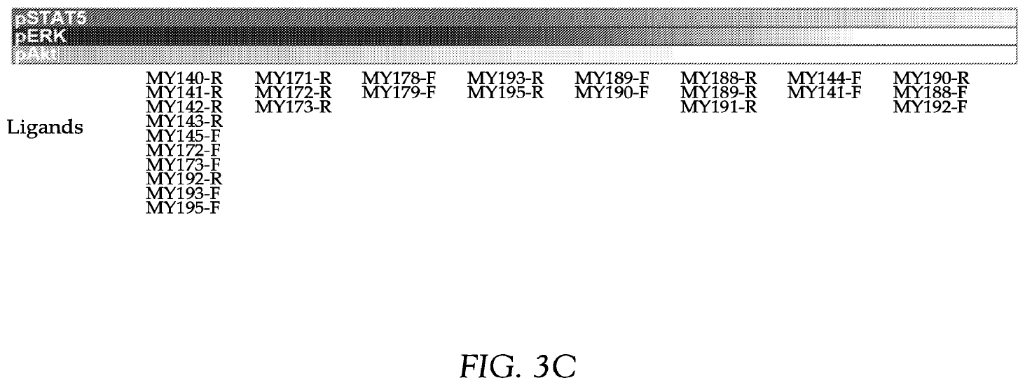

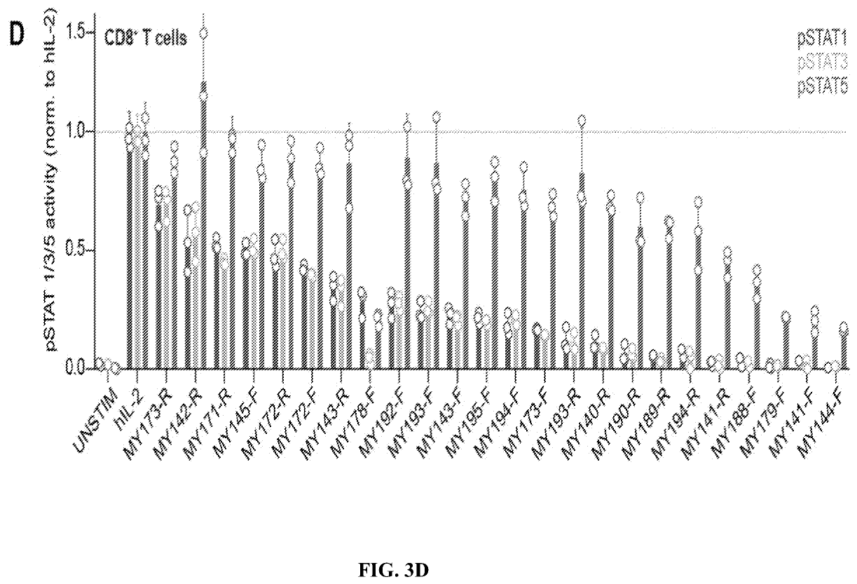

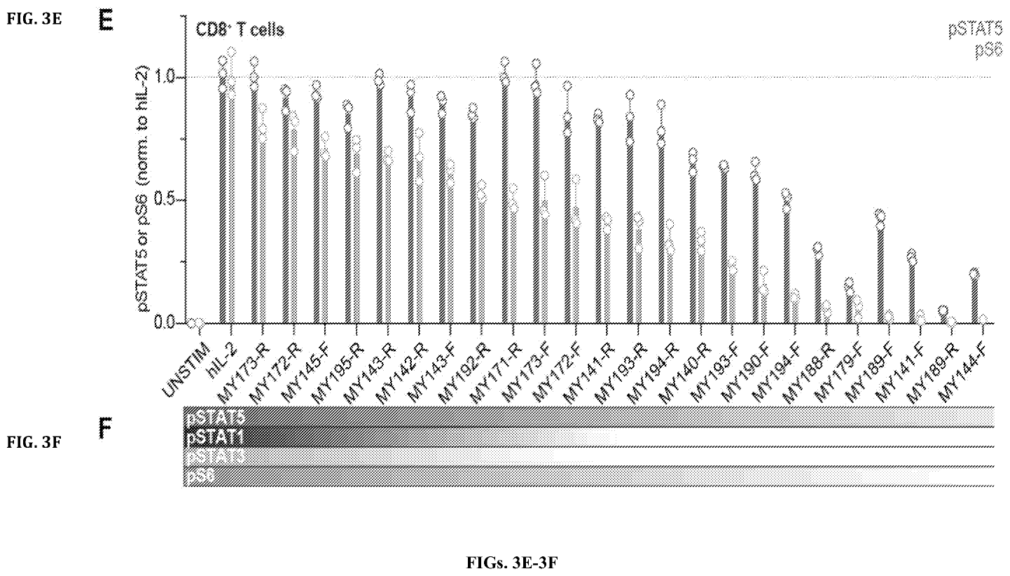

The inventors examined the principal membrane-proximal outputs of IL-2 signaling: activation of pSTAT5, pERK, and PI3K/pAkt. For kinetics studies, YT-1 cells were stimulated with saturating concentration of hIL-2 or surrogate ligands for varying amounts of time (FIG. 3A). For dose-response studies, the inventors varied the concentration of ligand and measured phosphorylation at a fixed time point (3 min.) corresponding to peak pERK and pAkt signal levels for hIL-2. Surrogate agonists elicited a range of behaviors: some similar to IL-2, while others showed delayed activation and reduced peak responses (MY189-F, MY190-F), and impaired activation of some, but not all pathways (MY178-F, MY179-F) (FIGS. 3A-3B). Grouping ligands according to their relative strengths of pSTAT5, pERK, and pAkt signaling (relative to hIL-2) revealed distinct classes of signal patterns (FIG. 3C). Notably, the relative strengths of pSTAT5/pERK/pAkt do not appear to be tightly coupled. Across all ligands, pSTAT5 appeared to be preferentially activated, trailed by pERK and then pAkt: pSTAT5 activity ≥ pERK ≥ pAkt.

IL-2 and IL-15 principally activate STAT5 but have also been shown to induce STAT1 and STAT3 activity (Delespine-Carmagnat et al., 2000; Ng and Cantrell, 1997), which are required for efficient maintenance of CD8⁺ memory T cells (Cui et al., 2011; Quigley et al., 2008; Siegel et al., 2011). The inventors measured STAT1, 3, and 5 phosphorylation after stimulation by IL-2 analogs. In pre-activated primary T and NK cells (FIG. 3D), ligand MY173-R was similar to IL-2 in its pSTAT1/3/5 balance. However, the remaining surrogate agonists favored dominant pSTAT5 signaling over pSTAT1 and pSTAT3. For example, in CD8⁺ T cells, MY193-R displayed ˜90% pSTAT5 activity, but only ˜25% pSTAT1 or pSTAT3 activity relative to hIL-2. MY193-R also showed a similar pSTAT bias in NK cells, where it evoked 45% of pSTAT5 activity and only 3% pSTAT1/3 activity. The inventors also found that in T cells, many surrogate ligands exhibited higher pSTAT5 activity relative to pS6, a substrate downstream of PI3K/Akt signaling (Ross and Cantrell, 2018) (FIG. 3E, 3F). In general, the biased pSTAT5 vs. pS6 ratio was not as pronounced as in the pSTAT5 vs. pSTAT1/3 ratio. In addition to MY173-R (the only ligand with a balanced pSTAT1/3/5 ratio), MY172-R, MY145-F, and MY195-F stimulated balanced levels of pSTAT5 and pS6 phosphorylation in T cells, whereas the remaining ligands favored pSTAT5 activity over pS6.

### Example 3—Crystallization of Vhhs Bound to IL-2Rβ Oric

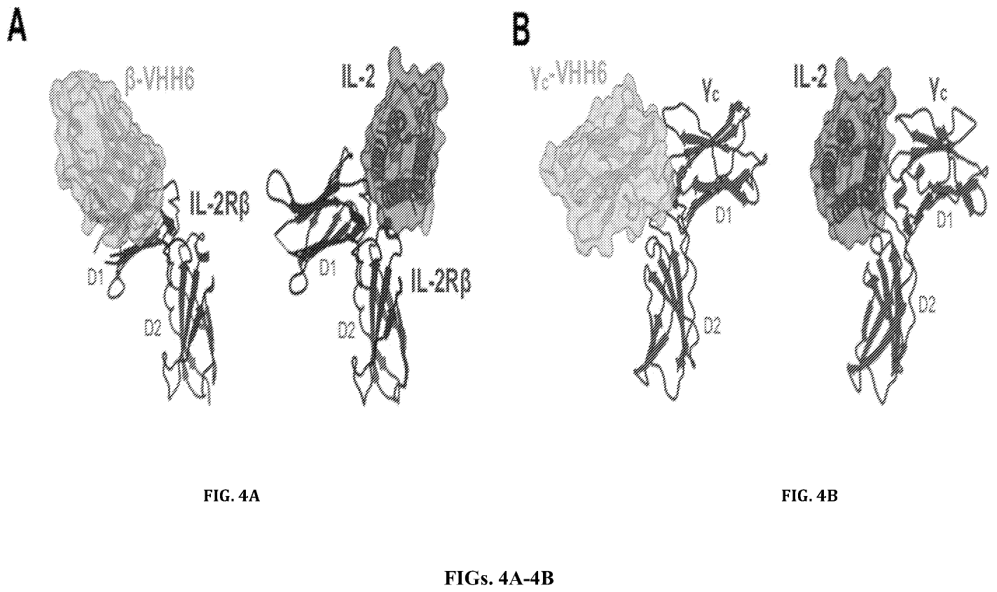

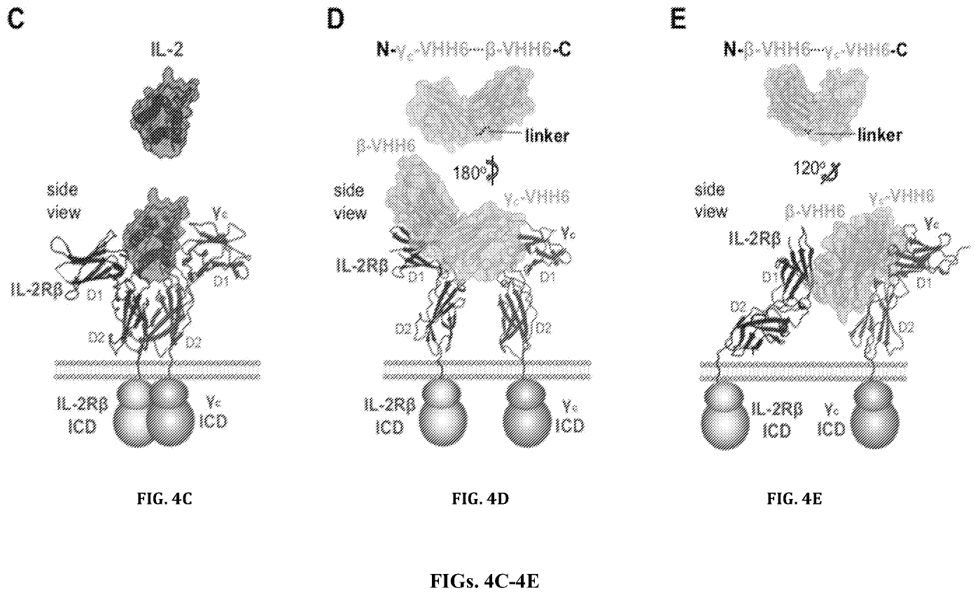

To gain insight into the structural basis for signaling differences, two different VHHs, one bound to IL-2Rβ and the other bound to γ_{C} were crystallized. With structures of the individual receptor:VHH complexes in hand the inventors modeled the complete dimeric receptor geometry since the short (2 a.a.) linker places constraints on the relative overall geometry of the two VHHs in the bispecific ligand. Due the presumed flexibility of the linker, the models were only intended to convey approximate relative differences in global dimer topologies as a result of the differing VHH epitopes on the ECDs. The IL-2Rβ:β-VHH6 complex was resolved at 1.9 Å and revealed that the β-VHH6 binds to the D1 domain of the receptor, as opposed to binding at the “elbow” of the D1-D2 juncture like IL-2 (Wang et al., 2005) (FIG. 4A and Table 1). The inventors resolved the γ_{C}:γ_{C}-VHH6 complex to 2.6 Å and found that the γ_{C}-VHH6 occupied a similar binding footprint on γ_{C} as IL-2 (FIG. 3B and Table 1). The inventors modeled the two (forward and reverse) orientations of ligands, β-VHH6-γ_{C}-VHH6 and γ_{C}-VHH6-β-VHH6. The two complexes predict significant differences from the IL-2 receptor heterodimer geometry in both distance and angular relationship between IL-2Rβ and γ_{C} (FIGS. 4C, 4D, and 4E). Surprisingly, it was observed that in YT-1 cells γ_{C}-VHH6-β-VHH6 (MY173-R) induced supraphysiologic STAT5 phosphorylation but normal ERK/Akt phosphorylation relative to hIL-2 (FIGS. 1D and 3C). The reverse orientation analog, β-VHH6-γ_{C}-VHH6 (MY173-F), had a similar signaling profile to IL-2 in YT-1 cells. Whereas MY173-R had a signaling profile like IL-2 in CD8⁺ T and NK cells, MY173-F had high pSTAT5 activity (˜70-100% of IL-2), and low pS6 and pSTAT1/3 activity (50% and ˜14-24%, respectively). These structures support the hypothesis that differences in receptor dimerization geometry strongly influence proximal signaling, likely through variations in the orientation and proximity of the JAK kinase domains to receptor ICDs.

### Example 4—Transcriptional Profiles of IL-2 Surrogate Ligands

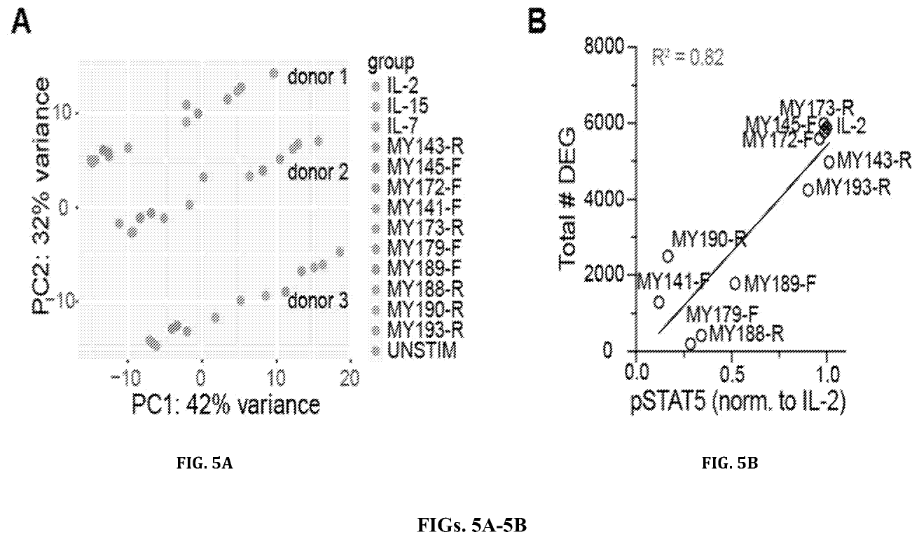

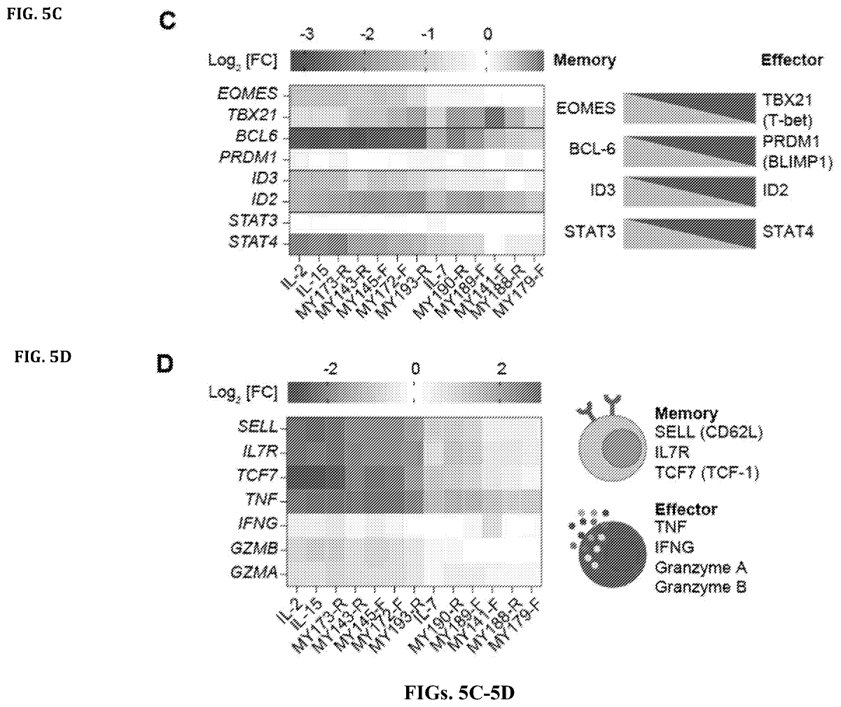

To characterize the transcriptional profiles induced by different signaling classes of IL-2 surrogate ligands on T cells, mRNA sequencing was performed. Principal component analysis (PCA) indicated that the IL-2 analogs had a concerted effect on gene expression across the PC1 axis (FIG. 5A). Within each donor, ligands MY173-R, MY143-R, MY145-F, and MY172-F clustered together with IL-2 and IL-15 (which are known to drive highly similar gene expression profiles (Ring et al., 2012)); MY141-F, MY188-R, and MY179-F clustered near the unstimulated sample, whereas MY190-R and MY193-R were close to IL-7, which is required for naïve and memory T cell homeostasis and opposes terminal effector T cell differentiation (FIG. 4A) (Shourian et al., 2019). These relationships were also preserved on a group level, as seen in the distance calculation matrix. Overall, a given ligand's pSTAT5 activity (on CD8⁺ T cells) was linearly correlated with its potency at regulating gene expression (R2=0.82, FIG. 5B).

An important function of IL-2 is to induce CD8⁺ T cell differentiation, so the inventors examined expression of pairs of transcription factors that exert opposing effects on T_{memory} VS. T_{effector} differentiation (FIG. 5C) (Kaech and Cui, 2012). Four pairs of transcription factors, EOMES/TBX21, BCL-6/PRDM1, ID3/ID2, and STAT3/STAT4, regulate the balance of memory vs. effector potential based on their relative expression ratios and/or activities (Kaech and Cui, 2012). We found that along with IL-2/IL-15, ligands MY173-R, MY143-R, MY145-F, and MY172-F downregulated EOMES but not TBX21 and BCL6 and not PRDM1, while upregulating ID2 but not ID3, and STAT4 but not STAT3. Taken together these four sets of ratios favor differentiation toward effector over memory cells (Kaech and Cui, 2012). Consistent with this, the same set of ligands downregulated markers of naïve and central memory cells (such as SELL, IL7R, and TCF7) while upregulating expression genes encoding the effector cytokines and cytolytic molecules TNFα, IFNγ, granzyme A, and granzyme B (FIG. 5D) (Kaech and Cui, 2012).

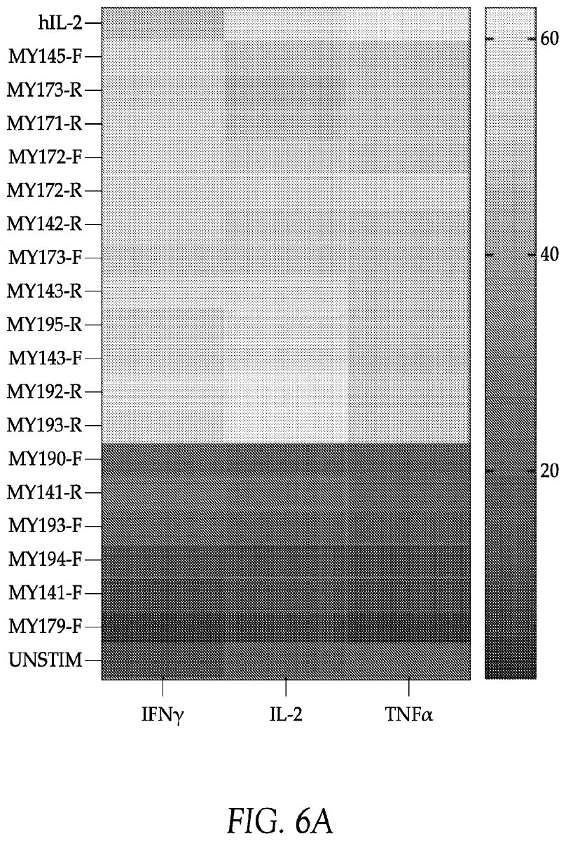

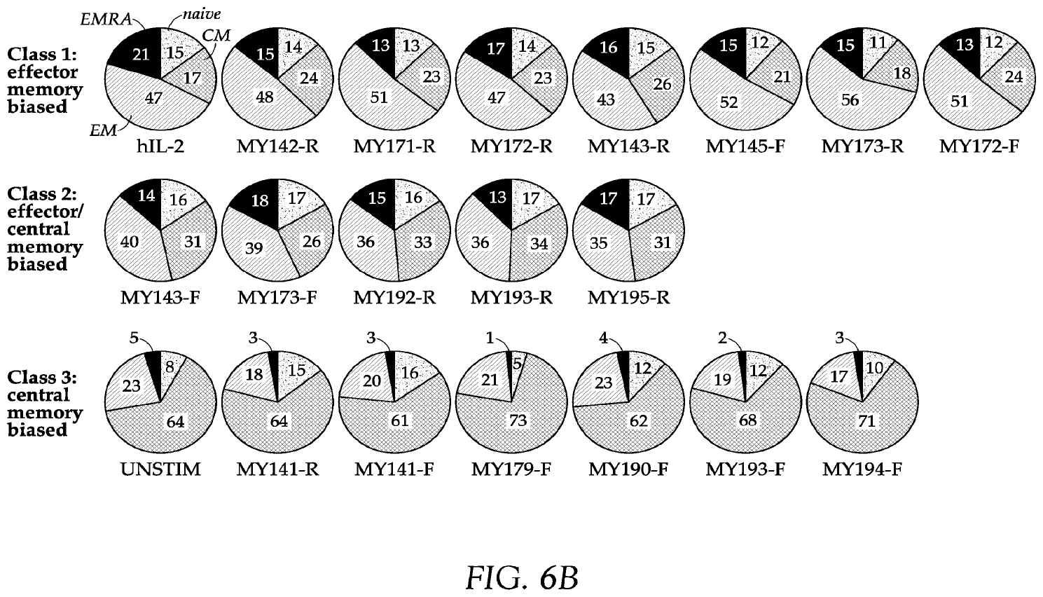

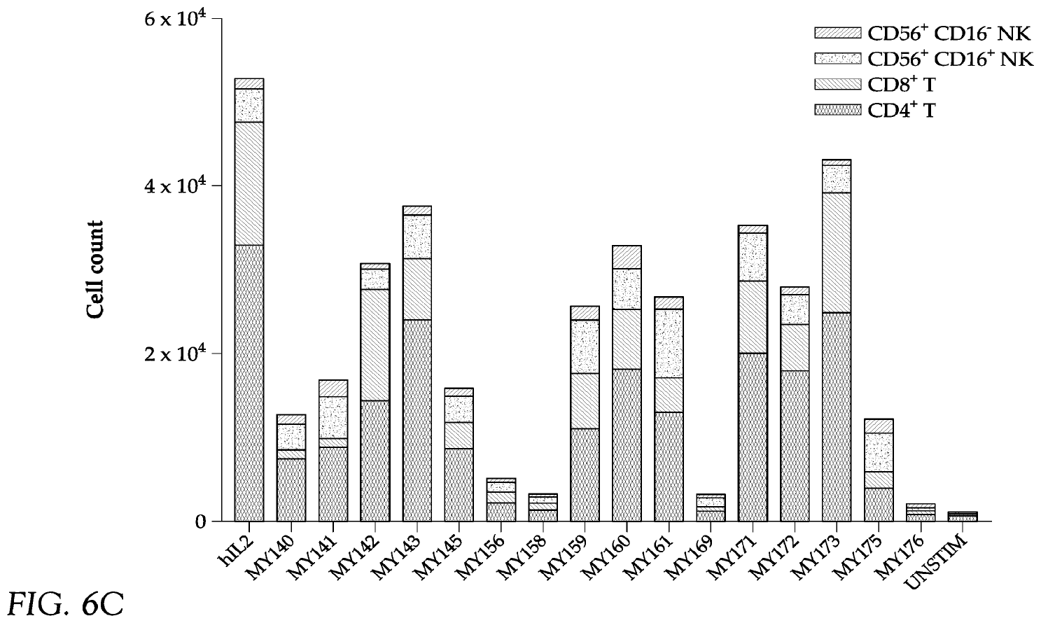

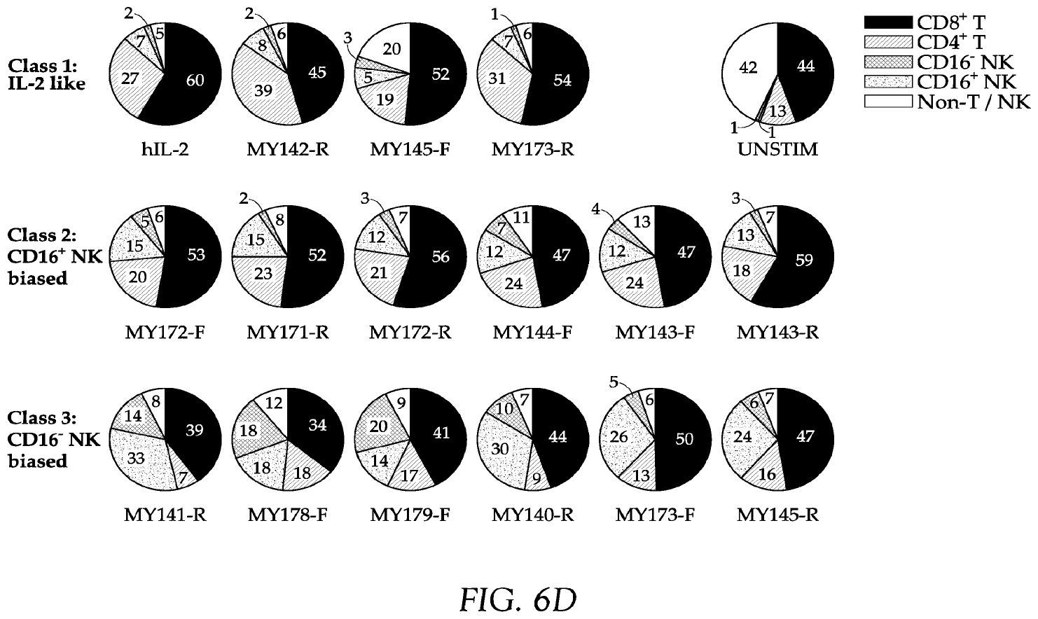

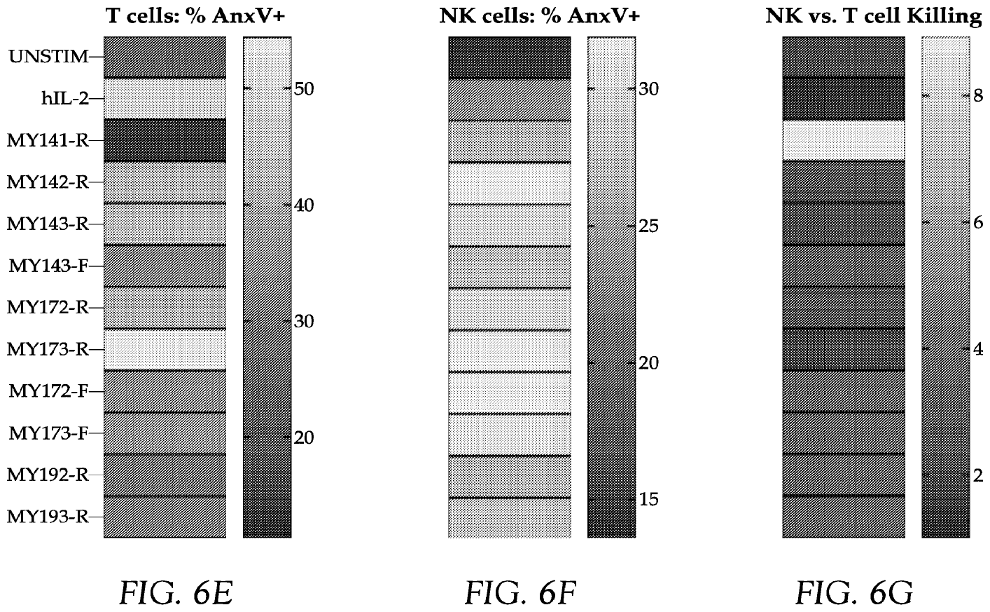

One of the principal roles of IL-2 is to direct the differentiation of naïve CD8⁺ T cells into memory and cytotoxic effector cells, thus the inventors also probed the ability of the surrogate ligands to orchestrate development of T cell memory. Cells were stained with surface antibodies to CCR7 and CD45RA to determine the distribution between naïve (Tₙ), central memory (T_{CM}), effector memory (T_{EM}), and more terminally differentiated effector memory CD45RA (T_{EMRA}) T cells (Maecker et al., 2012). Ligands spanned a broad range of differentiation potential, ranging from IL-2-like in distribution to central and effector memory biased, entirely CD8 selective, or nonfunctional despite triggering pSTAT5 signaling (FIG. 6B).

### Example 5—Profiles of Cytokine Expression

The expression of cytokines important for cytolytic function was profiled (FIG. 6A). Ligands that supported T cell expansion were tightly correlated with acquisition of proliferative and cytotoxic cytokine production (IL-2, TNFα, IFNγ). MY173-R supported equivalent T cell proliferation to IL-2, and the resultant cells had a CD8⁺ memory distribution phenotype (FIG. 6B, top row). However, relative to hIL-2, a higher proportion of MY173-R-cultured cells produced IFNγ, with a lower proportion making IL-2 and TNFα (FIG. 6A). Another differentiation phenotype is represented by MY173-F and MY193-R. These ligands evoked relatively high levels of pSTAT5 activity (˜70-90% of IL-2) but had lower levels of pSTAT1/3 activity (<30% of hIL-2; FIG. 2D) and promoted lower levels of proliferation as compared to hIL-2 (FIG. 6B, middle row). Relative to IL-2 treatment, MY173-F and MY193-R drove higher proportions of central memory cells in addition to supporting effector memory differentiation, while inducing less T_{EMRA} cells. A higher fraction of MY193-R treated cells produced IL-2 with a lower percentage of TNFα producers, consistent with a central memory phenotype (FIG. 6A). A third category of ligands, which includes MY141-F, were strongly central memory dominant (FIG. 6B, bottom row).

One clear IL-2 dependent functional readout is cytolysis of target cells. To test this, preactivated human T cells transduced with the A3A T cell receptor (TCR), which recognizes the MAGE-3A peptide presented by HLA-A*01 on A375 melanoma cells (Cameron et al., 2013; Linette et al., 2013) were used. The IL-2 surrogate ligands supported T cell cytotoxic function to varying degrees, largely matching their ability to support CD8⁺ T cell proliferation and generate effector cytokines (FIG. 6E).

IL-2 and IL-15 are also known to support NK cell expansion and arm them with cytotoxic function (Wu et al., 2017). To assess proliferation, the inventors expanded PBMCs with 100 nM hIL-2 or IL-2 surrogates for 14d, then profiled T and NK cell types using surface antibody staining (FIG. 6C). Culture with IL-2 supported the highest level of total cell expansion, while 12 of the surrogate agonists increased the proportion of NK cells in the population relative to IL-2-treated cells (FIG. 6D), indicating an NK bias. Ligands which supported 24-50% of total cell number produced an expanded fraction of CD16⁺ NK cells (˜3-5 fold increased relative to IL-2), which marks cytolytic NK cells (Cooper et al., 2001). Ligands MY141-R and MY178-F produced 7- and 9-fold expanded fractions of CD16-NK cells, which are thought to be specialized for cytokine production (Cooper et al., 2001).

The cytotoxic capacity of NK cells cultured with IL-2 or analogs was directly measured. Surprisingly, a subset of IL-2 surrogate agonists including MY173-R and MY173-F supported annexin positivity rates that were ˜23-45% higher than that produced by IL-2 or IL-15 (FIG. 6F). The NK vs. T cell bias in cell expansion and survival (FIG. 6E) suggested that the surrogate ligands might also preferentially drive the ability of NK cells to acquire cytotoxicity over that of T cells. To measure this, we normalized cytotoxicity values for surrogate ligands to that of hIL-2 in NK cells and in T cells, then plotted the normalized NK-to-T cell killing ratio (FIG. 6G). The IL-2 analogs of the present disclosure all exhibited bias toward NK-mediated killing (ratio >1), from slight NK bias (MY173-R, ratio=˜ 1.4) to moderate (most ligands, including MY173-F, ratio ˜2-3) to highly NK selective (MY141-R, ratio ˜9).

### Example 6—Type I Interferon Systems (IFN)

A similar strategy as described above was also used to create surrogate agonists in the Type I interferon (IFN) system using a collection of VHH and scFv binders to human IFNAR1 and IFNAR2, fused via 2 a.a. or 5 a.a linkers (FIG. 7A). A subset of binders were selected for SPR analysis, and bound to their corresponding receptor with high affinities (FIGS. 8A and 8B). Despite their high affinities, an initial 66-member screening matrix (11 IFNAR1 binders×6 IFNAR2 binders) in the IFNAR1-IFNAR2 orientation produced only 12 active hits, yielding an approximate 18% hit rate (FIG. 7A). These 12 hits were then expressed in the reverse (IFNAR2-IFNAR1) orientation and screened, none of which had measurable activity. A subset of active molecules, termed “Human Interferon Surrogates” (HIS) 1-7, were selected for further studies (FIG. 7A). The HIS ligands induced dose-dependent pSTAT1 activation, the hallmark STAT activated by Type I IFNs, on YT-1 and A549 (lung epithelial) cell lines, as well as on human PBMCs, exhibiting partial agonist Eₘₐₓ relative to the natural cytokine human IFNω (FIGS. 7β-D). Since Type I IFNs also activate additional STATs, STAT1-STAT6 phosphorylation was profiled. The inventors observed reduced pSTAT1 activation relative to IFNω but equivalent pSTAT2 and pSTAT3 activation on both on the NK cell line YT-1 and A549 cells (FIGS. 7E-F). Thus, the surrogate IFN ligands display signaling bias for pSTAT activation relative to human IFNω.

Since Type I IFNs are a critical viral defense mechanism, the surrogate ligands were tested as to whether they exhibited antiviral activity on A549 cells infected with Sendai virus (SeV). All HIS ligands showed similar inhibition of SeV replication as IFNω, despite their reduced pSTAT1 activation (FIG. 7G). HIS agonists also inhibited SARS-COV-2 replication in A549 cells expressing human ACE2 receptor, as measured with an antiviral assay using recombinant SARS-COV-2 engineered to express nanoluciferase (Hou et al., 2020) (FIG. 7H). Additionally, HIS agonists inhibited SARS-COV-2 replication in primary human airway cells (FIG. 8C). After 24 hr. pretreatment, we observed a potent dose-dependent antiviral effect on SARS-COV-2 replication. Interestingly, the antiviral potency of HIS ligands varied based on the identity of the IFNAR1 binder. Whereas all 4 ligands using the “3F11” scFv (HIS1-HIS4) exhibited potent antiviral activity, ⅔ ligands with the “A1” VHH (HIS5-7) (VHH “A1” was identified from a commercially available yeast-displayed VHH library (https://www.kerafast.com/item/1770/yeast-display-nanobody-library-nblib) had poor activity (FIGS. 7A and 7H) suggesting that the magnitude and/or composition of the generated antiviral response is guided by VHH or scFv structure-activity relationships, similar to observations from the IL-2 system.

Type I IFNs exhibit antiviral ability by inducing interferon stimulated genes (ISGs), and the surrogate IFN ligands compared with IFNω showed biased induction of ISGs. Specifically, the surrogates maintained high levels of antiviral gene expression but induced lower levels of pro-inflammatory and pro-apoptotic gene expression (FIGS. 7I and 8D). In human primary airway epithelial cells, HIS ligands induced high levels of the antiviral genes MX1 and OAS1 with minimal induction of pro-inflammatory genes CXCL9 and CXCL10 (FIG. 7J). Moreover, HIS agonists effectively inhibited SeV replication in PBMCs while barely inducing pro-inflammatory cytokine expression (FIGS. 7K-L).

Another functional property of type I IFNs is anti-proliferative activity, and the inventors observed less pro-apoptotic gene induction by HIS agonists. Consistent with this ISG bias, HIS agonists did not suppress cell proliferation as much as IFNω in primary airway epithelial cells (FIG. 7M). Taken together, these ligands have biased ISG induction, which contributes to preserved antiviral activity but restrained anti-proliferative and pro-inflammatory effects. Collectively, these data demonstrate that surrogate IFN agonists are exquisitely potent antiviral agents against SARS-COV-2 and could be further explored as potential medical countermeasures for COVID-19, as well as for other viruses.

### Example 7—IL-2Rβ and IL-10Rβ

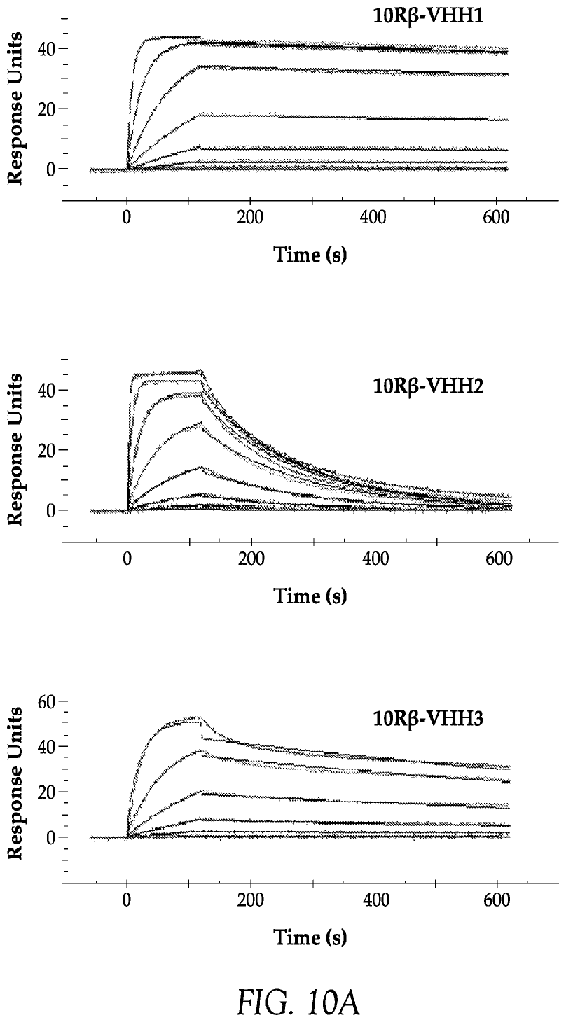

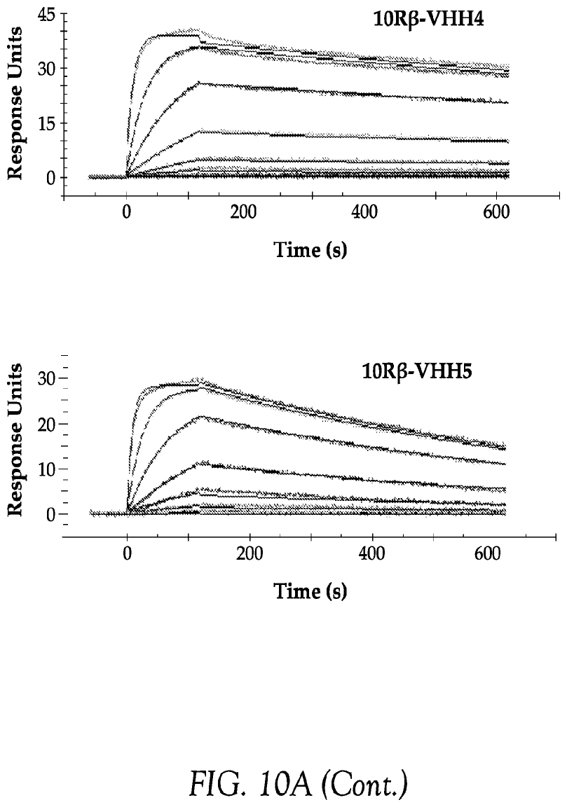

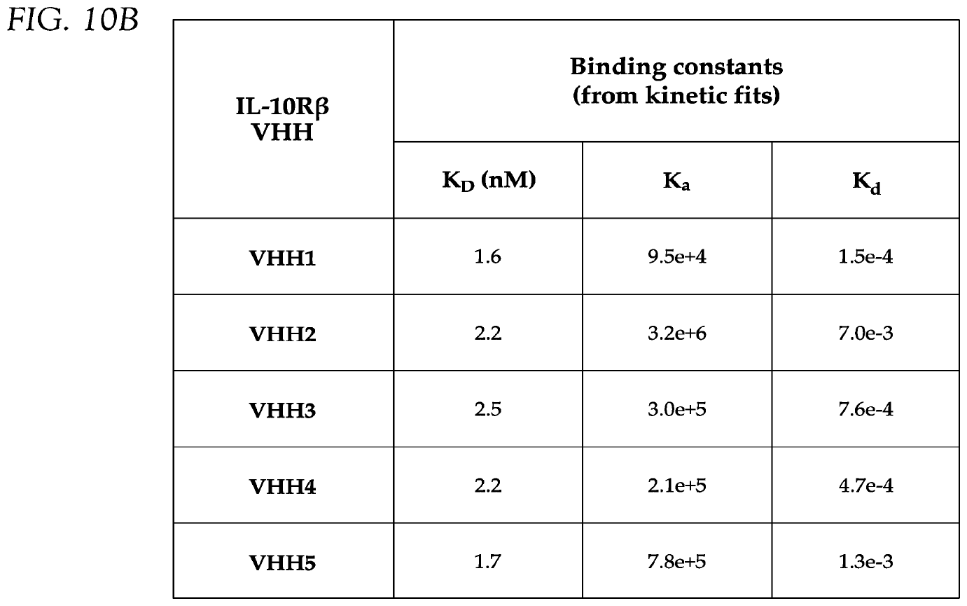

The availability of a large collection of VHH and scFv binders to cytokine receptor ECDs enabled the inventors to reach beyond natural pairings of cytokine receptors that are driven by natural cytokines, to explore enforced proximity of novel cytokine receptor heterodimer pairs that might elicit new types of signals. To this end, since IL-2Rβ and IL-10Rβ are both expressed on T and NK cells, a series of bispecific ligands were designed to induce an “synthetic” IL-2Rβ/IL-10Rβ heterodimer on cells (FIG. 9A). In principle, this would create a heterodimeric entity on the cell surface with the potential for JAK1/TYK2 transphosphorylation and activation of pSTAT5 (via IL-2Rβ) and pSTAT3 (via IL-10Rβ). Although surrogate IL-10Rβ VHHs bound to IL-10Rβ with low nM affinities (FIGS. 10A-10B) and IL-2Rβ VHHs produced agonists when paired with γ_{c} VHHs, only four active agonist “hits” (out of 30 attempted combinations, ˜13% hit rate) of bispecific agonists through induced proximity between IL-2Rβ and IL-10Rβ (FIG. 9B). These four ligands showed partial agonism of pSTAT5 relative to hIL-2 (FIG. 9B), but none stimulated measurable STAT3 phosphorylation. One of the most potent, 10Rβ1-2Rβ6, was selected for further optimization. In order to enhance the agonist strength of one of the partial agonist ligands, a panel of linker length-modulated 10Rβ1-2Rβ6 molecules (FIG. 9C) elicited pSTAT5 Eₘₐₓ activity from very low (16 a.a. linker) to full agonism equivalent to IL-2 (0 a.a. linker), demonstrating the “tunability” of the surrogate system (FIG. 9C). The most potent variant, 10Rβ1-2Rβ6 (0 a.a.) was selected and also a C-terminally Fc-fused 10Rβ1-2Rβ6 was tested versus 10Rβ1-2Rβ6 in pSTAT5 signaling in human primary T cells (FIGS. 9D and 9E). The Fc fusion more than doubled the Eₘₐₓ relative to the monomeric 10Rβ1-2Rβ6 (FIG. 9E), likely through avidity enhancement.

Given the critical role of STATS activity in T cell proliferation, the 10Rβ1-2Rβ6 dimerizers were tested as to whether they could drive primary T cells to proliferate. Monomeric and Fc-linked 10Rβ1-2Rβ6 induced expansion of CD8⁺ T cells but not CD4⁺ T cells (FIGS. 9F and 9G). Stimulation of naive T cells with these ligands in a differentiation assay resulted in a greater fraction of central memory T cells in CD8⁺ T cells than CD4⁺ T cells, which is more similar to the actions of IL-7 than to IL-2 (FIG. 9H). The 10Rβ1-2Rβ6 and 10Rβ1-2Rβ6-Fc agonists also potentiated CD8⁺ T cell degranulation, IFNγ production, and activation in the A3A-MAGE 3A TCR:pMHC system. On primary NK cells, 10Rβ1-2Rβ6 and 10Rβ1-2Rβ6-Fc induced different extents of STAT5 phosphorylation (FIG. 9I) and proliferation (FIG. 9J). 10Rβ1-2Rβ6 and 10Rβ1-286-Fc promoted the lytic activity of NKL cells in a NK cytotoxicity assay against K562 tumor cells and an NK ADCC assay against rituximab-treated Raji tumor cells (FIGS. 9K and 9L). To assess the effect of these ligands on NK degranulation and activation, NK cells were co-cultured with K562 cells. Treatment with 10Rβ1-2Rβ6 and 10Rβ1-2Rβ6-Fc robustly enhanced CD107 (LAMP-1) surface expression and production of IFNγ and MIP-1B in both primary NK cells or NKL cells (FIGS. 9M-9O). The results show that IL-10Rβ/IL-2Rβ agonists preferentially act on CD8⁺ T cells and NK cells versus IL-2, and that the novel IL-10Rβ/IL-2Rβ heterodimer signal more closely resembles an IL-2 receptor partial agonist than an IL-10-mediated partial given its pSTAT5 bias.

### Example 8—Materials and Methods for Examples 1-5

### Camel Immunization

Human IL-2Rβ ECD (a.a. 27-240), human γC ECD (a.a. 23-262), human IL-10Rβ ECD (a.a. 20-220), human IFNAR1 ECD (a.a. 28-436), and IFNAR2 ECD (a.a. 27-243) were expressed as Fc fusions in HEK293F cells and purified by protein A affinity chromatography. Purified receptor ECDs were mixed with Freund's adjuvant, then individually injected into healthy Bactrian camels (Camelus bactrianus). After the seventh immunization, antiserum titer reached 1.0×10⁵ (indicating a strong immune response) and we collected 100 mL of peripheral blood for phage display library construction. All camel experiments were performed in compliance with ethics guidelines approved by Shanghai Science and Technology Committee (STCSM).

### VHH Library Construction

Following isolation of peripheral blood lymphocytes (PBLs) from immunized camels, RNA was extracted, cDNAs were reverse transcribed, and VHHs were amplified by two-step nested PCR. Purified VHH fragments were subcloned into the phage-display phagemid pMECS and used to construct the phage display libraries. The quality of libraries was evaluated by size and insertion rate. Insertion rate was calculated by randomly screening 24 clones per library and determining insertion size by PCR amplification.

### VHH Library Selection

VHHs specific for IL-2Rβ, γ_{c}, IL-10Rβ, IFNAR1, and IFNAR2 were selected from phage-display libraries using target proteins and enriched by three consecutive rounds of bio-panning with the infection of VCSM13 helper phages. Three hundred individual colonies were randomly selected from the enriched pool and positive clones were identified using periplasmic extract ELISA (PE-ELISA).

### Protein Expression

VHH were fused using a 2-8 a.a. linker, and VHH-scFv were fused via an 8a.a. Gly-Ser linker. VHH and scFv fusions were cloned into a pD649 mammalian expression vector (ATUM DNA 2.0), which carries an HA secretion signal peptide and a C-terminal 6-His tag. Proteins were expressed in Expi293F cells (Thermo Fisher Scientific) for 5-7 days according to manufacturer protocols, isolated using Ni2+ affinity chromatography, then further fractionated over a Superdex 200 increase column equilibrated with 20 mM HEPES (pH 7.4) and 150 mM NaCl.

### Cell Culture

CD45⁺ YT-1 cells (Kuziel et al., 1993) and human PBMC were isolated from LRS chambers (Stanford Blood Center), were maintained at 37° C. in a 5% CO₂ humidified chamber, and cultured in complete RPMI medium (RPMIc) containing 10% FBS and supplemented with 25 mM HEPES, 2 mM pyruvate, 4 mM GlutaMAX, non-essential amino acids, and penicillin-streptomycin (all cell cultured reagents were purchased from Gibco). Prior to stimulation for pERK and pAkt studies, cells were starved in serum-free RPMI for 1-2 hours. Primary cells were rested overnight without cytokine before measuring signaling.

Normal human primary bronchial/tracheal epithelial cells were purchased from ATCC (PCS-300-010) and grown in Airway Epithelial Cell Basal Media (ATCC PCS-300-030) supplemented with Bronchial/Tracheal Epithelial Cell Growth Kit components (ATCC PCS-300-040) following manufacturer's instructions. A549 cells were maintained in complete DMEM medium containing 10% FBS and supplemented with 25 mM HEPES, 2 mM sodium pyruvate, 4 mM GlutaMAX, and penicillin-streptomycin.

RNA-seq experiments: T cells were pre-activated for 4d with α-CD3/CD28, washed, and rested overnight without stimulation. The following day, CD8⁺ T cells were purified using MACS (CD8⁺ T cell isolation kit, Miltenyi Biotec), then stimulated with 100 nM natural cytokine (hIL-2, hIL-7, hIL-15) or surrogate ligand for 24 hr. at 37° C. Total RNA from 1-2 million cells per condition was extracted using an RNeasy micro kit (Qiagen). For each condition, we performed 3 biological replicates, representing samples from 3 independent donors. cDNA library preparation and RNA sequencing were performed by Novogene. cDNA libraries were loaded onto an Illumina NovaSeq 6000 sequencer, PE150 platform. Reference genome and gene model annotation files were downloaded from the genome website browser (NCBI/UCSC/Ensembl) directly. Paired-end clean reads were aligned to the reference genome using STAR software, and differential expression analysis was conducted using the DESeq2 R package (Love et al., 2014). Data (raw and processed) are deposited under GEO accession record GSE183436.

NK experiments: Primary NK cells (from a mixed human PBMC population) were pre-activated for 5-7d with 2 μg/mL plate-bound α-NKD30 (Biolegend) with 9 nM hIL-15 (R&D) in RPMIc media. Following activation, cells were rested for 1d in RPMIc without stimulation, then plated into 96-well microplates in the presence of hIL-2 or IL-2 surrogate ligands. Media and ligand were refreshed every 3-4d. Cells were analyzed at the indicated time points for cytokine profiling and cytotoxicity.

### T-Cell Proliferation

PBMC were pre-activated for 3-4d with 2.5 μg/mL plate-bound α-CD3 (clone OKT3, Biolegend) and 5 μg/mL soluble α-CD28 (Biolegend), then rested for 1d in RPMIc without stimulation. Cells were loaded with 5 uM CellTrace Violet, then plated in 96-well format in media containing 100 nM hIL-2 or surrogate ligands. Live CD4⁺ and CD8⁺ T cells were enumerated using cell surface antibodies and propidium iodide exclusion after 3-5d in culture.

### T-Cell Differentiation

Naïve pan-T cells were isolated to >90% purity from 5-10E+7 cryopreserved PBMCs using an EasySep Human Naïve Pan T Cell Isolation Kit (STEMCELL technologies). Cells were preactivated using 2 μg/mL plate-bound α-CD3 (clone OKT3, Biolegend) and 1 μg/mL soluble α-CD28 (Biolegend) in RPMIc for 4d. Prior to differentiation, cells were washed and rested in RPMIc without stimulation for 1d, then plated into 96-well microplates with 100 nM hIL-2 or IL-2 surrogate ligands. Media and ligand were refreshed after 4d. Cells were analyzed for T memory surface markers or cytokine profiling at 8-10d post differentiation.

### Crystallography

For IL-2R nanobody crystallography, hIL-2Rβ extracellular domain (a.a. 27-233) and hyc extracellular domain (a.a. 55-254) were cloned into the pAcGP67a baculoviral vector carrying an N-terminal GP64 signal sequence and C-terminal 6×His tag. Baculovirus was produced by transfection of Sf9 insect cells with Cellfectin II (Gibco) and Sapphire Baculovirus DNA (Allele) followed by viral amplification in Sf9 cells. Protein was expressed in T. ni cells infected for 48-72 h. For γ_{c} expression, cells were infected in the presence of the endoplasmic reticulum mannosidase I (ERM1) inhibitor, kifunensine (Toronto Research Chemicals). Protein was purified by Ni-NTA affinity chromatography followed by size exclusion chromatography (SEC) using a Superdex S75 increase column (Cytiva). For nanobody expression, sequences were cloned into the pD649 vector with an N-terminal HA signal peptide and C-terminal AviTag and 6×His tag. Nanobodies were expressed by transient transfection in Expi293F cells (Gibco) using an ExpiFectamine 293 Transfection Kit (Gibco) according to manufacturer's protocols. Nanobodies were purified by Ni-NTA affinity chromatography and S75 SEC.

For γ_{c}:γ_{c}-VHH6 crystallography, γ_{c} and γ_{c}-VHH6 were complexed for 4 hours at 4° C. in the presence of carboxypeptidase A (Sigma), carboxypeptidase B (Sigma), and endoglycosidase H (EndoH) in HBS, pH6.8. The complex was purified by S75 SEC and concentrated to 12.9 mg/mL. Crystals were grown in a solution of 2M ammonium sulfate, 0.2M BIS-Tris pH5.5 and flash cooled in liquid nitrogen with the addition of 30% gelycerol as cryoprotectant. Diffraction data were collected at Stanford Linear Accelerator SSRL beamline 12-1. Data were indexed, integrated, and scaled using the XDS package (Kabsch, 2010). The structure was solved by molecular replacement using PHASER with hyc (PDB: 2B5I) (Wang et al., 2005) and a nanobody with loop deletions (PDB: 5LHR) (Kromann-Hansen et al., 2017). The final model was built by iterative rounds of model building in COOT (Emsley et al., 2010) and refinement in PHENIX (Liebschner et al., 2019). All crystallographic software was installed and configured by SBGrid (Morin et al., 2013).

For IL-2Rβ crystallography, IL-2Rβ and IL-2Rβ-VHH6 were methylated with borane dimethylamine complex (Sigma) and paraformaldehyde (Electron Microscopy Sciences) overnight at 4° C. according to previously established protocols (Walter et al., 2006) in the presence of carboxypeptidase A and B (Sigma). The following morning, the reaction was quenched with 200 mM Tris pH8.0 and purified by S75 SEC. The complex was concentrated to 11.7 mg/mL and crystallized in a solution of 0.23M ammonium sulfate, 0.08M BisTris pH5.5 and 23% PEG3350. Crystals were cryoprotected with 20% PEG 400 and flash cooled in liquid nitrogen. Diffraction data were collected at Stanford Linear Accelerator (SSRL 12-2) and processed as described for the γ_{c} structure other than that molecular replacement was performed with IL-2Rβ (PDB: 2B5I) (Wang et al., 2005).

For both structures, data refinement and statistics can be found in Table 1 and were deposited in the RSCB protein databank with accession codes PDB: 7S2R (γ_{c}:γ_{c}-VHH6) and PDB: 7S2S (IL-2Rβ:β-VHH6).

qRT-PCR

RNA was extracted with an RNeasy Plus kit (QIAGEN), converted to cDNA by a RT-PCR reaction (iSCRIPT reverse transcription kit, Bio-rad), and ISG induction relative to the untreated controls and normalized to GAPDH levels were measured by the PowerTrack SYBR green qPCR assay system (Thermo Fisher Scientific) on a StepOnePlus instrument (Thermo Fisher Scientific). The qRT-PCR primers are listed below:

GAPDH-F:
(SEQ ID NO: 111)
ACAACTTTGGTATCGTGGAAGG
GAPDH-R:
(SEQ ID NO: 112)
GCCATCACGCCACAGTTTC
MX1-F:
(SEQ ID NO: 113)
GTTTCCGAAGTGGACATCGCA
MX1-R:
(SEQ ID NO: 114)
CTGCACAGGTTGTTCTCAGC
OAS1-F:
(SEQ ID NO: 115)
TGTCCAAGGTGGTAAAGGGTG
OAS1-R:
(SEQ ID NO: 116)
CCGGCGATTTAACTGATCCTG
CXCL9-F:
(SEQ ID NO: 117)
CCAACCAAGGGACTATCCACC
CXCL9-R:
(SEQ ID NO: 118)
CCTTCACATCTGCTGAATCTGG
CXCL10-F:
(SEQ ID NO: 119)
GTGGCATTCAAGGAGTACCTC
CXCL10-R:
(SEQ ID NO: 120)
TGATGGCCTTCGATTCTGGATT
CCL2-F:
(SEQ ID NO: 121)
CAGCCAGATGCAATCAATGCC
CCL2-R:
(SEQ ID NO: 122)
TGGAATCCTGAACCCACTTCT
CCL3-F:
(SEQ ID NO: 123)
AGTTCTCTGCATCACTTGCTG
CCL3-R:
(SEQ ID NO: 124)
CGGCTTCGCTTGGTTAGGAA
IFIT1-F:
(SEQ ID NO: 125)
TCAGGTCAAGGATAGTCTGGAG
IFIT1-R:
(SEQ ID NO: 126)
AGGTTGTGTATTCCCACACTGTA
IFITM1-F:
(SEQ ID NO: 127)
CCAAGGTCCACCGTGATTAAC
IFITM1-R:
(SEQ ID NO: 128)
ACCAGTTCAAGAAGAGGGTGTT
TRAIL-F:
(SEQ ID NO: 129)
TGCGTGCTGATCGTGATCTTC
TRAIL-R:
(SEQ ID NO: 130)
GCTCGTTGGTAAAGTACACGTA
SeV-F:
(SEQ ID NO: 131)
GACGCGAGTTATGTGTTTGC
SeV-R:
(SEQ ID NO: 132)
TTCCACGCTCTCTTGGATCT

### Anti-Proliferative Activity Essay

Human primary bronchial/tracheal epithelial cells were seeded at 1,000 cells/well in 96-well plates. The following day media was replaced with surrogate ligand or IFN□ containing media. 3 days post IFN treatment cell density was measured using CellTiter-Glo (Promega) according to the manufacturer's protocol.

### A549-hACE2 SARS-COV-2 Antiviral Assay.

96-well plates were seeded with 20,000 A549-hACE2 cells/well. A549 is a human lung epithelial cell line stably expressing the SARS-COV-2 receptor, hACE2, to facilitate efficient infection for antiviral assays (Hou et al., 2020). Culture medium was removed 24 hr. post-seeding, and a 9-point agonist dose-response (top concentration 1000 nM, 10-fold steps) was prepared in “infection medium” (DMEM (Gibco), 5% fetal bovine serum (Hyclone), 1× anti/anti (antibiotic, antimycotic, Gibco). Cells were transported to Biosafety Level 3 after 24 hr. treatment with agonists, at which point cells were infected with recombinant SARS-COV-2 engineered to express nanoluciferase at a multiplicity of infection of 0.25. After incubation for 1 hr. at 37° C., input virus was removed, cells were washed once with infection medium and 100 μL fresh infection medium was added. As a positive control, a similar dose-response of recombinant human IFNω was employed. As a negative control, the monomeric hIFNAR1-specific VHH “A1” was employed, which should not facilitate the dimerization of the type I interferon receptor subunits. After 48 hr. of infection, levels of virus replication were measured by Promega NanoGlo assay measured on a Promega GloMax Luminometer. Similarly treated uninfected sister plates were generated in order to gauge potential cytotoxicity by Promega CellTiter Glo assay read on a Promega GloMax Luminometer.

### NK Cell Culture and Stimulation

PBMCs were isolated by ficoll density gradient centrifugation and resuspended in RPMI supplemented with 10% FBS, 1% L-glutamine, 1% HEPES, 1% MEM Non-Essential Amino Acids Solution, 1% sodium pyruvate, and 1% penicillin streptomycin. NK cells were stimulated with hIL-18 (100 ng/mL, R&D), hIL-15 (20 ng/mL, R&D), and hIL-12 (10 ng/ml, BioLegend) for 18 hr, washed 3 times, then cultured in cRPMI for 2 days.

NKL cells were cultured in cRPMI containing 100 IU human IL-2, with media and IL-2 changes every other day.

### NK Killing Assay, NK Degranulation and NK Activation

NKL cells were rested in cytokine-free media for 2 days. The rested NKL cells were preincubated with 100 nM surrogate ligand or hIL-2 for 12 h. K562 cells were labeled with 15 μM Calcein-AM (BioLegend) for 30 min at 37° C. The NKL cells were cocultured with 10,000 K562 cells at indicated effector: target ratios for 4 h at 37° C. in V bottom 96 well plate. The supernatants were transferred to a new 96 well plate and measured using a Spectramax Gemini dual-scanning microplate. (excitation filter: 485±9 nm; band-pass filter: 530±9 nm).

For degranulation and activation of NKL or primary NK cells, cells were rested and pre-stimulated with surrogate agonists for 12 h. K562 cells were labeled with 1 μM CellTrace Violet (Thermo Fisher) for 20 min at 37° C. NK cells were co-cultured with K562 cells for 4 h in the presence of FITC-CD107 antibody (BioLegend), GolgiStop and GolgiPlug (BD). The cells were surface stained with NK markers and CD69 antibody for 30 min on ice. IFNγ staining was performed by following the intracellular staining protocol (Invitrogen). The samples were analyzed via flow cytometry.

TABLE 1
Crystallographic data collection and refinement statistics.

| | IL2Rβ:β-VHH6 | γ_{c}:γ_{c}-VHH6 |
|---|---|---|
| Wavelength | 0.9795 | 0.9795 |
| Resolution range | 42.05-1.932 (2.001-1.932) | 44.15-2.49 (2.579-2.49) |
| Space group | P 1 | I 41 2 2 |
| Unit cell | 42.698 69.505 91.721 | 120.082 120.082 |
| | 99.566 99.854 90.191 | 237.38 90 90 90 |
| Total reflections | 263616 (25045) | 367179 (37470) |
| Unique | 73786 (7211) | 30736 (3015) |
| reflections | | |
| Completeness (%) | 95.93 (93.19) | 99.10 (96.36) |
| Mean I/sigma(I) | 7.79 (1.00) | 4.27 (0.41) |
| Wilson B-factor | 31.57 | 75.85 |
| R-meas | 0.1185 (1.429) | 0.3262 (7.646) |
| R-pim | 0.06211 (0.7516) | 0.09613 (2.147) |
| CC1/2 | 0.996 (0.587) | 0.983 (0.331) |
| CC* | 0.999 (0.86) | 0.996 (0.705) |
| Reflections used | 73632 (7104) | 30519 (2912) |
| in refinement | | |
| Reflections used | 2007 (192) | 2301 (218) |
| for R-free | | |
| R-work | 0.2123 (0.3496) | 0.2459 (0.5002) |
| R-free | 0.2464 (0.3599) | 0.2715 (0.4672) |
| Number of non- | 5660 | 2841 |
| hydrogen atoms | | |
| macromolecules | 5171 | 2683 |
| ligands | 295 | 132 |
| solvent | 194 | 26 |
| Protein residues | 657 | 333 |
| RMS(bonds) | 0.004 | 0.003 |
| RMS(angles) | 0.71 | 0.56 |
| Ramachandran | 96.18 | 98.18 |
| favored (%) | | |
| Ramachandran | 3.82 | 1.82 |
| allowed (%) | | |
| Ramachandran | 0.00 | 0.00 |
| outliers (%) | | |
| Rotamer | 1.28 | 2.42 |
| outliers (%) | | |
| Clashscore | 3.19 | 5.94 |
| Average B-factor | 54.65 | 96.32 |
| macromolecules | 54.63 | 95.24 |
| ligands | 64.65 | 119.87 |
| solvent | 39.98 | 87.57 |
| Number of | 23 | 14 |
| TLS groups | | |

### Statistics for the Highest-Resolution Shell are Shown in Parentheses.

INFORMAL SEQUENCE LISTING

| SEQ | | | |
|---|---|---|---|
| ID | Module | Description | |
| NO | Name | (Module) | Sequence |
| 1 | 2Rβ-VHH1 | VHH Binding | QVQLQESGGGSVQAGGSLRLSCVTSGYTYSSANMAWFRQAPGKEREGVAII |
| | | Module | TPSGRATTYADSVKGRFTISRDNAANTLYLQMNSLKPEDTAMYYCAADTPP |
| | | | YSGLWYAERTYNYWGQGTQVTVSS |
| 2 | 2Rβ-VHH3 | VHH Binding | QVQLQESGGGSVQAGGSLRLSCTASGFTFDDEDMGWYRQAPGNECELVS |
| | | Module | SIGSLGRRYYADSVKDRFAISQDNAKNTVYLQMNSLKPEDTAVYYCAAT |
| | | | KGGSWLDSILASCQGAFGYWGQGTQVTVSS |
| 3 | 2Rβ-VHH4 | VHH Binding | QVQLQESGGGSVQAGGSLRLSCAASGSTSCSSVMRWYRQAPGKEREFVSS |
| | | Module | INSDRRTVYADSVKGRFTISQDNAKSTLYLQMNSLKAEDTATYYCQRELY |
| | | | GDSWCQGNYWGQGTQVTVSS |
| 4 | 2Rβ-VHH6 | VHH Binding | QVQLQESGGGSVQAGGSLRLSCAASSYTISSVCMGWFRQAPGKEREGVA |
| | | Module | GIAPDGSTGYGDSVKGRFTISKDNAKNTLYLQMNSLKPEDTAMYYCAAA |
| | | | SPGRCFLPRTALEPALYYNWGQGTQVTVSS |
| 5 | γ_{c}-VHH3 | VHH Binding | QVQLQESGGGSVQAGGSLRLSCAASGYTYSKNWYMGWFRQTPGKEREG |
| | | Module | VAVIAYDDWPTYADSVKGRFTISKDNTKNTLYLQMNSLKPEDTAMYYCA |
| | | | ARQLGGDYCYFPNLSRFCYNYWGQGTQVTVSS |
| 6 | γ_{c}-VHH4 | VHH Binding | QVQLQESGGGSVQAGGSLRLSCTASGFTFNEANHMGWYRQAPGNECELV |
| | | Module | STISSDGTTYYPDSVKGRFTISQDNAKKTAFLQMNSLKPEDTAVYYCAAD |
| | | | QSRRGSLCLGQGTQVTVSS |
| 7 | γ_{c}-VHH6 | VHH Binding | QVQLQESGGGSVQAGGSLRLSCAASGYTYRDYYMGWFRQAPGREREGV |
| | | Module | ASIYTRGSREGSTRYSSSVEGRFTITLDTAKNTLYLQMNSLKPEDTAMYYC |
| | | | AADDRTWLPRVQLGGPRENEYNYWGQGTQVTVSS |

Human IL-2 surrogate agonists: sequences of active molecules from initial screen
(refers to FIG. 1D left, β-γ_{c} Forward orientation)

| | | | |
|---|---|---|---|
| 8 | MY144-F | 2Rβ-VHH1 | QVQLQESGGGSVQAGGSLRLSCVTSGYTYSSANMAWFRQAPGKEREGVA |
| | | γ_{c}-VHH4 | IITPSGRATTYADSVKGRFTISRDNAANTLYLQMNSLKPEDTAMYYCAAD |
| | | | TPPYSGLWYAERTYNYWGQGTQVTVSSSFQVQLQESGGGSVQAGGSLRL |
| | | | SCTASGFTFNEANHMGWYRQAPGNECELVSTISSDGTTYYPDSVKGRFTIS |
| | | | QDNAKKTAFLQMNSLKPEDTAVYYCAADQSRRGSLCLGQGTQVTVSS |
| 9 | MY141-F | 2Rβ-VHH3 | QVQLQESGGGSVQAGGSLRLSCTASGFTFDDEDMGWYRQAPGNECELVS |
| | | γ_{c}-VHH3 | SIGSLGRRYYADSVKDRFAISQDNAKNTVYLQMNSLKPEDTAVYYCAAT |
| | | | KGGSWLDSILASCQGAFGYWGQGTQVTVSSSFQVQLQESGGGSVQAGGS |
| | | | LRLSCAASGYTYSKNWYMGWFRQTPGKEREGVAVIAYDDWPTYADSVK |
| | | | GRFTISKDNTKNTLYLQMNSLKPEDTAMYYCAARQLGGDYCYFPNLSRFC |
| | | | YNYWGQGTQVTVSS |
| 10 | MY145-F | 2Rβ-VHH3 | QVQLQESGGGSVQAGGSLRLSCTASGFTFDDEDMGWYRQAPGNECELVS |
| | | γ_{c}-VHH4 | SIGSLGRRYYADSVKDRFAISQDNAKNTVYLQMNSLKPEDTAVYYCAAT |
| | | | KGGSWLDSILASCQGAFGYWGQGTQVTVSSSFQVQLQESGGGSVQAGGS |
| | | | LRLSCTASGFTFNEANHMGWYRQAPGNECELVSTISSDGTTYYPDSVKGR |
| | | | FTISQDNAKKTAFLQMNSLKPEDTAVYYCAADQSRRGSLCLGQGTQVTVS |
| | | | S |
| 11 | MY143-F | 2Rβ-VHH3 | QVQLQESGGGSVQAGGSLRLSCTASGFTFDDEDMGWYRQAPGNECELVS |
| | | γ_{c}-VHH6 | SIGSLGRRYYADSVKDRFAISQDNAKNTVYLQMNSLKPEDTAVYYCAAT |
| | | | KGGSWLDSILASCQGAFGYWGQGTQVTVSSSFQVQLQESGGGSVQAGGS |
| | | | LRLSCAASGYTYRDYYMGWFRQAPGREREGVASIYTRGSREGSTRYSSSV |
| | | | EGRFTITLDTAKNTLYLQMNSLKPEDTAMYYCAADDRTWLPRVQLGGPR |
| | | | ENEYNYWGQGTQVTVSS |
| 12 | MY189-F | 2Rβ-VHH3 | QVQLQESGGGSVQAGGSLRLSCTASGFTFDDEDMGWYRQAPGNECELVS |
| | | γ_{c}-scFv1 (P1A3) | SIGSLGRRYYADSVKDRFAISQDNAKNTVYLQMNSLKPEDTAVYYCAAT |
| | | | KGGSWLDSILASCQGAFGYWGQGTQVTVSSGGGGTSASQVQLQQWGAG |
| | | | LLKPSETLSLTCAVYGGSFSGYYWSWIRQPPGKGLEWIGEINHSGSTNYNP |
| | | | SLKSRATISVDTSKNQFSLKLSSVTAADTAVYYCATSPGGYSGGYFQHWG |
| | | | QGTLVTVSSGGGGSGGGGSGGGGSDVVMTQSPLSLPVTPGEPASISCRSSQ |
| | | | SLLHSNGYNYLDWYLQKPGQSPQLLIYLGSNRDSGVPDRFSGSGSGTDFTL |
| | | | KISRVEAEDVGVYYCMQGTHWPWTFGQGTKVEIK |
| 13 | MY193-F | 2Rβ-VHH3 | QVQLQESGGGSVQAGGSLRLSCTASGFTFDDEDMGWYRQAPGNECELVS |
| | | γ_{c}-scFv2 (P2B9) | SIGSLGRRYYADSVKDRFAISQDNAKNTVYLQMNSLKPEDTAVYYCAAT |
| | | | KGGSWLDSILASCQGAFGYWGQGTQVTVSSGGGGTSASQVQLQESGPGL |
| | | | VKPSETLSLTCTVSGGSISSSSYYWGWIRQPPGKGLEWIGSIYYSGSTYYNP |
| | | | SLKSRVTISVDTSKNQFSLKLSSVTAADTAVYYCAGDILTGYALDYWGQG |
| | | | TLVTVSSGGGGSGGGGSGGGGSSYELTQPPSMSVSPGQTARITCSGDALPK |
| | | | QFAFWYQQKPGQAPVLVIYKDTERPSGIPERFSGSSSGTTVTLTITGVQAED |
| | | | EADYYCQSPDSSGTVEVFGGGTKLTVL |
| 14 | MY178-F | 2Rβ-VHH4 | QVQLQESGGGSVQAGGSLRLSCAASGSTSCSSVMRWYRQAPGKEREFVSS |
| | | γ_{c}-VHH4 | INSDRRTVYADSVKGRFTISQDNAKSTLYLQMNSLKAEDTATYYCQRELY |
| | | | GDSWCQGNYWGQGTQVTVSSSFQVQLQESGGGSVQAGGSLRLSCTASGF |
| | | | TFNEANHMGWYRQAPGNECELVSTISSDGTTYYPDSVKGRFTISQDNAKK |
| | | | TAFLQMNSLKPEDTAVYYCAADQSRRGSLCLGQGTQVTVSS |
| 15 | MY179-F | 2Rβ-VHH4 | QVQLQESGGGSVQAGGSLRLSCAASGSTSCSSVMRWYRQAPGKEREFVSS |
| | | γ_{c}-VHH6 | INSDRRTVYADSVKGRFTISQDNAKSTLYLQMNSLKAEDTATYYCQRELY |
| | | | GDSWCQGNYWGQGTQVTVSSSFQVQLQESGGGSVQAGGSLRLSCAASG |
| | | | YTYRDYYMGWFRQAPGREREGVASIYTRGSREGSTRYSSSVEGRFTITLDT |
| | | | AKNTLYLQMNSLKPEDTAMYYCAADDRTWLPRVQLGGPRENEYNYWG |
| | | | QGTQVTVSS |
| 16 | MY190-F | 2Rβ-VHH4 | QVQLQESGGGSVQAGGSLRLSCAASGSTSCSSVMRWYRQAPGKEREFVSS |
| | | γ_{c}-scFv1 (P1A3) | INSDRRTVYADSVKGRFTISQDNAKSTLYLQMNSLKAEDTATYYCQRELY |
| | | | GDSWCQGNYWGQGTQVTVSSGGGGTSASQVQLQQWGAGLLKPSETLSL |
| | | | TCAVYGGSFSGYYWSWIRQPPGKGLEWIGEINHSGSTNYNPSLKSRATISV |
| | | | DTSKNQFSLKLSSVTAADTAVYYCATSPGGYSGGYFQHWGQGTLVTVSS |
| | | | GGGGSGGGGSGGGGSDVVMTQSPLSLPVTPGEPASISCRSSQSLLHSNGYN |
| | | | YLDWYLQKPGQSPQLLIYLGSNRDSGVPDRFSGSGSGTDFTLKISRVEAED |
| | | | VGVYYCMQGTHWPWTFGQGTKVEIK |
| 17 | MY194-F | 2Rβ-VHH4 | QVQLQESGGGSVQAGGSLRLSCAASGSTSCSSVMRWYRQAPGKEREFVSS |
| | | γ_{c}-scFv2 (P2B9) | INSDRRTVYADSVKGRFTISQDNAKSTLYLQMNSLKAEDTATYYCQRELY |
| | | | GDSWCQGNYWGQGTQVTVSSGGGGTSASQVQLQESGPGLVKPSETLSLT |
| | | | CTVSGGSISSSSYYWGWIRQPPGKGLEWIGSIYYSGSTYYNPSLKSRVTISV |
| | | | DTSKNQFSLKLSSVTAADTAVYYCAGDILTGYALDYWGQGTLVTVSSGG |
| | | | GGSGGGGSGGGGSSYELTQPPSMSVSPGQTARITCSGDALPKQFAFWYQQ |
| | | | KPGQAPVLVIYKDTERPSGIPERFSGSSSGTTVTLTITGVQAEDEADYYCQS |
| | | | PDSSGTVEVFGGGTKLTVL |
| 18 | MY172-F | 2Rβ-VHH6 | QVQLQESGGGSVQAGGSLRLSCAASSYTISSVCMGWFRQAPGKEREGVA |
| | | γ_{c}-VHH4 | GIAPDGSTGYGDSVKGRFTISKDNAKNTLYLQMNSLKPEDTAMYYCAAA |
| | | | SPGRCFLPRTALEPALYYNWGQGTQVTVSSSFQVQLQESGGGSVQAGGSL |
| | | | RLSCTASGFTFNEANHMGWYRQAPGNECELVSTISSDGTTYYPDSVKGRF |
| | | | TISQDNAKKTAFLQMNSLKPEDTAVYYCAADQSRRGSLCLGQGTQVTVSS |
| 19 | MY173-F | 2Rβ-VHH6 | QVQLQESGGGSVQAGGSLRLSCAASSYTISSVCMGWFRQAPGKEREGVA |
| | | γ_{c}-VHH6 | GIAPDGSTGYGDSVKGRFTISKDNAKNTLYLQMNSLKPEDTAMYYCAAA |
| | | | SPGRCFLPRTALEPALYYNWGQGTQVTVSSSFQVQLQESGGGSVQAGGSL |
| | | | RLSCAASGYTYRDYYMGWFRQAPGREREGVASIYTRGSREGSTRYSSSVE |
| | | | GRFTITLDTAKNTLYLQMNSLKPEDTAMYYCAADDRTWLPRVQLGGPRE |
| | | | NEYNYWGQGTQVTVSS |

Human IL-2 surrogate agonists: sequences of active molecules from initial screen
(refers to FIG. 1D right, γ_{c}-β Reverse orientation)

| | | | γ_{c}-VHH-IL2Rβ-VHH (Reverse) |
|---|---|---|---|
| | | | orientation, 2 or 8 a.a. Linker |
| 20 | MY140-R | γ_{c}-VHH3 | QVQLQESGGGSVQAGGSLRLSCAASGYTYSKNWYMGWFRQTPGKEREG |
| | | 2Rβ-VHH1 | VAVIAYDDWPTYADSVKGRFTISKDNTKNTLYLQMNSLKPEDTAMYYCA |
| | | | ARQLGGDYCYFPNLSRFCYNYWGQGTQVTVSSSFQVQLQESGGGSVQAG |
| | | | GSLRLSCVTSGYTYSSANMAWFRQAPGKEREGVAIITPSGRATTYADSVK |
| | | | GRFTISRDNAANTLYLQMNSLKPEDTAMYYCAADTPPYSGLWYAERTYN |
| | | | YWGQGTQVTVSS |
| 21 | MY142-R | γ_{c}-VHH6 | QVQLQESGGGSVQAGGSLRLSCAASGYTYRDYYMGWFRQAPGREREGV |
| | | 2Rβ-VHH1 | ASIYTRGSREGSTRYSSSVEGRFTITLDTAKNTLYLQMNSLKPEDTAMYYC |
| | | | AADDRTWLPRVQLGGPRENEYNYWGQGTQVTVSSSFQVQLQESGGGSV |
| | | | QAGGSLRLSCVTSGYTYSSANMAWFRQAPGKEREGVAIITPSGRATTYAD |
| | | | SVKGRFTISRDNAANTLYLQMNSLKPEDTAMYYCAADTPPYSGLWYAER |
| | | | TYNYWGQGTQVTVSS |
| 22 | MY188-R | γ_{c}-scFv1 (P1A3) | QVQLQQWGAGLLKPSETLSLTCAVYGGSFSGYYWSWIRQPPGKGLEWIG |
| | | 2Rβ-VHH1 | EINHSGSTNYNPSLKSRATISVDTSKNQFSLKLSSVTAADTAVYYCATSPG |
| | | | GYSGGYFQHWGQGTLVTVSSGGGGSGGGGSGGGGSDVVMTQSPLSLPVT |
| | | | PGEPASISCRSSQSLLHSNGYNYLDWYLQKPGQSPQLLIYLGSNRDSGVPD |
| | | | RFSGSGSGTDFTLKISRVEAEDVGVYYCMQGTHWPWTFGQGTKVEIKGG |
| | | | GGTSASQVQLQESGGGSVQAGGSLRLSCVTSGYTYSSANMAWFRQAPGK |
| | | | EREGVAIITPSGRATTYADSVKGRFTISRDNAANTLYLQMNSLKPEDTAMY |
| | | | YCAADTPPYSGLWYAERTYNYWGQGTQVTVSS |
| 23 | MY192-R | γ_{c}-scFv2 (P2B9) | QVQLQESGPGLVKPSETLSLTCTVSGGSISSSSYYWGWIRQPPGKGLEWIG |
| | | 2Rβ-VHH1 | SIYYSGSTYYNPSLKSRVTISVDTSKNQFSLKLSSVTAADTAVYYCAGDILT |
| | | | GYALDYWGQGTLVTVSSGGGGSGGGGSGGGGSSYELTQPPSMSVSPGQT |
| | | | ARITCSGDALPKQFAFWYQQKPGQAPVLVIYKDTERPSGIPERFSGSSSGTT |
| | | | VTLTITGVQAEDEADYYCQSPDSSGTVEVFGGGTKLTVLGGGGTSASQV |
| | | | QLQESGGGSVQAGGSLRLSCVTSGYTYSSANMAWFRQAPGKEREGVAIIT |
| | | | PSGRATTYADSVKGRFTISRDNAANTLYLQMNSLKPEDTAMYYCAADTPP |
| | | | YSGLWYAERTYNYWGQGTQVTVSS |
| 24 | MY141-R | γ_{c}-VHH3 | QVQLQESGGGSVQAGGSLRLSCAASGYTYSKNWYMGWFRQTPGKEREG |
| | | 2Rβ-VHH3 | VAVIAYDDWPTYADSVKGRFTISKDNTKNTLYLQMNSLKPEDTAMYYCA |
| | | | ARQLGGDYCYFPNLSRFCYNYWGQGTQVTVSSSFQVQLQESGGGSVQAG |
| | | | GSLRLSCTASGFTFDDEDMGWYRQAPGNECELVSSIGSLGRRYYADSVKD |
| | | | RFAISQDNAKNTVYLQMNSLKPEDTAVYYCAATKGGSWLDSILASCQGA |
| | | | FGYWGQGTQVTVSS |
| 25 | MY145-R | γ_{c}-VHH4 | QVQLQESGGGSVQAGGSLRLSCTASGFTFNEANHMGWYRQAPGNECELV |
| | | 2Rβ-VHH3 | STISSDGTTYYPDSVKGRFTISQDNAKKTAFLQMNSLKPEDTAVYYCAAD |
| | | | QSRRGSLCLGQGTQVTVSSSFQVQLQESGGGSVQAGGSLRLSCTASGFTF |
| | | | DDEDMGWYRQAPGNECELVSSIGSLGRRYYADSVKDRFAISQDNAKNTV |
| | | | YLQMNSLKPEDTAVYYCAATKGGSWLDSILASCQGAFGYWGQGTQVTV |
| | | | SS |
| 26 | MY143-R | γ_{c}-VHH6 | QVQLQESGGGSVQAGGSLRLSCAASGYTYRDYYMGWFRQAPGREREGV |
| | | 2Rβ-VHH3 | ASIYTRGSREGSTRYSSSVEGRFTITLDTAKNTLYLQMNSLKPEDTAMYYC |
| | | | AADDRTWLPRVQLGGPRENEYNYWGQGTQVTVSSSFQVQLQESGGGSV |
| | | | QAGGSLRLSCTASGFTFDDEDMGWYRQAPGNECELVSSIGSLGRRYYADS |
| | | | VKDRFAISQDNAKNTVYLQMNSLKPEDTAVYYCAATKGGSWLDSILASC |
| | | | QGAFGYWGQGTQVTVSS |
| 27 | MY189-R | γ_{c}-scFv1 (P1A3) | QVQLQQWGAGLLKPSETLSLTCAVYGGSFSGYYWSWIRQPPGKGLEWIG |
| | | 2Rβ-VHH3 | EINHSGSTNYNPSLKSRATISVDTSKNQFSLKLSSVTAADTAVYYCATSPG |
| | | | GYSGGYFQHWGQGTLVTVSSGGGGSGGGGSGGGGSDVVMTQSPLSLPVT |
| | | | PGEPASISCRSSQSLLHSNGYNYLDWYLQKPGQSPQLLIYLGSNRDSGVPD |
| | | | RFSGSGSGTDFTLKISRVEAEDVGVYYCMQGTHWPWTFGQGTKVEIKGG |
| | | | GGTSASQVQLQESGGGSVQAGGSLRLSCTASGFTFDDEDMGWYRQAPGN |
| | | | ECELVSSIGSLGRRYYADSVKDRFAISQDNAKNTVYLQMNSLKPEDTAVY |
| | | | YCAATKGGSWLDSILASCQGAFGYWGQGTQVTVSS |
| 28 | MY193-R | γ_{c}-scFv2 (P2B9) | QVQLQESGPGLVKPSETLSLTCTVSGGSISSSSYYWGWIRQPPGKGLEWIG |
| | | 2Rβ-VHH3 | SIYYSGSTYYNPSLKSRVTISVDTSKNQFSLKLSSVTAADTAVYYCAGDILT |
| | | | GYALDYWGQGTLVTVSSGGGGSGGGGSGGGGSSYELTQPPSMSVSPGQT |
| | | | ARITCSGDALPKQFAFWYQQKPGQAPVLVIYKDTERPSGIPERFSGSSSGTT |
| | | | VTLTITGVQAEDEADYYCQSPDSSGTVEVFGGGTKLTVLGGGGTSASQV |
| | | | QLQESGGGSVQAGGSLRLSCTASGFTFDDEDMGWYRQAPGNECELVSSIG |
| | | | SLGRRYYADSVKDRFAISQDNAKNTVYLQMNSLKPEDTAVYYCAATKGG |
| | | | SWLDSILASCQGAFGYWGQGTQVTVSS |
| 29 | MY190-R | γ_{c}-scFv1 (P1A3) | QVQLQQWGAGLLKPSETLSLTCAVYGGSFSGYYWSWIRQPPGKGLEWIG |
| | | 2Rβ-VHH4 | EINHSGSTNYNPSLKSRATISVDTSKNQFSLKLSSVTAADTAVYYCATSPG |
| | | | GYSGGYFQHWGQGTLVTVSSGGGGSGGGGSGGGGSDVVMTQSPLSLPVT |
| | | | PGEPASISCRSSQSLLHSNGYNYLDWYLQKPGQSPQLLIYLGSNRDSGVPD |
| | | | RFSGSGSGTDFTLKISRVEAEDVGVYYCMQGTHWPWTFGQGTKVEIKGG |
| | | | GGTSASQVQLQESGGGSVQAGGSLRLSCAASGSTSCSSVMRWYRQAPGK |
| | | | EREFVSSINSDRRTVYADSVKGRFTISQDNAKSTLYLQMNSLKAEDTATYY |
| | | | CQRELYGDSWCQGNYWGQGTQVTVSS |
| 30 | MY194-R | γ_{c}-scFv2 (P2B9) | QVQLQESGPGLVKPSETLSLTCTVSGGSISSSSYYWGWIRQPPGKGLEWIG |
| | | 2Rβ-VHH4 | SIYYSGSTYYNPSLKSRVTISVDTSKNQFSLKLSSVTAADTAVYYCAGDILT |
| | | | GYALDYWGQGTLVTVSSGGGGSGGGGSGGGGSSYELTQPPSMSVSPGQT |
| | | | ARITCSGDALPKQFAFWYQQKPGQAPVLVIYKDTERPSGIPERFSGSSSGTT |
| | | | VTLTITGVQAEDEADYYCQSPDSSGTVEVFGGGTKLTVLGGGGTSASQV |
| | | | QLQESGGGSVQAGGSLRLSCAASGSTSCSSVMRWYRQAPGKEREFVSSIN |
| | | | SDRRTVYADSVKGRFTISQDNAKSTLYLQMNSLKAEDTATYYCQRELYG |
| | | | DSWCQGNYWGQGTQVTVSS |
| 31 | MY171-R | γ_{c}-VHH3 | QVQLQESGGGSVQAGGSLRLSCAASGYTYSKNWYMGWFRQTPGKEREG |
| | | 2Rβ-VHH6 | VAVIAYDDWPTYADSVKGRFTISKDNTKNTLYLQMNSLKPEDTAMYYCA |
| | | | ARQLGGDYCYFPNLSRFCYNYWGQGTQVTVSSSFQVQLQESGGGSVQAG |
| | | | GSLRLSCAASSYTISSVCMGWFRQAPGKEREGVAGIAPDGSTGYGDSVKG |
| | | | RFTISKDNAKNTLYLQMNSLKPEDTAMYYCAAASPGRCFLPRTALEPALY |
| | | | YNWGQGTQVTVSS |
| 32 | MY172-R | γ_{c}-VHH4 | QVQLQESGGGSVQAGGSLRLSCTASGFTFNEANHMGWYRQAPGNECELV |
| | | 2Rβ-VHH6 | STISSDGTTYYPDSVKGRFTISQDNAKKTAFLQMNSLKPEDTAVYYCAAD |
| | | | QSRRGSLCLGQGTQVTVSSGFQVQLQESGGGSVQAGGSLRLSCAASSYTIS |
| | | | SVCMGWFRQAPGKEREGVAGIAPDGSTGYGDSVKGRFTISKDNAKNTLY |
| | | | LQMNSLKPEDTAMYYCAAASPGRCFLPRTALEPALYYNWGQGTQVTVSS |
| 33 | MY173-R | γ_{c}-VHH6 | QVQLQESGGGSVQAGGSLRLSCAASGYTYRDYYMGWFRQAPGREREGV |
| | | 2Rβ-VHH6 | ASIYTRGSREGSTRYSSSVEGRFTITLDTAKNTLYLQMNSLKPEDTAMYYC |
| | | | AADDRTWLPRVQLGGPRENEYNYWGQGTQVTVSSSFQVQLQESGGGSV |
| | | | QAGGSLRLSCAASSYTISSVCMGWFRQAPGKEREGVAGIAPDGSTGYGDS |
| | | | VKGRFTISKDNAKNTLYLQMNSLKPEDTAMYYCAAASPGRCFLPRTALEP |
| | | | ALYYNWGQGTQVTVSS |
| 34 | MY191-R | γ_{c}-scFv1 (P1A3) | QVQLQQWGAGLLKPSETLSLTCAVYGGSFSGYYWSWIRQPPGKGLEWIG |
| | | 2Rβ-VHH6 | EINHSGSTNYNPSLKSRATISVDTSKNQFSLKLSSVTAADTAVYYCATSPG |
| | | | GYSGGYFQHWGQGTLVTVSSGGGGSGGGGSGGGGSDVVMTQSPLSLPVT |
| | | | PGEPASISCRSSQSLLHSNGYNYLDWYLQKPGQSPQLLIYLGSNRDSGVPD |
| | | | RFSGSGSGTDFTLKISRVEAEDVGVYYCMQGTHWPWTFGQGTKVEIKGG |
| | | | GGTSASQVQLQESGGGSVQAGGSLRLSCAASSYTISSVCMGWFRQAPGKE |
| | | | REGVAGIAPDGSTGYGDSVKGRFTISKDNAKNTLYLQMNSLKPEDTAMY |
| | | | YCAAASPGRCFLPRTALEPALYYNWGQGTQVTVSS |
| 35 | MY195-R | γ_{c}-scFv2 (P2B9) | QVQLQESGPGLVKPSETLSLTCTVSGGSISSSSYYWGWIRQPPGKGLEWIG |
| | | 2Rβ-VHH6 | SIYYSGSTYYNPSLKSRVTISVDTSKNQFSLKLSSVTAADTAVYYCAGDILT |
| | | | GYALDYWGQGTLVTVSSGGGGSGGGGSGGGGSSYELTQPPSMSVSPGQT |
| | | | ARITCSGDALPKQFAFWYQQKPGQAPVLVIYKDTERPSGIPERFSGSSSGTT |
| | | | VTLTITGVQAEDEADYYCQSPDSSGTVEVFGGGTKLTVLGGGGTSASQV |
| | | | QLQESGGGSVQAGGSLRLSCAASSYTISSVCMGWFRQAPGKEREGVAGIA |
| | | | PDGSTGYGDSVKGRFTISKDNAKNTLYLQMNSLKPEDTAMYYCAAASPG |
| | | | RCFLPRTALEPALYYNWGQGTQVTVSS |

Human Type I IFN surrogate agonists: sequences of individual VHH binding modules

| | | |
|---|---|---|
| 36 | IFNAR1-VHH1 | QVQLQESGGGSVQAGGSLRLSCASSGYTYSNNCMGWFRQAPGKEREGVA |
| | | AIYTGGGSTYYADSVKGRFTTSQDNAKNTVYLQMNSLKPEDTAMYYCAA |
| | | VESRTYCTPDALKRFGYRGQGTQVTVSS |
| 37 | IFNAR1-VHH5 | QVQLQESGGGSVQAGGSLRLSCACEASRYTYSSNCMGWFRQTPGKEREG |
| | | VAAISTASGSTYYADSVKGRFTISQDNAKNTVYLQMNSLKPEDTAMYYC |
| | | AVVESSTYCTPDALKRFGYWGQGTQVTVSS |
| 38 | IFNAR1-VHH6 | QVQLQESGGGSVQAGGSLRLSCAVSGYGNRNYYSGWFRQAPGKGREGV |
| | | AVIDTYGDIRYGDFVKGRFTISKDSAKNTLYLQMNNLSPEDSAMYYCAAS |
| | | RGYYRNYALREDEYAYWGQGTQVTVSS |
| 39 | IFNAR1-VHHA1 | QVQLQESGGGLVQAGGSLRLSCAASGNIFSYDYMGWYRQAPGKEREFVA |
| | | GITVGGSTNYADSVKGRFTISRDNAKNTVYLQMNSLKPEDTAVYYCAAW |
| | | WYDSGYFAYWGQGTQVTVSS |
| 40 | IFNAR1-VHHA5 | QVQLQESGGGLVQAGGSLRLSCAASGTIFLFDYMGWYRQAPGKEREFVA |
| | | GIARGASTNYADSVKGRFTISRDNAKNTVYLQMNSLKPEDTAVYYCAVLP |
| | | YLYFPPDGDPANYLDYWGQGTQVTVSS |
| 41 | IFNAR1-VHHA11 | QVQLQESGGGLVQAGGSLRLSCAASGSIFTVYEMGWYRQAPGKEREFVA |
| | | SISYGASTYYADSVKGRFTISRDNAKNTVYLQMNSLKPEDTAVYYCAVVA |
| | | YVTDQWVGYYTPRYAYWGQGTQVTVSS |
| 42 | IFNAR1-VHHB4 | QVQLQESGGGLVQAGGSLRLSCAASGNIFPYYGMGWYRQAPGKERELVA |
| | | GIAIGASTNYADSVKGRFTISRDNAKNTVYLQMNSLKPEDTAVYYCAVIL |
| | | GPYNGNINYYLYWGQGTQVTVSS |
| 43 | IFNAR1-VHHC1 | QVQLQESGGGLVQAGGSLRLSCAASGTISKPWGMGWYRQAPGKEREFVA |
| | | TIDGGSTTNYADSVKGRFTISRDNAKNTVYLQMNSLKPEDTAVYYCAAV |
| | | YADLDVVLYYHIYWGQGTQVTVSS |
| 44 | IFNAR1-VHHH7 | QVQLQESGGGLVQAGGSLRLSCAASGYIFYPFGMGWYRQAPGKEREFVA |
| | | GINDGTTTNYADSVKGRFTISRDNAKNTVYLQMNSLKPEDTAVYYCAVV |
| | | YTNGPARTRGDNYYHIYWGQGTQVTVSS |
| 45 | IFNAR2-VHH1 | QVQLQESGGGSVQPGGSLRLSCAASGFTFSNYIMSWVRQAPGKGLEWVS |
| | | AISGGGNTYYTDSVKGRFTISRDNAKNTLYLQLNSLKTEDTAMYYCAQN |
| | | WWVAGRSPNPPGQGTQVTVSS |
| 46 | IFNAR2-VHH2 | QVQLQESGGGLVQPGGSLRLSCAASGFTFSDYIMTWVRQAPGKGLEWVS |
| | | SINNGGRSTYYADSVKGRFTISRDNAKNTLYLQLNSLKIEDTAMYYCARD |
| | | VTCWGARTGCRVSGGTQVTVSS |
| 47 | IFNAR2-VHH3 | QVQLQESGGGLVQPGGSLRLSCAASEFTFSEYSMKWVRQPPGKGLEWVS |
| | | TISPSGGTTRYAESVKGRFTISRDNAKNTLYLQLNSLKTEDTAMYYCSVAD |
| | | WSVTQRRGQGTQVTVSS |
| 48 | IFNAR2-VHH5 | QVQLQESGGGLVQPGGSLTLSCAASGFTFRNHIMSWVRQAPGKGLEWVS |
| | | AISSGGGNTYYADSVKGRFTISRDNAKSTLYLQLNSLKTEDTAMYYCTKD |
| | | WARPPYSDFLAPQAQGTQVTVSS |
| 49 | IFNAR2-VHH8 | QVQLQESGGGSVQAGGSLRLSCAASGFYGTYSMGWFRQAPGKEREGVAH |
| | | IESDGSTRYADSMKGRFTVSKDNAKKILYLQMNSLKPEDTAVYYCAADPR |
| | | PYFRTWYERARYGGQGTQVTVSS |
| 50 | IFNAR2-VHH10 | QVQLQESGGGLVQPGGSLRLSCAASGFTFSNHIMSWVRQAPGKGLEWVS |
| | | AINSGGGSTYYRDSVKGRFTISRDNAKNTLYLQLNSLKTEDTAMYYCAKD |
| | | WWRYGSDKPPGQGTQVTVSS |

Human Type I IFN surrogate agonists: sequences
of active molecules from initial screen (refers to FIG. 11A)

| | | | IFNAR1-IFNAR2 orientation |
|---|---|---|---|
| | | | Linker = 5 a.a. (GTSAS) |
| 51 | This hit was | IFNAR1-scFv | QVQLQQWGAGLLKPSETLSLTCAVYGGSFSGYFWSWIRQPPGKGLEWIGE |
| | optimized by | 3F11 | IDHSGKTNYNPSLKSRVTISVDTSKNQVSLKLSSVTAADTAVYYCARESKY |
| | shortening the | IFNAR2-VHH1 | YFGLDVWGQGTTVTVTSGGGGSGGGGSGGGGSAIQLTQSPSSLSASVGDR |
| | linker to 2 a.a. | | VTITCRASQGIYSVLAWYQQKPGKTPKLLIYDASRLESGVPSRFSGSGSGT |
| | The short | | DFTLTISSLQPEDFATYYCQQFNSYITFGQGTRLEIKGTSASQVQLQESGGG |
| | linker variant | | SVQPGGSLRLSCAASGFTFSNYIMSWVRQAPGKGLEWVSAISGGGNTYYT |
| | is named | | DSVKGRFTISRDNAKNTLYLQLNSLKTEDTAMYYCAQNWWVAGRSPNPP |
| | “HIS1”. | | GQGTQVTVSS |
| 52 | This hit was | IFNAR1-scFv | QVQLQQWGAGLLKPSETLSLTCAVYGGSFSGYFWSWIRQPPGKGLEWIGE |
| | optimized by | 3F11 | IDHSGKTNYNPSLKSRVTISVDTSKNQVSLKLSSVTAADTAVYYCARESKY |
| | shortening the | IFNAR2-VHH2 | YFGLDVWGQGTTVTVTSGGGGSGGGGSGGGGSAIQLTQSPSSLSASVGDR |
| | linker to 2 a.a. | | VTITCRASQGIYSVLAWYQQKPGKTPKLLIYDASRLESGVPSRFSGSGSGT |
| | The short | | DFTLTISSLQPEDFATYYCQQFNSYITFGQGTRLEIKGTSASQVQLQESGGG |
| | linker variant | | LVQPGGSLRLSCAASGFTFSDYIMTWVRQAPGKGLEWVSSINNGGRSTYY |
| | is named | | ADSVKGRFTISRDNAKNTLYLQLNSLKIEDTAMYYCARDVTCWGARTGC |
| | “HIS2”. | | RVSGGTQVTVSS |
| 53 | | IFNAR1-scFv | QVQLQQWGAGLLKPSETLSLTCAVYGGSFSGYFWSWIRQPPGKGLEWIGE |
| | | 3F11 | IDHSGKTNYNPSLKSRVTISVDTSKNQVSLKLSSVTAADTAVYYCARESKY |
| | | IFNAR2-VHH3 | YFGLDVWGQGTTVTVTSGGGGSGGGGSGGGGSAIQLTQSPSSLSASVGDR |
| | | | VTITCRASQGIYSVLAWYQQKPGKTPKLLIYDASRLESGVPSRFSGSGSGT |
| | | | DFTLTISSLQPEDFATYYCQQFNSYITFGQGTRLEIKGTSASQVQLQESGGG |
| | | | LVQPGGSLRLSCAASEFTFSEYSMKWVRQPPGKGLEWVSTISPSGGTTRYA |
| | | | ESVKGRFTISRDNAKNTLYLQLNSLKTEDTAMYYCSVADWSVTQRRGQG |
| | | | TQVTVSS |
| 54 | | IFNAR1-scFv | QVQLQQWGAGLLKPSETLSLTCAVYGGSFSGYFWSWIRQPPGKGLEWIGE |
| | | 3F11 | IDHSGKTNYNPSLKSRVTISVDTSKNQVSLKLSSVTAADTAVYYCARESKY |
| | | IFNAR2-VHH5 | YFGLDVWGQGTTVTVTSGGGGSGGGGSGGGGSAIQLTQSPSSLSASVGDR |
| | | | VTITCRASQGIYSVLAWYQQKPGKTPKLLIYDASRLESGVPSRFSGSGSGT |
| | | | DFTLTISSLQPEDFATYYCQQFNSYITFGQGTRLEIKGTSASQVQLQESGGG |
| | | | LVQPGGSLTLSCAASGFTFRNHIMSWVRQAPGKGLEWVSAISSGGGNTYY |
| | | | ADSVKGRFTISRDNAKSTLYLQLNSLKTEDTAMYYCTKDWARPPYSDFLA |
| | | | PQAQGTQVTVSS |
| 55 | “HIS3” | IFNAR1-scFv | QVQLQQWGAGLLKPSETLSLTCAVYGGSFSGYFWSWIRQPPGKGLEWIGE |
| | | 3F11 | IDHSGKTNYNPSLKSRVTISVDTSKNQVSLKLSSVTAADTAVYYCARESKY |
| | | IFNAR2-VHH8 | YFGLDVWGQGTTVTVTSGGGGSGGGGSGGGGSAIQLTQSPSSLSASVGDR |
| | | | VTITCRASQGIYSVLAWYQQKPGKTPKLLIYDASRLESGVPSRFSGSGSGT |
| | | | DFTLTISSLQPEDFATYYCQQFNSYITFGQGTRLEIKGTSASQVQLQESGGG |
| | | | SVQAGGSLRLSCAASGFYGTYSMGWFRQAPGKEREGVAHIESDGSTRYA |
| | | | DSMKGRFTVSKDNAKKILYLQMNSLKPEDTAVYYCAADPRPYFRTWYER |
| | | | ARYGGQGTQVTVSS |
| 56 | This hit was | IFNAR1-scFv | QVQLQQWGAGLLKPSETLSLTCAVYGGSFSGYFWSWIRQPPGKGLEWIGE |
| | optimized by | 3F11 | IDHSGKTNYNPSLKSRVTISVDTSKNQVSLKLSSVTAADTAVYYCARESKY |
| | shortening the | IFNAR2-VHH10 | YFGLDVWGQGTTVTVTSGGGGSGGGGSGGGGSAIQLTQSPSSLSASVGDR |
| | linker to 2 a.a. | | VTITCRASQGIYSVLAWYQQKPGKTPKLLIYDASRLESGVPSRFSGSGSGT |
| | The short | | DFTLTISSLQPEDFATYYCQQFNSYITFGQGTRLEIKGTSASQVQLQESGGG |
| | linker variant | | LVQPGGSLRLSCAASGFTFSNHIMSWVRQAPGKGLEWVSAINSGGGSTYY |
| | is named | | RDSVKGRFTISRDNAKNTLYLQLNSLKTEDTAMYYCAKDWWRYGSDKPP |
| | “HIS4”. | | GQGTQVTVSS |
| | | | IFNAR1-IFNAR2 orientation |
| | | | Linker = 5 a.a. (GTSAS) |
| 57 | “HIS5” | IFNAR1-VHHA1 | QVQLQESGGGLVQAGGSLRLSCAASGNIFSYDYMGWYRQAPGKEREFVA |
| | | IFNAR2-VHH1 | GITVGGSTNYADSVKGRFTISRDNAKNTVYLQMNSLKPEDTAVYYCAAW |
| | | | WYDSGYFAYWGQGTQVTVSSGTSASQVQLQESGGGSVQPGGSLRLSCAA |
| | | | SGFTFSNYIMSWVRQAPGKGLEWVSAISGGGNTYYTDSVKGRFTISRDNA |
| | | | KNTLYLQLNSLKTEDTAMYYCAQNWWVAGRSPNPPGQGTQVTVSS |
| 58 | | IFNAR1-VHHA1 | QVQLQESGGGLVQAGGSLRLSCAASGNIFSYDYMGWYRQAPGKEREFVA |
| | | IFNAR2-VHH2 | GITVGGSTNYADSVKGRFTISRDNAKNTVYLQMNSLKPEDTAVYYCAAW |
| | | | WYDSGYFAYWGQGTQVTVSSGTSASQVQLQESGGGLVQPGGSLRLSCAA |
| | | | SGFTFSDYIMTWVRQAPGKGLEWVSSINNGGRSTYYADSVKGRFTISRDN |
| | | | AKNTLYLQLNSLKIEDTAMYYCARDVTCWGARTGCRVSGGTQVTVSS |
| 59 | This hit was | IFNAR1-VHHA1 | QVQLQESGGGLVQAGGSLRLSCAASGNIFSYDYMGWYRQAPGKEREFVA |
| | optimized by | IFNAR2-VHH3 | GITVGGSTNYADSVKGRFTISRDNAKNTVYLQMNSLKPEDTAVYYCAAW |
| | shortening the | | WYDSGYFAYWGQGTQVTVSSGTSASQVQLQESGGGLVQPGGSLRLSCAA |
| | linker to 2 a.a. | | SEFTFSEYSMKWVRQPPGKGLEWVSTISPSGGTTRYAESVKGRFTISRDNA |
| | The short | | KNTLYLQLNSLKTEDTAMYYCSVADWSVTQRRGQGTQVTVSS |
| | linker variant | | |
| | is named | | |
| | “HIS6”. | | |
| 60 | | IFNAR1-VHHA1 | QVQLQESGGGLVQAGGSLRLSCAASGNIFSYDYMGWYRQAPGKEREFVA |
| | | IFNAR2-VHH5 | GITVGGSTNYADSVKGRFTISRDNAKNTVYLQMNSLKPEDTAVYYCAAW |
| | | | WYDSGYFAYWGQGTQVTVSSGTSASQVQLQESGGGLVQPGGSLTLSCAA |
| | | | SGFTFRNHIMSWVRQAPGKGLEWVSAISSGGGNTYYADSVKGRFTISRDN |
| | | | AKSTLYLQLNSLKTEDTAMYYCTKDWARPPYSDFLAPQAQGTQVTVSS |
| 61 | | IFNAR1-VHHA1 | QVQLQESGGGLVQAGGSLRLSCAASGNIFSYDYMGWYRQAPGKEREFVA |
| | | IFNAR2-VHH8 | GITVGGSTNYADSVKGRFTISRDNAKNTVYLQMNSLKPEDTAVYYCAAW |
| | | | WYDSGYFAYWGQGTQVTVSSGTSASQVQLQESGGGSVQAGGSLRLSCA |
| | | | ASGFYGTYSMGWFRQAPGKEREGVAHIESDGSTRYADSMKGRFTVSKDN |
| | | | AKKILYLQMNSLKPEDTAVYYCAADPRPYFRTWYERARYGGQGTQVTVS |
| | | | S |
| 62 | “HIS7” | IFNAR1-VHHA1 | QVQLQESGGGLVQAGGSLRLSCAASGNIFSYDYMGWYRQAPGKEREFVA |
| | | IFNAR2-VHH10 | GITVGGSTNYADSVKGRFTISRDNAKNTVYLQMNSLKPEDTAVYYCAAW |
| | | | WYDSGYFAYWGQGTQVTVSSGTSASQVQLQESGGGLVQPGGSLRLSCAA |
| | | | SGFTFSNHIMSWVRQAPGKGLEWVSAINSGGGSTYYRDSVKGRFTISRDN |
| | | | AKNTLYLQLNSLKTEDTAMYYCAKDWWRYGSDKPPGQGTQVTVSS |

Human Type I IFN surrogate agonists:
sequences of molecules selected for functional studies
(refers to FIGS. 11A and 11B-M)

| | | | |
|---|---|---|---|
| 63 | HIS1 | IFNAR1-scFv | QVQLQQWGAGLLKPSETLSLTCAVYGGSFSGYFWSWIRQPPGKGLEWIGE |
| | | 3F11 | IDHSGKTNYNPSLKSRVTISVDTSKNQVSLKLSSVTAADTAVYYCARESKY |
| | | IFNAR2-VHH1 | YFGLDVWGQGTTVTVTSGGGGSGGGGSGGGGSAIQLTQSPSSLSASVGDR |
| | | | VTITCRASQGIYSVLAWYQQKPGKTPKLLIYDASRLESGVPSRFSGSGSGT |
| | | | DFTLTISSLQPEDFATYYCQQFNSYITFGQGTRLEIKGSQVQLQESGGGSVQ |
| | | | PGGSLRLSCAASGFTFSNYIMSWVRQAPGKGLEWVSAISGGGNTYYTDSV |
| | | | KGRFTISRDNAKNTLYLQLNSLKTEDTAMYYCAQNWWVAGRSPNPPGQG |
| | | | TQVTVSS |
| 64 | HIS2 | IFNAR1-scFv | QVQLQQWGAGLLKPSETLSLTCAVYGGSFSGYFWSWIRQPPGKGLEWIGE |
| | | 3F11 | IDHSGKTNYNPSLKSRVTISVDTSKNQVSLKLSSVTAADTAVYYCARESKY |
| | | IFNAR2-VHH2 | YFGLDVWGQGTTVTVTSGGGGSGGGGSGGGGSAIQLTQSPSSLSASVGDR |
| | | | VTITCRASQGIYSVLAWYQQKPGKTPKLLIYDASRLESGVPSRFSGSGSGT |
| | | | DFTLTISSLQPEDFATYYCQQFNSYITFGQGTRLEIKGSQVQLQESGGGLVQ |
| | | | PGGSLRLSCAASGFTFSDYIMTWVRQAPGKGLEWVSSINNGGRSTYYADS |
| | | | VKGRFTISRDNAKNTLYLQLNSLKIEDTAMYYCARDVTCWGARTGCRVS |
| | | | GGTQVTVSS |
| 65 | HIS3 | IFNAR1-scFv | QVQLQQWGAGLLKPSETLSLTCAVYGGSFSGYFWSWIRQPPGKGLEWIGE |
| | | 3F11 | IDHSGKTNYNPSLKSRVTISVDTSKNQVSLKLSSVTAADTAVYYCARESKY |
| | | IFNAR2-VHH8 | YFGLDVWGQGTTVTVTSGGGGSGGGGSGGGGSAIQLTQSPSSLSASVGDR |
| | | | VTITCRASQGIYSVLAWYQQKPGKTPKLLIYDASRLESGVPSRFSGSGSGT |
| | | | DFTLTISSLQPEDFATYYCQQFNSYITFGQGTRLEIKGTSASQVQLQESGGG |
| | | | SVQAGGSLRLSCAASGFYGTYSMGWFRQAPGKEREGVAHIESDGSTRYA |
| | | | DSMKGRFTVSKDNAKKILYLQMNSLKPEDTAVYYCAADPRPYFRTWYER |
| | | | ARYGGQGTQVTVSS |
| 66 | HIS4 | IFNAR1-scFv | QVQLQQWGAGLLKPSETLSLTCAVYGGSFSGYFWSWIRQPPGKGLEWIGE |
| | | 3F11 | IDHSGKTNYNPSLKSRVTISVDTSKNQVSLKLSSVTAADTAVYYCARESKY |
| | | IFNAR2-VHH10 | YFGLDVWGQGTTVTVTSGGGGSGGGGSGGGGSAIQLTQSPSSLSASVGDR |
| | | | VTITCRASQGIYSVLAWYQQKPGKTPKLLIYDASRLESGVPSRFSGSGSGT |
| | | | DFTLTISSLQPEDFATYYCQQFNSYITFGQGTRLEIKGSQVQLQESGGGLVQ |
| | | | PGGSLRLSCAASGFTFSNHIMSWVRQAPGKGLEWVSAINSGGGSTYYRDS |
| | | | VKGRFTISRDNAKNTLYLQLNSLKTEDTAMYYCAKDWWRYGSDKPPGQ |
| | | | GTQVTVSS |
| 67 | HIS5 | IFNAR1-VHHA1 | QVQLQESGGGLVQAGGSLRLSCAASGNIFSYDYMGWYRQAPGKEREFVA |
| | | IFNAR2-VHH1 | GITVGGSTNYADSVKGRFTISRDNAKNTVYLQMNSLKPEDTAVYYCAAW |
| | | | WYDSGYFAYWGQGTQVTVSSGTSASQVQLQESGGGSVQPGGSLRLSCAA |
| | | | SGFTFSNYIMSWVRQAPGKGLEWVSAISGGGNTYYTDSVKGRFTISRDNA |
| | | | KNTLYLQLNSLKTEDTAMYYCAQNWWVAGRSPNPPGQGTQVTVSS |
| 68 | HIS6 | IFNAR1-VHHA1 | QVQLQESGGGLVQAGGSLRLSCAASGNIFSYDYMGWYRQAPGKEREFVA |
| | | IFNAR2-VHH3 | GITVGGSTNYADSVKGRFTISRDNAKNTVYLQMNSLKPEDTAVYYCAAW |
| | | | WYDSGYFAYWGQGTQVTVSSGSQVQLQESGGGLVQPGGSLRLSCAASEF |
| | | | TFSEYSMKWVRQPPGKGLEWVSTISPSGGTTRYAESVKGRFTISRDNAKN |
| | | | TLYLQLNSLKTEDTAMYYCSVADWSVTQRRGQGTQVTVSS |
| 69 | HIS7 | IFNAR1-VHHA1 | QVQLQESGGGLVQAGGSLRLSCAASGNIFSYDYMGWYRQAPGKEREFVA |
| | | IFNAR2-VHH10 | GITVGGSTNYADSVKGRFTISRDNAKNTVYLQMNSLKPEDTAVYYCAAW |
| | | | WYDSGYFAYWGQGTQVTVSSGTSASQVQLQESGGGLVQPGGSLRLSCAA |
| | | | SGFTFSNHIMSWVRQAPGKGLEWVSAINSGGGSTYYRDSVKGRFTISRDN |
| | | | AKNTLYLQLNSLKTEDTAMYYCAKDWWRYGSDKPPGQGTQVTVSS |

Human IL-2Rβ/IL-10Rβ surrogate agonists: sequences of individual VHH binding modules

| | | |
|---|---|---|
| 70 | 2Rβ-VHH1 | QVQLQESGGGSVQAGGSLRLSCVTSGYTYSSANMAWFRQAPGKEREGVA |
| | | IITPSGRATTYADSVKGRFTISRDNAANTLYLQMNSLKPEDTAMYYCAAD |
| | | TPPYSGLWYAERTYNYWGQGTQVTVSS |
| 71 | 2Rβ-VHH3 | QVQLQESGGGSVQAGGSLRLSCTASGFTFDDEDMGWYRQAPGNECELVS |
| | | SIGSLGRRYYADSVKDRFAISQDNAKNTVYLQMNSLKPEDTAVYYCAAT |
| | | KGGSWLDSILASCQGAFGYWGQGTQVTVSS |
| 72 | 2Rβ-VHH6 | QVQLQESGGGSVQAGGSLRLSCAASSYTISSVCMGWFRQAPGKEREGVA |
| | | GIAPDGSTGYGDSVKGRFTISKDNAKNTLYLQMNSLKPEDTAMYYCAAA |
| | | SPGRCFLPRTALEPALYYNWGQGTQVTVSS |
| 73 | 10Rβ-VHH1 | QVQLQESGGGSVQAGGSLRLSCAARYTYSTYCMGWFRQAPGKEREGVA |
| | | AIDSDGSTSYADSVKGRFTISKDNAKNTLYLQMNSLKPEDTAMYYCASDA |
| | | DCDLGGSWSTRGQGTQVTVSS |
| 74 | 10Rβ-VHH2 | QVQLQESGGGSVQTGGSLRLSCAASQVNMNSVGWFRQAPGKAREGVATI |
| | | SPSGGATYYTDSVKGRFTISRDNAKNTVYLQMNSLNAEDTAMYYCAADG |
| | | GRDWWLLRPQTFSYWGQGTQVTVSS |
| 75 | 10Rβ-VHH3 | QVQLQESGGGSVQVGGSLRLSCAASGYTYSNYCMGWFRQAPGKEREGV |
| | | ATIDGDGSTRYADSVKGRFTISKDNAKNTLYLQMHSLKPEDTAMYYCAA |
| | | DFALCDPTVVAHTDFGYWGQGTQVTVSS |
| 76 | 10Rβ-VHH4 | QVQLQESGGGSVQAGGSLRLSCAASGYTYSSYCMGWFRQASGKEREGVA |
| | | TIDSDGSTRYADSVKGRFTISKDNAKNTLYLQMNSLKPEDTAMYYCAADL |
| | | ADCAPSIYSDYDVAHWGQGTQVTVSS |
| 77 | 10Rβ-VHH5 | QVQLQESGGGSVQAGGSLRLSCAASGYTYSSYCMGWFRQAPGKEREGVA |
| | | AIDSDGSTSYADSVKGRFTISKDNAKNTLYLQMNSLKPEDTAMYYCAAEP |
| | | WCTSQGRGRESAEFGYWGQGTQVTVSS |

Human IL-2Rβ/IL-10Rβ surrogate agonists:
sequences of active molecules from initial screen (refers to FIG. 13B)

| | | |
|---|---|---|
| 78 | 10Rβ-VHH1 | QVQLQESGGGSVQAGGSLRLSCAARYTYSTYCMGWFRQAPGKEREGVA |
| | 2Rβ-VHH6 | AIDSDGSTSYADSVKGRFTISKDNAKNTLYLQMNSLKPEDTAMYYCASDA |
| | | DCDLGGSWSTRGQGTQVTVSSGGGGTSASQVQLQESGGGSVQAGGSLRL |
| | | SCAASSYTISSVCMGWFRQAPGKEREGVAGIAPDGSTGYGDSVKGRFTISK |
| | | DNAKNTLYLQMNSLKPEDTAMYYCAAASPGRCFLPRTALEPALYYNWG |
| | | QGTQVTVSS |
| 79 | 10Rβ-VHH3 | QVQLQESGGGSVQVGGSLRLSCAASGYTYSNYCMGWFRQAPGKEREGV |
| | 2Rβ-VHH6 | ATIDGDGSTRYADSVKGRFTISKDNAKNTLYLQMHSLKPEDTAMYYCAA |
| | | DFALCDPTVVAHTDFGYWGQGTQVTVSSGGGGTSASQVQLQESGGGSV |
| | | QAGGSLRLSCAASSYTISSVCMGWFRQAPGKEREGVAGIAPDGSTGYGDS |
| | | VKGRFTISKDNAKNTLYLQMNSLKPEDTAMYYCAAASPGRCFLPRTALEP |
| | | ALYYNWGQGTQVTVSS |
| 80 | 10Rβ-VHH4 | QVQLQESGGGSVQAGGSLRLSCAASGYTYSSYCMGWFRQASGKEREGVA |
| | 2Rβ-VHH1 | TIDSDGSTRYADSVKGRFTISKDNAKNTLYLQMNSLKPEDTAMYYCAADL |
| | | ADCAPSIYSDYDVAHWGQGTQVTVSSGGGGTSASQVQLQESGGGSVQA |
| | | GGSLRLSCVTSGYTYSSANMAWFRQAPGKEREGVAIITPSGRATTYADSV |
| | | KGRFTISRDNAANTLYLQMNSLKPEDTAMYYCAADTPPYSGLWYAERTY |
| | | NYWGQGTQVTVSS |
| 81 | 2Rβ-VHH3 | QVQLQESGGGSVQAGGSLRLSCTASGFTFDDEDMGWYRQAPGNECELVS |
| | 10Rβ-VHH1 | SIGSLGRRYYADSVKDRFAISQDNAKNTVYLQMNSLKPEDTAVYYCAAT |
| | | KGGSWLDSILASCQGAFGYWGQGTQVTVSSGGGGTSASQVQLQESGGGS |
| | | VQAGGSLRLSCAARYTYSTYCMGWFRQAPGKEREGVAAIDSDGSTSYAD |
| | | SVKGRFTISKDNAKNTLYLQMNSLKPEDTAMYYCASDADCDLGGSWSTR |
| | | GQGTQVTVSS |

Human IL-2Rβ/IL-10Rβ surrogate agonists:
sequences of linker-modulated 10Rβ1-2Rβ6 constructs (refers to FIG. 13C)

| | Linker length | | |
|---|---|---|---|
| | (a.a.) | | |
| | Linker | | |
| | sequence | | 10Rβ-VHH1-2Rβ-VHH6 orientation |
| 82 | 0 | 10Rβ-VHH1 | QVQLQESGGGSVQAGGSLRLSCAARYTYSTYCMGWFRQAPGKEREGVA |
| | | 2Rβ-VHH6 | AIDSDGSTSYADSVKGRFTISKDNAKNTLYLQMNSLKPEDTAMYYCASDA |
| | | (0 a.a.) | DCDLGGSWSTRGQGTQVTVSSQVQLQESGGGSVQAGGSLRLSCAASSYTI |
| | | | SSVCMGWFRQAPGKEREGVAGIAPDGSTGYGDSVKGRFTISKDNAKNTL |
| | | | YLQMNSLKPEDTAMYYCAAASPGRCFLPRTALEPALYYNWGQGTQVTVS |
| | | | S |
| 83 | 4 | 10Rβ-VHH1 | QVQLQESGGGSVQAGGSLRLSCAARYTYSTYCMGWFRQAPGKEREGVA |
| | GGGS | 2Rβ-VHH6 | AIDSDGSTSYADSVKGRFTISKDNAKNTLYLQMNSLKPEDTAMYYCASDA |
| | | (4 a.a.) | DCDLGGSWSTRGQGTQVTVSSGGGSQVQLQESGGGSVQAGGSLRLSCAA |
| | | | SSYTISSVCMGWFRQAPGKEREGVAGIAPDGSTGYGDSVKGRFTISKDNA |
| | | | KNTLYLQMNSLKPEDTAMYYCAAASPGRCFLPRTALEPALYYNWGQGTQ |
| | | | VTVSS |
| 84 | 8 | 10Rβ-VHH1 | QVQLQESGGGSVQAGGSLRLSCAARYTYSTYCMGWFRQAPGKEREGVA |
| | GGGGTSAS | 2Rβ-VHH6 | AIDSDGSTSYADSVKGRFTISKDNAKNTLYLQMNSLKPEDTAMYYCASDA |
| | | (8 a.a.) | DCDLGGSWSTRGQGTQVTVSSGGGGTSASQVQLQESGGGSVQAGGSLRL |
| | | | SCAASSYTISSVCMGWFRQAPGKEREGVAGIAPDGSTGYGDSVKGRFTISK |
| | | | DNAKNTLYLQMNSLKPEDTAMYYCAAASPGRCFLPRTALEPALYYNWG |
| | | | QGTQVTVSS |
| 85 | 12 | 10Rβ-VHH1 | QVQLQESGGGSVQAGGSLRLSCAARYTYSTYCMGWFRQAPGKEREGVA |
| | GGGSGGGG | 2Rβ-VHH6 | AIDSDGSTSYADSVKGRFTISKDNAKNTLYLQMNSLKPEDTAMYYCASDA |
| | TSAS | (12 a.a.) | DCDLGGSWSTRGQGTQVTVSSGGGSGGGGTSASQVQLQESGGGSVQAG |
| | | | GSLRLSCAASSYTISSVCMGWFRQAPGKEREGVAGIAPDGSTGYGDSVKG |
| | | | RFTISKDNAKNTLYLQMNSLKPEDTAMYYCAAASPGRCFLPRTALEPALY |
| | | | YNWGQGTQVTVSS |
| 86 | 16 | 10Rβ-VHH1 | QVQLQESGGGSVQAGGSLRLSCAARYTYSTYCMGWFRQAPGKEREGVA |
| | GGGSGGGS | 2Rβ-VHH6 | AIDSDGSTSYADSVKGRFTISKDNAKNTLYLQMNSLKPEDTAMYYCASDA |
| | GGGGTSAS | (16 a.a.) | DCDLGGSWSTRGQGTQVTVSSGGGSGGGSGGGGTSASQVQLQESGGGS |
| | | | VQAGGSLRLSCAASSYTISSVCMGWFRQAPGKEREGVAGIAPDGSTGYGD |
| | | | SVKGRFTISKDNAKNTLYLQMNSLKPEDTAMYYCAAASPGRCFLPRTALE |
| | | | PALYYNWGQGTQVTVSS |

2Rβ-10Rβ surrogate agonists: sequence of 10Rβ1-2Rβ6-Fc construct (refers to FIGS. 13D-O)

| | Linker length | | |
|---|---|---|---|
| | (a.a.) | | |
| | Linker | | |
| | sequence | | 10Rβ-VHH1-2Rβ-VHH6-Fc |
| 87 | 0 | 10Rβ-VHH1 | QVQLQESGGGSVQAGGSLRLSCAARYTYSTYCMGWFRQAPGKEREGVA |
| | | 2Rβ-VHH6-Fc | AIDSDGSTSYADSVKGRFTISKDNAKNTLYLQMNSLKPEDTAMYYCASDA |
| | | (0 a.a.) | DCDLGGSWSTRGQGTQVTVSSQVQLQESGGGSVQAGGSLRLSCAASSY |
| | | | TISSVCMGWFRQAPGKEREGVAGIAPDGSTGYGDSVKGRFTISKDNA |
| | | | KNTLYLQMNSLKPEDTAMYYCAAASPGRCFLPRTALEPALYYNWGQ |
| | | | GTQVTVSSEPKSCDKTHTCPPCPAPELLGGPSVFLFPPKPKDTLMISRTPEV |
| | | | TCVVVDVSHEDPEVKFNWYVDGVEVHNAKTKPREEQYNSTYRVVSVLT |
| | | | VLHQDWLNGKEYKCKVSNKALPAPIEKTISKAKGQPREPQVYTLPPSREE |
| | | | MTKNQVSLTCLVKGFYPSDIAVEWESNGQPENNYKTTPPVLDSDGSFFLY |
| | | | SKLTVDKSRWQQGNVFSCSVMHEALHNHYTQKSLSLSPGK |

Human IL-2 surrogate agonists: sequences of active molecules assembled in alternative formats

| Zipper Module Name | | Protein sequence | |
|---|---|---|---|
| 88 | 2Rβ-VHH1 | QVQLQESGGGSVQAGGSLRLSCVTSGYTYSSANMAWFRQAPGKEREGVA | |
| | Acidic Zipper | IITPSGRATTYADSVKGRFTISRDNAANTLYLQMNSLKPEDTAMYYCAAD | |
| | | TPPYSGLWYAERTYNYWGQGTQVTVSSGSCGAQLEKELQALEKENAQLE | |
| | | WELQALEKELAQ | |
| 89 | 2Rβ-VHH3 | QVQLQESGGGSVQAGGSLRLSCTASGFTFDDEDMGWYRQAPGNECELVS | |
| | Acidic Zipper | SIGSLGRRYYADSVKDRFAISQDNAKNTVYLQMNSLKPEDTAVYYCAAT | |
| | | KGGSWLDSILASCQGAFGYWGQGTQVTVSSGSCGAQLEKELQALEKENA | |
| | | QLEWELQALEKELAQ | |
| 90 | 2Rβ-VHH6 | QVQLQESGGGSVQAGGSLRLSCAASSYTISSVCMGWFRQAPGKEREGVA | |
| | Acidic Zipper | GIAPDGSTGYGDSVKGRFTISKDNAKNTLYLQMNSLKPEDTAMYYCAAA | |
| | | SPGRCFLPRTALEPALYYNWGQGTQVTVSSGSCGAQLEKELQALEKENAQ | |
| | | LEWELQALEKELAQ | |
| 91 | γ_{c}-VHH3 | QVQLQESGGGSVQAGGSLRLSCAASGYTYSKNWYMGWFRQTPGKEREG | |
| | Basic Zipper | VAVIAYDDWPTYADSVKGRFTISKDNTKNTLYLQMNSLKPEDTAMYYCA | |
| | | ARQLGGDYCYFPNLSRFCYNYWGQGTQVTVSSGSCGAQLKKKLQALKK | |
| | | KNAQLKWKLQALKKKLAQ | |
| 92 | γ_{c}-VHH4 | QVQLQESGGGSVQAGGSLRLSCTASGFTFNEANHMGWYRQAPGNECELV | |
| | Basic Zipper | STISSDGTTYYPDSVKGRFTISQDNAKKTAFLQMNSLKPEDTAVYYCAAD | |
| | | QSRRGSLCLGQGTQVTVSSGSCGAQLKKKLQALKKKNAQLKWKLQALK | |
| | | KKLAQ | |
| 93 | γ_{c}-VHH6 | QVQLQESGGGSVQAGGSLRLSCAASGYTYRDYYMGWFRQAPGREREGV | |
| | Basic Zipper | ASIYTRGSREGSTRYSSSVEGRFTITLDTAKNTLYLQMNSLKPEDTAMYYC | |
| | | AADDRTWLPRVQLGGPRENEYNYWGQGTQVTVSSGSCGAQLKKKLQAL | |
| | | KKKNAQLKWKLQALKKKLAQ | |
| Name | Module combo | Protein sequence | |
| MY142-zip | 2Rβ-VHH1 | See SEQ ID 88-93. The indicated acidic and | |
| | Acidic Zipper | basic zipper combos were co-expressed | |
| | γ_{c}-VHH3 | and self-assemble as heterodimers. | |
| | Basic Zipper | | |
| MY141-zip | 2Rβ-VHH3 | See SEQ ID 88-93. The indicated acidic and | |
| | Acidic Zipper | basic zipper combos were co-expressed | |
| | γ_{c}-VHH3 | and self-assemble as heterodimers. | |
| | Basic Zipper | | |
| MY144-zip | 2Rβ-VHH1 | See SEQ ID 88-93. The indicated acidic and | |
| | Acidic Zipper | basic zipper combos were co-expressed | |
| | γ_{c}-VHH4 | and self-assemble as heterodimers. | |
| | Basic Zipper | | |
| MY173-zip | 2Rβ-VHH6 | See SEQ ID 88-93. The indicated acidic and | |
| | Acidic Zipper | basic zipper combos were co-expressed | |
| | γ_{c}-VHH6 | and self-assemble as heterodimers. | |
| | Basic Zipper | | |
| 94 | MY170β | 2Rβ-VHH6 | QVQLQESGGGSVQAGGSLRLSCAASSYTISSVCMGWFRQAPGKEREGVA |
| | | γ_{c}-VHH3 | GIAPDGSTGYGDSVKGRFTISKDNAKNTLYLQMNSLKPEDTAMYYCAAA |
| | | 2Rβ-VHH6 | SPGRCFLPRTALEPALYYNWGQGTQVTVSSSFQVQLQESGGGSVQAGGSL |
| | | | RLSCAASGYTYSKNWYMGWFRQTPGKEREGVAVIAYDDWPTYADSVKG |
| | | | RFTISKDNTKNTLYLQMNSLKPEDTAMYYCAARQLGGDYCYFPNLSRFCY |
| | | | NYWGQGTQVTVSSSFQVQLQESGGGSVQAGGSLRLSCAASSYTISSVCMG |
| | | | WFRQAPGKEREGVAGIAPDGSTGYGDSVKGRFTISKDNAKNTLYLQMNS |
| | | | LKPEDTAMYYCAAASPGRCFLPRTALEPALYYNWGQGTQVTVSS |
| 95 | MY179γ | γ_{c}-VHH6 | QVQLQESGGGSVQAGGSLRLSCAASGYTYRDYYMGWFRQAPGREREGV |
| | | 2Rβ-VHH4 | ASIYTRGSREGSTRYSSSVEGRFTITLDTAKNTLYLQMNSLKPEDTAMYYC |
| | | γ_{c}-VHH6 | AADDRTWLPRVQLGGPRENEYNYWGQGTQVTVSSSFQVQLQESGGGSV |
| | | | QAGGSLRLSCAASGSTSCSSVMRWYRQAPGKEREFVSSINSDRRTVYADS |
| | | | VKGRFTISQDNAKSTLYLQMNSLKAEDTATYYCQRELYGDSWCQGNYW |
| | | | GQGTQVTVSSSFQVQLQESGGGSVQAGGSLRLSCAASGYTYRDYYMGWF |
| | | | RQAPGREREGVASIYTRGSREGSTRYSSSVEGRFTITLDTAKNTLYLQMNS |
| | | | LKPEDTAMYYCAADDRTWLPRVQLGGPRENEYNYWGQGTQVTVSS |
| 96 | MY176β | 2Rβ-VHH4 | QVQLQESGGGSVQAGGSLRLSCAASGSTSCSSVMRWYRQAPGKEREFVSS |
| | | γ_{c}-VHH6 | INSDRRTVYADSVKGRFTISQDNAKSTLYLQMNSLKAEDTATYYCQRELY |
| | | 2Rβ-VHH4 | GDSWCQGNYWGQGTQVTVSSSFQVQLQESGGGSVQAGGSLRLSCAASG |
| | | | YTYRDYYMGWFRQAPGREREGVASIYTRGSREGSTRYSSSVEGRFTITLDT |
| | | | AKNTLYLQMNSLKPEDTAMYYCAADDRTWLPRVQLGGPRENEYNYWG |
| | | | QGTQVTVSSSFQVQLQESGGGSVQAGGSLRLSCAASGSTSCSSVMRWYR |
| | | | QAPGKEREFVSSINSDRRTVYADSVKGRFTISQDNAKSTLYLQMNSLKAE |
| | | | DTATYYCQRELYGDSWCQGNYWGQGTQVTVSS |

Mouse Type I IFN surrogate agonists: sequences of individual VHH binding modules

| SEQ ID NO://Module name | | Protein sequence |
|---|---|---|
| 97 | mIFNAR1-VHH2 | QVQLQESGGGSVQAGGSLRLSCATSGNTVTRSCMAWFRQGPGKQREGVA |
| | | TVASDGSTWYAASVKGRFTISEDNAKNTLYLQMNSLKPEDTAMYYCAAT |
| | | SPGRGCSNLYISPYWGQGTQVTVSS |
| 98 | mIFNAR2-VHH2 | QVQLQESGGGLVQPGGSLRLSCAASGFTFSNYWMYWVRQAPGKGLEWV |
| | | SRINQWGSLTGYADSVKGRFTISRDNAKNMLYLQLNNLKAEDTAMYYCT |
| | | RGNSLQPTSQGTQVTVSS |
| 99 | mIFNAR2-VHH3 | QVQLQESGGGLVQPGGSLRLSCAASGFTFSSSAMNWVRQAPGKGLEWVS |
| | | SISSGGISKFYADSVKGRFTISRDNAKNILYLQMNSLKVEDTGVYNCVPSTS |
| | | CMGRYCSRRDYWGQGTQVTVSS |
| 100 | mIFNAR2-VHH5 | QVQLQESGGGSVETGGSLRLSCAASGFTFSSYWMYWVRQAPGKGLEWVS |
| | | RINQWGSLTGYADSVKGRFTISRDNAKNTVYLQMNNLAAEDTAVYYCAT |
| | | GLAGTIDQRPRGQGTQVTVSS |
| 101 | mIFNAR2-VHH7 | QVQLQESGGGLVQPGGSLRLSCAASGFTFSSYWMYWVRQAPGKGLEWV |
| | | SRINQWGSLTGYADSVKGRFTISRDNAKNTVYLQMNSLKPEDTAMYYCA |
| | | RSRDPYTGGSWAPPLHFSEYVYWGQGTQVTVSS |

Mouse Type I IFN surrogate agonists: sequences of active molecules

| | | mIFNAR1-mIFNAR2 orientation |
|---|---|---|
| Module combo | | Linker = 5 a.a. (GTSAS) |
| 102 | mIFNAR1-VHH2 | QVQLQESGGGSVQAGGSLRLSCATSGNTVTRSCMAWFRQGPGKQREGVA |
| | mIFNAR2-VHH2 | TVASDGSTWYAASVKGRFTISEDNAKNTLYLQMNSLKPEDTAMYYCAAT |
| | | SPGRGCSNLYISPYWGQGTQVTVSSGTSASQVQLQESGGGLVQPGGSLRL |
| | | SCAASGFTFSNYWMYWVRQAPGKGLEWVSRINQWGSLTGYADSVKGRF |
| | | TISRDNAKNMLYLQLNNLKAEDTAMYYCTRGNSLQPTSQGTQVTVSS |
| 103 | mIFNAR1-VHH2 | QVQLQESGGGSVQAGGSLRLSCATSGNTVTRSCMAWFRQGPGKQREGVA |
| | mIFNAR2-VHH3 | TVASDGSTWYAASVKGRFTISEDNAKNTLYLQMNSLKPEDTAMYYCAAT |
| | | SPGRGCSNLYISPYWGQGTQVTVSSGTSASQVQLQESGGGLVQPGGSLRL |
| | | SCAASGFTFSSSAMNWVRQAPGKGLEWVSSISSGGISKFYADSVKGRFTIS |
| | | RDNAKNILYLQMNSLKVEDTGVYNCVPSTSCMGRYCSRRDYWGQGTQV |
| | | TVSS |
| 104 | mIFNAR1-VHH2 | QVQLQESGGGSVQAGGSLRLSCATSGNTVTRSCMAWFRQGPGKQREGVA |
| | mIFNAR2-VHH5 | TVASDGSTWYAASVKGRFTISEDNAKNTLYLQMNSLKPEDTAMYYCAAT |
| | | SPGRGCSNLYISPYWGQGTQVTVSSGTSASQVQLQESGGGSVETGGSLRL |
| | | SCAASGFTFSSYWMYWVRQAPGKGLEWVSRINQWGSLTGYADSVKGRF |
| | | TISRDNAKNTVYLQMNNLAAEDTAVYYCATGLAGTIDQRPRGQGTQVTV |
| | | SS |
| 105 | mIFNAR1-VHH2 | QVQLQESGGGSVQAGGSLRLSCATSGNTVTRSCMAWFRQGPGKQREGVA |
| | mIFNAR2-VHH7 | TVASDGSTWYAASVKGRFTISEDNAKNTLYLQMNSLKPEDTAMYYCAAT |
| | | SPGRGCSNLYISPYWGQGTQVTVSSGTSASQVQLQESGGGLVQPGGSLRL |
| | | SCAASGFTFSSYWMYWVRQAPGKGLEWVSRINQWGSLTGYADSVKGRF |
| | | TISRDNAKNTVYLQMNSLKPEDTAMYYCARSRDPYTGGSWAPPLHFSEY |
| | | VYWGQGTQVTVSS |

### REFERENCES

1. Berraondo, P., Sanmamed, M. F., Ochoa, M. C., Etxeberria, I., Aznar, M. A., Pérez-Gracia, J. L., Rodríguez-Ruiz, M. E., Ponz-Sarvise, M., Castañón, E., and Melero, I. (2018). Cytokines in clinical cancer immunotherapy. British Journal of Cancer 120, 6-15.
2. Cameron, B. J., Gerry, A. B., Dukes, J., Harper, J. V., Kannan, V., Bianchi, F. C., Grand, F., Brewer, J. E., Gupta, M., Plesa, G., et al. (2013). Identification of a Titin-derived HLA-A1-presented peptide as a cross-reactive target for engineered MAGE A3-directed T cells. Sci Transl Med 5, 197ra103.
3. Cardarelli, J. M., Witte, A., and Srinivasan, M. (2010). Interferon Alpha Receptor 1 Antibodies and Their Uses (U.S. Pat. No. 7,662,381B2). 1-58. U.S. Patent and Trademark Office.
4. Cooper, M. A., Fehniger, T. A., and Caligiuri, M. A. (2001). The biology of human natural killer-cell subsets. Trends Immunol. 22, 633-640.
5. Cui, W., Liu, Y., Weinstein, J. S., Craft, J., and Kaech, S. M. (2011). An interleukin-21-interleukin-10-STAT3 pathway is critical for functional maturation of memory CD8⁺ T cells. Immunity 35, 792-805.
6. Delespine-Carmagnat, M., Bouvier, G., and Bertoglio, J. (2000). Association of STAT1, STAT3 and STAT5 proteins with the IL-2 receptor involves different subdomains of the IL-2 receptor I chain. Eur. J. Immunol. 30, 59-68.
7. Emsley, P., Lohkamp, B., Scott, W. G., and Cowtan, K. (2010). Features and development of Coot. Acta Crystallogr D Biol Crystallogr 66, 486-501.
8. Glassman, C. R., Su, L., Majri-Morrison, S. S., Winkelmann, H., Mo, F., Li, P., Perez-Cruz, M., Ho, P. P., Koliesnik, I., Nagy, N., et al. (2021a). Calibration of cell-intrinsic interleukin-2 response thresholds guides design of a regulatory T cell biased agonist. Elife 10.
9. Glassman, C. R., Mathiharan, Y. K., Jude, K. M., Su, L., Panova, O., Lupardus, P. J., Spangler, J. B., Ely, L. K., Thomas, C., Skiniotis, G., et al. (2021b). Structural basis for IL-12 and IL-23 receptor sharing reveals a gateway for shaping actions on T versus NK cells. Cell 184, 983-999.e24.
10. Harbury, P. B., Zhang, T. Kim, P. S. and Alber, T. A switch between two-, three-, and four-stranded coiled coils in GCN4 leucine zipper mutants. (1993). Science.; 262 (5138): 1401-7.
11. Harris, K. E., Lorentsen, K. J., Malik-Chaudhry, H. K., Loughlin, K., Basappa, H. M., Hartstein, S., Ahmil, G., Allen, N. S., Avanzino, B. C., Balasubramani, A., et al. (2021). A bispecific antibody agonist of the IL-2 heterodimeric receptor preferentially promotes in vivo expansion of CD8 and NK cells. Sci Rep 11.
12. Hou, Y. J., Chiba, S., Halfmann, P., Ehre, C., Kuroda, M., Dinnon, K. H., Leist, S. R., Schäfer, A., Nakajima, N., Takahashi, K., et al. (2020). SARS-COV-2 D614G variant exhibits efficient replication ex vivo and transmission in vivo. Science 370, 1464-1468.
13. Kabsch, W. (2010). XDS. Acta Crystallogr D Biol Crystallogr 66, 125-132.
14. Kaech, S. M., and Cui, W. (2012). Transcriptional control of effector and memory CD8+ T cell differentiation. Nat. Rev. Immunol. 12, 749-761.
15. Kromann-Hansen, T., Louise Lange, E., Peter Sørensen, H., Hassanzadeh-Ghassabeh, G., Huang, M., Jensen, J. K., Muyldermans, S., Declerck, P. J., Komives, E. A., and Andreasen, P. A. (2017). Discovery of a novel conformational equilibrium in urokinase-type plasminogen activator. Sci Rep 7, 3385-11.
16. Kuziel, W. A., Ju, G., Grdina, T. A., and Greene, W. C. (1993). Unexpected effects of the IL-2 receptor alpha subunit on high affinity IL-2 receptor assembly and function detected with a mutant IL-2 analog. J Immunol 150, 3357-3365.
17. Leonard, W. J., Lin, J.-A., and O'Shea, J. J. (2019). The γc Family of Cytokines: Basic Biology to Therapeutic Ramifications. Immunity 50, 832-850.
18. Levin, A. M., Bates, D. L., Ring, A. M., Krieg, C., Lin, J. T., Su, L., Moraga, I., Raeber, M. E., Bowman, G. R., Novick, P., et al. (2012). Exploiting a natural conformational switch to engineer an interleukin-2 “superkine.” Nature 484, 529-533.
19. Li, S., Gong M., Zhao, F., Shao, J., Xie, Y., Zhang, Y. and Chang, H. (2018). Type I Interferons: Distinct Biological Activities and Current Applications for Viral Infection. Cell Physiol Biochem. 2018; 51 (5): 2377-2396.
20. Liberzon, A., Birger, C., Thorvaldsdóttir, H., Ghandi, M., Mesirov, J. P., and Tamayo, P. (2015). The Molecular Signatures Database (MSigDB) hallmark gene set collection. Cell Syst 1, 417-425.
21. Liebschner, D., Afonine, P. V., Baker, M. L., Bunkóczi, G., Chen, V. B., Croll, T. I., Hintze, B., Hung, L. W., Jain, S., McCoy, A. J., et al. (2019). Macromolecular structure determination using X-rays, neutrons and electrons: recent developments in Phenix. Acta Crystallogr D Struct Biol 75, 861-877.
22. Lin, J.-X., and Leonard, W. J. (2018). The Common Cytokine Receptor y Chain Family of Cytokines. Cold Spring Harb Perspect Biol 10, a028449.
23. Linette, G. P., Stadtmauer, E. A., Maus, M. V., Rapoport, A. P., Levine, B. L., Emery, L., Litzky, L., Bagg, A., Carreno, B. M., Cimino, P. J., et al. (2013). Cardiovascular toxicity and titin cross-reactivity of affinity-enhanced T cells in myeloma and melanoma. 122 6, 863-871.
24. Love, M. I., Huber, W., and Anders, S. (2014). Moderated estimation of fold change and dispersion for RNA-seq data with DESeq2. Genome Biol 15, 550-21.
25 Maecker, H. T., McCoy, J. P., and Nussenblatt, R. (2012). Standardizing immunophenotyping for the Human Immunology Project. Nat. Rev. Immunol. 12, 191-200.
26. Mansurov, A., Lauterbach, A., Budina, E., Alpar, A. T., Hubbell, J. A., and Ishihara, J. (2021). Immunoengineering approaches for cytokine therapy. Am. J. Physiol., Cell Physiol. 321, C369-C383.
27. Mendoza, J. L., Escalante, N. K., Jude, K. M., Bellon, J. S., Su, L., Horton, T. M., Tsutsumi, N., Berardinelli, S. J., Haltiwanger, R. S., Pichler, J., et al. (2019). Structure of the IFNγ receptor complex guides design of biased agonists. Nature 567, 56-60.
28. Metzemaekers, M., Vanheule, V., Janssens R., Struyf, S. and Proost, P. (2018). Overview of the Mechanisms that May Contribute to the Non-Redundant Activities of Interferon-Inducible CXC Chemokine Receptor 3 Ligands. Front Immunol. 8:1970.
29 Mitra, S., Ring, A. M., Amarnath, S., Spangler, J. B., Li, P., Ju, W., Fischer, S., Oh, J., Spolski, R., Weiskopf, K., et al. (2015). Interleukin-2 Activity Can Be Fine Tuned with Engineered Receptor Signaling Clamps. Immunity 42, 826-838.
30. Miyazaki, T., Kawahara, A., Fujii, H., Nakagawa, Y., Minami, Y., Liu, Z. J., Oishi, I., Silvennoinen, O., Witthuhn, B. A., Ihle, J. N., et al. (1994). Functional Activation of Jak1 and Jak3 by Selective Association with IL-2 Receptor Subunits. Science 266, 1045-1047.
31. Mohan, K., Ueda, G., Kim, A. R., Jude, K. M., Fallas, J. A., Guo, Y., Hafer, M., Miao, Y., Saxton, R. A., Piehler, J., et al. (2019). Topological control of cytokine receptor signaling induces differential effects in hematopoiesis. Science 364.
32. Moraga, I., Wernig, G., Wilmes, S., Gryshkova, V., Richter, C. P., Hong, W.-J., Sinha, R., Guo, F., Fabionar, H., Wehrman, T. S., et al. (2015). Tuning Cytokine Receptor Signaling by Re-orienting Dimer Geometry with Surrogate Ligands. Cell 160, 1196-1208.
33 Morin, A., Eisenbraun, B., Key, J., Sanschagrin, P. C., Timony, M. A., Ottaviano, M., and Sliz, P. (2013). Collaboration gets the most out of software. Elife 2, e01456.
34. Ng, C. T., Mendoza, J. L., Garcia, K. C., and Oldstone, M. B. (2016). Alpha and Beta Type 1 Interferon Signaling: Passage for Diverse Biologic Outcomes. Cell 164, 349-352.
35. Ng, J., and Cantrell, D. (1997). STAT3 is a serine kinase target in T lymphocytes. Interleukin 2 and T cell antigen receptor signals converge upon serine 727. J. Biol. Chem. 272, 24542-24549.
36 Ouyang, W., and O'Garra, A. (2019). IL-10 Family Cytokines IL-10 and IL-22: from Basic Science to Clinical Translation. Immunity 50, 871-891.
37. Overwijk, W. W., Tagliaferri, M. A., and Zalevsky, J. (2021). Engineering IL-2 to Give New Life to T Cell Immunotherapy. Annu Rev Med 72, 281-311.
38. Piehler, J., Thomas, C., Garcia, K. C., and Schreiber, G. (2012). Structural and dynamic determinants of type I interferon receptor assembly and their functional interpretation. Immunol. Rev. 250, 317-334.
39. Quigley, M., Huang, X., and Yang, Y. (2008). STAT1 Signaling in CD8 T Cells Is Required for Their Clonal Expansion and Memory Formation Following Viral Infection In Vivo. J Immunol 180, 2158-2164.
40. Reid, G. S. D., S. Bharya, H-G Klingemann, and K. R. Schultz. (2002). Differential killing of pre-B acute lymphoblastic leukaemia cells by activated NK cells and the NK-92 ci cell line. Clin. Exp. Immunol. 129 (2), 265-71.
41. Ring, A. M., Lin, J.-X., Feng, D., Mitra, S., Rickert, M., Bowman, G. R., Pande, V. S., Li, P., Moraga, I., Spolski, R., et al. (2012). Mechanistic and structural insight into the functional dichotomy between IL-2 and IL-15. Nat. Immunol. 13, 1187-1195.
42 Ross, S. H., and Cantrell, D. A. (2018). Signaling and Function of Interleukin-2 in T Lymphocytes. 43. Russell, S. M., Johnston, J. A., Noguchi, M., Kawamura, M., Bacon C M, Friedmann, M., Berg, M., McVicar, D. W., Witthuhn, B. A., Silvennoinen, O., et al. (1994). Interaction of IL-2Rb and gammac Chains with Jak1 and Jak3: Implication for XSCID and SCID.

### Science 266.

44. Saxton, R. A., Tsutsumi, N., Su, L. L., Abhiraman, G. C., Mohan, K., Henneberg, L. T., Aduri, N. G., Gati, C., and Garcia, K. C. (2021). Structure-based decoupling of the pro- and anti-inflammatory functions of interleukin-10. Science 371.
45. Shoichet, B. K., and Kobilka, B. K. (2012). Structure-based drug screening for G-protein-coupled receptors. Trends Pharmacol. Sci. 33, 268-272.
46 Shourian, M., Beltra, J.-C., Bourdin, B., and Decaluwe, H. (2019). Common gamma chain cytokines and CD8 T cells in cancer. Semin. Immunol. 42, 101307.
47. Siegel, A. M., Heimall, J., Freeman, A. F., Hsu, A. P., Brittain, E., Brenchley, J. M., Douek, D. C., Fahle, G. H., Cohen, J. I., Holland, S. M., et al. (2011). A critical role for STAT3 transcription factor signaling in the development and maintenance of human T cell memory. Immunity 35, 806-818.
48. Silva, D.-A., Yu, S., Ulge, U. Y., Spangler, J. B., Jude, K. M., Labão-Almeida, C., Ali, L. R., Quijano-Rubio, A., Ruterbusch, M., Leung, I., et al. (2019). De novo design of potent and selective mimics of IL-2 and IL-15. Nature 565, 186-191.
49 Smith, J. S., Lefkowitz, R. J., and Rajagopal, S. (2018). Biased signalling: from simple switches to allosteric microprocessors. Nature Reviews Drug Discovery 17, 243-260.
50 Spangler, J. B., Moraga, I., Mendoza, J. L., and Garcia, K. C. (2015). Insights into cytokine-receptor interactions from cytokine engineering. Annu. Rev. Immunol. 33, 139-167.
51. Stroud, R. M., and Wells, J. A. (2004). Mechanistic diversity of cytokine receptor signaling across cell membranes. Sci STKE 2004, re7.
52. Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc Natl Acad Sci USA 102, 15545-15550.
53 Thomas, C., Moraga, I., Levin, D., Krutzik, P. O., Podoplelova, Y., Trejo, A., Lee, C., Yarden, G., Vleck, S. E., Glenn, J. S., et al. (2011). Structural Linkage between Ligand Discrimination and Receptor Activation by Type I Interferons. Cell 146, 621-632.
54. Walter, T. S., Meier, C., Assenberg, R., Au, K.-F., Ren, J., Verma, A., Nettleship, J. E., Owens, R. J., Stuart, D. I., and Grimes, J. M. (2006). Lysine methylation as a routine rescue strategy for protein crystallization. Structure 14, 1617-1622.
55. Wang, C.-I., Brauer, P., Yeo, S. P., Tan, H. C., and Connelly, J. E. (2016). IL2Rbeta/common gamma chain antibodies (US20160367664A1). 1-173. U.S. Patent and Trademark Office.
56 Wang, X., Rickert, M., and Garcia, K. C. (2005). Structure of the quaternary complex of interleukin-2 with its alpha, beta, and gammac receptors. Science 310, 1159-1163.
57. Wu, Y., Tian, Z., and Wei, H. (2017). Developmental and Functional Control of Natural Killer Cells by Cytokines. Front Immunol 8, 1-18.
58. Xue, H. H., Fink, D. W. J., Zhang, X., Qin, J., Turck, C. W., and Leonard, W. J. (2002). Serine phosphorylation of Stat5 proteins in lymphocytes stimulated with IL-2. International Immunology 14, 1263-1271.
59. Yang, H. G., M. C. Kang, T. Y. Kim, I. Hwang, H. T. Jin, Y. C. Sung, Ki-Seong Eom, S. W. Kim. (2019). Discovery of a novel natural killer cell line with distinct immunostimulatory and proliferative potential as an alternative platform for cancer immunotherapy. J. Immunother Cancer 24, 7 (1): 138.

What is claimed is:

1. An engineered polypeptide comprising a single-chain bispecific ligand wherein a first specificity of the ligand is to IFNAR1 and a second specificity of the ligand is to IFNAR2 and wherein the engineered polypeptide is a cytokine agonist.

2. The engineered polypeptide of claim 1, wherein the single-chain bispecific ligand comprises in first nanobody specific to IFNAR1 and a second nanobody specific to IFNAR2.

3. The engineered polypeptide of claim 1, wherein the single-chain bispecific ligand comprises an amino acid sequence at least 80% identical to the amino acid sequence set forth in SEQ. ID. No.: 51-69.

4. The engineered polypeptide of claim 1, wherein the single-chain bispecific ligand comprises an amino acid sequence at least 90% identical to the amino acid sequence set forth in SEQ. ID. No.: 51-69.

5. The engineered polypeptide of claim 1, wherein the single-chain bispecific ligand comprises an amino acid sequence at least 95% identical to the amino acid sequence set forth in SEQ. ID. No.: 51-69.

6. The engineered polypeptide of claim 1, wherein the single-chain bispecific ligand comprises an amino acid sequence that is the amino acid sequence set forth in SEQ. ID. No.: 51-69.

7. The engineered polypeptide of claim 2, wherein the first nanobody comprises an amino acid sequence that is the amino acid sequence set forth in SEQ. ID. No.: 36-44.

8. The engineered polypeptide of claim 2, wherein the second nanobody comprises an amino acid sequence that is the amino acid sequence set forth in SEQ. ID. No.: 45-50.

9. The engineered polypeptide of any one of claims 1-8, wherein the single-chain bispecific ligand is a dimerizing-ligand for an IFNAR1/IFNAR2 receptor heterodimer.

10. The engineered polypeptide of any one of claims 1-9, wherein the engineered polypeptide is capable of inducing phosphorylation of STAT1, or STAT2 or STAT3 or a combination thereof in vitro.

11. The engineered polypeptide of any one of claims 1-9 wherein the engineered polypeptide is capable of inducing phosphorylation of STAT1, or STAT2 or STAT3 or a combination thereof in vivo.

12. The engineered polypeptide of any one of claims 1-11, wherein the engineered polypeptide is capable of inhibiting SARS-COV-2 replication.

13. The engineered polypeptide of claim 12, wherein the engineered polypeptide is capable of inhibiting SARS-COV-2 replication in vitro.

14. The engineered polypeptide of claim 12, wherein the engineered polypeptide is capable of inhibiting SARS-COV-2 replication in vivo.

15. The engineered polypeptide of claim 12, wherein the engineered polypeptide is capable of inhibiting SARS-COV-2 replication in a cell without inducing the expression of pro-inflammatory cytokines.

16. The engineered polypeptide of claim 12, wherein the engineered polypeptide is capable of inhibiting SARS-COV-2 replication in a cell without inducing the expression of anti-proliferative cytokines.

17. The engineered polypeptide of claim 15 or 16, wherein the cell is a mammalian cell.

18. The engineered polypeptide of claim 17 wherein the mammalian cell is a human cell.

19. The engineered polypeptide of any one of claims 2-18, wherein the first nanobody and the second nanobody are linked by linker.

20. The engineered polypeptide of claim 19, wherein the linker is a peptide linker.

21. A cell comprising the engineered polypeptide of any one of claims 1-20.

22. A composition comprising the engineered polypeptide of any one of claims 1-20.

23. A pharmaceutical composition comprising a pharmaceutically acceptable excipient and the engineered polypeptide of any one of claims 1-20.

24. A nucleic acid molecule encoding the engineered polypeptide of any one of claims 1-20.

25. An expression construct comprising the engineered polypeptide of any one of claims 1-20.

26. A method for identifying surrogate cytokine agonists, the method comprises:
providing nanobodies or scFvs against a first target cytokine receptor and against a second target cytokine receptor; and
linking a nanobody or scFv against the first target cytokine receptor with a nanobody or scFv against the second target cytokine receptor thereby identifying a surrogate cytokine agonist.

27. The method of of claim 26, wherein the method further comprises screening for induction of downstream signaling activity.

28. The method of of claim 27, wherein the method comprises screening for induction of STAT1, STAT2, STAT3, STAT5, STAT6, Akt, S6, or ERK activity or combinations thereof.

29. A method for identifying surrogate agonists for a cell surface receptor wherein the cell surface receptor comprises a first component and a second component, the method comprising:
assembling one or more antibody domains to form a bispecific ligand, wherein a first specificity of the ligand is to the first component and a second specificity of the ligand is to the second component; and
identifying surrogate agonists for the receptor.

30. The method of claim 29, wherein the cell surface receptor is a dimeric receptor.

31. The method of claim 30, wherein the dimeric receptor is an RTK, a cytokine or an IgSF receptor.

32. The method of any preceding claims, wherein the first component and the second component are identical and the ligand is monospecific.

33. The method of claim 29, wherein the receptor is a trimeric receptor and further comprises a third component.

34. The method of claim 33, wherein the trimeric receptor is a death receptor such as TNF receptor-1, CD95 (Fas), TRAMP, TRAIL-R1, or TRAIL-R2.

35. The method of any preceding claim, wherein the antibody domain is a VHH or scFv.

36. The method of any preceding claim, wherein the ligand is a single chain homodimer, a heterodimer, or an Fc fusion.

37. The method of any preceding claim, wherein the ligand is a multi-chain agonist comprised of fusions to oligomeric zippers.

38. The method of any preceding claim, wherein the surrogate agonist is a cytokine agonist or an IFN agonist.

39. The method of any preceding claim, wherein the method further comprises employing screening of the differential induction of interferon stimulated genes (ISGs) as a metric for surrogate IFN activity.

40. The method of claim 39, wherein the ISG is MX1, OAS1, IFIT1, IFITM1, TRAIL, CXCL10, ISG15, CH25CH, cGAS, BST2, and NCOA7ISG.

41. The method of any one preceding claim, wherein the ligand is a dimerizing ligand for the receptor.

42. The method of any one of claims 33 to 41, wherein the ligand homodimerizes the first, second and third component of the receptor.


SEQUENCE LISTING (132 sequences)

SEQ ID NO: 1 - protein, 126 aa, synthetic construct
  REGION 1..126: note = VHH Binding Module 2Rbeta-VHH1
QVQLQESGGGSVQAGGSLRLSCVTSGYTYSSANMAWFRQAPGKEREGVAIITPSGRATTYADSVKGRFTISRDNAANTLYLQMNSLKPEDTAMYYCAADTPPYSGLWYAERTYNYWGQGTQVTVSS

SEQ ID NO: 2 - protein, 128 aa, synthetic construct
  REGION 1..128: note = VHH Binding Module 2Rbeta-VHH3
QVQLQESGGGSVQAGGSLRLSCTASGFTFDDEDMGWYRQAPGNECELVSSIGSLGRRYYADSVKDRFAISQDNAKNTVYLQMNSLKPEDTAVYYCAATKGGSWLDSILASCQGAFGYWGQGTQVTVSS

SEQ ID NO: 3 - protein, 120 aa, synthetic construct
  REGION 1..120: note = VHH Binding Module 2Rbeta-VHH4
QVQLQESGGGSVQAGGSLRLSCAASGSTSCSSVMRWYRQAPGKEREFVSSINSDRRTVYADSVKGRFTISQDNAKSTLYLQMNSLKAEDTATYYCQRELYGDSWCQGNYWGQGTQVTVSS

SEQ ID NO: 4 - protein, 128 aa, synthetic construct
  REGION 1..128: note = VHH Binding Module 2Rbeta-VHH6
QVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSS

SEQ ID NO: 5 - protein, 129 aa, synthetic construct
  REGION 1..129: note = VHH Binding Module gamma_c-VHH3
QVQLQESGGGSVQAGGSLRLSCAASGYTYSKNWYMGWFRQTPGKEREGVAVIAYDDWPTYADSVKGRFTISKDNTKNTLYLQMNSLKPEDTAMYYCAARQLGGDYCYFPNLSRFCYNYWGQGTQVTVSS

SEQ ID NO: 6 - protein, 118 aa, synthetic construct
  REGION 1..118: note = VHH Binding Module gamma_c-VHH4
QVQLQESGGGSVQAGGSLRLSCTASGFTFNEANHMGWYRQAPGNECELVSTISSDGTTYYPDSVKGRFTISQDNAKKTAFLQMNSLKPEDTAVYYCAADQSRRGSLCLGQGTQVTVSS

SEQ ID NO: 7 - protein, 133 aa, synthetic construct
  REGION 1..133: note = VHH Binding Module gamma_c-VHH6
QVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSS

SEQ ID NO: 8 - protein, 246 aa, synthetic construct
  REGION 1..246: note = 2Rbeta-VHH1 gamma_c-VHH4
QVQLQESGGGSVQAGGSLRLSCVTSGYTYSSANMAWFRQAPGKEREGVAIITPSGRATTYADSVKGRFTISRDNAANTLYLQMNSLKPEDTAMYYCAADTPPYSGLWYAERTYNYWGQGTQVTVSSSFQVQLQESGGGSVQAGGSLRLSCTASGFTFNEANHMGWYRQAPGNECELVSTISSDGTTYYPDSVKGRFTISQDNAKKTAFLQMNSLKPEDTAVYYCAADQSRRGSLCLGQGTQVTVSS

SEQ ID NO: 9 - protein, 259 aa, synthetic construct
  REGION 1..259: note = 2Rbeta-VHH3 gamma_c-VHH3
QVQLQESGGGSVQAGGSLRLSCTASGFTFDDEDMGWYRQAPGNECELVSSIGSLGRRYYADSVKDRFAISQDNAKNTVYLQMNSLKPEDTAVYYCAATKGGSWLDSILASCQGAFGYWGQGTQVTVSSSFQVQLQESGGGSVQAGGSLRLSCAASGYTYSKNWYMGWFRQTPGKEREGVAVIAYDDWPTYADSVKGRFTISKDNTKNTLYLQMNSLKPEDTAMYYCAARQLGGDYCYFPNLSRFCYNYWGQGTQVTVSS

SEQ ID NO: 10 - protein, 248 aa, synthetic construct
  REGION 1..248: note = 2Rbeta-VHH3 gamma_c-VHH4
QVQLQESGGGSVQAGGSLRLSCTASGFTFDDEDMGWYRQAPGNECELVSSIGSLGRRYYADSVKDRFAISQDNAKNTVYLQMNSLKPEDTAVYYCAATKGGSWLDSILASCQGAFGYWGQGTQVTVSSSFQVQLQESGGGSVQAGGSLRLSCTASGFTFNEANHMGWYRQAPGNECELVSTISSDGTTYYPDSVKGRFTISQDNAKKTAFLQMNSLKPEDTAVYYCAADQSRRGSLCLGQGTQVTVSS

SEQ ID NO: 11 - protein, 263 aa, synthetic construct
  REGION 1..263: note = 2Rbeta-VHH3 gamma_c-VHH6
QVQLQESGGGSVQAGGSLRLSCTASGFTFDDEDMGWYRQAPGNECELVSSIGSLGRRYYADSVKDRFAISQDNAKNTVYLQMNSLKPEDTAVYYCAATKGGSWLDSILASCQGAFGYWGQGTQVTVSSSFQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSS

SEQ ID NO: 12 - protein, 383 aa, synthetic construct
  REGION 1..383: note = 2Rbeta-VHH3 gamma_c-scFv1 (P1A3)
QVQLQESGGGSVQAGGSLRLSCTASGFTFDDEDMGWYRQAPGNECELVSSIGSLGRRYYADSVKDRFAISQDNAKNTVYLQMNSLKPEDTAVYYCAATKGGSWLDSILASCQGAFGYWGQGTQVTVSSGGGGTSASQVQLQQWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTLVTVSSGGGGSGGGGSGGGGSDVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIK

SEQ ID NO: 13 - protein, 379 aa, synthetic construct
  REGION 1..379: note = 2Rbeta-VHH3 gamma_c-scFv2 (P2B9)
QVQLQESGGGSVQAGGSLRLSCTASGFTFDDEDMGWYRQAPGNECELVSSIGSLGRRYYADSVKDRFAISQDNAKNTVYLQMNSLKPEDTAVYYCAATKGGSWLDSILASCQGAFGYWGQGTQVTVSSGGGGTSASQVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAGDILTGYALDYWGQGTLVTVSSGGGGSGGGGSGGGGSSYELTQPPSMSVSPGQTARITCSGDALPKQFAFWYQQKPGQAPVLVIYKDTERPSGIPERFSGSSSGTTVTLTITGVQAEDEADYYCQSPDSSGTVEVFGGGTKLTVL

SEQ ID NO: 14 - protein, 240 aa, synthetic construct
  REGION 1..240: note = 2Rbeta-VHH4 gamma_c-VHH4
QVQLQESGGGSVQAGGSLRLSCAASGSTSCSSVMRWYRQAPGKEREFVSSINSDRRTVYADSVKGRFTISQDNAKSTLYLQMNSLKAEDTATYYCQRELYGDSWCQGNYWGQGTQVTVSSSFQVQLQESGGGSVQAGGSLRLSCTASGFTFNEANHMGWYRQAPGNECELVSTISSDGTTYYPDSVKGRFTISQDNAKKTAFLQMNSLKPEDTAVYYCAADQSRRGSLCLGQGTQVTVSS

SEQ ID NO: 15 - protein, 255 aa, synthetic construct
  REGION 1..255: note = 2Rbeta-VHH4 gamma_c-VHH6
QVQLQESGGGSVQAGGSLRLSCAASGSTSCSSVMRWYRQAPGKEREFVSSINSDRRTVYADSVKGRFTISQDNAKSTLYLQMNSLKAEDTATYYCQRELYGDSWCQGNYWGQGTQVTVSSSFQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSS

SEQ ID NO: 16 - protein, 375 aa, synthetic construct
  REGION 1..375: note = 2Rbeta-VHH4 gamma_c-scFv1 (P1A3)
QVQLQESGGGSVQAGGSLRLSCAASGSTSCSSVMRWYRQAPGKEREFVSSINSDRRTVYADSVKGRFTISQDNAKSTLYLQMNSLKAEDTATYYCQRELYGDSWCQGNYWGQGTQVTVSSGGGGTSASQVQLQQWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTLVTVSSGGGGSGGGGSGGGGSDVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIK

SEQ ID NO: 17 - protein, 371 aa, synthetic construct
  REGION 1..371: note = 2Rbeta-VHH4 gamma_c-scFv2 (P2B9)
QVQLQESGGGSVQAGGSLRLSCAASGSTSCSSVMRWYRQAPGKEREFVSSINSDRRTVYADSVKGRFTISQDNAKSTLYLQMNSLKAEDTATYYCQRELYGDSWCQGNYWGQGTQVTVSSGGGGTSASQVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAGDILTGYALDYWGQGTLVTVSSGGGGSGGGGSGGGGSSYELTQPPSMSVSPGQTARITCSGDALPKQFAFWYQQKPGQAPVLVIYKDTERPSGIPERFSGSSSGTTVTLTITGVQAEDEADYYCQSPDSSGTVEVFGGGTKLTVL

SEQ ID NO: 18 - protein, 248 aa, synthetic construct
  REGION 1..248: note = 2Rbeta-VHH6 gamma_c-VHH4
QVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSSSFQVQLQESGGGSVQAGGSLRLSCTASGFTFNEANHMGWYRQAPGNECELVSTISSDGTTYYPDSVKGRFTISQDNAKKTAFLQMNSLKPEDTAVYYCAADQSRRGSLCLGQGTQVTVSS

SEQ ID NO: 19 - protein, 263 aa, synthetic construct
  REGION 1..263: note = 2Rbeta-VHH6 gamma_c-VHH6
QVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSSSFQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSS

SEQ ID NO: 20 - protein, 257 aa, synthetic construct
  REGION 1..257: note = gamma_c-VHH3 2Rbeta-VHH1
QVQLQESGGGSVQAGGSLRLSCAASGYTYSKNWYMGWFRQTPGKEREGVAVIAYDDWPTYADSVKGRFTISKDNTKNTLYLQMNSLKPEDTAMYYCAARQLGGDYCYFPNLSRFCYNYWGQGTQVTVSSSFQVQLQESGGGSVQAGGSLRLSCVTSGYTYSSANMAWFRQAPGKEREGVAIITPSGRATTYADSVKGRFTISRDNAANTLYLQMNSLKPEDTAMYYCAADTPPYSGLWYAERTYNYWGQGTQVTVSS

SEQ ID NO: 21 - protein, 261 aa, synthetic construct
  REGION 1..261: note = gamma_c-VHH6 2Rbeta-VHH1
QVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSSFQVQLQESGGGSVQAGGSLRLSCVTSGYTYSSANMAWFRQAPGKEREGVAIITPSGRATTYADSVKGRFTISRDNAANTLYLQMNSLKPEDTAMYYCAADTPPYSGLWYAERTYNYWGQGTQVTVSS

SEQ ID NO: 22 - protein, 381 aa, synthetic construct
  REGION 1..381: note = gamma_c-scFv1 (P1A3) 2Rbeta-VHH1
QVQLQQWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTLVTVSSGGGGSGGGGSGGGGSDVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIKGGGGTSASQVQLQESGGGSVQAGGSLRLSCVTSGYTYSSANMAWFRQAPGKEREGVAIITPSGRATTYADSVKGRFTISRDNAANTLYLQMNSLKPEDTAMYYCAADTPPYSGLWYAERTYNYWGQGTQVTVSS

SEQ ID NO: 23 - protein, 377 aa, synthetic construct
  REGION 1..377: note = gamma_c-scFv2 (P2B9) 2Rbeta-VHH1
QVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAGDILTGYALDYWGQGTLVTVSSGGGGSGGGGSGGGGSSYELTQPPSMSVSPGQTARITCSGDALPKQFAFWYQQKPGQAPVLVIYKDTERPSGIPERFSGSSSGTTVTLTITGVQAEDEADYYCQSPDSSGTVEVFGGGTKLTVLGGGGTSASQVQLQESGGGSVQAGGSLRLSCVTSGYTYSSANMAWFRQAPGKEREGVAIITPSGRATTYADSVKGRFTISRDNAANTLYLQMNSLKPEDTAMYYCAADTPPYSGLWYAERTYNYWGQGTQVTVSS

SEQ ID NO: 24 - protein, 259 aa, synthetic construct
  REGION 1..259: note = gamma_c-VHH3 2Rbeta-VHH3
QVQLQESGGGSVQAGGSLRLSCAASGYTYSKNWYMGWFRQTPGKEREGVAVIAYDDWPTYADSVKGRFTISKDNTKNTLYLQMNSLKPEDTAMYYCAARQLGGDYCYFPNLSRFCYNYWGQGTQVTVSSSFQVQLQESGGGSVQAGGSLRLSCTASGFTFDDEDMGWYRQAPGNECELVSSIGSLGRRYYADSVKDRFAISQDNAKNTVYLQMNSLKPEDTAVYYCAATKGGSWLDSILASCQGAFGYWGQGTQVTVSS

SEQ ID NO: 25 - protein, 248 aa, synthetic construct
  REGION 1..248: note = gamma_c-VHH4 2Rbeta-VHH3
QVQLQESGGGSVQAGGSLRLSCTASGFTFNEANHMGWYRQAPGNECELVSTISSDGTTYYPDSVKGRFTISQDNAKKTAFLQMNSLKPEDTAVYYCAADQSRRGSLCLGQGTQVTVSSSFQVQLQESGGGSVQAGGSLRLSCTASGFTFDDEDMGWYRQAPGNECELVSSIGSLGRRYYADSVKDRFAISQDNAKNTVYLQMNSLKPEDTAVYYCAATKGGSWLDSILASCQGAFGYWGQGTQVTVSS

SEQ ID NO: 26 - protein, 263 aa, synthetic construct
  REGION 1..263: note = gamma_c-VHH6 2Rbeta-VHH3
QVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSSFQVQLQESGGGSVQAGGSLRLSCTASGFTFDDEDMGWYRQAPGNECELVSSIGSLGRRYYADSVKDRFAISQDNAKNTVYLQMNSLKPEDTAVYYCAATKGGSWLDSILASCQGAFGYWGQGTQVTVSS

SEQ ID NO: 27 - protein, 383 aa, synthetic construct
  REGION 1..383: note = gamma_c-scFv1 (P1A3) 2Rbeta-VHH3
QVQLQQWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTLVTVSSGGGGSGGGGSGGGGSDVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIKGGGGTSASQVQLQESGGGSVQAGGSLRLSCTASGFTFDDEDMGWYRQAPGNECELVSSIGSLGRRYYADSVKDRFAISQDNAKNTVYLQMNSLKPEDTAVYYCAATKGGSWLDSILASCQGAFGYWGQGTQVTVSS

SEQ ID NO: 28 - protein, 379 aa, synthetic construct
  REGION 1..379: note = gamma_c-scFv2 (P2B9) 2Rbeta-VHH3
QVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAGDILTGYALDYWGQGTLVTVSSGGGGSGGGGSGGGGSSYELTQPPSMSVSPGQTARITCSGDALPKQFAFWYQQKPGQAPVLVIYKDTERPSGIPERFSGSSSGTTVTLTITGVQAEDEADYYCQSPDSSGTVEVFGGGTKLTVLGGGGTSASQVQLQESGGGSVQAGGSLRLSCTASGFTFDDEDMGWYRQAPGNECELVSSIGSLGRRYYADSVKDRFAISQDNAKNTVYLQMNSLKPEDTAVYYCAATKGGSWLDSILASCQGAFGYWGQGTQVTVSS

SEQ ID NO: 29 - protein, 375 aa, synthetic construct
  REGION 1..375: note = gamma_c-scFv1 (P1A3) 2Rbeta-VHH4
QVQLQQWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTLVTVSSGGGGSGGGGSGGGGSDVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIKGGGGTSASQVQLQESGGGSVQAGGSLRLSCAASGSTSCSSVMRWYRQAPGKEREFVSSINSDRRTVYADSVKGRFTISQDNAKSTLYLQMNSLKAEDTATYYCQRELYGDSWCQGNYWGQGTQVTVSS

SEQ ID NO: 30 - protein, 371 aa, synthetic construct
  REGION 1..371: note = gamma_c-scFv2 (P2B9) 2Rbeta-VHH4
QVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAGDILTGYALDYWGQGTLVTVSSGGGGSGGGGSGGGGSSYELTQPPSMSVSPGQTARITCSGDALPKQFAFWYQQKPGQAPVLVIYKDTERPSGIPERFSGSSSGTTVTLTITGVQAEDEADYYCQSPDSSGTVEVFGGGTKLTVLGGGGTSASQVQLQESGGGSVQAGGSLRLSCAASGSTSCSSVMRWYRQAPGKEREFVSSINSDRRTVYADSVKGRFTISQDNAKSTLYLQMNSLKAEDTATYYCQRELYGDSWCQGNYWGQGTQVTVSS

SEQ ID NO: 31 - protein, 259 aa, synthetic construct
  REGION 1..259: note = gamma_c-VHH3 2Rbeta-VHH6
QVQLQESGGGSVQAGGSLRLSCAASGYTYSKNWYMGWFRQTPGKEREGVAVIAYDDWPTYADSVKGRFTISKDNTKNTLYLQMNSLKPEDTAMYYCAARQLGGDYCYFPNLSRFCYNYWGQGTQVTVSSSFQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSS

SEQ ID NO: 32 - protein, 248 aa, synthetic construct
  REGION 1..248: note = gamma_c-VHH4 2Rbeta-VHH6
QVQLQESGGGSVQAGGSLRLSCTASGFTFNEANHMGWYRQAPGNECELVSTISSDGTTYYPDSVKGRFTISQDNAKKTAFLQMNSLKPEDTAVYYCAADQSRRGSLCLGQGTQVTVSSGFQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSS

SEQ ID NO: 33 - protein, 263 aa, synthetic construct
  REGION 1..263: note = gamma_c-VHH6 2Rbeta-VHH6
QVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSSFQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSS

SEQ ID NO: 34 - protein, 383 aa, synthetic construct
  REGION 1..383: note = gamma_c-scFv1 (P1A3) 2Rbeta-VHH6
QVQLQQWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTLVTVSSGGGGSGGGGSGGGGSDVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIKGGGGTSASQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSS

SEQ ID NO: 35 - protein, 379 aa, synthetic construct
  REGION 1..379: note = gamma_c-scFv2 (P2B9) 2Rbeta-VHH6
QVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAGDILTGYALDYWGQGTLVTVSSGGGGSGGGGSGGGGSSYELTQPPSMSVSPGQTARITCSGDALPKQFAFWYQQKPGQAPVLVIYKDTERPSGIPERFSGSSSGTTVTLTITGVQAEDEADYYCQSPDSSGTVEVFGGGTKLTVLGGGGTSASQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSS

SEQ ID NO: 36 - protein, 126 aa, synthetic construct
  REGION 1..126: note = IFNAR1-VHH 1
QVQLQESGGGSVQAGGSLRLSCASSGYTYSNNCMGWFRQAPGKEREGVAAIYTGGGSTYYADSVKGRFTTSQDNAKNTVYLQMNSLKPEDTAMYYCAAVESRTYCTPDALKRFGYRGQGTQVTVSS

SEQ ID NO: 37 - protein, 128 aa, synthetic construct
  REGION 1..128: note = IFNAR1-VHH 5
QVQLQESGGGSVQAGGSLRLSCACEASRYTYSSNCMGWFRQTPGKEREGVAAISTASGSTYYADSVKGRFTISQDNAKNTVYLQMNSLKPEDTAMYYCAVVESSTYCTPDALKRFGYWGQGTQVTVSS

SEQ ID NO: 38 - protein, 125 aa, synthetic construct
  REGION 1..125: note = IFNAR1-VHH 6
QVQLQESGGGSVQAGGSLRLSCAVSGYGNRNYYSGWFRQAPGKGREGVAVIDTYGDIRYGDFVKGRFTISKDSAKNTLYLQMNNLSPEDSAMYYCAASRGYYRNYALREDEYAYWGQGTQVTVSS

SEQ ID NO: 39 - protein, 118 aa, synthetic construct
  REGION 1..118: note = IFNAR1-VHH A1
QVQLQESGGGLVQAGGSLRLSCAASGNIFSYDYMGWYRQAPGKEREFVAGITVGGSTNYADSVKGRFTISRDNAKNTVYLQMNSLKPEDTAVYYCAAWWYDSGYFAYWGQGTQVTVSS

SEQ ID NO: 40 - protein, 126 aa, synthetic construct
  REGION 1..126: note = IFNAR1-VHH A5
QVQLQESGGGLVQAGGSLRLSCAASGTIFLFDYMGWYRQAPGKEREFVAGIARGASTNYADSVKGRFTISRDNAKNTVYLQMNSLKPEDTAVYYCAVLPYLYFPPDGDPANYLDYWGQGTQVTVSS

SEQ ID NO: 41 - protein, 126 aa, synthetic construct
  REGION 1..126: note = IFNAR1-VHH A11
QVQLQESGGGLVQAGGSLRLSCAASGSIFTVYEMGWYRQAPGKEREFVASISYGASTYYADSVKGRFTISRDNAKNTVYLQMNSLKPEDTAVYYCAVVAYVTDQWVGYYTPRYAYWGQGTQVTVSS

SEQ ID NO: 42 - protein, 122 aa, synthetic construct
  REGION 1..122: note = IFNAR1-VHH B4
QVQLQESGGGLVQAGGSLRLSCAASGNIFPYYGMGWYRQAPGKERELVAGIAIGASTNYADSVKGRFTISRDNAKNTVYLQMNSLKPEDTAVYYCAVILGPYNGNINYYLYWGQGTQVTVSS

SEQ ID NO: 43 - protein, 122 aa, synthetic construct
  REGION 1..122: note = IFNAR1-VHH C1
QVQLQESGGGLVQAGGSLRLSCAASGTISKPWGMGWYRQAPGKEREFVATIDGGSTTNYADSVKGRFTISRDNAKNTVYLQMNSLKPEDTAVYYCAAVYADLDVVLYYHIYWGQGTQVTVSS

SEQ ID NO: 44 - protein, 126 aa, synthetic construct
  REGION 1..126: note = IFNAR1-VHH H7
QVQLQESGGGLVQAGGSLRLSCAASGYIFYPFGMGWYRQAPGKEREFVAGINDGTTTNYADSVKGRFTISRDNAKNTVYLQMNSLKPEDTAVYYCAVVYTNGPARTRGDNYYHIYWGQGTQVTVSS

SEQ ID NO: 45 - protein, 119 aa, synthetic construct
  REGION 1..119: note = IFNAR2-VHH 1
QVQLQESGGGSVQPGGSLRLSCAASGFTFSNYIMSWVRQAPGKGLEWVSAISGGGNTYYTDSVKGRFTISRDNAKNTLYLQLNSLKTEDTAMYYCAQNWWVAGRSPNPPGQGTQVTVSS

SEQ ID NO: 46 - protein, 121 aa, synthetic construct
  REGION 1..121: note = IFNAR2-VHH 2
QVQLQESGGGLVQPGGSLRLSCAASGFTFSDYIMTWVRQAPGKGLEWVSSINNGGRSTYYADSVKGRFTISRDNAKNTLYLQLNSLKIEDTAMYYCARDVTCWGARTGCRVSGGTQVTVSS

SEQ ID NO: 47 - protein, 117 aa, synthetic construct
  REGION 1..117: note = IFNAR2-VHH 3
QVQLQESGGGLVQPGGSLRLSCAASEFTFSEYSMKWVRQPPGKGLEWVSTISPSGGTTRYAESVKGRFTISRDNAKNTLYLQLNSLKTEDTAMYYCSVADWSVTQRRGQGTQVTVSS

SEQ ID NO: 48 - protein, 122 aa, synthetic construct
  REGION 1..122: note = IFNAR2-VHH 5
QVQLQESGGGLVQPGGSLTLSCAASGFTFRNHIMSWVRQAPGKGLEWVSAISSGGGNTYYADSVKGRFTISRDNAKSTLYLQLNSLKTEDTAMYYCTKDWARPPYSDFLAPQAQGTQVTVSS

SEQ ID NO: 49 - protein, 122 aa, synthetic construct
  REGION 1..122: note = IFNAR2-VHH 8
QVQLQESGGGSVQAGGSLRLSCAASGFYGTYSMGWFRQAPGKEREGVAHIESDGSTRYADSMKGRFTVSKDNAKKILYLQMNSLKPEDTAVYYCAADPRPYFRTWYERARYGGQGTQVTVSS

SEQ ID NO: 50 - protein, 119 aa, synthetic construct
  REGION 1..119: note = IFNAR2-VHH 10
QVQLQESGGGLVQPGGSLRLSCAASGFTFSNHIMSWVRQAPGKGLEWVSAINSGGGSTYYRDSVKGRFTISRDNAKNTLYLQLNSLKTEDTAMYYCAKDWWRYGSDKPPGQGTQVTVSS

SEQ ID NO: 51 - protein, 363 aa, synthetic construct
  REGION 1..363: note = IFNAR1-scFv 3F11 IFNAR2-VHH 1
QVQLQQWGAGLLKPSETLSLTCAVYGGSFSGYFWSWIRQPPGKGLEWIGEIDHSGKTNYNPSLKSRVTISVDTSKNQVSLKLSSVTAADTAVYYCARESKYYFGLDVWGQGTTVTVTSGGGGSGGGGSGGGGSAIQLTQSPSSLSASVGDRVTITCRASQGIYSVLAWYQQKPGKTPKLLIYDASRLESGVPSRFSGSGSGTDFTLTISSLQPEDFATYYCQQFNSYITFGQGTRLEIKGTSASQVQLQESGGGSVQPGGSLRLSCAASGFTFSNYIMSWVRQAPGKGLEWVSAISGGGNTYYTDSVKGRFTISRDNAKNTLYLQLNSLKTEDTAMYYCAQNWWVAGRSPNPPGQGTQVTVSS

SEQ ID NO: 52 - protein, 365 aa, synthetic construct
  REGION 1..365: note = IFNAR1-scFv 3F11 IFNAR2-VHH 2
QVQLQQWGAGLLKPSETLSLTCAVYGGSFSGYFWSWIRQPPGKGLEWIGEIDHSGKTNYNPSLKSRVTISVDTSKNQVSLKLSSVTAADTAVYYCARESKYYFGLDVWGQGTTVTVTSGGGGSGGGGSGGGGSAIQLTQSPSSLSASVGDRVTITCRASQGIYSVLAWYQQKPGKTPKLLIYDASRLESGVPSRFSGSGSGTDFTLTISSLQPEDFATYYCQQFNSYITFGQGTRLEIKGTSASQVQLQESGGGLVQPGGSLRLSCAASGFTFSDYIMTWVRQAPGKGLEWVSSINNGGRSTYYADSVKGRFTISRDNAKNTLYLQLNSLKIEDTAMYYCARDVTCWGARTGCRVSGGTQVTVSS

SEQ ID NO: 53 - protein, 361 aa, synthetic construct
  REGION 1..361: note = IFNAR1-scFv 3F11 IFNAR2-VHH 3
QVQLQQWGAGLLKPSETLSLTCAVYGGSFSGYFWSWIRQPPGKGLEWIGEIDHSGKTNYNPSLKSRVTISVDTSKNQVSLKLSSVTAADTAVYYCARESKYYFGLDVWGQGTTVTVTSGGGGSGGGGSGGGGSAIQLTQSPSSLSASVGDRVTITCRASQGIYSVLAWYQQKPGKTPKLLIYDASRLESGVPSRFSGSGSGTDFTLTISSLQPEDFATYYCQQFNSYITFGQGTRLEIKGTSASQVQLQESGGGLVQPGGSLRLSCAASEFTFSEYSMKWVRQPPGKGLEWVSTISPSGGTTRYAESVKGRFTISRDNAKNTLYLQLNSLKTEDTAMYYCSVADWSVTQRRGQGTQVTVSS

SEQ ID NO: 54 - protein, 366 aa, synthetic construct
  REGION 1..366: note = IFNAR1-scFv 3F11 IFNAR2-VHH 5
QVQLQQWGAGLLKPSETLSLTCAVYGGSFSGYFWSWIRQPPGKGLEWIGEIDHSGKTNYNPSLKSRVTISVDTSKNQVSLKLSSVTAADTAVYYCARESKYYFGLDVWGQGTTVTVTSGGGGSGGGGSGGGGSAIQLTQSPSSLSASVGDRVTITCRASQGIYSVLAWYQQKPGKTPKLLIYDASRLESGVPSRFSGSGSGTDFTLTISSLQPEDFATYYCQQFNSYITFGQGTRLEIKGTSASQVQLQESGGGLVQPGGSLTLSCAASGFTFRNHIMSWVRQAPGKGLEWVSAISSGGGNTYYADSVKGRFTISRDNAKSTLYLQLNSLKTEDTAMYYCTKDWARPPYSDFLAPQAQGTQVTVSS

SEQ ID NO: 55 - protein, 366 aa, synthetic construct
  REGION 1..366: note = IFNAR1-scFv 3F11 IFNAR2-VHH 8
QVQLQQWGAGLLKPSETLSLTCAVYGGSFSGYFWSWIRQPPGKGLEWIGEIDHSGKTNYNPSLKSRVTISVDTSKNQVSLKLSSVTAADTAVYYCARESKYYFGLDVWGQGTTVTVTSGGGGSGGGGSGGGGSAIQLTQSPSSLSASVGDRVTITCRASQGIYSVLAWYQQKPGKTPKLLIYDASRLESGVPSRFSGSGSGTDFTLTISSLQPEDFATYYCQQFNSYITFGQGTRLEIKGTSASQVQLQESGGGSVQAGGSLRLSCAASGFYGTYSMGWFRQAPGKEREGVAHIESDGSTRYADSMKGRFTVSKDNAKKILYLQMNSLKPEDTAVYYCAADPRPYFRTWYERARYGGQGTQVTVSS

SEQ ID NO: 56 - protein, 363 aa, synthetic construct
  REGION 1..363: note = IFNAR1-scFv 3F11 IFNAR2-VHH 10
QVQLQQWGAGLLKPSETLSLTCAVYGGSFSGYFWSWIRQPPGKGLEWIGEIDHSGKTNYNPSLKSRVTISVDTSKNQVSLKLSSVTAADTAVYYCARESKYYFGLDVWGQGTTVTVTSGGGGSGGGGSGGGGSAIQLTQSPSSLSASVGDRVTITCRASQGIYSVLAWYQQKPGKTPKLLIYDASRLESGVPSRFSGSGSGTDFTLTISSLQPEDFATYYCQQFNSYITFGQGTRLEIKGTSASQVQLQESGGGLVQPGGSLRLSCAASGFTFSNHIMSWVRQAPGKGLEWVSAINSGGGSTYYRDSVKGRFTISRDNAKNTLYLQLNSLKTEDTAMYYCAKDWWRYGSDKPPGQGTQVTVSS

SEQ ID NO: 57 - protein, 242 aa, synthetic construct
  REGION 1..242: note = IFNAR1-VHH A1 IFNAR2-VHH 1
QVQLQESGGGLVQAGGSLRLSCAASGNIFSYDYMGWYRQAPGKEREFVAGITVGGSTNYADSVKGRFTISRDNAKNTVYLQMNSLKPEDTAVYYCAAWWYDSGYFAYWGQGTQVTVSSGTSASQVQLQESGGGSVQPGGSLRLSCAASGFTFSNYIMSWVRQAPGKGLEWVSAISGGGNTYYTDSVKGRFTISRDNAKNTLYLQLNSLKTEDTAMYYCAQNWWVAGRSPNPPGQGTQVTVSS

SEQ ID NO: 58 - protein, 244 aa, synthetic construct
  REGION 1..244: note = IFNAR1-VHH A1 IFNAR2-VHH 2
QVQLQESGGGLVQAGGSLRLSCAASGNIFSYDYMGWYRQAPGKEREFVAGITVGGSTNYADSVKGRFTISRDNAKNTVYLQMNSLKPEDTAVYYCAAWWYDSGYFAYWGQGTQVTVSSGTSASQVQLQESGGGLVQPGGSLRLSCAASGFTFSDYIMTWVRQAPGKGLEWVSSINNGGRSTYYADSVKGRFTISRDNAKNTLYLQLNSLKIEDTAMYYCARDVTCWGARTGCRVSGGTQVTVSS

SEQ ID NO: 59 - protein, 240 aa, synthetic construct
  REGION 1..240: note = IFNAR1-VHH A1 IFNAR2-VHH 3
QVQLQESGGGLVQAGGSLRLSCAASGNIFSYDYMGWYRQAPGKEREFVAGITVGGSTNYADSVKGRFTISRDNAKNTVYLQMNSLKPEDTAVYYCAAWWYDSGYFAYWGQGTQVTVSSGTSASQVQLQESGGGLVQPGGSLRLSCAASEFTFSEYSMKWVRQPPGKGLEWVSTISPSGGTTRYAESVKGRFTISRDNAKNTLYLQLNSLKTEDTAMYYCSVADWSVTQRRGQGTQVTVSS

SEQ ID NO: 60 - protein, 245 aa, synthetic construct
  REGION 1..245: note = IFNAR1-VHH A1 IFNAR2-VHH 5
QVQLQESGGGLVQAGGSLRLSCAASGNIFSYDYMGWYRQAPGKEREFVAGITVGGSTNYADSVKGRFTISRDNAKNTVYLQMNSLKPEDTAVYYCAAWWYDSGYFAYWGQGTQVTVSSGTSASQVQLQESGGGLVQPGGSLTLSCAASGFTFRNHIMSWVRQAPGKGLEWVSAISSGGGNTYYADSVKGRFTISRDNAKSTLYLQLNSLKTEDTAMYYCTKDWARPPYSDFLAPQAQGTQVTVSS

SEQ ID NO: 61 - protein, 245 aa, synthetic construct
  REGION 1..245: note = IFNAR1-VHH A1 IFNAR2-VHH 8
QVQLQESGGGLVQAGGSLRLSCAASGNIFSYDYMGWYRQAPGKEREFVAGITVGGSTNYADSVKGRFTISRDNAKNTVYLQMNSLKPEDTAVYYCAAWWYDSGYFAYWGQGTQVTVSSGTSASQVQLQESGGGSVQAGGSLRLSCAASGFYGTYSMGWFRQAPGKEREGVAHIESDGSTRYADSMKGRFTVSKDNAKKILYLQMNSLKPEDTAVYYCAADPRPYFRTWYERARYGGQGTQVTVSS

SEQ ID NO: 62 - protein, 242 aa, synthetic construct
  REGION 1..242: note = IFNAR1-VHH A1 IFNAR2-VHH 10
QVQLQESGGGLVQAGGSLRLSCAASGNIFSYDYMGWYRQAPGKEREFVAGITVGGSTNYADSVKGRFTISRDNAKNTVYLQMNSLKPEDTAVYYCAAWWYDSGYFAYWGQGTQVTVSSGTSASQVQLQESGGGLVQPGGSLRLSCAASGFTFSNHIMSWVRQAPGKGLEWVSAINSGGGSTYYRDSVKGRFTISRDNAKNTLYLQLNSLKTEDTAMYYCAKDWWRYGSDKPPGQGTQVTVSS

SEQ ID NO: 63 - protein, 360 aa, synthetic construct
  REGION 1..360: note = IFNAR1-scFv 3F11 IFNAR2-VHH 1
QVQLQQWGAGLLKPSETLSLTCAVYGGSFSGYFWSWIRQPPGKGLEWIGEIDHSGKTNYNPSLKSRVTISVDTSKNQVSLKLSSVTAADTAVYYCARESKYYFGLDVWGQGTTVTVTSGGGGSGGGGSGGGGSAIQLTQSPSSLSASVGDRVTITCRASQGIYSVLAWYQQKPGKTPKLLIYDASRLESGVPSRFSGSGSGTDFTLTISSLQPEDFATYYCQQFNSYITFGQGTRLEIKGSQVQLQESGGGSVQPGGSLRLSCAASGFTFSNYIMSWVRQAPGKGLEWVSAISGGGNTYYTDSVKGRFTISRDNAKNTLYLQLNSLKTEDTAMYYCAQNWWVAGRSPNPPGQGTQVTVSS

SEQ ID NO: 64 - protein, 362 aa, synthetic construct
  REGION 1..362: note = IFNAR1-scFv 3F11 IFNAR2-VHH 2
QVQLQQWGAGLLKPSETLSLTCAVYGGSFSGYFWSWIRQPPGKGLEWIGEIDHSGKTNYNPSLKSRVTISVDTSKNQVSLKLSSVTAADTAVYYCARESKYYFGLDVWGQGTTVTVTSGGGGSGGGGSGGGGSAIQLTQSPSSLSASVGDRVTITCRASQGIYSVLAWYQQKPGKTPKLLIYDASRLESGVPSRFSGSGSGTDFTLTISSLQPEDFATYYCQQFNSYITFGQGTRLEIKGSQVQLQESGGGLVQPGGSLRLSCAASGFTFSDYIMTWVRQAPGKGLEWVSSINNGGRSTYYADSVKGRFTISRDNAKNTLYLQLNSLKIEDTAMYYCARDVTCWGARTGCRVSGGTQVTVSS

SEQ ID NO: 65 - protein, 366 aa, synthetic construct; identical to SEQ ID NO: 55
  REGION 1..366: note = IFNAR1-scFv 3F11 IFNAR2-VHH 8

SEQ ID NO: 66 - protein, 360 aa, synthetic construct
  REGION 1..360: note = IFNAR1-scFv 3F11 IFNAR2-VHH 10
QVQLQQWGAGLLKPSETLSLTCAVYGGSFSGYFWSWIRQPPGKGLEWIGEIDHSGKTNYNPSLKSRVTISVDTSKNQVSLKLSSVTAADTAVYYCARESKYYFGLDVWGQGTTVTVTSGGGGSGGGGSGGGGSAIQLTQSPSSLSASVGDRVTITCRASQGIYSVLAWYQQKPGKTPKLLIYDASRLESGVPSRFSGSGSGTDFTLTISSLQPEDFATYYCQQFNSYITFGQGTRLEIKGSQVQLQESGGGLVQPGGSLRLSCAASGFTFSNHIMSWVRQAPGKGLEWVSAINSGGGSTYYRDSVKGRFTISRDNAKNTLYLQLNSLKTEDTAMYYCAKDWWRYGSDKPPGQGTQVTVSS

SEQ ID NO: 67 - protein, 242 aa, synthetic construct; identical to SEQ ID NO: 57
  REGION 1..242: note = IFNAR1-VHH A1 IFNAR2-VHH 1

SEQ ID NO: 68 - protein, 237 aa, synthetic construct
  REGION 1..237: note = IFNAR1-VHH A1 IFNAR2-VHH 3
QVQLQESGGGLVQAGGSLRLSCAASGNIFSYDYMGWYRQAPGKEREFVAGITVGGSTNYADSVKGRFTISRDNAKNTVYLQMNSLKPEDTAVYYCAAWWYDSGYFAYWGQGTQVTVSSGSQVQLQESGGGLVQPGGSLRLSCAASEFTFSEYSMKWVRQPPGKGLEWVSTISPSGGTTRYAESVKGRFTISRDNAKNTLYLQLNSLKTEDTAMYYCSVADWSVTQRRGQGTQVTVSS

SEQ ID NO: 69 - protein, 242 aa, synthetic construct; identical to SEQ ID NO: 62
  REGION 1..242: note = IFNAR1-VHH A1 IFNAR2-VHH 10

SEQ ID NO: 70 - protein, 126 aa, synthetic construct; identical to SEQ ID NO: 1
  REGION 1..126: note = 2Rbeta-VHH1

SEQ ID NO: 71 - protein, 128 aa, synthetic construct; identical to SEQ ID NO: 2
  REGION 1..128: note = 2Rbeta-VHH3

SEQ ID NO: 72 - protein, 128 aa, synthetic construct; identical to SEQ ID NO: 4
  REGION 1..128: note = 2Rbeta-VHH6

SEQ ID NO: 73 - protein, 119 aa, synthetic construct
  REGION 1..119: note = 10Rbeta-VHH1
QVQLQESGGGSVQAGGSLRLSCAARYTYSTYCMGWFRQAPGKEREGVAAIDSDGSTSYADSVKGRFTISKDNAKNTLYLQMNSLKPEDTAMYYCASDADCDLGGSWSTRGQGTQVTVSS

SEQ ID NO: 74 - protein, 123 aa, synthetic construct
  REGION 1..123: note = 10Rbeta-VHH2
QVQLQESGGGSVQTGGSLRLSCAASQVNMNSVGWFRQAPGKAREGVATISPSGGATYYTDSVKGRFTISRDNAKNTVYLQMNSLNAEDTAMYYCAADGGRDWWLLRPQTFSYWGQGTQVTVSS

SEQ ID NO: 75 - protein, 125 aa, synthetic construct
  REGION 1..125: note = 10Rbeta-VHH3
QVQLQESGGGSVQVGGSLRLSCAASGYTYSNYCMGWFRQAPGKEREGVATIDGDGSTRYADSVKGRFTISKDNAKNTLYLQMHSLKPEDTAMYYCAADFALCDPTVVAHTDFGYWGQGTQVTVSS

SEQ ID NO: 76 - protein, 125 aa, synthetic construct
  REGION 1..125: note = 10Rbeta-VHH4
QVQLQESGGGSVQAGGSLRLSCAASGYTYSSYCMGWFRQASGKEREGVATIDSDGSTRYADSVKGRFTISKDNAKNTLYLQMNSLKPEDTAMYYCAADLADCAPSIYSDYDVAHWGQGTQVTVSS

SEQ ID NO: 77 - protein, 126 aa, synthetic construct
  REGION 1..126: note = 10Rbeta-VHH5
QVQLQESGGGSVQAGGSLRLSCAASGYTYSSYCMGWFRQAPGKEREGVAAIDSDGSTSYADSVKGRFTISKDNAKNTLYLQMNSLKPEDTAMYYCAAEPWCTSQGRGRESAEFGYWGQGTQVTVSS

SEQ ID NO: 78 - protein, 255 aa, synthetic construct
  REGION 1..255: note = 10Rbeta-VHH1 2Rbeta-VHH6
QVQLQESGGGSVQAGGSLRLSCAARYTYSTYCMGWFRQAPGKEREGVAAIDSDGSTSYADSVKGRFTISKDNAKNTLYLQMNSLKPEDTAMYYCASDADCDLGGSWSTRGQGTQVTVSSGGGGTSASQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSS

SEQ ID NO: 79 - protein, 261 aa, synthetic construct
  REGION 1..261: note = 10Rbeta-VHH3 2Rbeta-VHH6
QVQLQESGGGSVQVGGSLRLSCAASGYTYSNYCMGWFRQAPGKEREGVATIDGDGSTRYADSVKGRFTISKDNAKNTLYLQMHSLKPEDTAMYYCAADFALCDPTVVAHTDFGYWGQGTQVTVSSGGGGTSASQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSS

SEQ ID NO: 80 - protein, 259 aa, synthetic construct
  REGION 1..259: note = 10Rbeta-VHH4 2Rbeta-VHH1
QVQLQESGGGSVQAGGSLRLSCAASGYTYSSYCMGWFRQASGKEREGVATIDSDGSTRYADSVKGRFTISKDNAKNTLYLQMNSLKPEDTAMYYCAADLADCAPSIYSDYDVAHWGQGTQVTVSSGGGGTSASQVQLQESGGGSVQAGGSLRLSCVTSGYTYSSANMAWFRQAPGKEREGVAIITPSGRATTYADSVKGRFTISRDNAANTLYLQMNSLKPEDTAMYYCAADTPPYSGLWYAERTYNYWGQGTQVTVSS

SEQ ID NO: 81 - protein, 255 aa, synthetic construct
  REGION 1..255: note = 2Rbeta-VHH3 10Rbeta-VHH1
QVQLQESGGGSVQAGGSLRLSCTASGFTFDDEDMGWYRQAPGNECELVSSIGSLGRRYYADSVKDRFAISQDNAKNTVYLQMNSLKPEDTAVYYCAATKGGSWLDSILASCQGAFGYWGQGTQVTVSSGGGGTSASQVQLQESGGGSVQAGGSLRLSCAARYTYSTYCMGWFRQAPGKEREGVAAIDSDGSTSYADSVKGRFTISKDNAKNTLYLQMNSLKPEDTAMYYCASDADCDLGGSWSTRGQGTQVTVSS

SEQ ID NO: 82 - protein, 247 aa, synthetic construct
  REGION 1..247: note = 10Rbeta-VHH1 2Rbeta-VHH6 (0a.a.)
QVQLQESGGGSVQAGGSLRLSCAARYTYSTYCMGWFRQAPGKEREGVAAIDSDGSTSYADSVKGRFTISKDNAKNTLYLQMNSLKPEDTAMYYCASDADCDLGGSWSTRGQGTQVTVSSQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSS

SEQ ID NO: 83 - protein, 251 aa, synthetic construct
  REGION 1..251: note = 10Rbeta-VHH1 2Rbeta-VHH6 (4a.a.)
QVQLQESGGGSVQAGGSLRLSCAARYTYSTYCMGWFRQAPGKEREGVAAIDSDGSTSYADSVKGRFTISKDNAKNTLYLQMNSLKPEDTAMYYCASDADCDLGGSWSTRGQGTQVTVSSGGGSQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSS

SEQ ID NO: 84 - protein, 255 aa, synthetic construct; identical to SEQ ID NO: 78
  REGION 1..255: note = 10Rbeta-VHH1 2Rbeta-VHH6 (8a.a.)

SEQ ID NO: 85 - protein, 259 aa, synthetic construct
  REGION 1..259: note = 10Rbeta-VHH1 2Rbeta-VHH6 (12a.a.)
QVQLQESGGGSVQAGGSLRLSCAARYTYSTYCMGWFRQAPGKEREGVAAIDSDGSTSYADSVKGRFTISKDNAKNTLYLQMNSLKPEDTAMYYCASDADCDLGGSWSTRGQGTQVTVSSGGGSGGGGTSASQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSS

SEQ ID NO: 86 - protein, 263 aa, synthetic construct
  REGION 1..263: note = 10Rbeta-VHH1 2Rbeta-VHH6 (16a.a.)
QVQLQESGGGSVQAGGSLRLSCAARYTYSTYCMGWFRQAPGKEREGVAAIDSDGSTSYADSVKGRFTISKDNAKNTLYLQMNSLKPEDTAMYYCASDADCDLGGSWSTRGQGTQVTVSSGGGSGGGSGGGGTSASQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSS

SEQ ID NO: 87 - protein, 479 aa, synthetic construct
  REGION 1..479: note = 10Rbeta-VHH1 2Rbeta-VHH6-Fc (0a.a.)
QVQLQESGGGSVQAGGSLRLSCAARYTYSTYCMGWFRQAPGKEREGVAAIDSDGSTSYADSVKGRFTISKDNAKNTLYLQMNSLKPEDTAMYYCASDADCDLGGSWSTRGQGTQVTVSSQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 160 aa, synthetic construct
  REGION 1..160: note = 2Rbeta-VHH1 Acidic Zipper
QVQLQESGGGSVQAGGSLRLSCVTSGYTYSSANMAWFRQAPGKEREGVAIITPSGRATTYADSVKGRFTISRDNAANTLYLQMNSLKPEDTAMYYCAADTPPYSGLWYAERTYNYWGQGTQVTVSSGSCGAQLEKELQALEKENAQLEWELQALEKELAQ

SEQ ID NO: 89 - protein, 162 aa, synthetic construct
  REGION 1..162: note = 2Rbeta-VHH3 Acidic Zipper
QVQLQESGGGSVQAGGSLRLSCTASGFTFDDEDMGWYRQAPGNECELVSSIGSLGRRYYADSVKDRFAISQDNAKNTVYLQMNSLKPEDTAVYYCAATKGGSWLDSILASCQGAFGYWGQGTQVTVSSGSCGAQLEKELQALEKENAQLEWELQALEKELAQ

SEQ ID NO: 90 - protein, 162 aa, synthetic construct
  REGION 1..162: note = 2Rbeta-VHH6 Acidic Zipper
QVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSSGSCGAQLEKELQALEKENAQLEWELQALEKELAQ

SEQ ID NO: 91 - protein, 163 aa, synthetic construct
  REGION 1..163: note = gamma_c-VHH3 Basic Zipper
QVQLQESGGGSVQAGGSLRLSCAASGYTYSKNWYMGWFRQTPGKEREGVAVIAYDDWPTYADSVKGRFTISKDNTKNTLYLQMNSLKPEDTAMYYCAARQLGGDYCYFPNLSRFCYNYWGQGTQVTVSSGSCGAQLKKKLQALKKKNAQLKWKLQALKKKLAQ

SEQ ID NO: 92 - protein, 152 aa, synthetic construct
  REGION 1..152: note = gamma_c-VHH4 Basic Zipper
QVQLQESGGGSVQAGGSLRLSCTASGFTFNEANHMGWYRQAPGNECELVSTISSDGTTYYPDSVKGRFTISQDNAKKTAFLQMNSLKPEDTAVYYCAADQSRRGSLCLGQGTQVTVSSGSCGAQLKKKLQALKKKNAQLKWKLQALKKKLAQ

SEQ ID NO: 93 - protein, 167 aa, synthetic construct
  REGION 1..167: note = gamma_c-VHH6 Basic Zipper
QVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGSCGAQLKKKLQALKKKNAQLKWKLQALKKKLAQ

SEQ ID NO: 94 - protein, 389 aa, synthetic construct
  REGION 1..389: note = 2Rbeta-VHH6 gamma_c-VHH3 2Rbeta-VHH6
QVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSSSFQVQLQESGGGSVQAGGSLRLSCAASGYTYSKNWYMGWFRQTPGKEREGVAVIAYDDWPTYADSVKGRFTISKDNTKNTLYLQMNSLKPEDTAMYYCAARQLGGDYCYFPNLSRFCYNYWGQGTQVTVSSSFQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSS

SEQ ID NO: 95 - protein, 390 aa, synthetic construct
  REGION 1..390: note = gamma_c-VHH6 2Rbeta-VHH4 gamma_c-VHH6
QVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSSFQVQLQESGGGSVQAGGSLRLSCAASGSTSCSSVMRWYRQAPGKEREFVSSINSDRRTVYADSVKGRFTISQDNAKSTLYLQMNSLKAEDTATYYCQRELYGDSWCQGNYWGQGTQVTVSSSFQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSS

SEQ ID NO: 96 - protein, 377 aa, synthetic construct
  REGION 1..377: note = 2Rbeta-VHH4 gamma_c-VHH6 2Rbeta-VHH4
QVQLQESGGGSVQAGGSLRLSCAASGSTSCSSVMRWYRQAPGKEREFVSSINSDRRTVYADSVKGRFTISQDNAKSTLYLQMNSLKAEDTATYYCQRELYGDSWCQGNYWGQGTQVTVSSSFQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSSFQVQLQESGGGSVQAGGSLRLSCAASGSTSCSSVMRWYRQAPGKEREFVSSINSDRRTVYADSVKGRFTISQDNAKSTLYLQMNSLKAEDTATYYCQRELYGDSWCQGNYWGQGTQVTVSS

SEQ ID NO: 97 - protein, 123 aa, synthetic construct
  REGION 1..123: note = mIFNAR1-VHH 2
QVQLQESGGGSVQAGGSLRLSCATSGNTVTRSCMAWFRQGPGKQREGVATVASDGSTWYAASVKGRFTISEDNAKNTLYLQMNSLKPEDTAMYYCAATSPGRGCSNLYISPYWGQGTQVTVSS

SEQ ID NO: 98 - protein, 115 aa, synthetic construct
  REGION 1..115: note = mIFNAR2-VHH 2
QVQLQESGGGLVQPGGSLRLSCAASGFTFSNYWMYWVRQAPGKGLEWVSRINQWGSLTGYADSVKGRFTISRDNAKNMLYLQLNNLKAEDTAMYYCTRGNSLQPTSQGTQVTVSS

SEQ ID NO: 99 - protein, 123 aa, synthetic construct
  REGION 1..123: note = mIFNAR2-VHH 3
QVQLQESGGGLVQPGGSLRLSCAASGFTFSSSAMNWVRQAPGKGLEWVSSISSGGISKFYADSVKGRFTISRDNAKNILYLQMNSLKVEDTGVYNCVPSTSCMGRYCSRRDYWGQGTQVTVSS

SEQ ID NO: 100 - protein, 119 aa, synthetic construct
  REGION 1..119: note = mIFNAR2-VHH 5
QVQLQESGGGSVETGGSLRLSCAASGFTFSSYWMYWVRQAPGKGLEWVSRINQWGSLTGYADSVKGRFTISRDNAKNTVYLQMNNLAAEDTAVYYCATGLAGTIDQRPRGQGTQVTVSS

SEQ ID NO: 101 - protein, 130 aa, synthetic construct
  REGION 1..130: note = mIFNAR2-VHH 7
QVQLQESGGGLVQPGGSLRLSCAASGFTFSSYWMYWVRQAPGKGLEWVSRINQWGSLTGYADSVKGRFTISRDNAKNTVYLQMNSLKPEDTAMYYCARSRDPYTGGSWAPPLHFSEYVYWGQGTQVTVSS

SEQ ID NO: 102 - protein, 243 aa, synthetic construct
  REGION 1..243: note = mIFNAR1-VHH 2 mIFNAR2-VHH 2
QVQLQESGGGSVQAGGSLRLSCATSGNTVTRSCMAWFRQGPGKQREGVATVASDGSTWYAASVKGRFTISEDNAKNTLYLQMNSLKPEDTAMYYCAATSPGRGCSNLYISPYWGQGTQVTVSSGTSASQVQLQESGGGLVQPGGSLRLSCAASGFTFSNYWMYWVRQAPGKGLEWVSRINQWGSLTGYADSVKGRFTISRDNAKNMLYLQLNNLKAEDTAMYYCTRGNSLQPTSQGTQVTVSS

SEQ ID NO: 103 - protein, 251 aa, synthetic construct
  REGION 1..251: note = mIFNAR1-VHH 2 mIFNAR2-VHH 3
QVQLQESGGGSVQAGGSLRLSCATSGNTVTRSCMAWFRQGPGKQREGVATVASDGSTWYAASVKGRFTISEDNAKNTLYLQMNSLKPEDTAMYYCAATSPGRGCSNLYISPYWGQGTQVTVSSGTSASQVQLQESGGGLVQPGGSLRLSCAASGFTFSSSAMNWVRQAPGKGLEWVSSISSGGISKFYADSVKGRFTISRDNAKNILYLQMNSLKVEDTGVYNCVPSTSCMGRYCSRRDYWGQGTQVTVSS

SEQ ID NO: 104 - protein, 247 aa, synthetic construct
  REGION 1..247: note = mIFNAR1-VHH 2 mIFNAR2-VHH 5
QVQLQESGGGSVQAGGSLRLSCATSGNTVTRSCMAWFRQGPGKQREGVATVASDGSTWYAASVKGRFTISEDNAKNTLYLQMNSLKPEDTAMYYCAATSPGRGCSNLYISPYWGQGTQVTVSSGTSASQVQLQESGGGSVETGGSLRLSCAASGFTFSSYWMYWVRQAPGKGLEWVSRINQWGSLTGYADSVKGRFTISRDNAKNTVYLQMNNLAAEDTAVYYCATGLAGTIDQRPRGQGTQVTVSS

SEQ ID NO: 105 - protein, 258 aa, synthetic construct
  REGION 1..258: note = mIFNAR1-VHH 2 mIFNAR2-VHH 7
QVQLQESGGGSVQAGGSLRLSCATSGNTVTRSCMAWFRQGPGKQREGVATVASDGSTWYAASVKGRFTISEDNAKNTLYLQMNSLKPEDTAMYYCAATSPGRGCSNLYISPYWGQGTQVTVSSGTSASQVQLQESGGGLVQPGGSLRLSCAASGFTFSSYWMYWVRQAPGKGLEWVSRINQWGSLTGYADSVKGRFTISRDNAKNTVYLQMNSLKPEDTAMYYCARSRDPYTGGSWAPPLHFSEYVYWGQGTQVTVSS

SEQ ID NO: 106 - protein, 4 aa, synthetic construct
  REGION 1..4: note = Polypeptide Linker
GGGS

SEQ ID NO: 107 - protein, 5 aa, synthetic construct
  REGION 1..5: note = Polypeptide Linker
GTSAS

SEQ ID NO: 108 - protein, 8 aa, synthetic construct
  REGION 1..8: note = Polypeptide Linker
GGGGTSAS

SEQ ID NO: 109 - protein, 12 aa, synthetic construct
  REGION 1..12: note = Polypeptide Linker
GGGSGGGGTSAS

SEQ ID NO: 110 - protein, 16 aa, synthetic construct
  REGION 1..16: note = Polypeptide Linker
GGGSGGGSGGGGTSAS

SEQ ID NO: 111 - protein, 22 aa, synthetic construct
  REGION 1..22: note = qRT-PCR primer GAPDH-F
ACAACTTTGGTATCGTGGAAGG

SEQ ID NO: 112 - protein, 19 aa, synthetic construct
  REGION 1..19: note = qRT-PCR primer GAPDH-R
GCCATCACGCCACAGTTTC

SEQ ID NO: 113 - protein, 21 aa, synthetic construct
  REGION 1..21: note = qRT-PCR primer MX1-F
GTTTCCGAAGTGGACATCGCA

SEQ ID NO: 114 - protein, 20 aa, synthetic construct
  REGION 1..20: note = qRT-PCR primer MX1-R
CTGCACAGGTTGTTCTCAGC

SEQ ID NO: 115 - protein, 21 aa, synthetic construct
  REGION 1..21: note = qRT-PCR primer OAS1-F
TGTCCAAGGTGGTAAAGGGTG

SEQ ID NO: 116 - protein, 21 aa, synthetic construct
  REGION 1..21: note = qRT-PCR primer OAS1-R
CCGGCGATTTAACTGATCCTG

SEQ ID NO: 117 - protein, 21 aa, synthetic construct
  REGION 1..21: note = qRT-PCR primer CXCL9-F
CCAACCAAGGGACTATCCACC

SEQ ID NO: 118 - protein, 22 aa, synthetic construct
  REGION 1..22: note = qRT-PCR primer CXCL9-R
CCTTCACATCTGCTGAATCTGG

SEQ ID NO: 119 - protein, 21 aa, synthetic construct
  REGION 1..21: note = qRT-PCR primer CXCL10-F
GTGGCATTCAAGGAGTACCTC

SEQ ID NO: 120 - protein, 21 aa, synthetic construct
  REGION 1..21: note = qRT-PCR primer CXCL10-R
GATGGCCTTCGATTCTGGATT

SEQ ID NO: 121 - protein, 21 aa, synthetic construct
  REGION 1..21: note = qRT-PCR primer CCL2-F
CAGCCAGATGCAATCAATGCC

SEQ ID NO: 122 - protein, 21 aa, synthetic construct
  REGION 1..21: note = qRT-PCR primer CCL2-R
TGGAATCCTGAACCCACTTCT

SEQ ID NO: 123 - protein, 21 aa, synthetic construct
  REGION 1..21: note = qRT-PCR primer CCL3-F
AGTTCTCTGCATCACTTGCTG

SEQ ID NO: 124 - protein, 20 aa, synthetic construct
  REGION 1..20: note = qRT-PCR primer CCL3-R
CGGCTTCGCTTGGTTAGGAA

SEQ ID NO: 125 - protein, 22 aa, synthetic construct
  REGION 1..22: note = qRT-PCR primer IFIT1-F
TCAGGTCAAGGATAGTCTGGAG

SEQ ID NO: 126 - protein, 23 aa, synthetic construct
  REGION 1..23: note = qRT-PCR primer IFIT1-R
AGGTTGTGTATTCCCACACTGTA

SEQ ID NO: 127 - protein, 21 aa, synthetic construct
  REGION 1..21: note = qRT-PCR primer IFITM1-F
CCAAGGTCCACCGTGATTAAC

SEQ ID NO: 128 - protein, 22 aa, synthetic construct
  REGION 1..22: note = qRT-PCR primer IFITM1-R
ACCAGTTCAAGAAGAGGGTGTT

SEQ ID NO: 129 - protein, 21 aa, synthetic construct
  REGION 1..21: note = qRT-PCR primer TRAIL-F
TGCGTGCTGATCGTGATCTTC

SEQ ID NO: 130 - protein, 22 aa, synthetic construct
  REGION 1..22: note = qRT-PCR primer TRAIL-R
GCTCGTTGGTAAAGTACACGTA

SEQ ID NO: 131 - protein, 20 aa, synthetic construct
  REGION 1..20: note = qRT-PCR primer SeV-F
GACGCGAGTTATGTGTTTGC

SEQ ID NO: 132 - protein, 20 aa, synthetic construct
  REGION 1..20: note = qRT-PCR primer SeV-R
TTCCACGCTCTCTTGGATCT